(19) United States
(12) Patent Application Publication
(10) Pub. No.: US 2024/0392289 A1
(43) Pub. Date: Nov. 28, 2024

(54) LIGAND CONJUGATES FOR DELIVERY OF THERAPEUTICALLY ACTIVE AGENTS

(71) Applicant: HEPAGENE THERAPEUTICS (HK) LIMITED, Hong Kong (CN)

(72) Inventors: Xiaodong Xu, Hong Kong (CN); Cheng Mo, Hong Kong (CN); Shijia Chen, Hong Kong (CN)

(73) Assignee: HEPAGENE THERAPEUTICS (HK) LIMITED, Hong Kong (CN)

(21) Appl. No.: 18/294,381

(22) PCT Filed: Aug. 4, 2022
(86) PCT No.: PCT/CN2022/110327

§ 371 Date: Feb. 1, 2024

(30) Foreign Application Priority Data
Aug. 4, 2021 (WO) PCT/CN2021/110556
Apr. 25, 2022 (WO) PCT/CN2022/088882

(51) Int. Cl. C12N 15/113; A61P 3/00

(52) U.S. Cl. CPC C12N 15/113; A61P 3/00; C12N 2310/14; C12N 2310/351; C12N 2320/32

(57) ABSTRACT

The present invention discloses compounds of Formula (I) as novel ligand conjugates, and use thereof for delivering therapeutically active agents. The compounds of the present invention have the advantages for in vitro and/or in vivo delivery of therapeutically active agents, e.g., iRNA agents.

### CROSS REFERENCE TO RELATED APPLICATIONS

This is the U.S. National Stage of International Application No. PCT/CN2022/110327, filed Aug. 4, 2022, which was published in English under PCT Article 21(2), herein incorporated by reference in its entirety, which in turn claims the benefit of International Application No. PCT/CN2021/110556, filed Aug. 4, 2021, and International Application No. PCT/CN2022/088882, filed Apr. 25, 2022.

### FILED OF INVENTION

The present invention relates to the field of delivering therapeutically active agents using ligand conjugates. Particularly, the present invention discloses novel ligand conjugates, which will have the advantages for the in vitro and/or in vivo delivery of therapeutically active agents, and use and compositions thereof.

### BACKGROUND OF THE INVENTION

RNA interference (RNAi) is an RNA-dependent gene silencing process that is controlled by the RNA-induced silencing complex (RISC) and is initiated by short double-stranded RNA molecules in a cell's cytoplasm, where they interact with the catalytic RISC component argonaute and regulate the expression of protein-coding genes. This natural mechanism for sequence-specific gene silencing makes it a promising strategy for therapeutic intervention.

Efficient delivery of RNAi compounds to the target organ in vivo requires specific targeting and substantial protection from the extracellular environment, particularly exonuclease. One method to achieve organ specificity is to conjugate a targeting ligand to a RNAi compound which selectively binds to a surface of membrane receptor highly abundant on the target tissue, and initiates endocytotic activity.

The asialoglycoprotein receptor (ASGPR) is a transmembrane receptor, which is primarily expressed on hepatocytes and minimally on extra-hepatic cells. ASGPR facilitates internalization by clathrin-mediated endocytosis and exhibits high affinity for carbohydrates or the like, e.g., galactose, N-acetylgalactosamine and glucose. These features make it specifically attractive for receptor-mediated drug delivery with minimum concerns of toxicity.

Liver disease (e.g., non-alcoholic fatty liver disease (NAFLD), non-alcoholic steatohepatitis (NASH), infection of Hepatitis B virus (HBV), liver fibrosis, and liver cirrhosis) is a heavy burden for modern society, and there is a huge unmet medical need in this area. Therefore, there is a clear need for the safe and effective therapeutically active agents to improve the life quality of the patients with liver disease.

Lots of ligand conjugates for delivering therapeutically active iRNA agents are reported in the literatures (see, for example, International Patent Application Publications Nos. WO2009/073809A2, WO2016/077321A1, WO2021/113851A2, WO2019/105419A1, WO2009/134487A2, WO2011/091396A1, WO2017/157899A1). Given the complex of drug discovery and the huge unmet medical need, it is meaningful to design new ligand conjugates for delivering therapeutically active agents.

### SUMMARY OF THE INVENTION

The present invention is directed to compounds which are novel ligand conjugates. The compounds of the present invention are efficient for delivering therapeutically active agents, such as iRNA agents, and thus useful in, e.g., modulating expression of a target gene in a cell and treating various disorders and conditions.

In one aspect, provided herein is a compound having the structure shown in Formula (I)

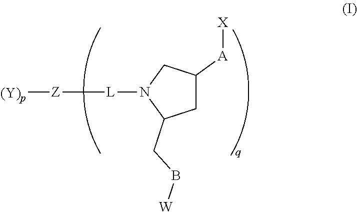

or a pharmaceutically acceptable salt or solvate thereof, wherein
A and B, for each occurrence, are each independently O, N(R^{N}), or S;
R^{N} is H or C₁₋₆ alkyl;
X and W, for each occurrence, are each independently H, a protecting group, a phosphate group, a phosphodiester group, an activated phosphate group, an activated phosphite group, a phosphoramidite, a solid support, —P(Z′)(Z″)O-nucleoside, —P(Z′)(Z″)O-oligonucleotide, a lipid, a PEG, a steroid, a polymer, a nucleotide, a nucleoside, an oligonucleotide, or a therapeutically active agent;
Z′ and Z″ are each independently O or S;
L is a covalent linker;
Z is a carbohydrate mimetic or a disaccharide, trisaccharide, or oligosaccharide;
each Y is independently -L′-T;
each T is independently a ligand selected from the group consisting of carbohydrate ligands, polypeptide ligands, and lipophile ligands;
each L′ is independently a covalent linker; and
p and q are each independently 1, 2, 3, 4, or 5.

In certain embodiments, Z is a carbohydrate mimetic of a monosaccharide.

In certain embodiments, Z is a carbohydrate mimetic of a monosaccharide selected from the group consisting of deoxysugars, aminosugars, N-glycosides, iminosugars, unsaturated sugars, carboxylated sugars, amidated sugars, fused cyclic sugars, and carbasugars of a monosaccharide.

In certain embodiments, Z is an iminosugar of a monosaccharide.

In certain embodiments, the monosaccharide is a terose, pentose, hexose, heptose, or octose.

In certain embodiments, Z has the structure:

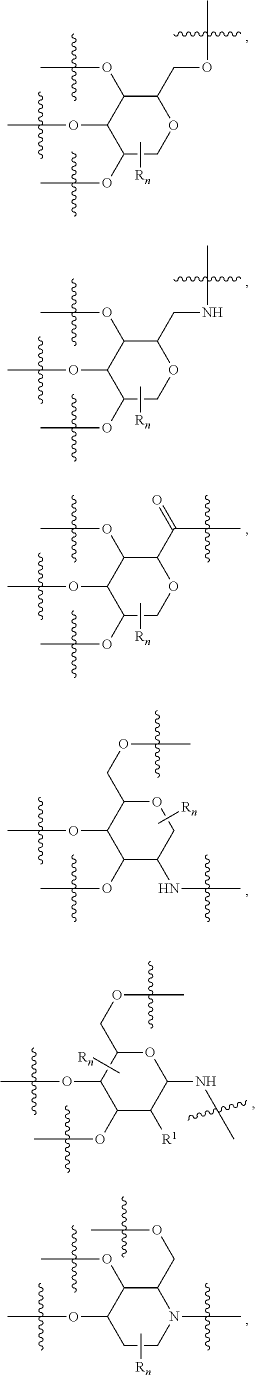

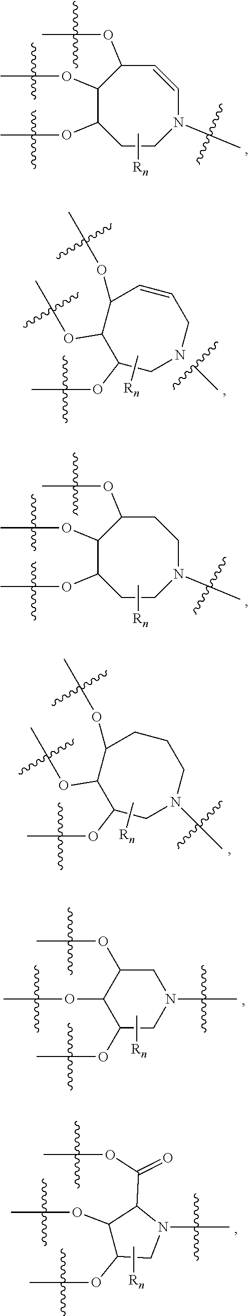

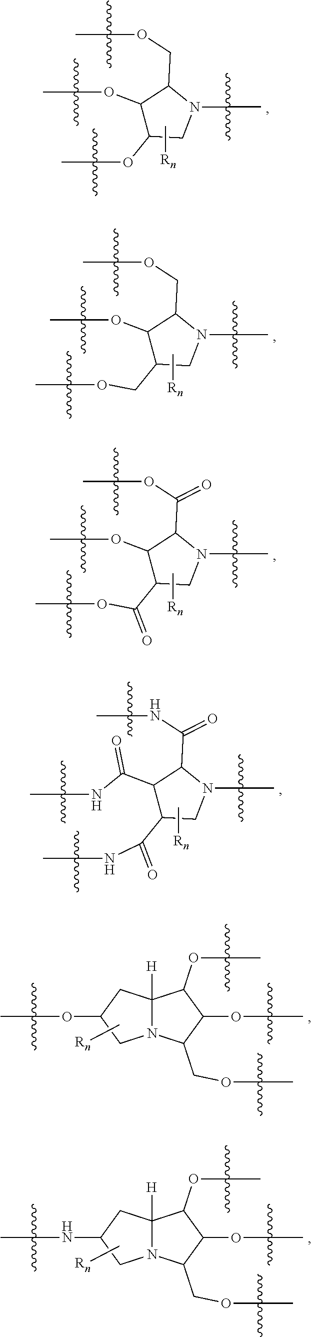

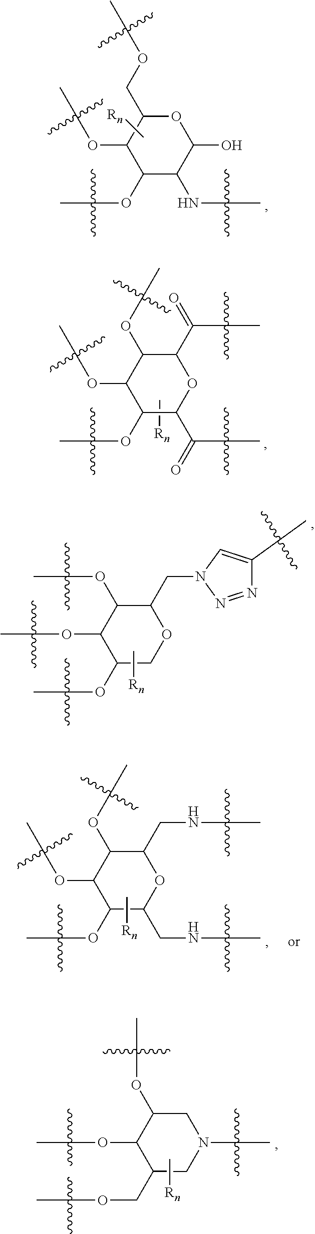

wherein
R¹ is H, C₁₋₆ alkyl, halogen or —NH(R²), wherein R² is H or acetyl;
each R is independently H, halogen, —CN, —C≡CH, —NH₂, —OC₁₋₆ alkyl, or C₁₋₆ alkyl, wherein said alkyl of —C₁₋₆alkyl and —OC₁₋₆alkyl is substituted with 0 to 5 halogen atoms; or two R together with the carbon to which they are attached form a C₃₋₆cycloalkyl or 3- to 6-membered heterocycloalkyl group, wherein said cycloalkyl of C₃₋₆cycloalkyl and heterocycloalkyl of 3- to 6-membered heterocycloalkyl is substituted with 0 to 5 halogen atoms; and
n is 0, 1, 2, or 3, as valency permits.

In certain embodiments, n is 0.

In certain embodiments, Z has the structure:

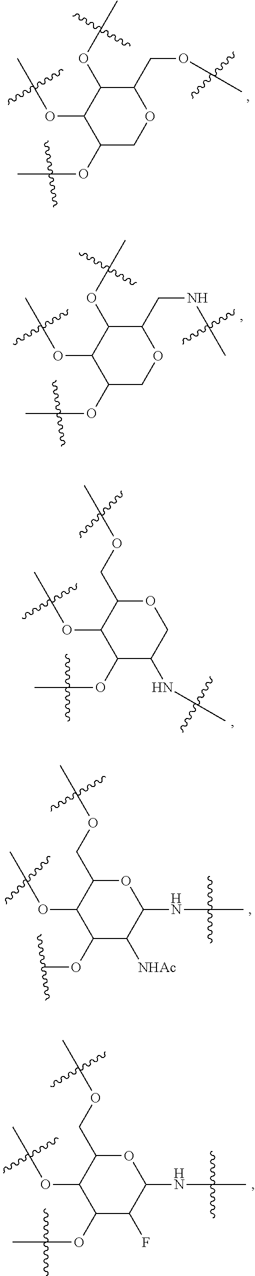 [SMILES: *OCC1OCC(O*)C(O*)C1O* ; *NCC1OCC(O*)C(O*)C1O* ; *NC1COC(CO*)C(O*)C1O* ; *NC1OC(CO*)C(O*)C(O*)C1NC(C)=O ; *NC1OC(CO*)C(O*)C(O*)C1F]

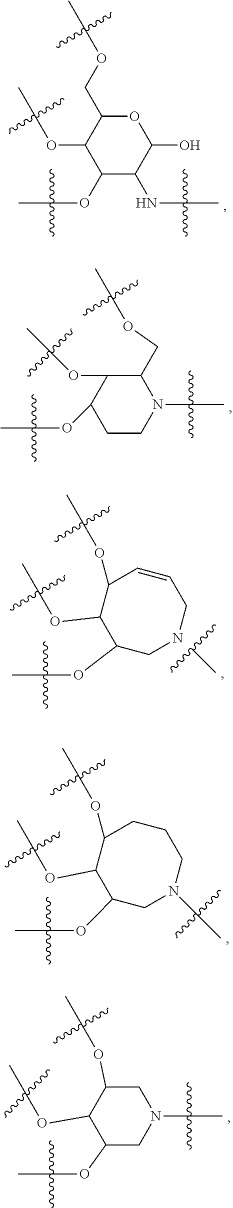

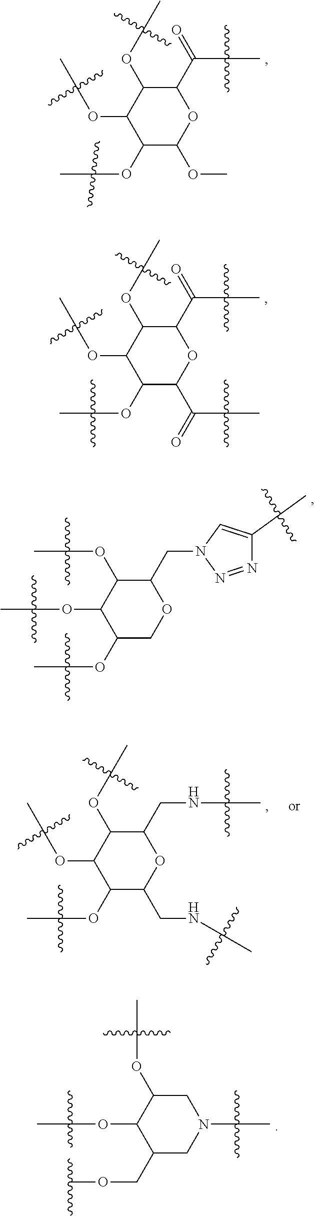 [SMILES: *OC1C(OC)OC(C(*)=O)C(O*)C1O* ; *OC1C(C(*)=O)OC(C(*)=O)C(O*)C1O* ; *OC1COC(Cn2cc(*)nn2)C(O*)C1O* ; *NCC1OC(CN*)C(O*)C(O*)C1O* ; *OCC1CN(*)CC(O*)C1O*]

In certain embodiments, (Y)ₚ—Z— has the structure:

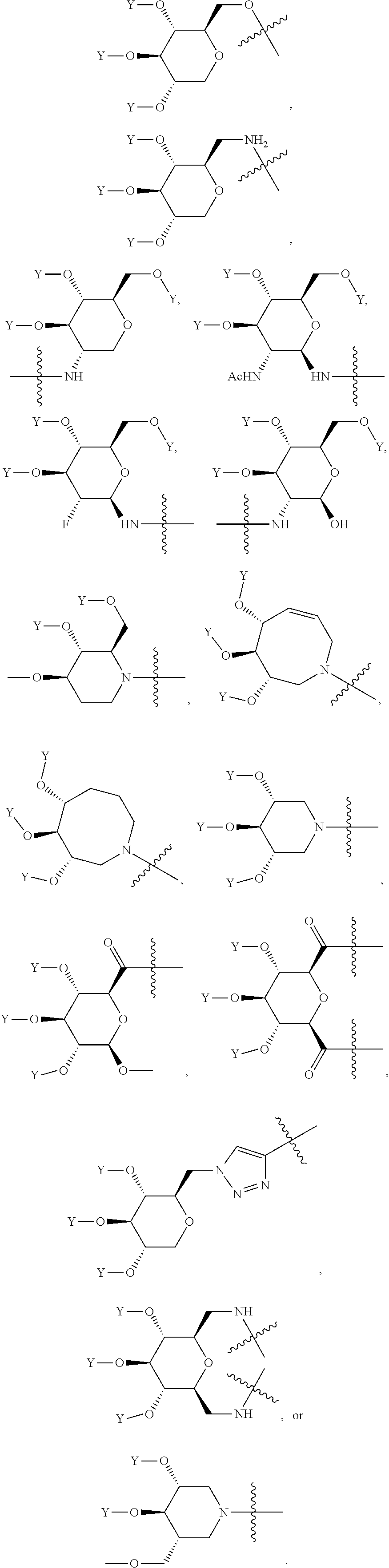

In certain embodiments, Z is a disaccharide, a trisaccharide, or a carbohydrate mimetic of disaccharide or trisaccharide.

In certain embodiments, Z is a disaccharide or a carbohydrate mimetic of disaccharide.

In certain embodiments, Z is a disaccharide selected from the group consisting of gentiobiose, isomaltose, melibiose, trehalose, sucrose, lactose, maltose, and cellobiose, or a carbohydrate mimetic thereof.

In certain embodiments, Z has the following structure:

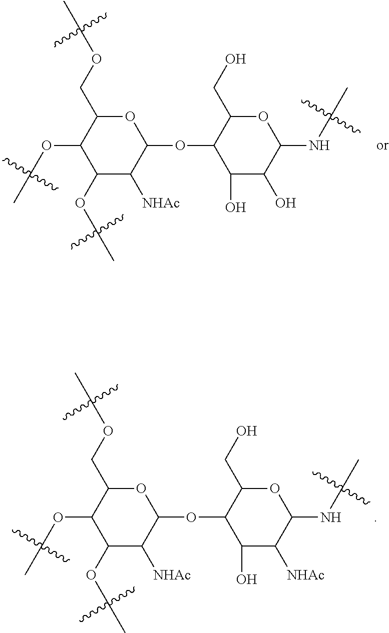

In certain embodiments, Z has the following structure:

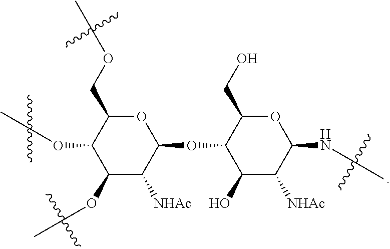 [SMILES: *N[C@@H]1O[C@H](CO)[C@@H](O[C@@H]2O[C@H](CO*)[C@@H](O*)[C@H](O*)[C@H]2NC(C)=O)[C@H](O)[C@H]1NC(C)=O]

In certain embodiments,
each

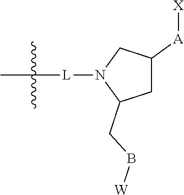

is independently a group having the following structure
   wherein

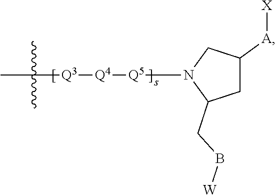

s is an integer from 1 to 20,
each Q³ is independently absent, —CO—, —NH—, —O—, —S—, —SO₂—, —OC(O)—, —C(O)O—, —NHC(O), —C(O)NH—, —CH₂—, —CH₂NH—, —NHCH₂—, —CH₂O—, or —OCH₂—,
each Q⁴ is independently absent, unsubstituted or substituted C₁₋₁₂ alkylene, unsubstituted or substituted C₂₋₁₂ alkenylene, unsubstituted or substituted C₂₋₁₂ alkynylene, unsubstituted or substituted C₂₋₁₂ heteroalkylene, unsubstituted or substituted 6- to 12-membered arylene, unsubstituted or substituted 5- to 12-membered heteroarylene, or unsubstituted or substituted 5- to 12-membered heterocyclylene, and
each Q⁵ is independently absent, —CO—, —NH—, —O—, —S—, —SO₂—, —CH₂—, —C(O)O—, —OC(O)—, —C(O)NH—, —NHC(O)—, —NH—CH(R^{a})—C(O)—, —C(O)—CH(R^{a})—NH—, —OP(O)(OH)O—, or —OP(S)(OH)O—, wherein each R^{a} is independently H or unsubstituted or substituted C₁₋₁₂ alkyl,
provided that at least one Q⁴ is present.

In certain embodiments,
s is an integer from 1 to 5,
each Q³ is independently absent, —CO—, —NH—, —O—, —OC(O)—, —C(O)O—, —NHC(O)—, —C(O)NH—, —CH₂—, —CH₂NH—, —NHCH₂—, —CH₂O—, or —OCH₂—,
each Q⁴ is independently absent, unsubstituted or substituted C₁₋₁₂ alkylene, unsubstituted or substituted C₂₋₁₂ alkenylene, or unsubstituted or substituted C₂₋₁₂ alkynylene, and
each Q⁵ is independently absent, —CO—, —NH—, —O—, —CH₂—, —C(O)O—, —OC(O)—, —C(O)NH—, or —NHC(O)—,
provided that at least one Q⁴ is present.

In certain embodiments,
s is 1 or 2,
each Q³ is independently absent, —CO—, —NH—, —CH₂—, or —NHC(O)—,
each Q⁴ is independently absent, or C₁₋₁₂ alkylene, and
each Q⁵ is independently absent, —CO—, —CH₂—, or —NHC(O)—,
provided that at least one Q⁴ is present.

In certain embodiments,
each

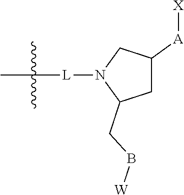

is independently a group having the structure
   wherein

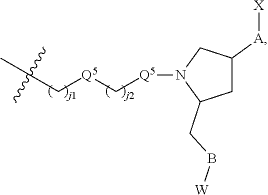

each Q⁵ is independently —CO—, —NH—, —O—, —CH₂—, —C(O)O—, —OC(O)—, —C(O)NH—, or —NHC(O)—, and
each of j1 and j2 is independently 0, 1, 2, 3, 4, 5, 6, 7, 8, 9, 10, 11, or 12.

In certain embodiments,
each

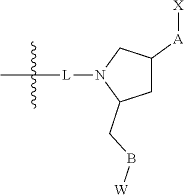

is independently a group having the following structure

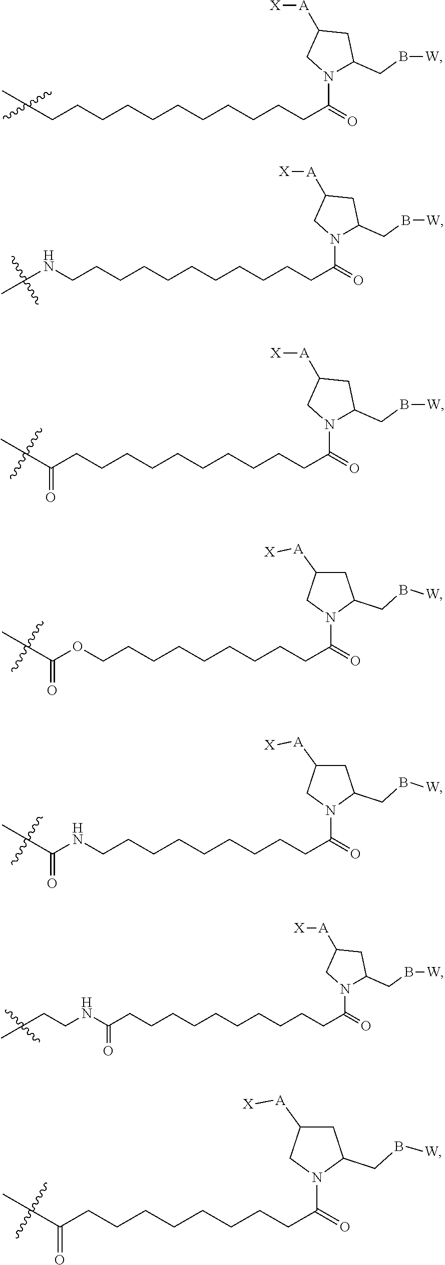

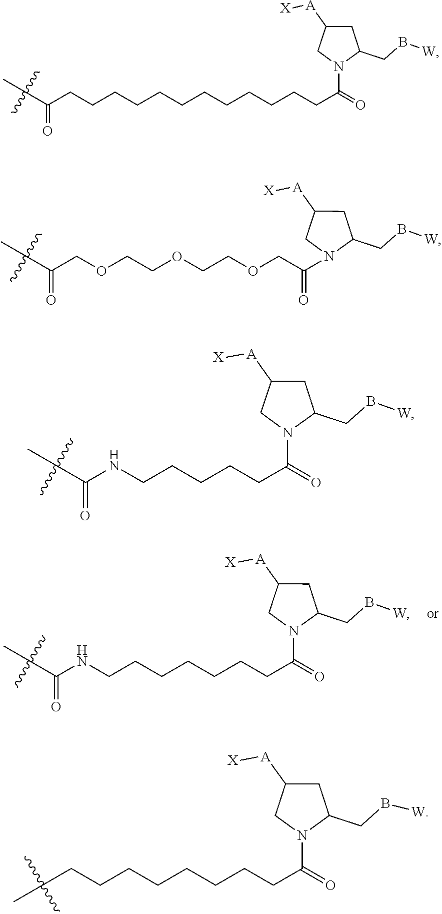

In certain embodiments, each -L′-T is independently a group having the following structure:

[-Q³-Q⁴-Q⁵]ₛ-Q⁶-T,

wherein
s is an integer from 0 to 20,
each of Q³ and Q⁶ is independently absent, —CO—, —NH—, —O—, —S—, —SO₂—, —OC(O)—, —C(O)O—, —NHC(O)—, —C(O)NH—, —CH₂—, —CH₂NH—, —NHCH₂—, —CH₂O—, or —OCH₂—,
each Q⁴ is independently absent, unsubstituted or substituted C₁₋₁₂ alkylene, unsubstituted or substituted C₂₋₁₂ alkenylene, unsubstituted or substituted C₂₋₁₂ alkynylene, unsubstituted or substituted C₂₋₁₂ heteroalkylene, unsubstituted or substituted 6- to 12-membered arylene, unsubstituted or substituted 5- to 12-membered heteroarylene, or unsubstituted or substituted 5- to 12-membered heterocyclylene, and
each Q⁵ is independently absent, —CO—, —NH—, —O—, —S—, —SO₂—, —CH₂—, —C(O)O—, —OC(O)—, —C(O)NH—, —NHC(O)—, —NH—CH(R^{a})—C(O)—, —C(O)—CH(R^{a})—NH—, —OP(O)(OH)O—, or —OP(S)(OH)O—, wherein each R^{a} is independently H or unsubstituted or substituted C₁₋₁₂ alkyl,
provided that at least one of Q³, Q⁴, Q⁵, and Q⁶ is present.

In certain embodiments, s is 1, 2, 3, 4, 5, 6, 7, 8, 9, or 10.

In certain embodiments, each -L′-T is independently a group of the following structure:

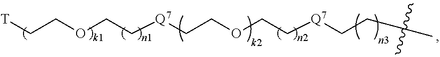

wherein
each Q⁷ is independently absent, —CO—, —NH—, —O—, —S—, —SO₂—, —OC(O)—, —C(O)O—, —NHC(O)—, —C(O)NH—, —CH₂—, —CH₂NH—, —NHCH₂—, —CH₂O—, or —OCH₂—,
each of k1 and k2 is independently 0, 1, 2, 3, 4, 5, 6, 7, 8, 9, or 10, and
each of n1, n2 and n3 is independently 1, 2, 3, 4, or 5.

In certain embodiments, each Q⁷ is independently —NHC(O)— or —C(O)NH—.

In certain embodiments, each -L′-T is independently a group of the following structure:

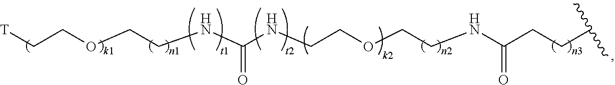

wherein
each of k1 and k2 is independently 0, 1, 2, or 3,
each of n1, n2 and n3 is independently 1, 2, 3, 4, or 5, and
one of t1 and t2 is 0 and anther of t1 and t2 is 1.

In certain embodiments, each -L′-T is independently a group having the following structure:

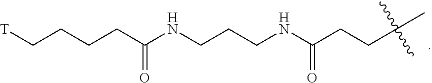

In certain embodiments, each -L′-T is independently a group of the following structure:

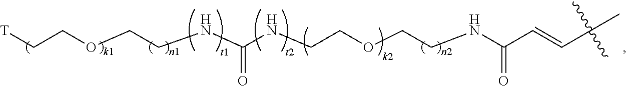

wherein
each of k1 and k2 is independently 0, 1, 2, or 3,
each of n1 and n2 is independently 1, 2, 3, 4, or 5, and
one of t1 and t2 is 0 and another of t1 and t2 is 1.

In certain embodiments, each -L′-T is independently a group having the following structure:

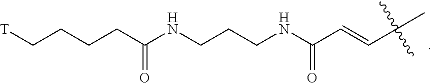

In certain embodiments, each T is independently a carbohydrate ligand.

In certain embodiments, each T is independently a carbohydrate ligand selected from the group consisting of N-acetyl-galactosamine (GalNAc), allose, altrose, arabinose, cladinose, erythrose, erythrulose, fructose, D-fucitol, L-fucitol, fucosamine, fucose, fuculose, galactosamine, D-galactosaminitol, galactose, glucosamine, N-acetyl-glucosamine, glucosaminitol, glucose, glucose-6-phosphate, gulose glyceraldehyde, L-glycero-D-mannos-heptose, glycerol, glycerone, gulose, idose, lyxose, mannosamine, mannose, mannose-6-phosphate, psicose, quinovose, quinovosamine, rhamnitol, rhamnosamine, rhamnose, ribose, ribulose, sedoheptulose, sorbose, tagatose, talose, tartaric acid, threose, xylose, and xylulose, in an unprotected or protected form.

In certain embodiments, each T is independently N-acetyl-galactosamine (GalNAc) or N-acetyl-galactosamine triacetate.

In certain embodiments, the therapeutically active agent is selected from the group consisting of an antisense oligonucleotide (ASO), a small interfering RNA (siRNA), a microRNA (miRNA), a microRNA mimic, an anti-miRNA oligonucleotide (AMO), a long non-coding RNA, a peptide nucleic acid (PNA), a helper lipid, and a phosphorodiamidate morpholino oligomer (PMO), wherein the nucleic acid is unmodified or modified.

In certain embodiments, the therapeutically active agent is a small interfering RNA (siRNA).

In certain embodiments, said compound comprises a structure selected from the group consisting of:

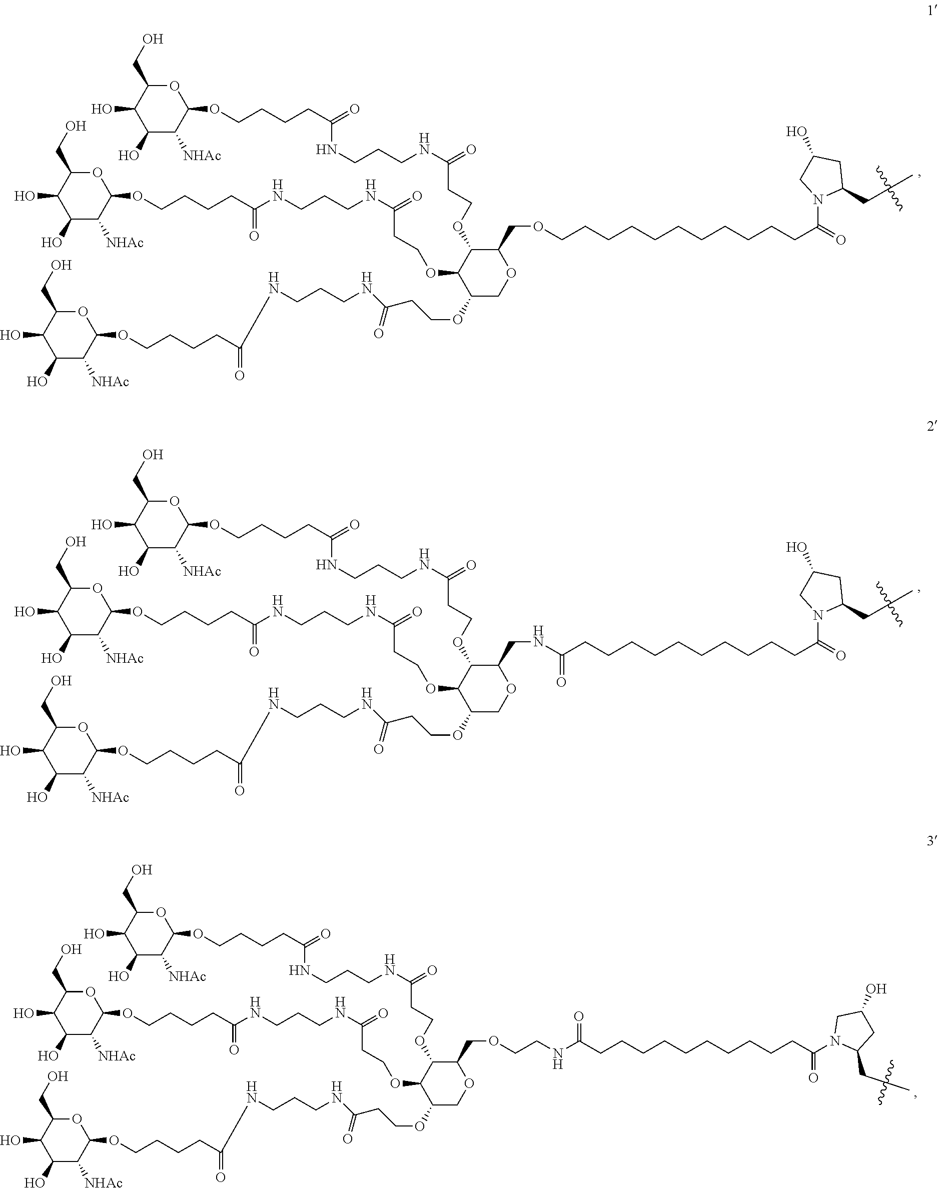

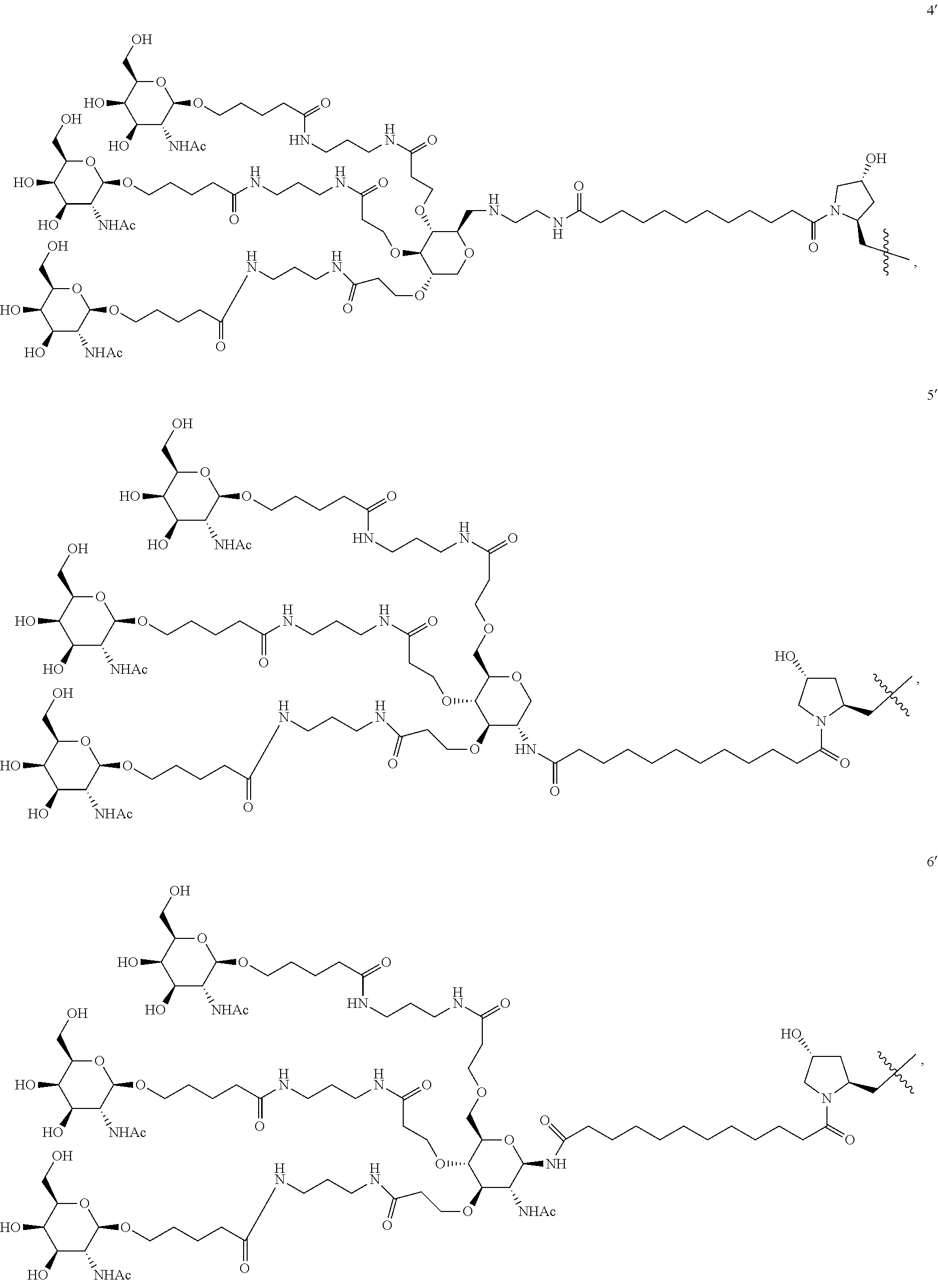

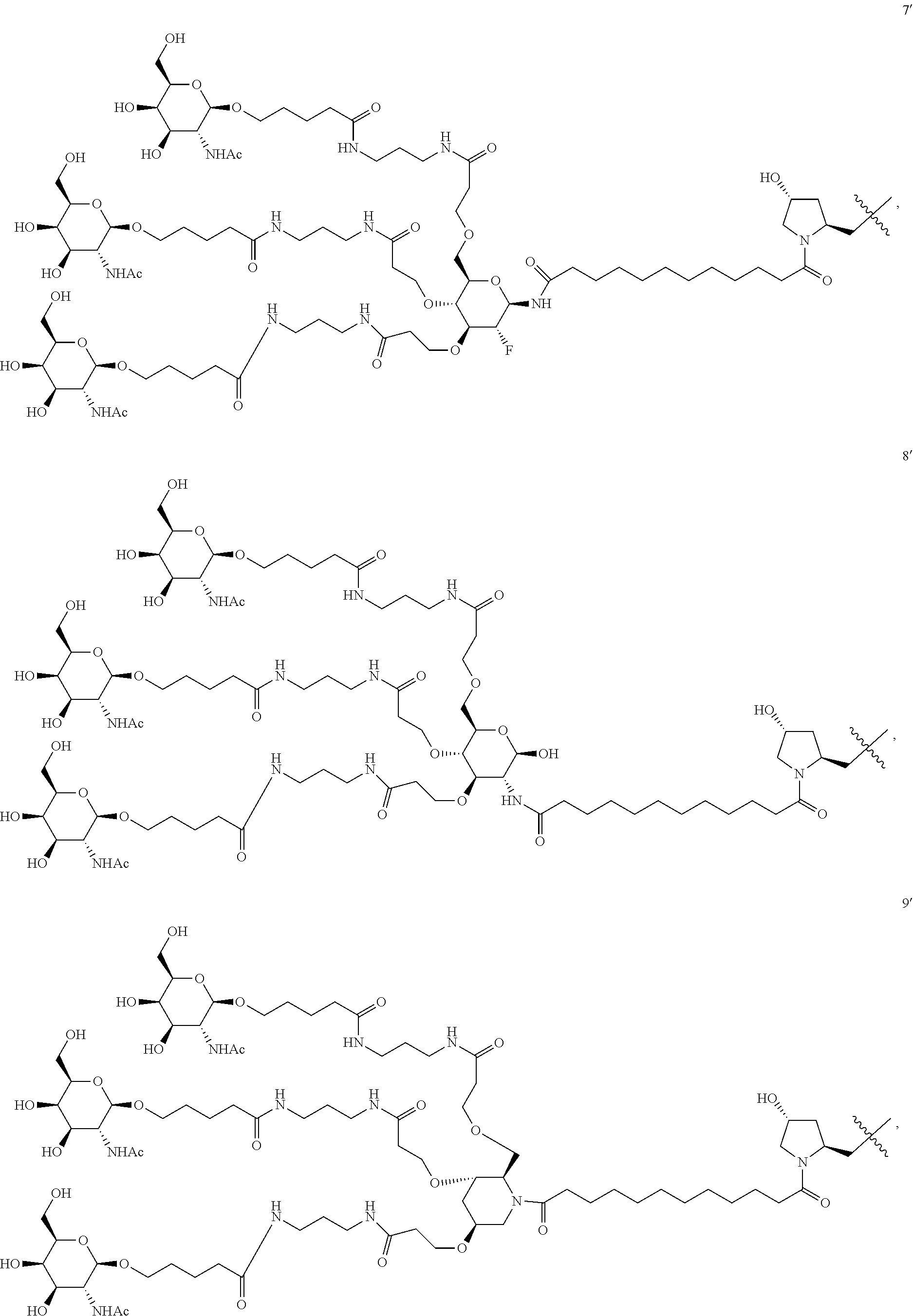

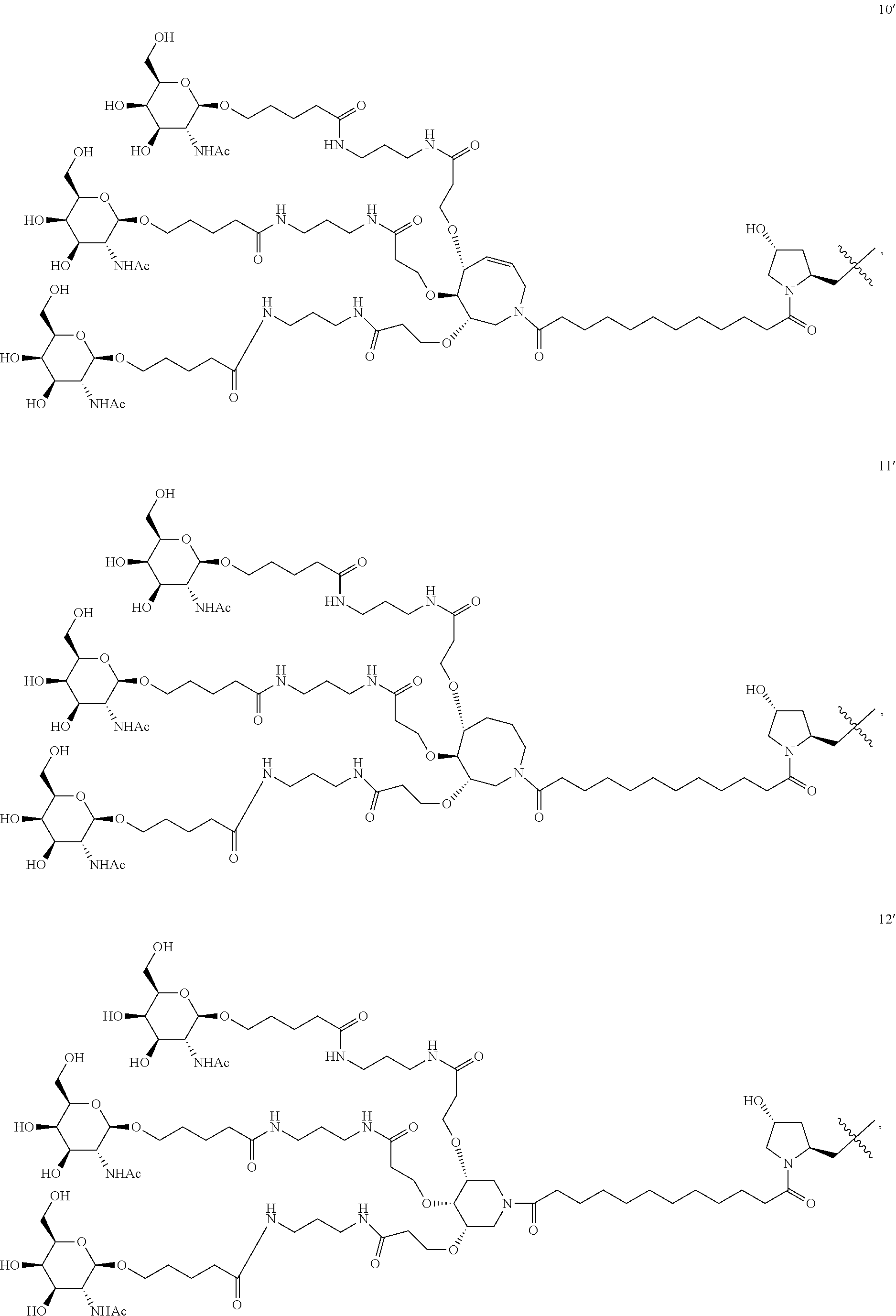

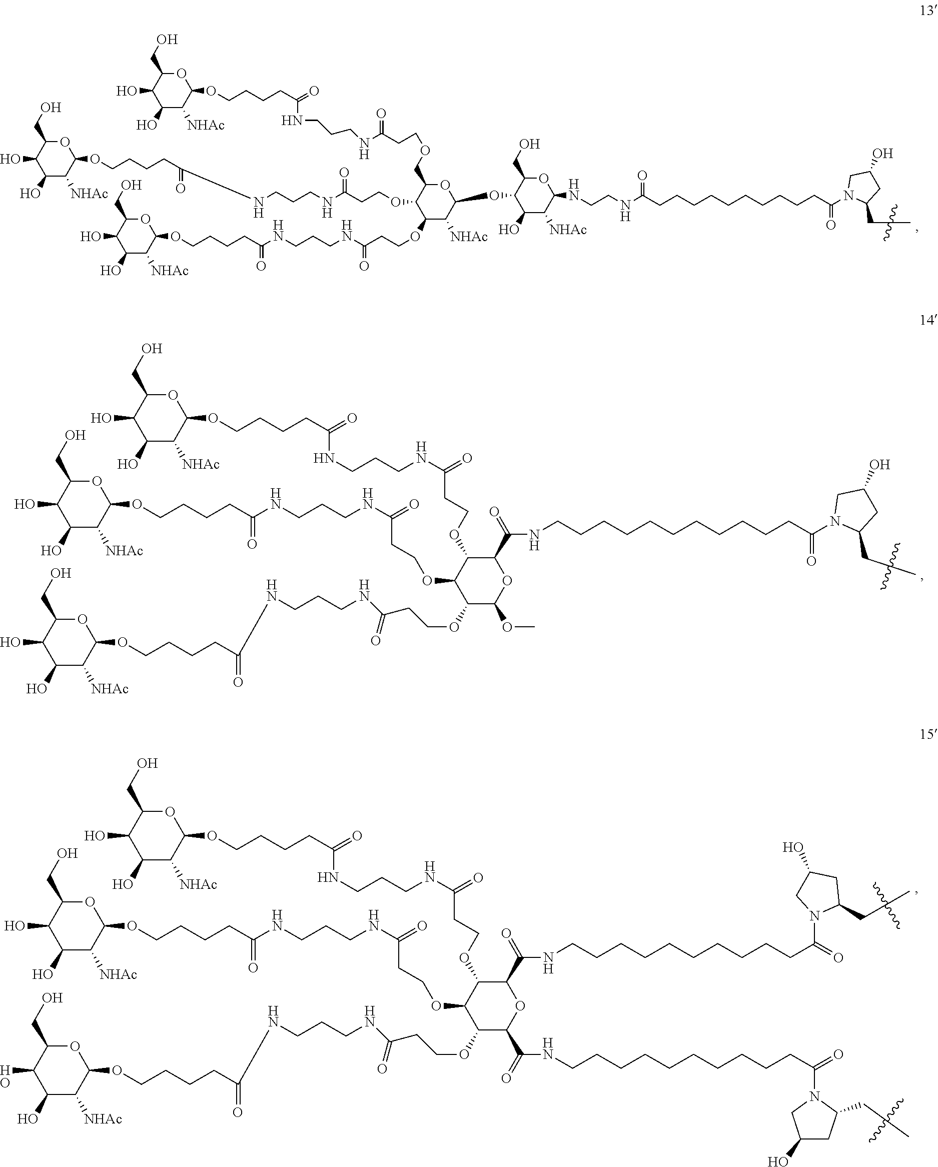

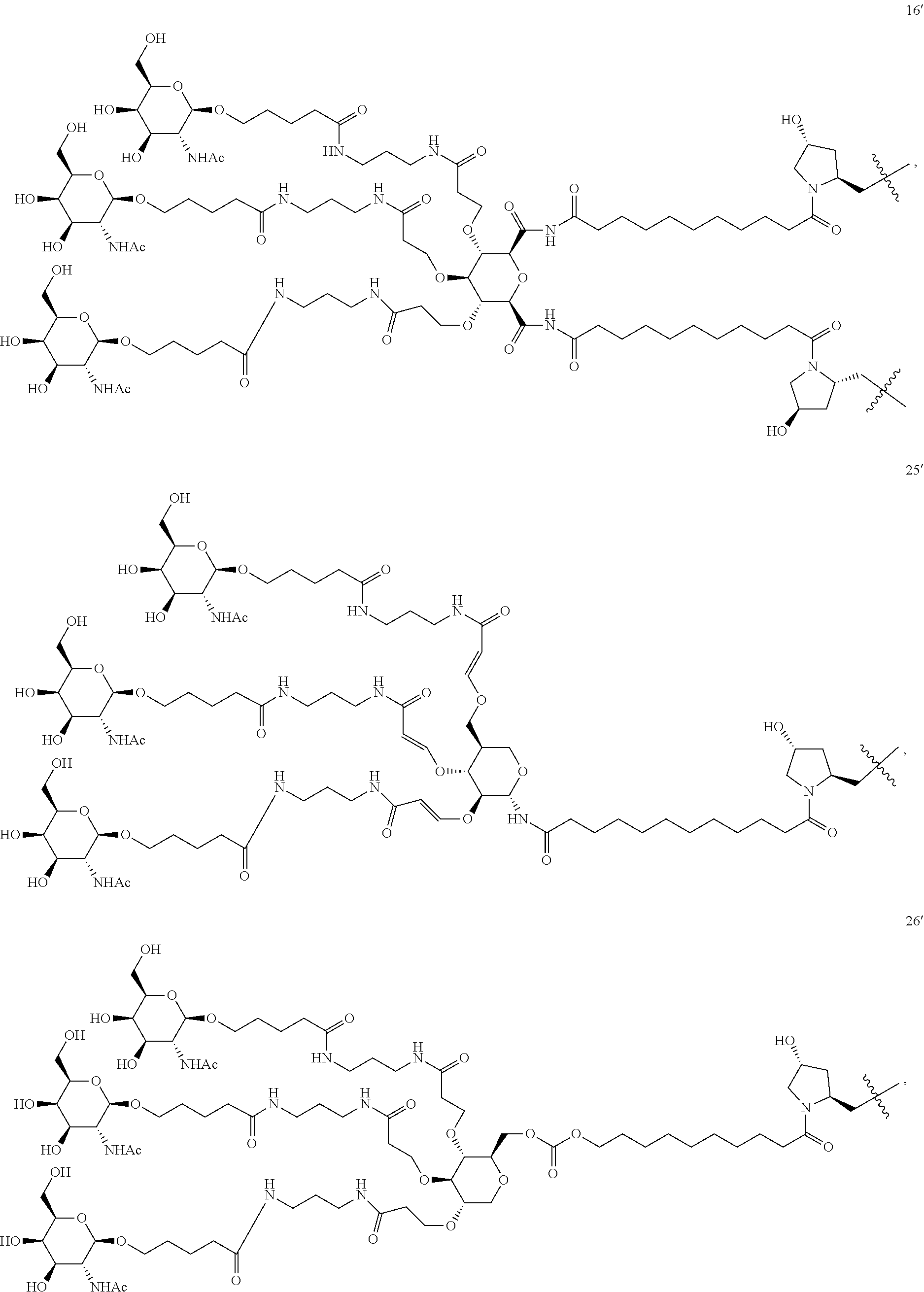

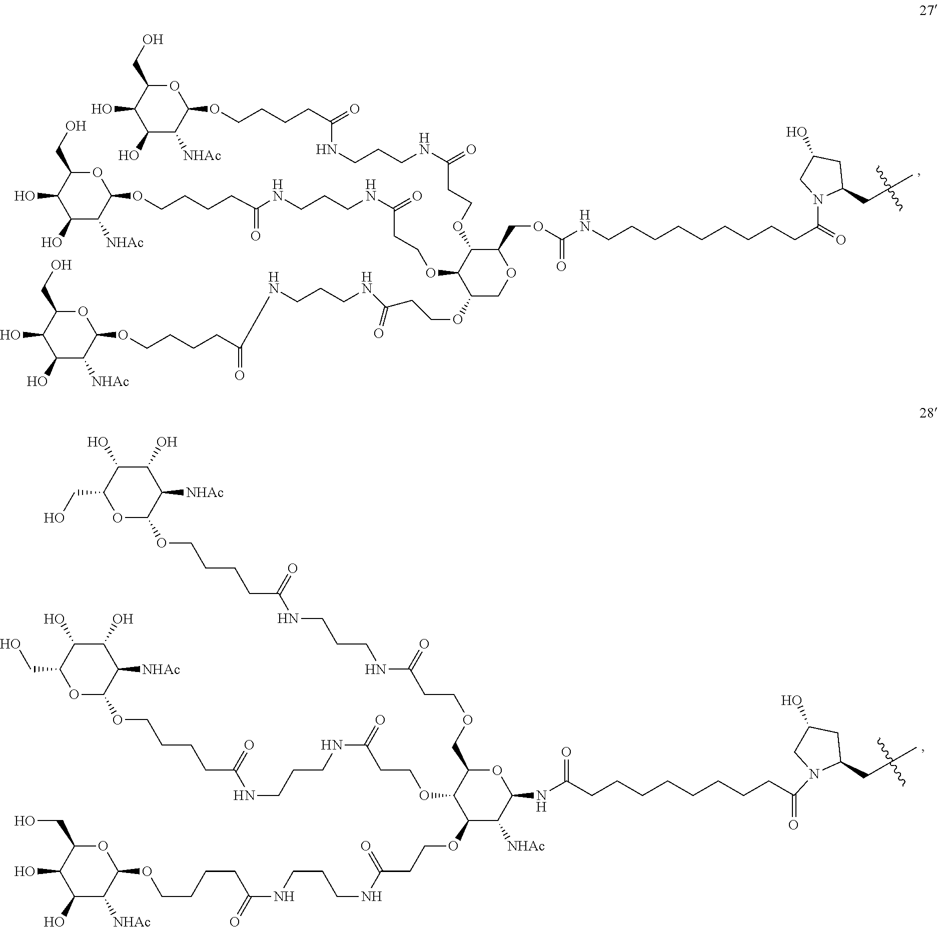

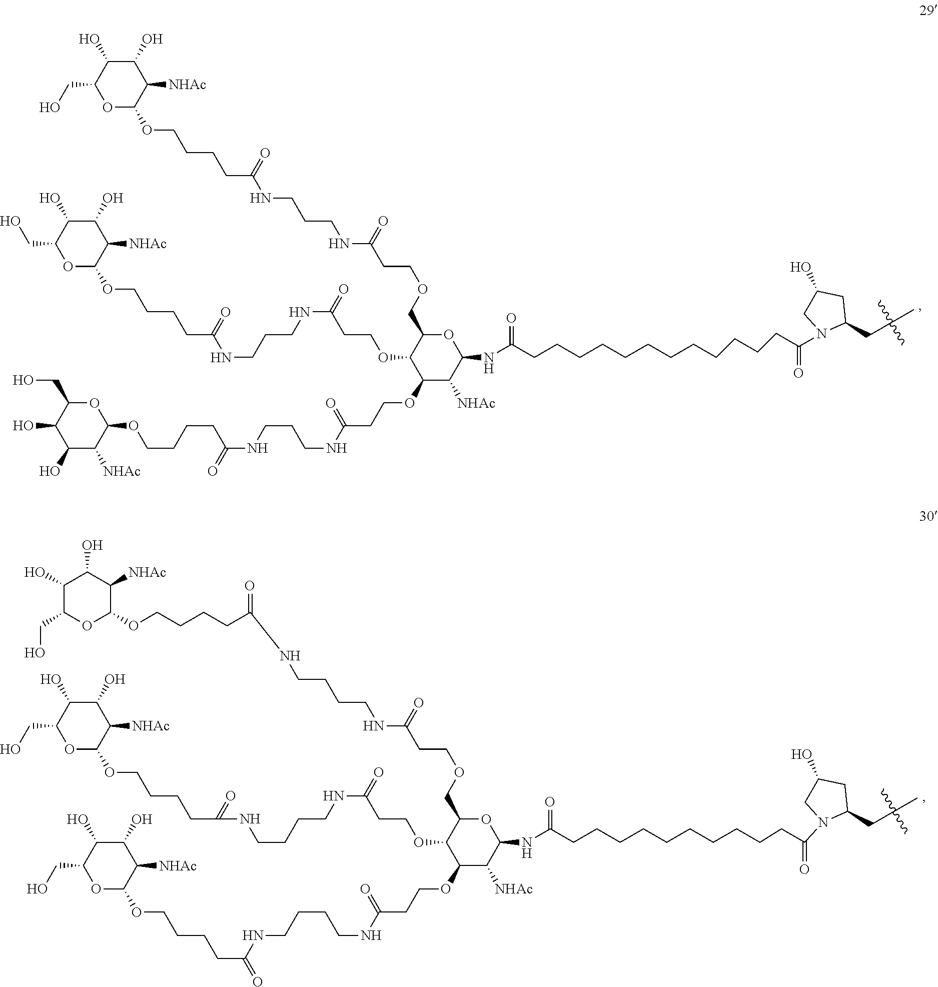

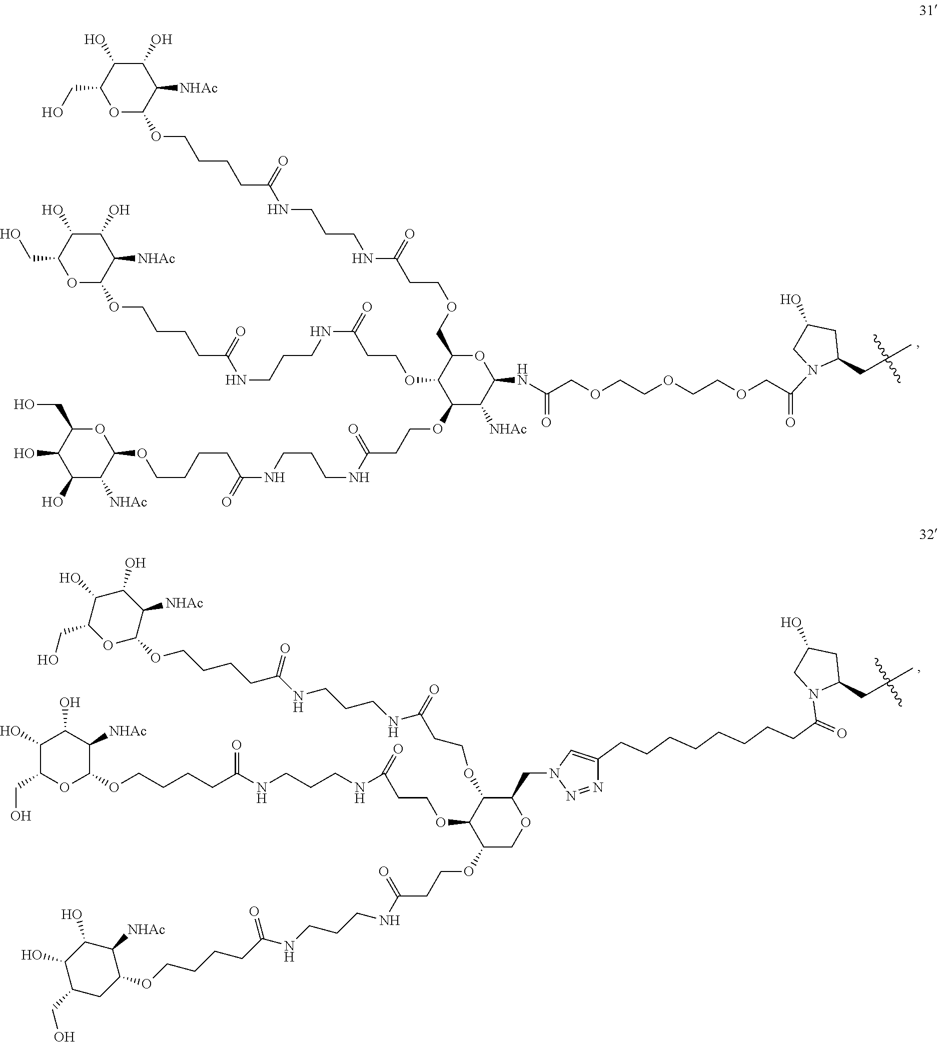

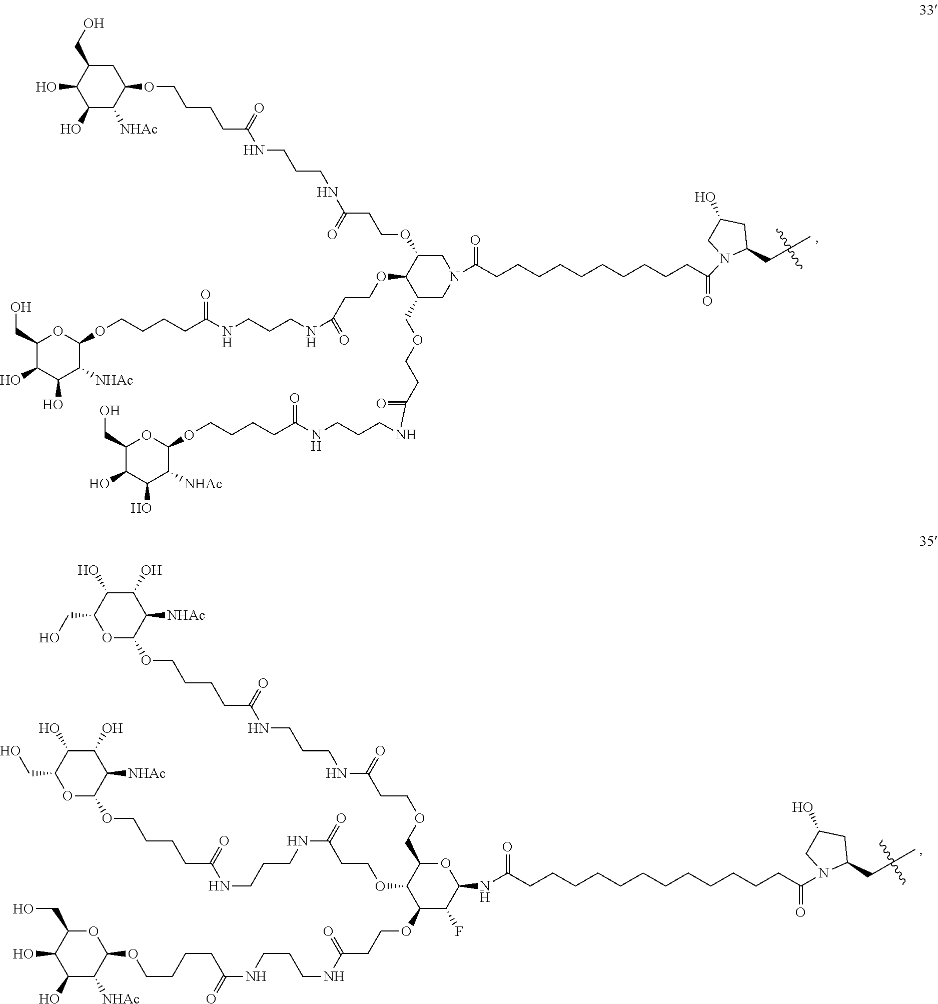

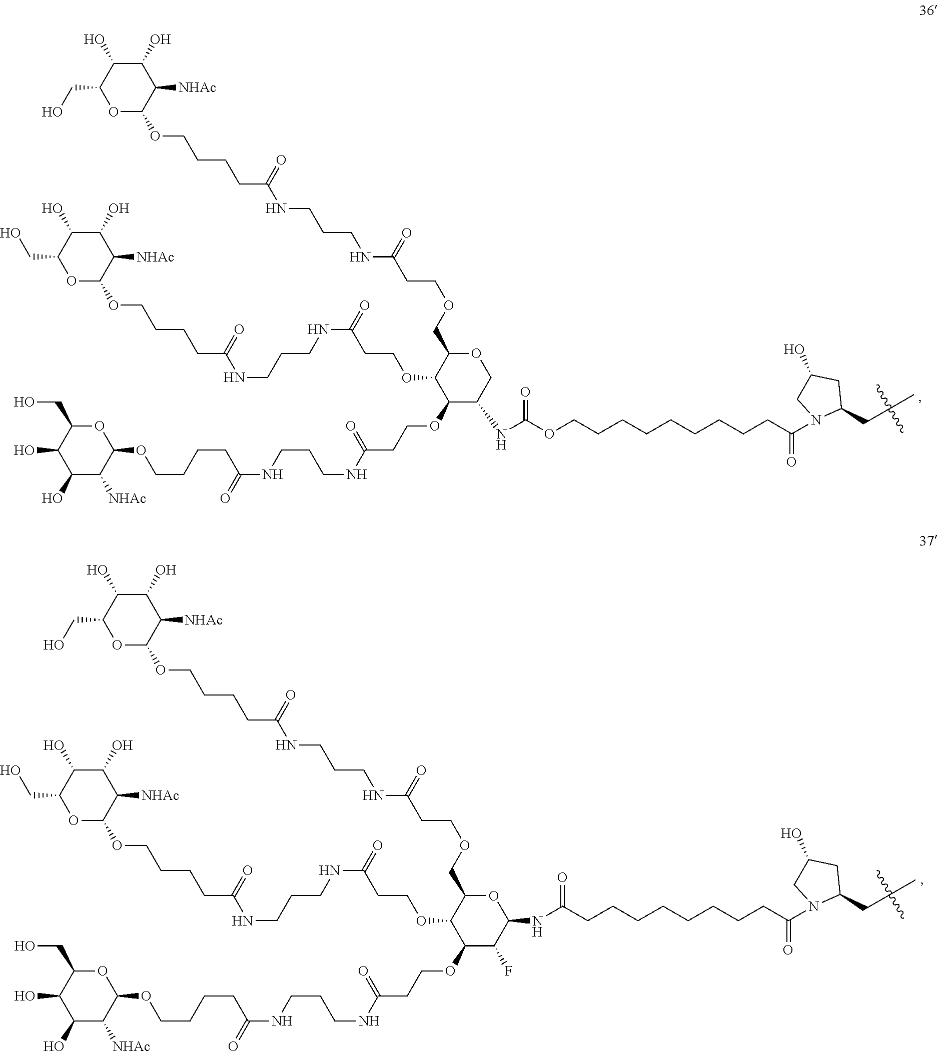

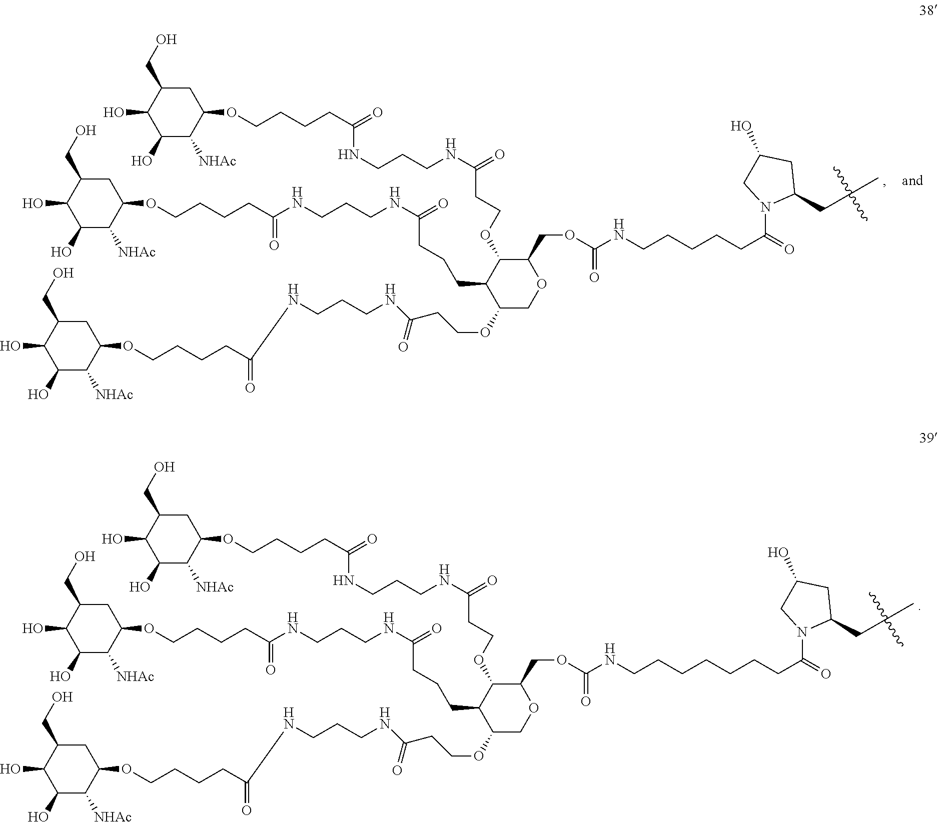

In certain embodiments, said compound comprises a structure selected from the group consisting of:

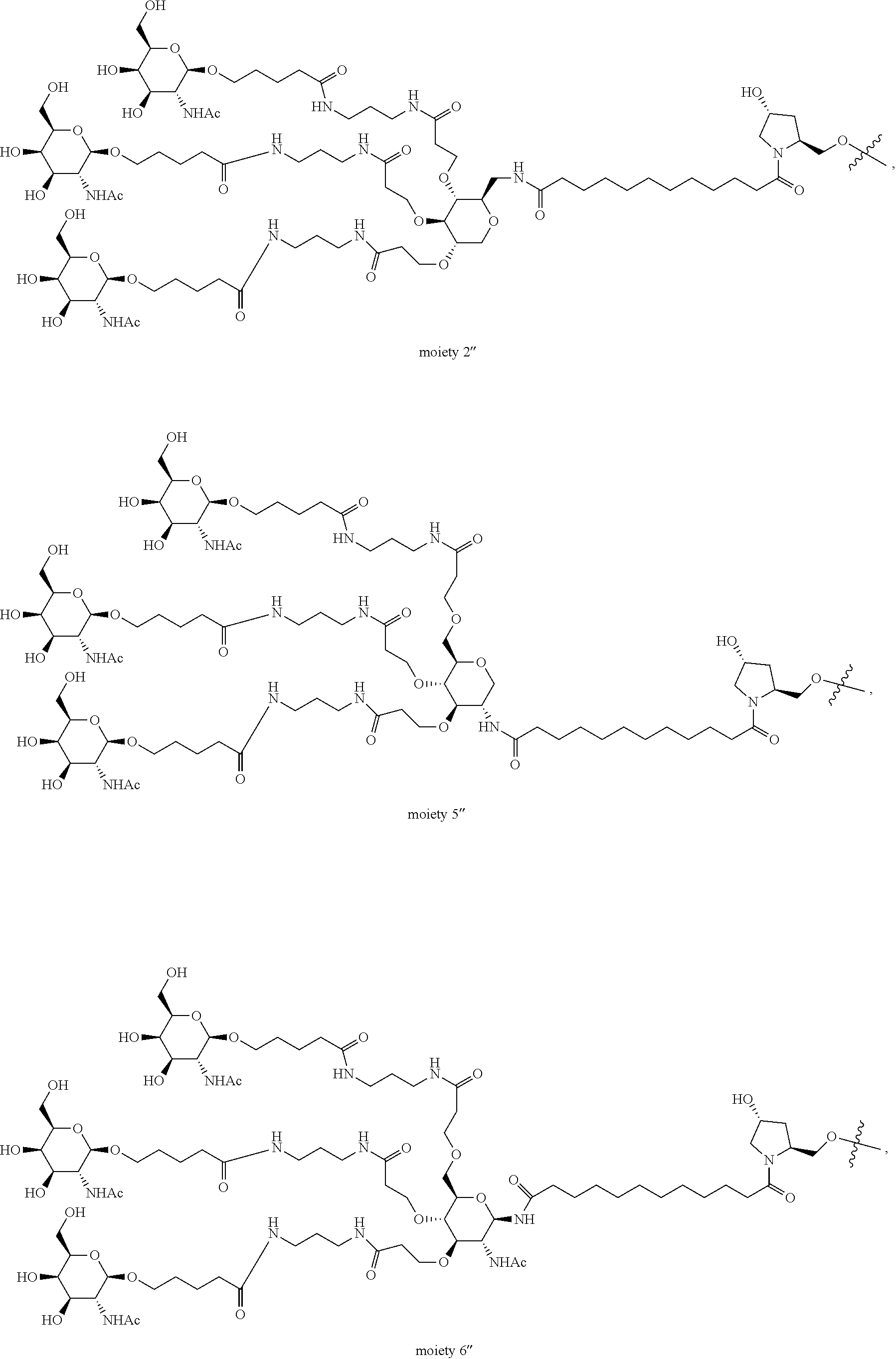

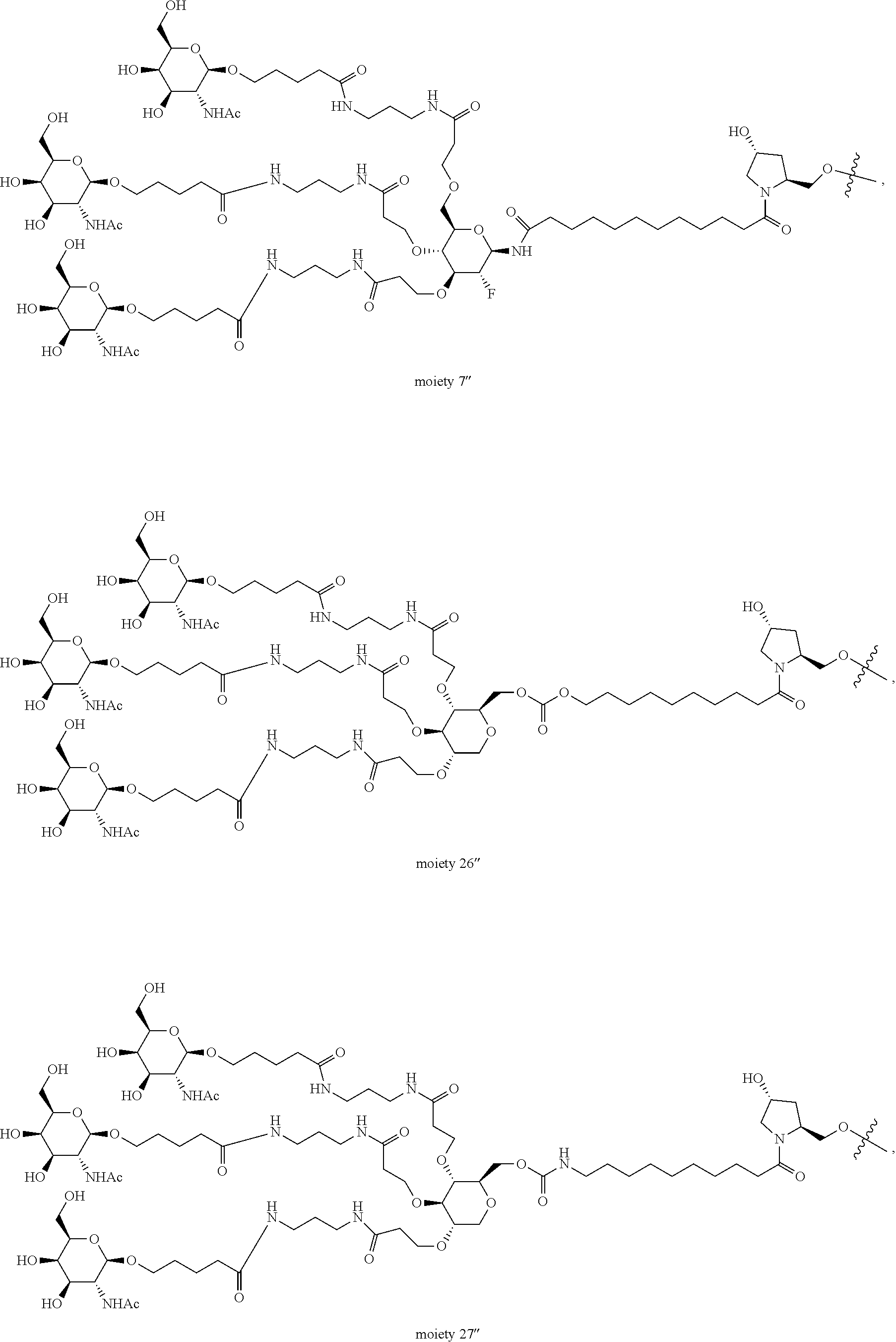

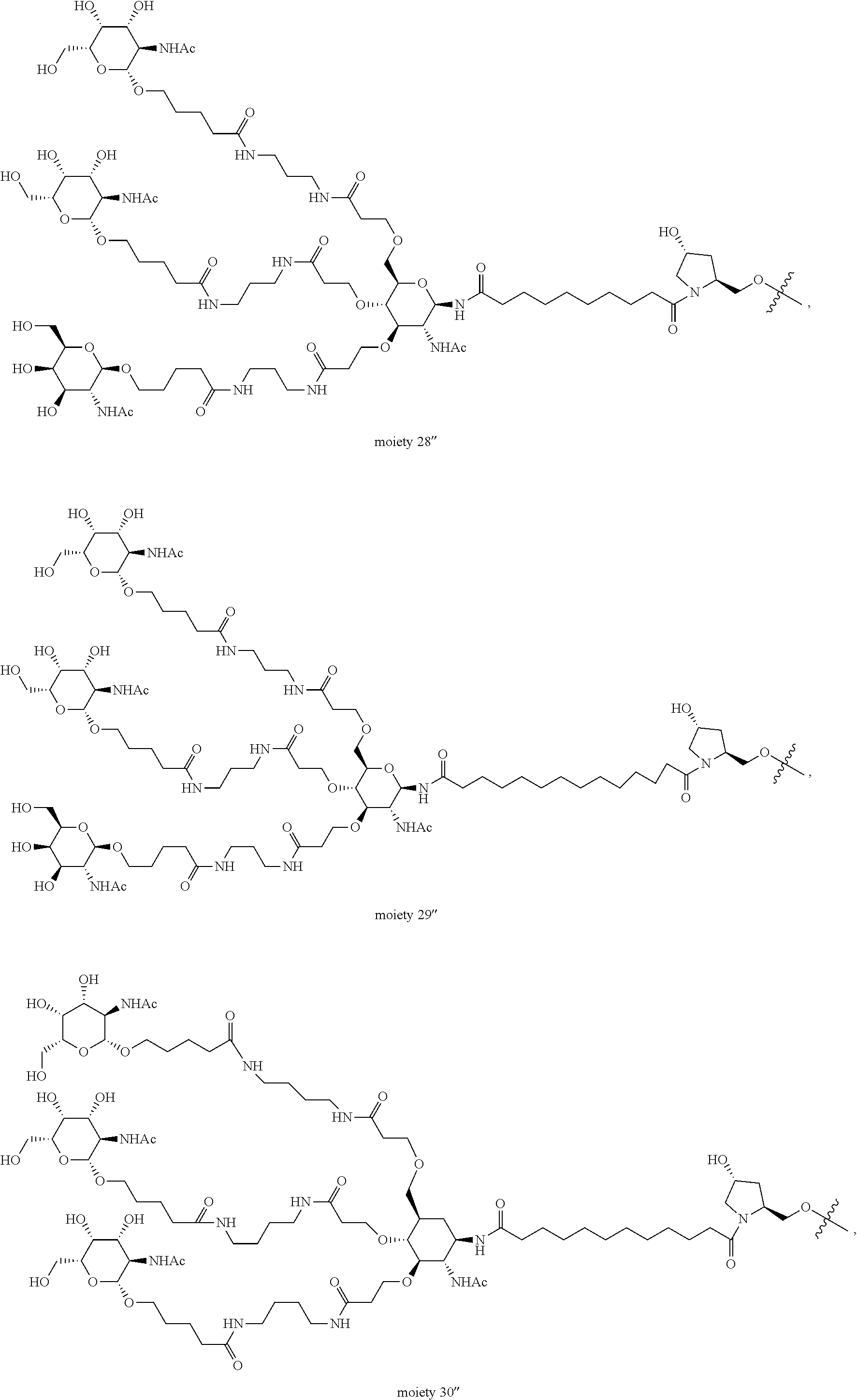

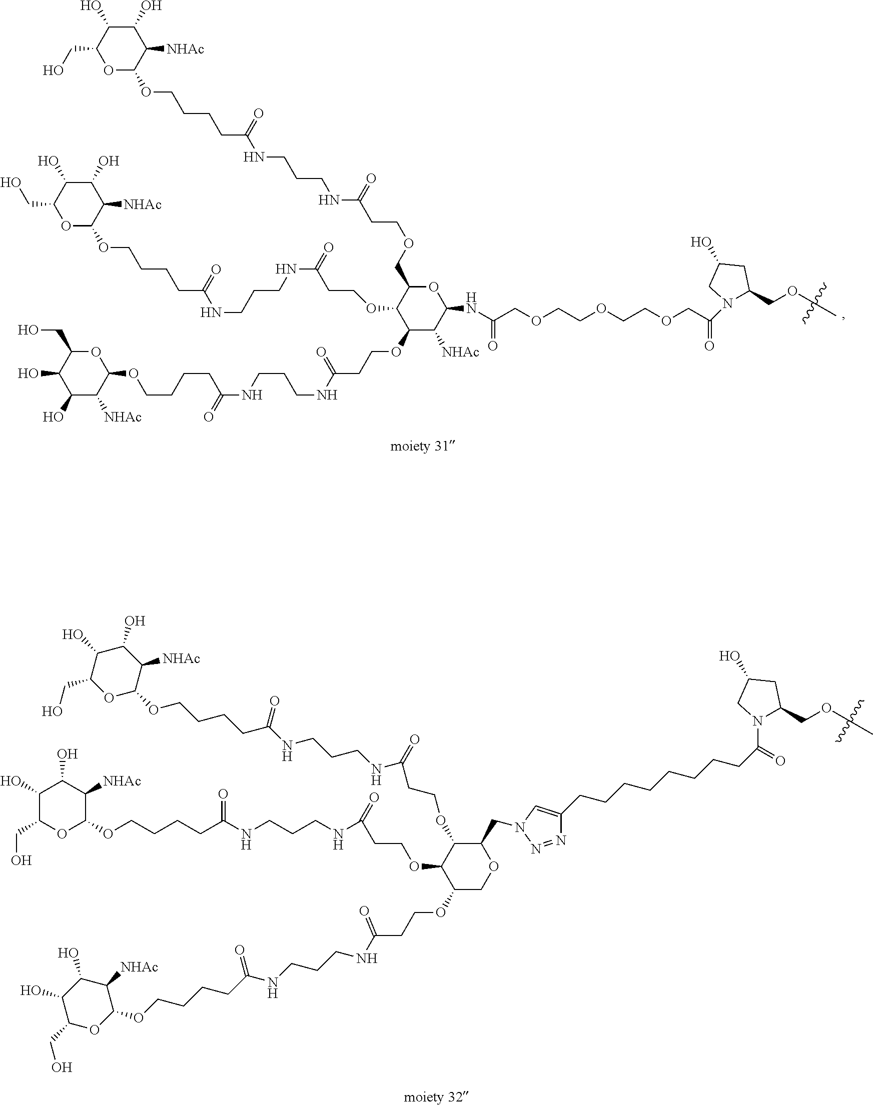

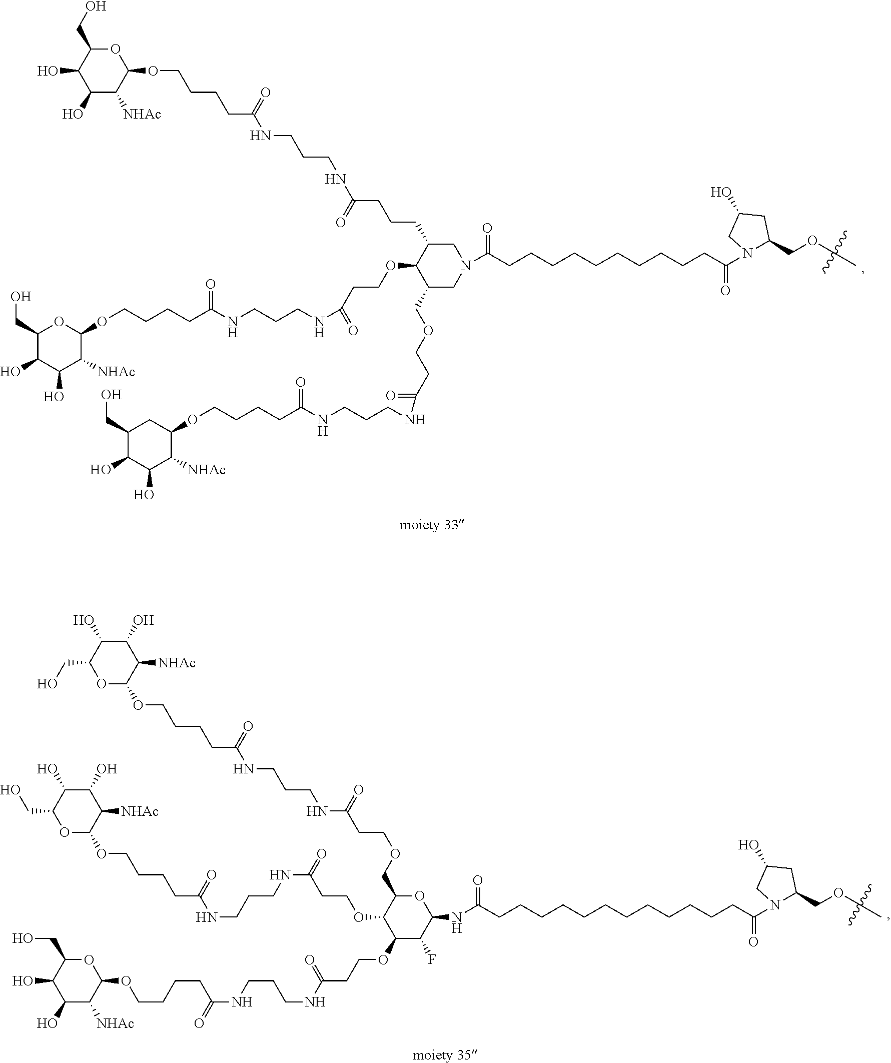

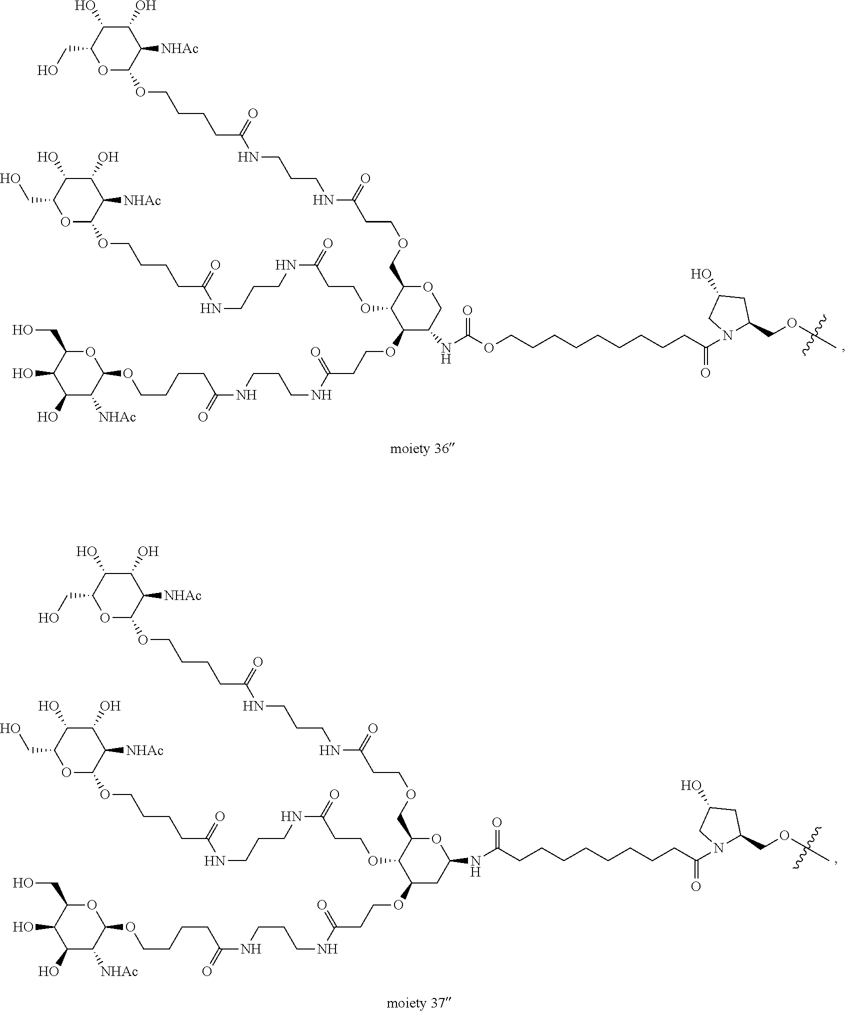

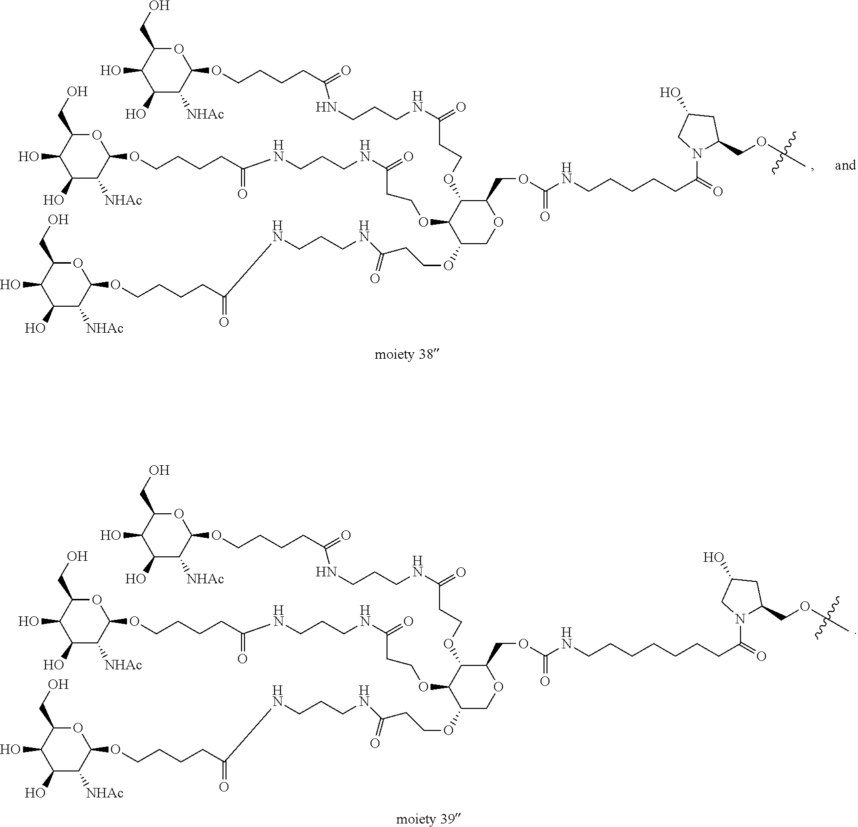

In certain embodiments, said compound is selected from a group consisting of the following compounds:

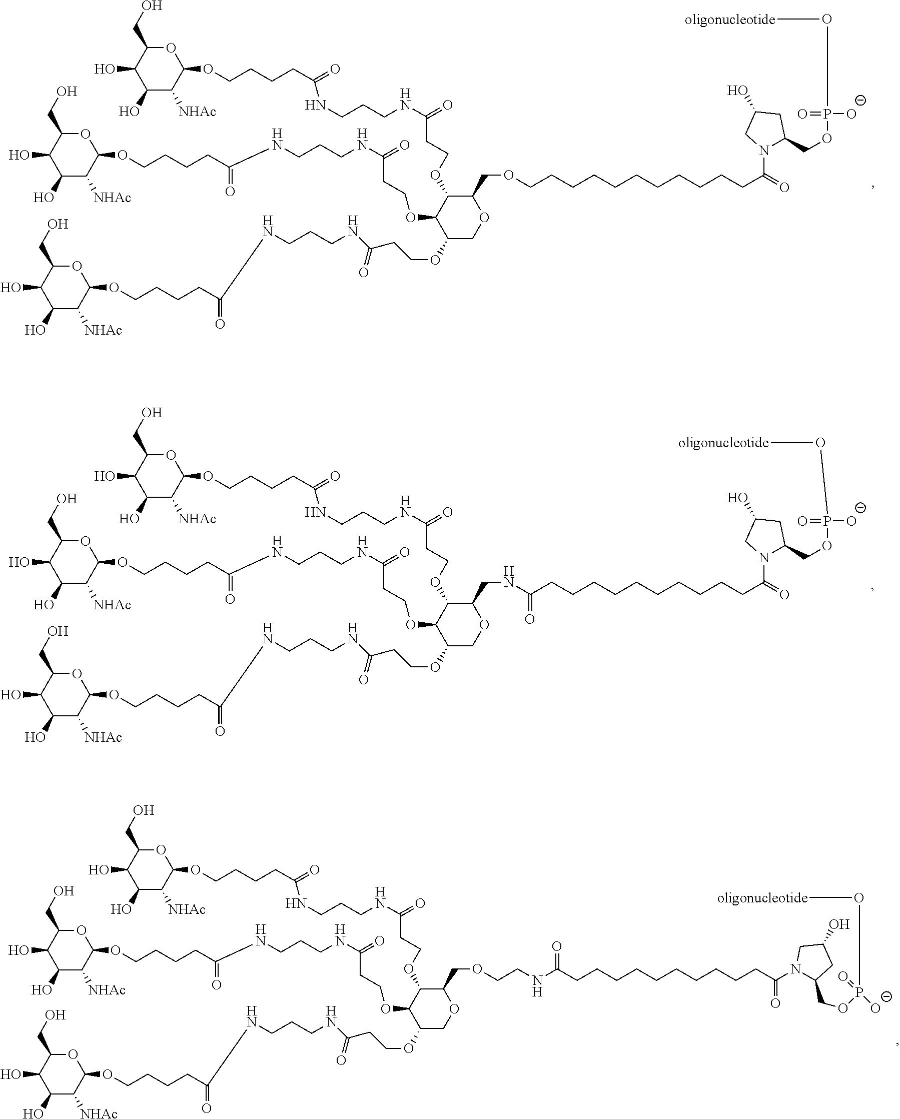

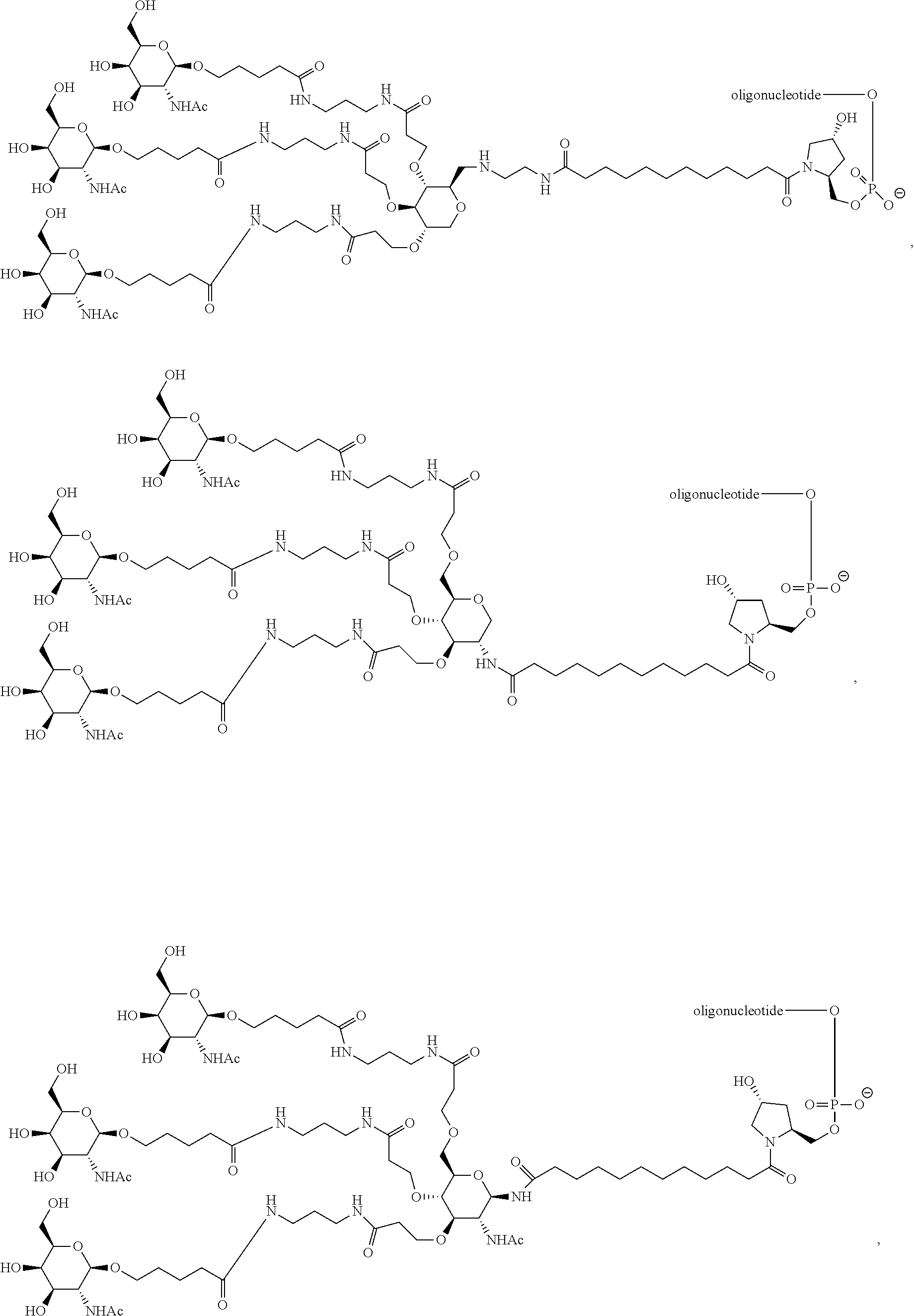

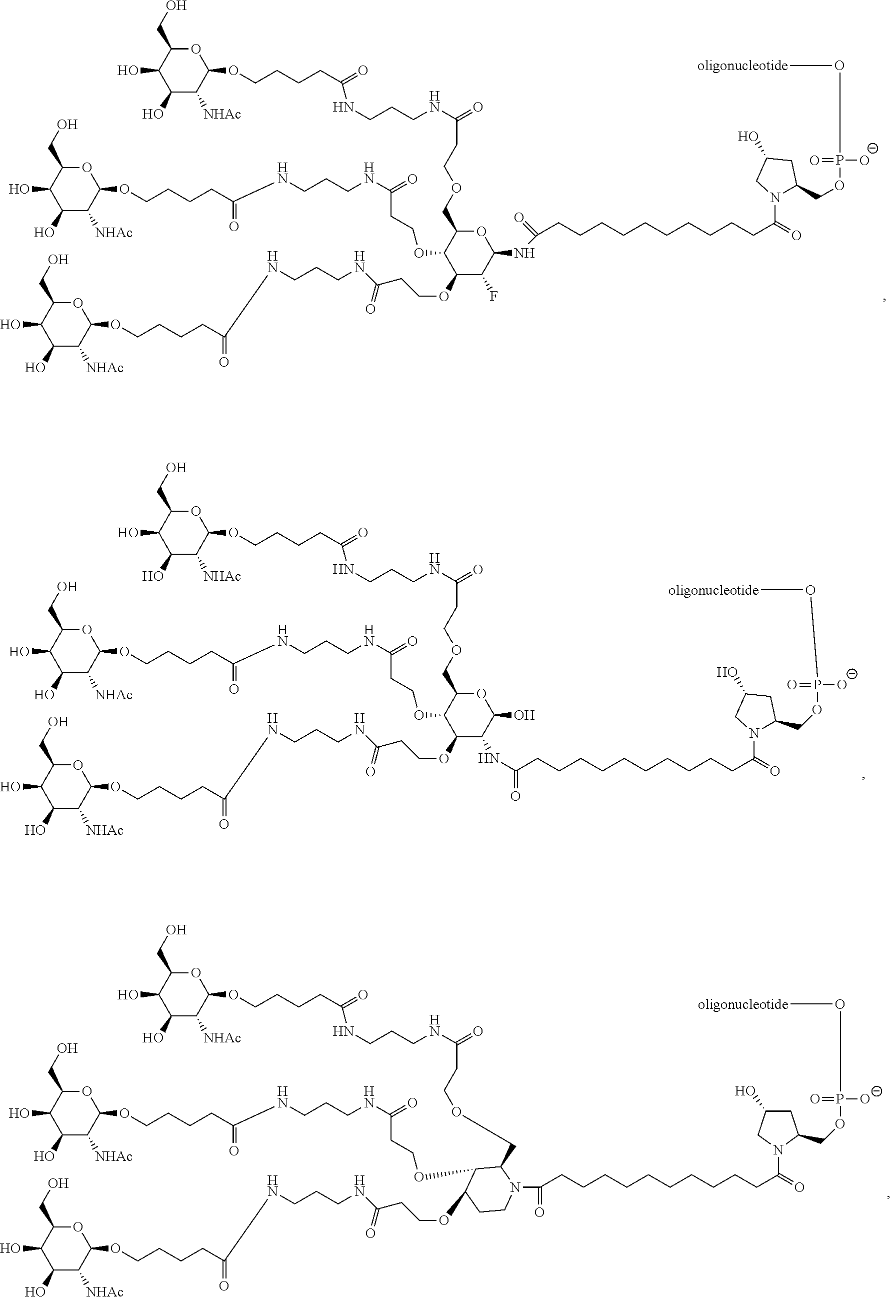

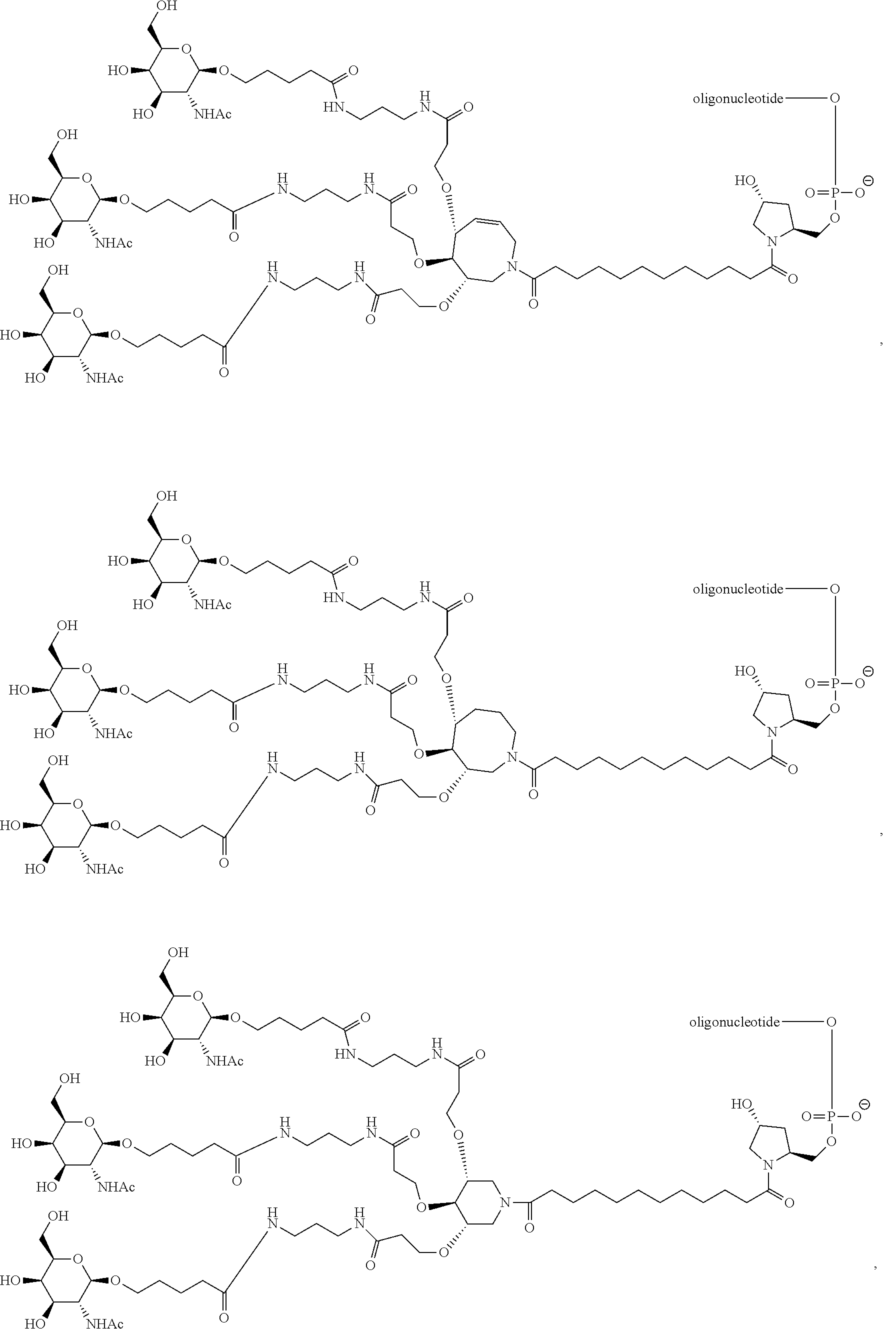

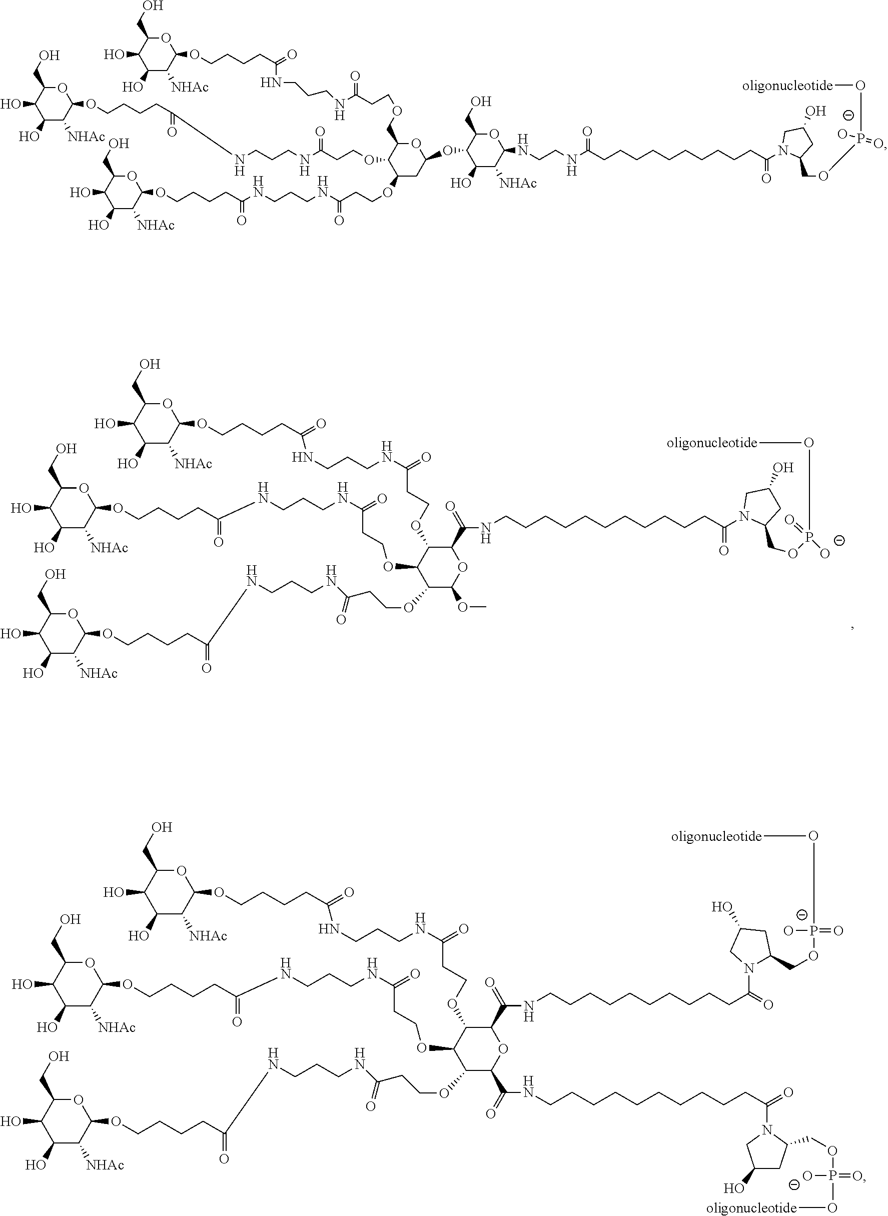

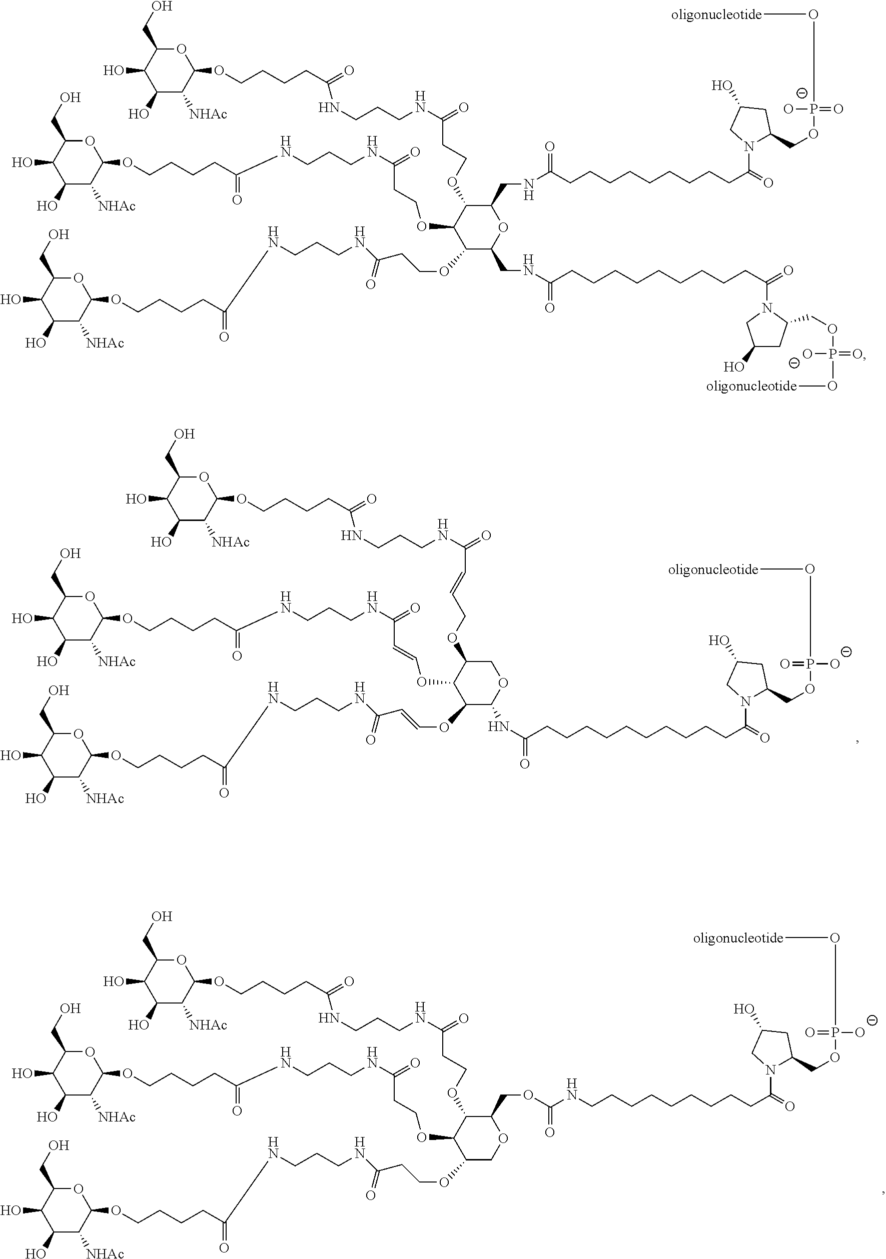

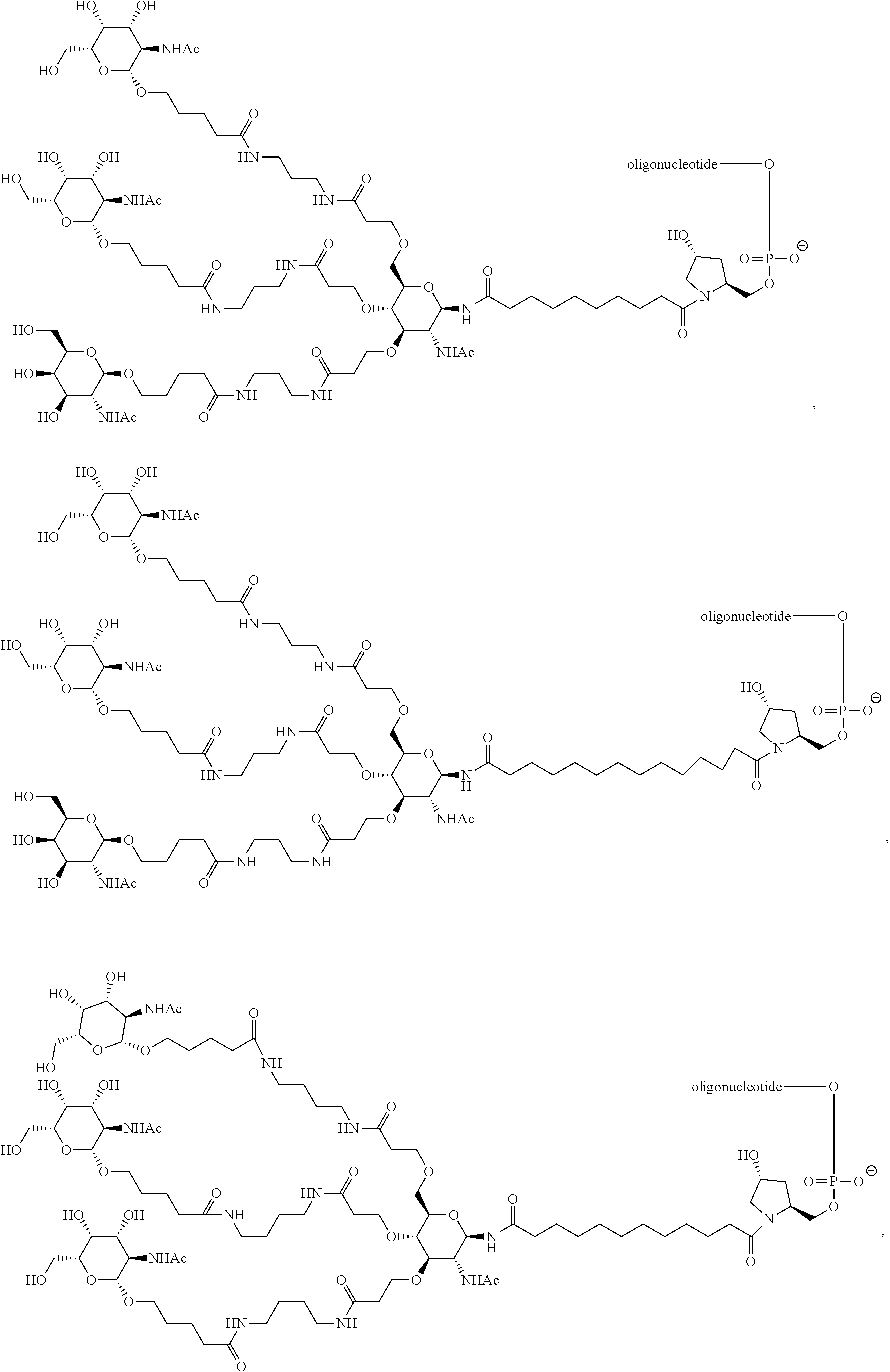

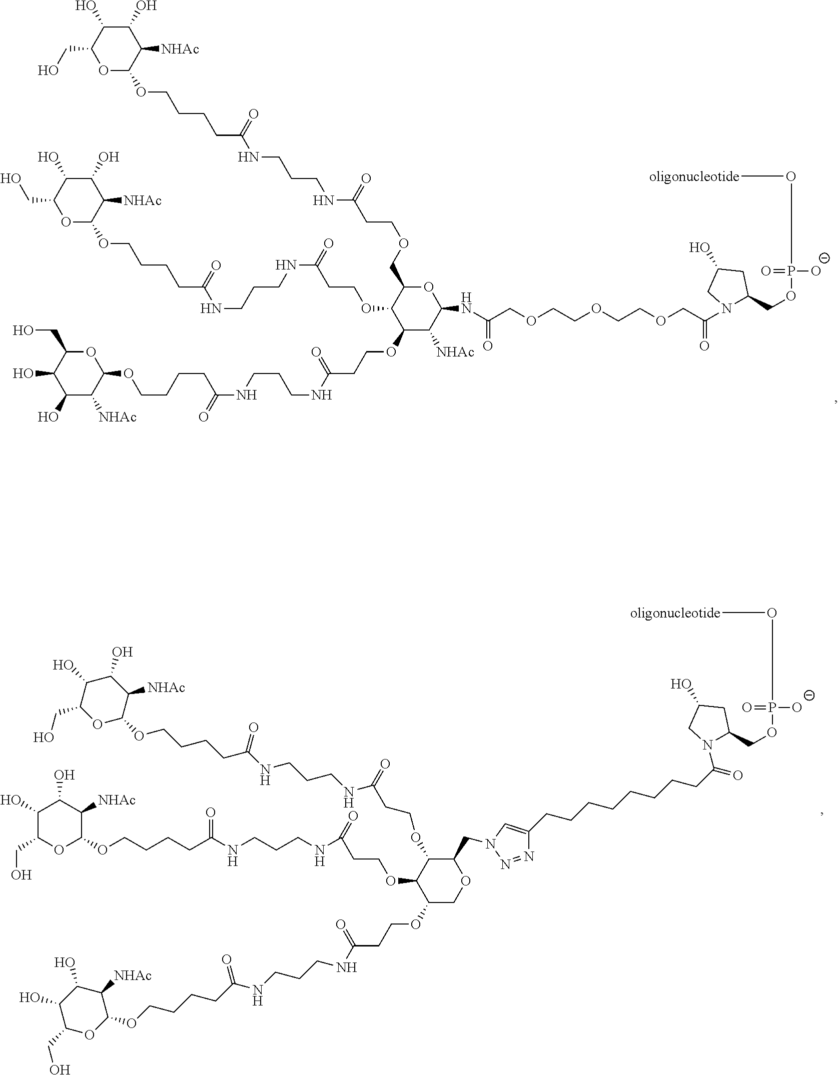

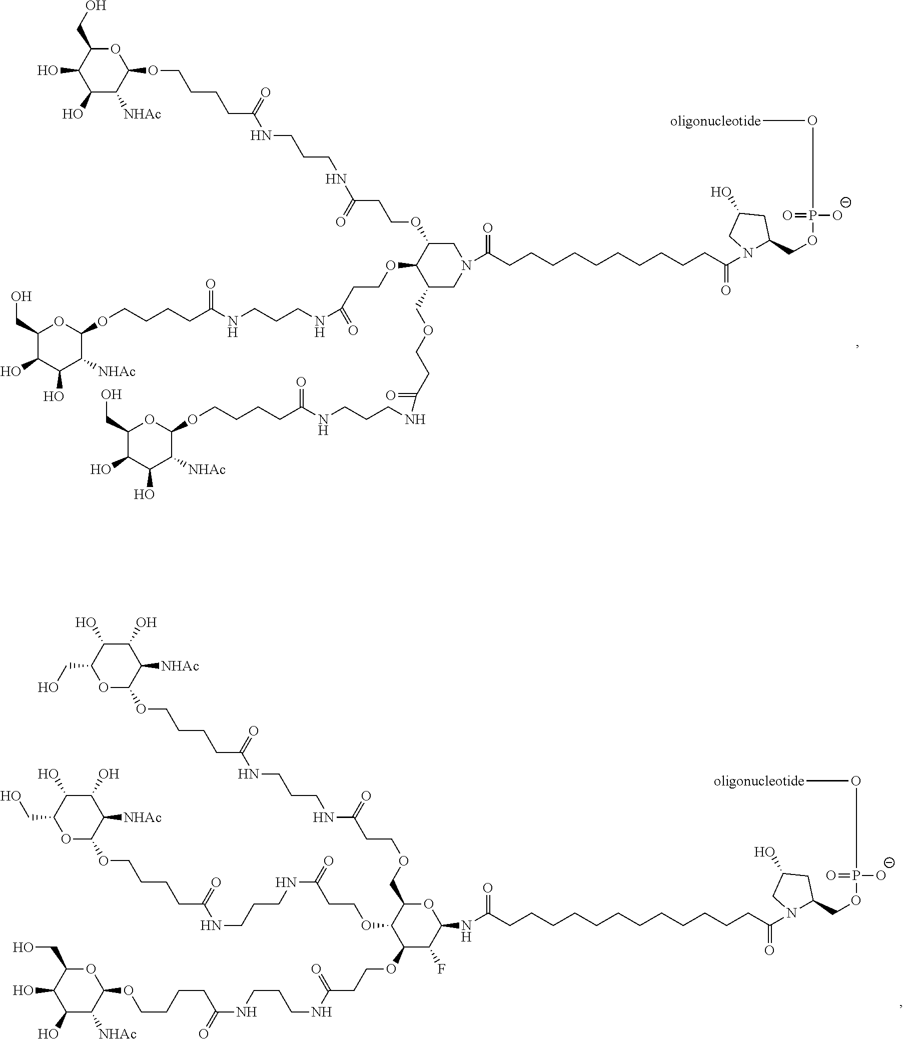

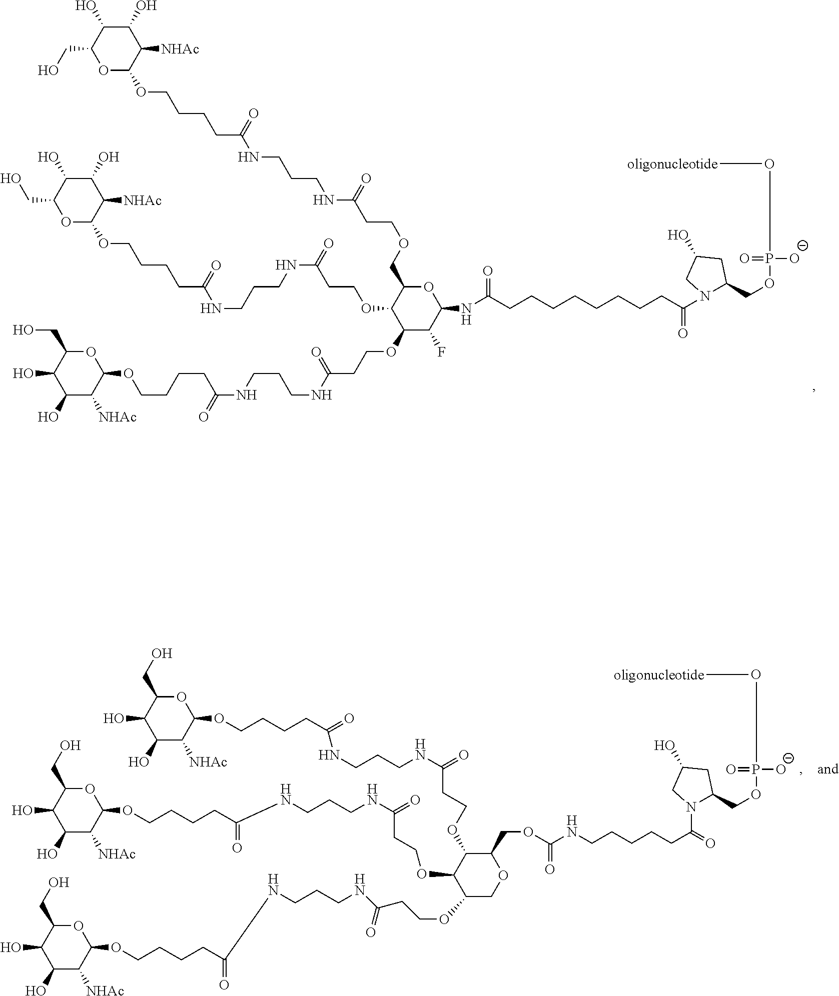

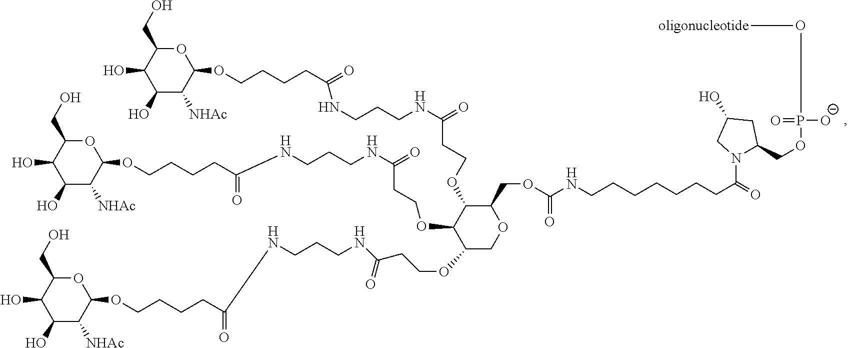

or a pharmaceutically acceptable salt or solvate thereof.

In certain embodiments, said compound is selected from a group consisting of the following compounds:

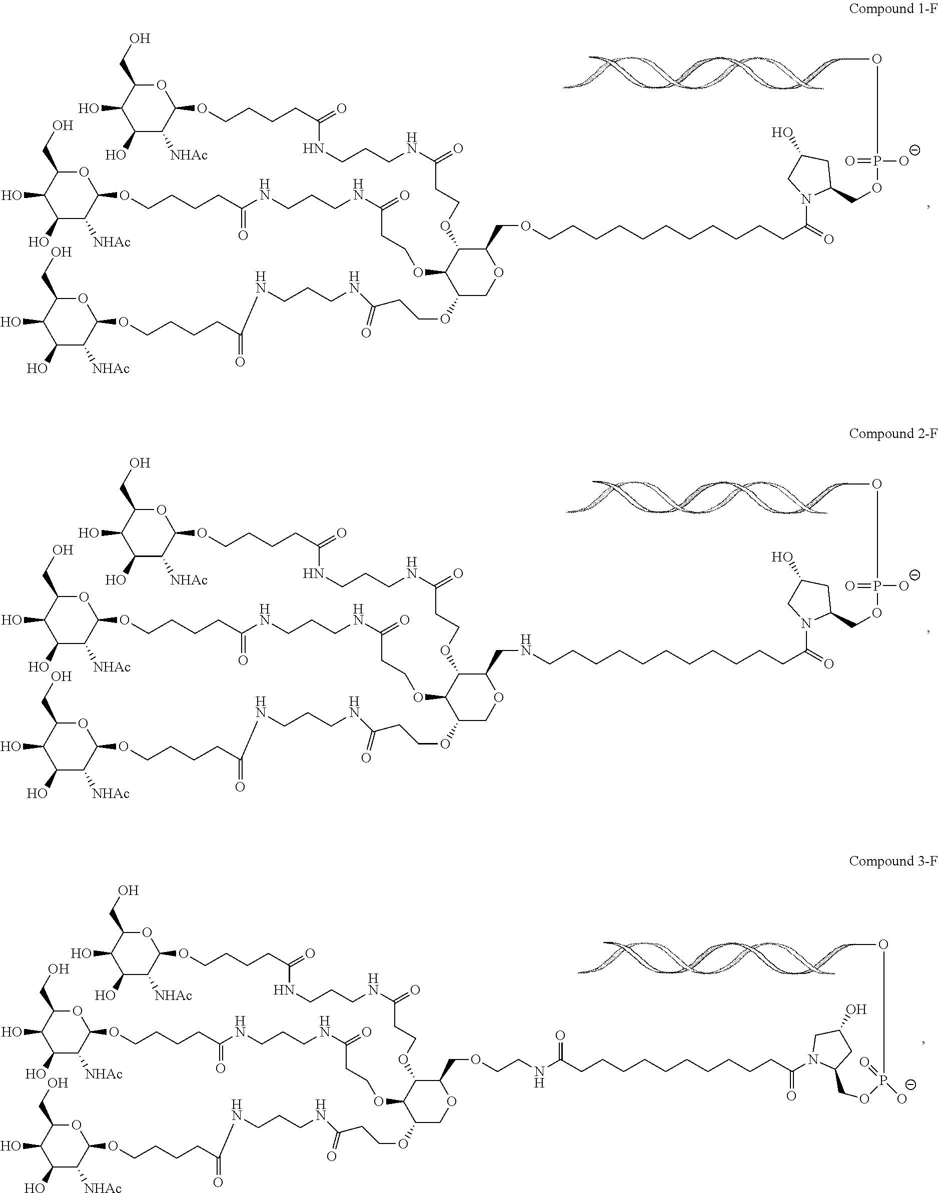

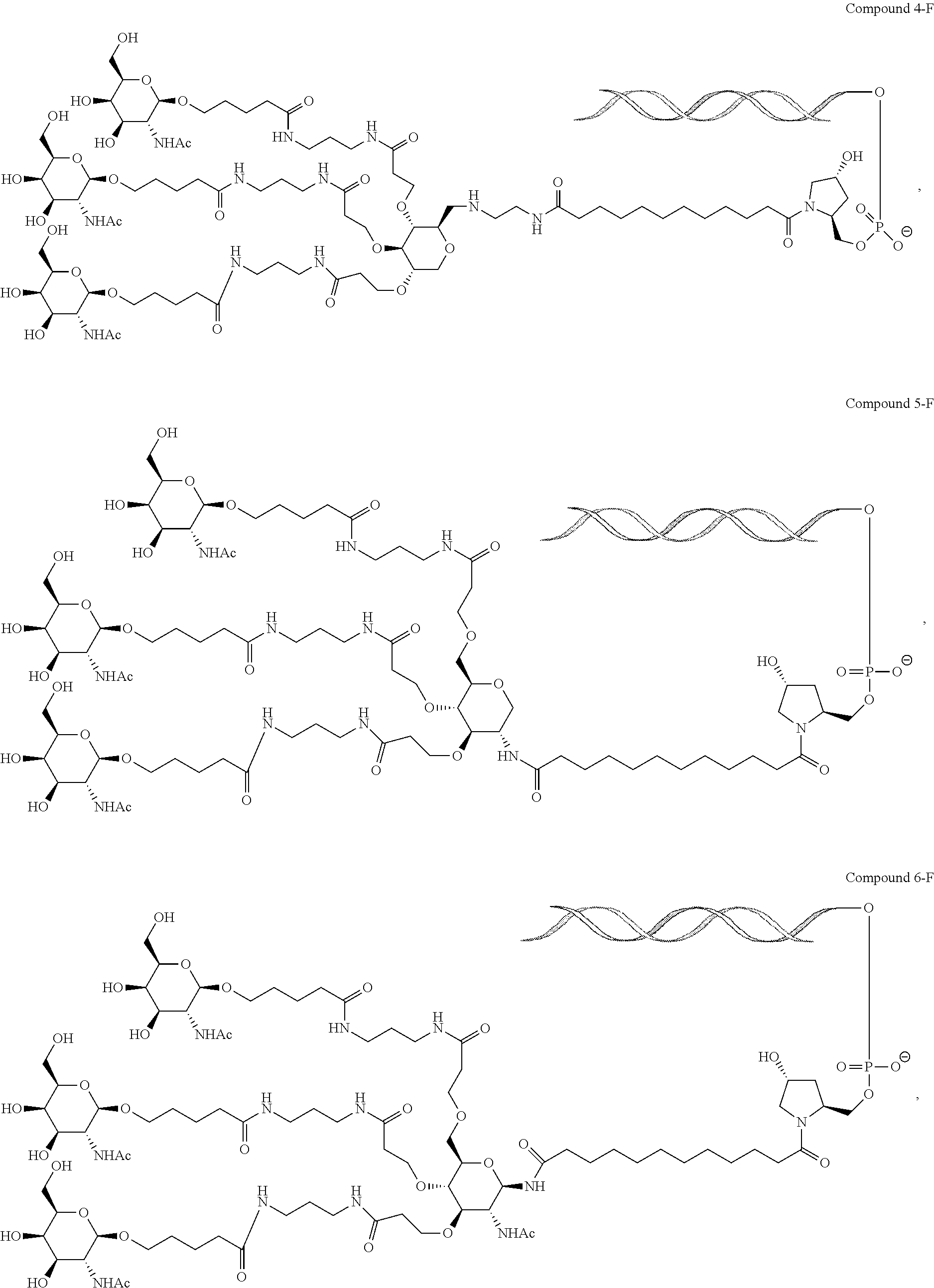

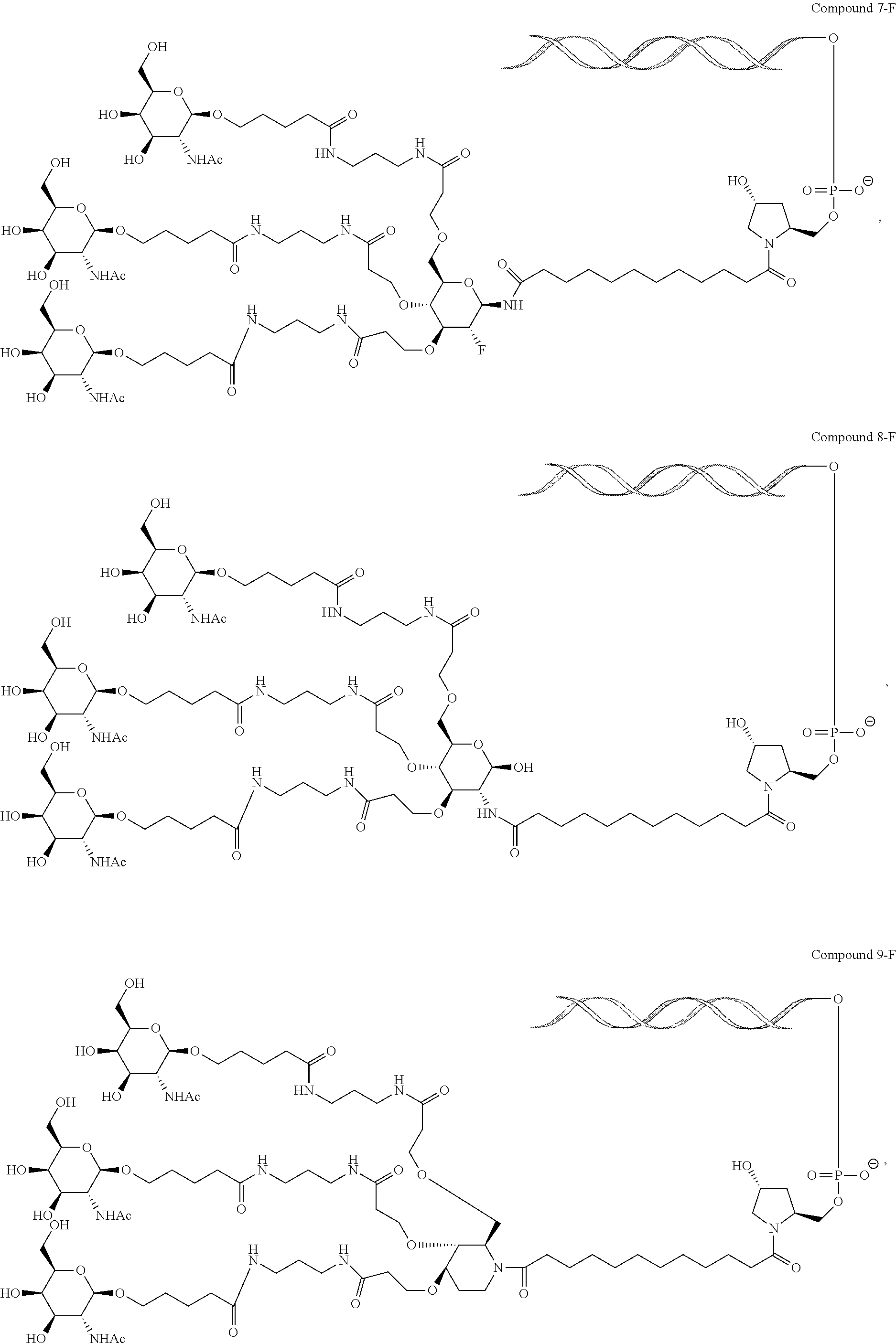

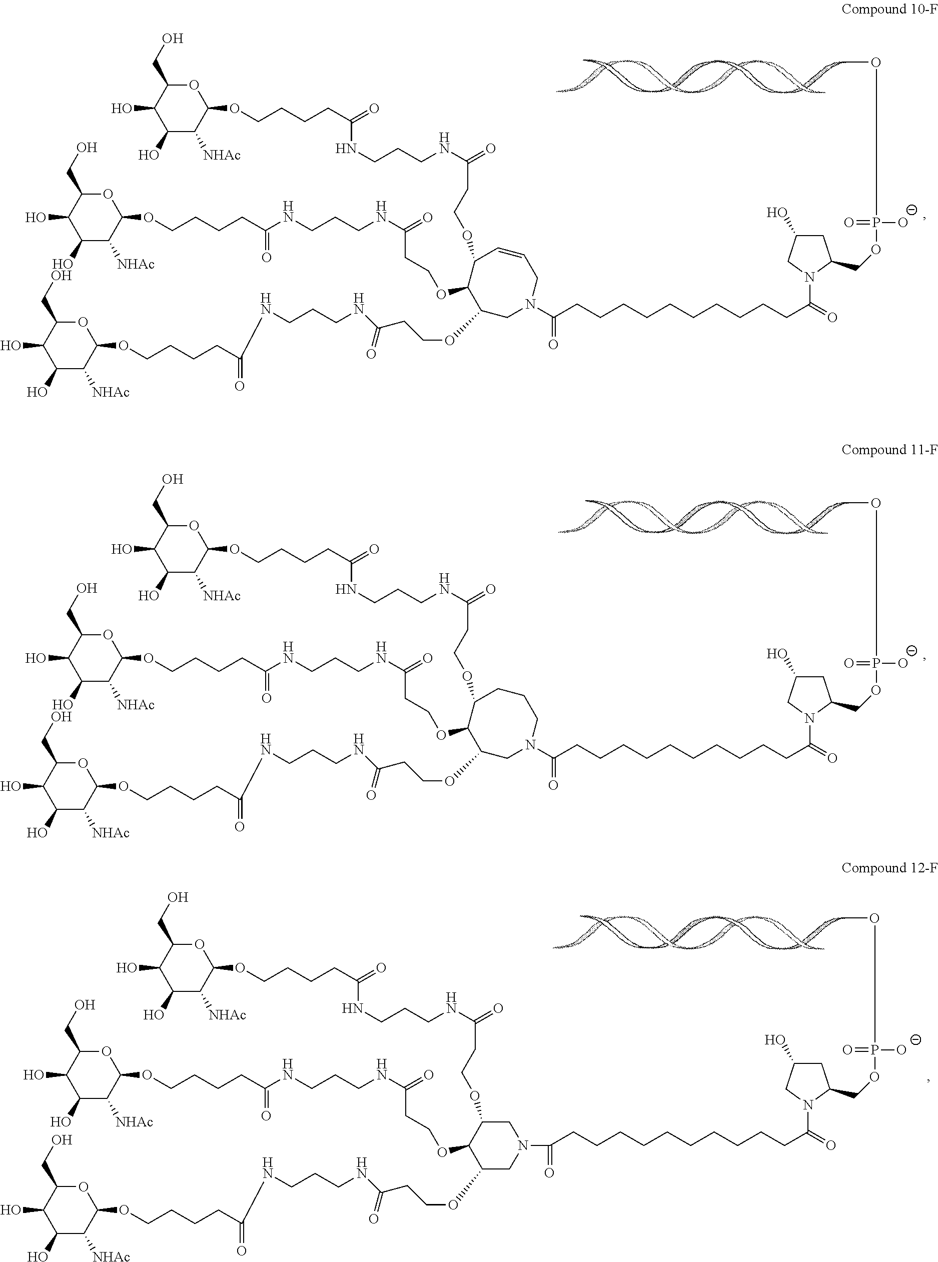

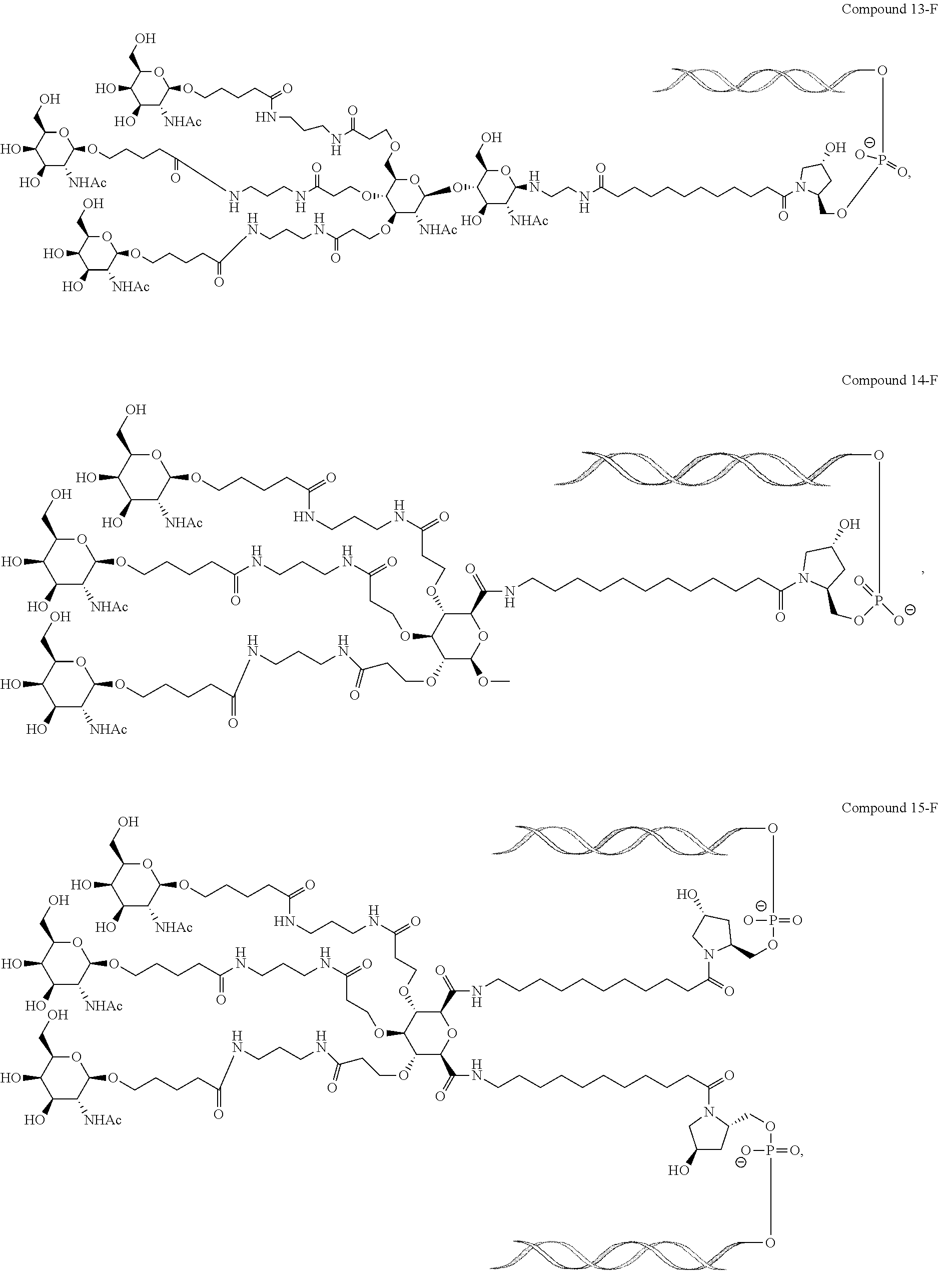

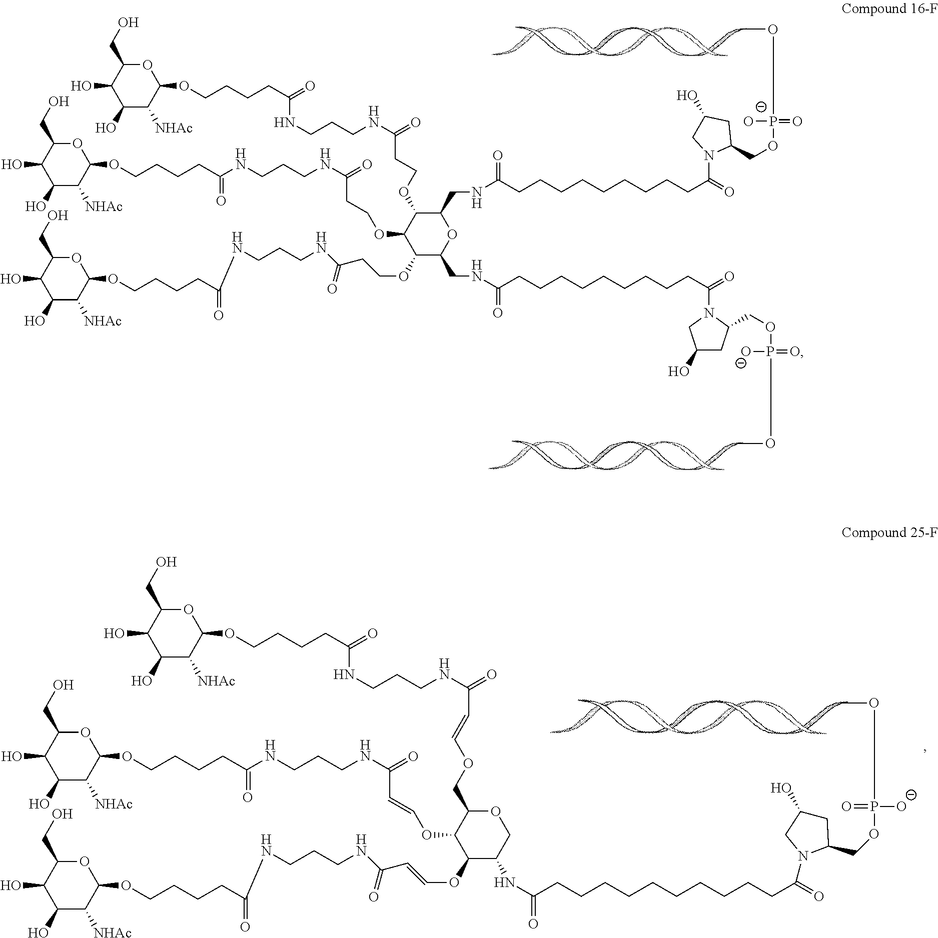

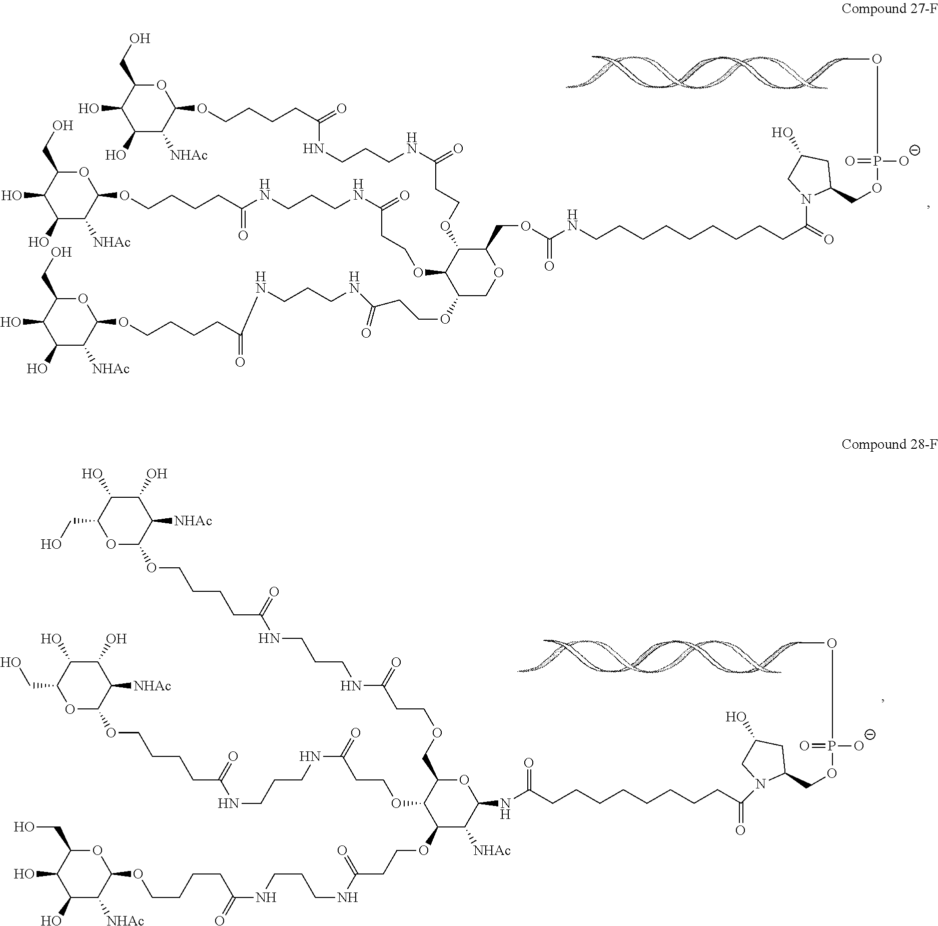

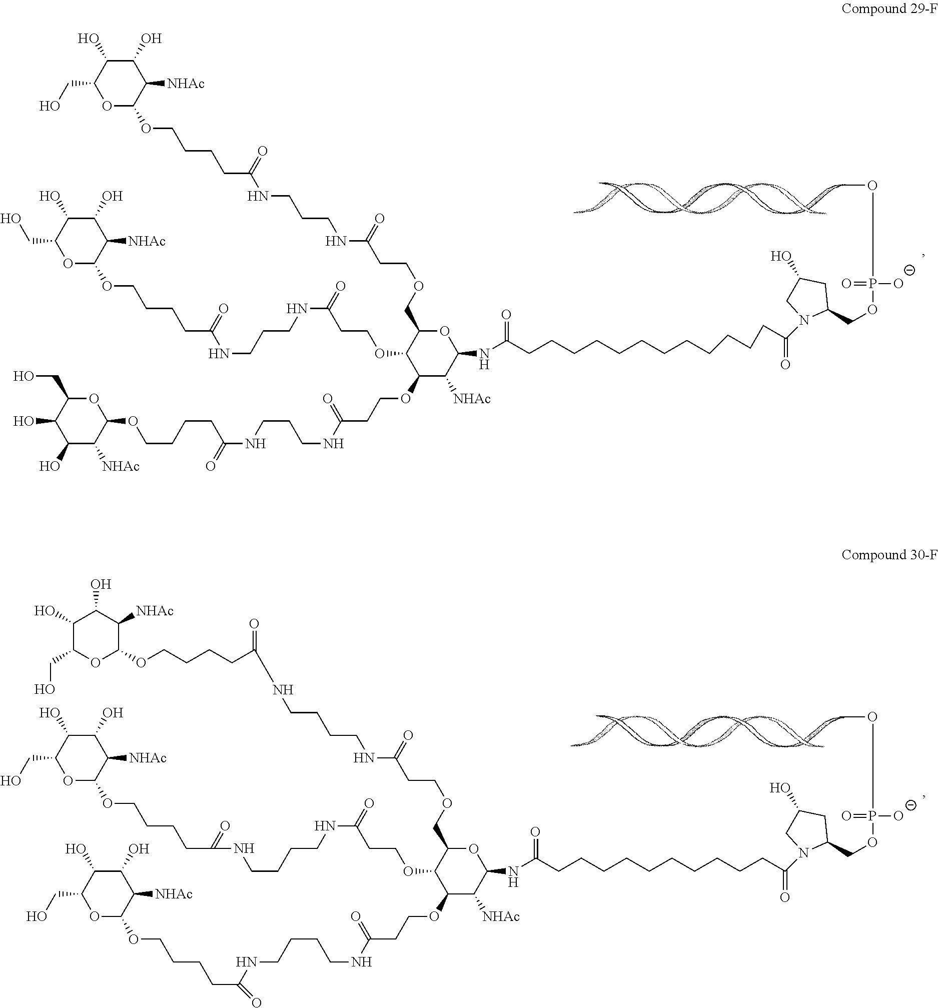

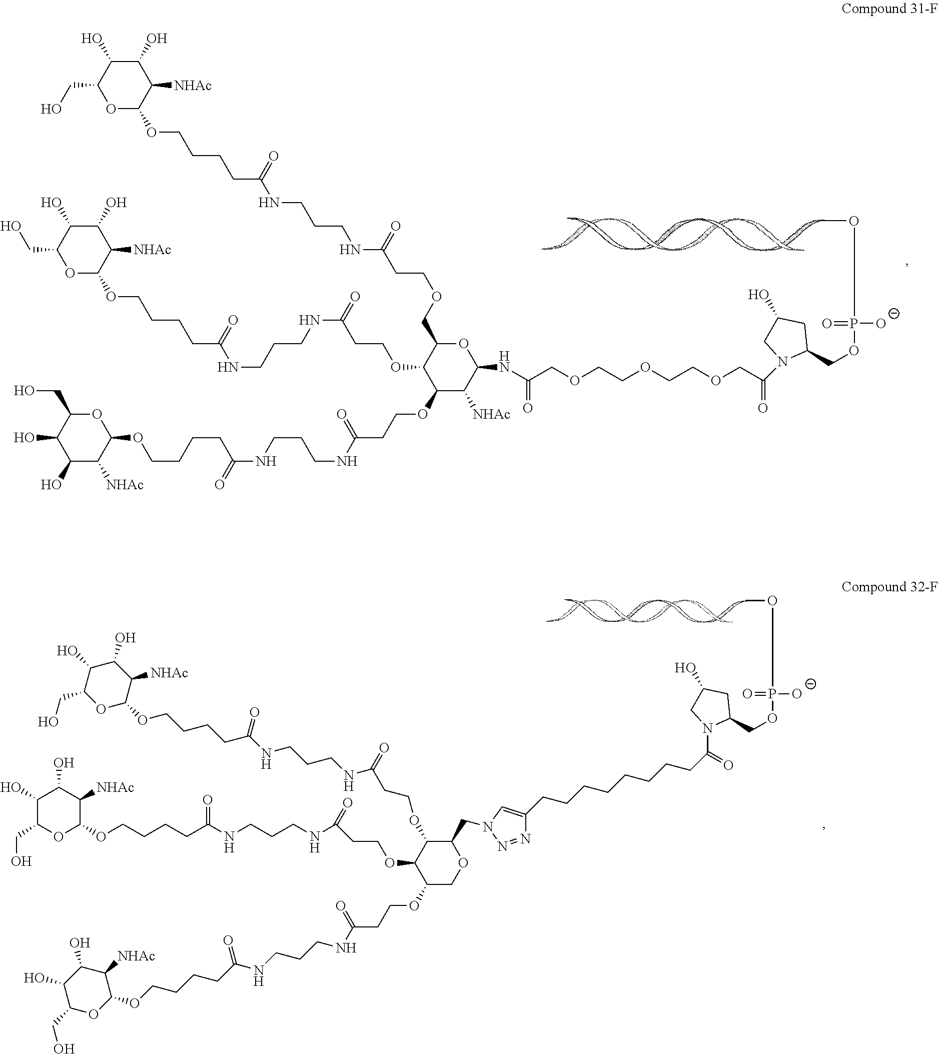

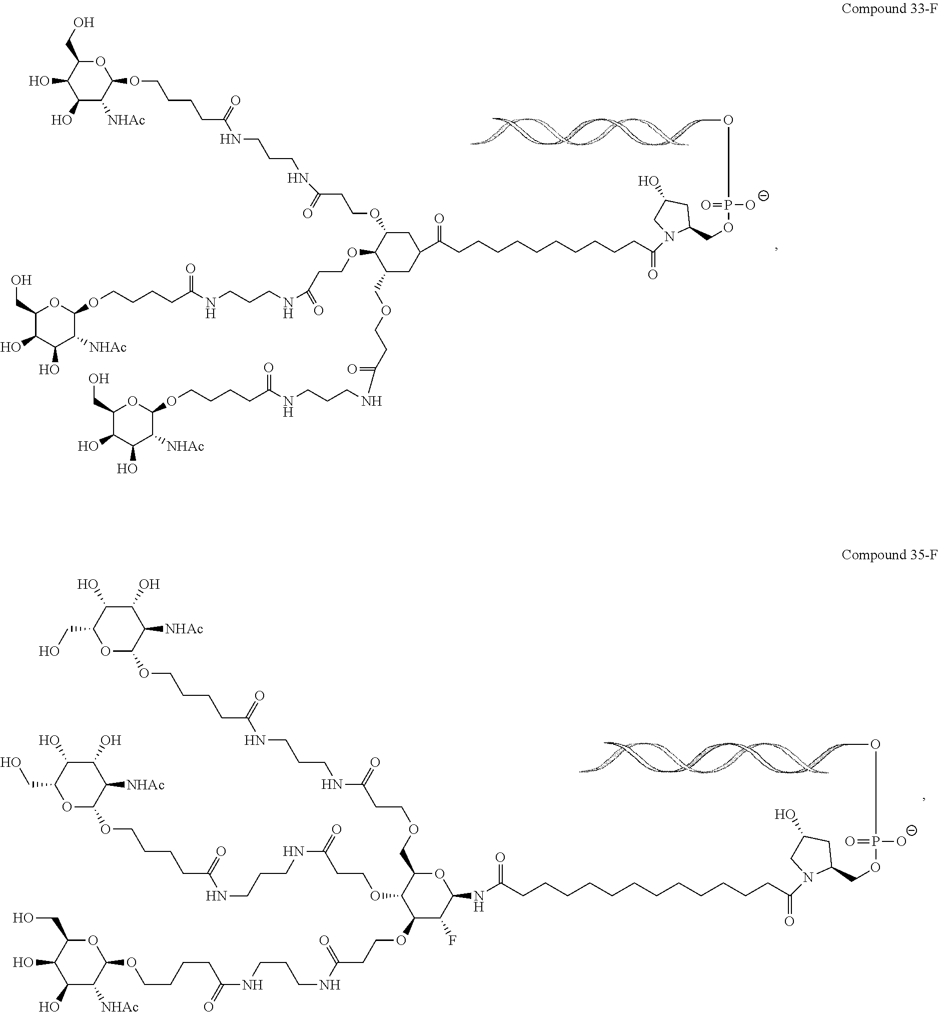

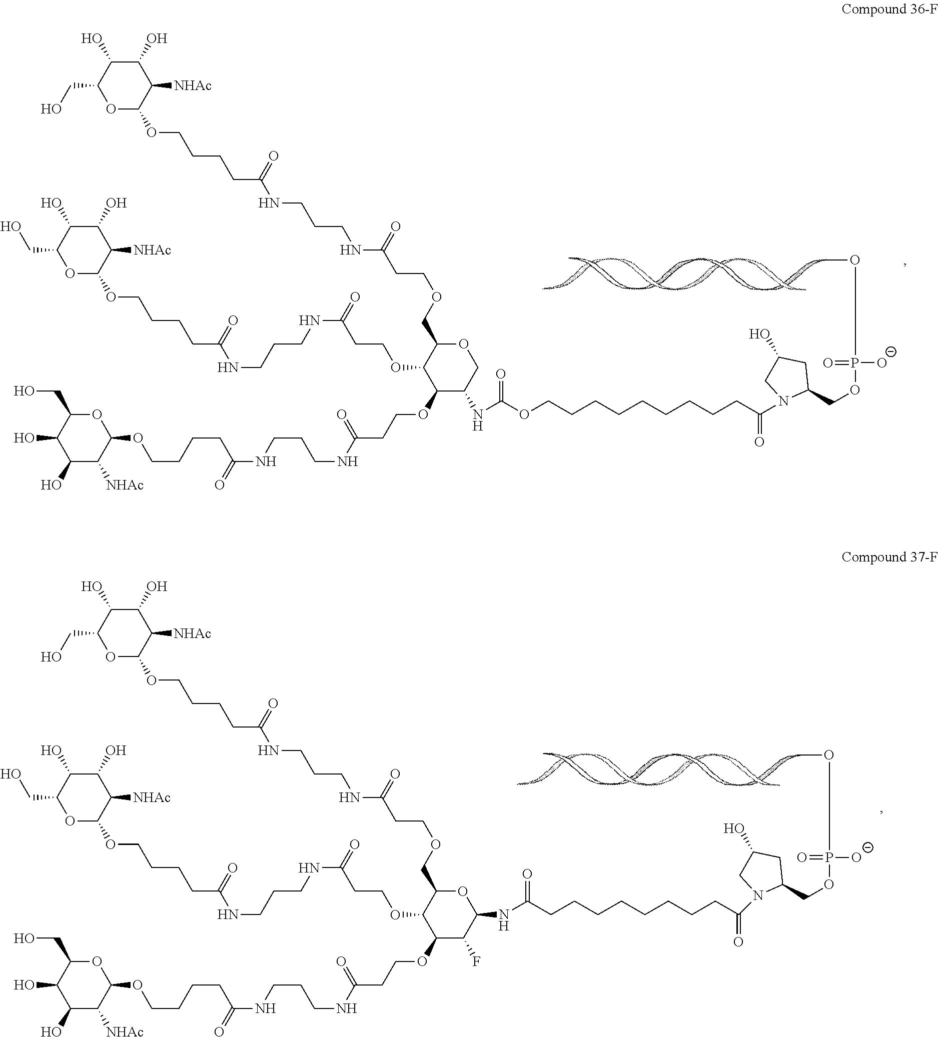

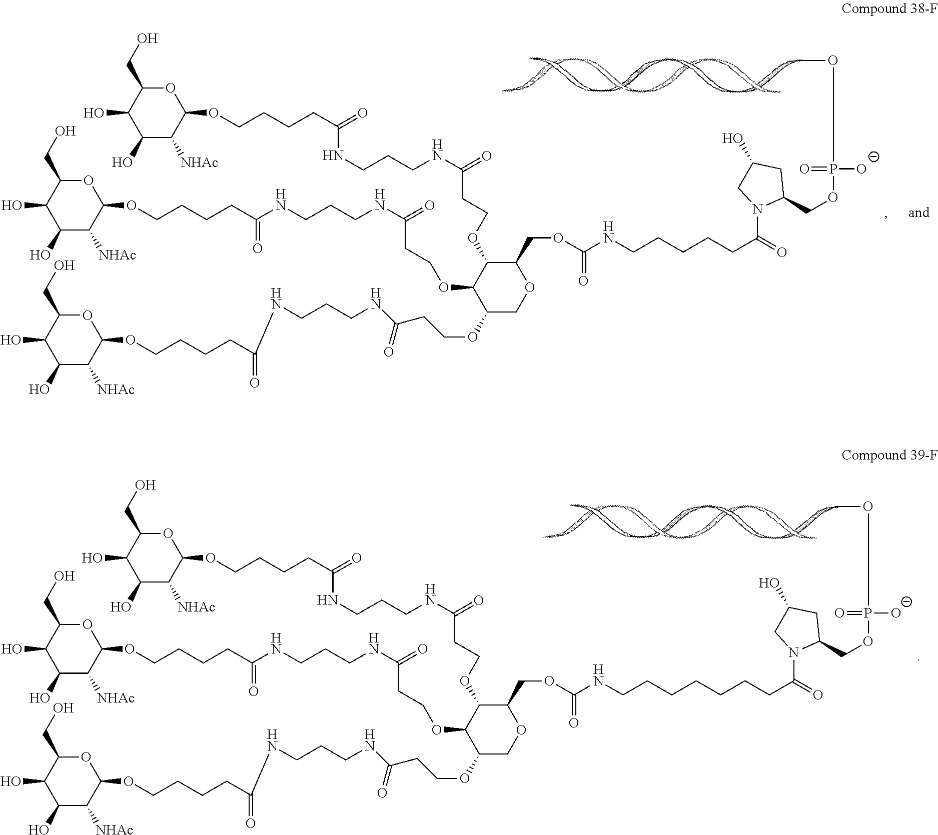

wherein [symbol] is an oligonucleotide (e.g., iRNA agents),
or a pharmaceutically acceptable salt or solvate thereof.

In one aspect, provided herein is a method of modulating the expression of a target gene in a cell, comprising delivering to said cell a compound of the invention or a pharmaceutically acceptable salt or solvate thereof.

In certain embodiments, the target gene is relevant to a liver disease.

In certain embodiments, the liver disease is selected from the group consisting of nonalcoholic fatty liver disease (NAFLD), nonalcoholic steatohepatitis (NASH), HBV infection, liver fibrosis, and liver cirrhosis.

In one aspect, provided herein is a pharmaceutical composition comprising a compound of the invention or a pharmaceutically acceptable salt or solvate thereof alone or in combination with a pharmaceutically acceptable carrier or excipient.

### BRIEF DESCRIPTION OF THE DRAWINGS

The summary, as well as the following detailed description, is further understood when read in conjunction with the appended drawings. However, the invention is not limited to the specific disclosure of the drawings. In the drawings:

FIG. 1 shows the activity of certain illustrated GalNAc-siRNA conjugates as provided herein.

### DETAILED DESCRIPTION OF THE INVENTION

Reference will now be made in detail to certain embodiments, examples of which are illustrated in the accompanying detailed description. While enumerated embodiments will be described, it should be understood that they are not intended to limit the invention to those embodiments. On the contrary, the invention is intended to cover all alternatives, modifications and equivalents which may be included within the scope of the present invention as defined by the claims.

### Definitions

The terms used herein have their ordinary meaning and the meaning of such terms is independent at each occurrence thereof. Nevertheless, unless otherwise stated, the following definitions apply throughout the specification and claims.

As used herein, the singular forms “a”, “an” and “the” include plural referents unless expressly stated to the contrary.

As used herein, the terms “comprise” and “include” are intended to specify the presence of stated features, integers, components, or steps, but they do not preclude the presence or addition of one or more other features, integers, components, steps, or groups thereof.

As used herein, the term “about” means approximately, in the region of, roughly, or around. When the term “about” is used in conjunction with a numerical range, it modifies that range by extending the boundaries above and below the numerical values set forth. In general, the term “about” is used herein to modify a numerical value above and below the given value by a variance of 20%, typically 10%, more typically 5%, and even more typically 1%. Sometimes, such a range can lie within the experimental error, type of standard methods used for the measurement and/or determination of a given value or range.

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modem Methods of Organic Synthesis, 3^{rd} Edition, Cambridge University Press, Cambridge, 1987.

All ranges cited herein are inclusive, unless expressly stated to the contrary.

When a range of values is listed, it is intended to encompass each value and sub-range within the range. For example, “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

When any variable occurs more than one time in any constituent or in Formula I or in any other formula depicting and describing compounds of the present invention, its definition at each occurrence is independent of its definition at every other occurrence. Also, combinations of substituents and/or variables are permissible only if such combinations result in stable compounds.

As used herein, the term “alkyl”, whether as part of another term or used independently, refers to an acyclic straight or branched chain saturated hydrocarbon group, which may be optionally substituted (i.e., unsubstituted or substituted) independently with one or more substituents described below. The term “Cᵢ₋ⱼ alkyl” refers to an alkyl having i to j carbon atoms. In certain embodiments, alkyl groups contain 1 to 12 carbon atoms. In certain embodiments, alkyl groups contain 1 to 11 carbon atoms. In certain embodiments, alkyl groups contain 1 to 11 carbon atoms, 1 to 10 carbon atoms, 1 to 9 carbon atoms, 1 to 8 carbon atoms, 1 to 7 carbon atoms, 1 to 6 carbon atoms, 1 to 5 carbon atoms, 1 to 4 carbon atoms, 1 to 3 carbon atoms, or 1 to 2 carbon atoms. Non-limiting examples of alkyl groups include methyl; ethyl; n- and iso-propyl; n-, sec-, iso- and tert-butyl; neopentyl, and the like. Alkyl groups may be optionally substituted, as valency permits, with one, two, three, or, in the case of alkyl groups of two carbons or more, four or more substituents independently selected from the group consisting of: alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thiol; silyl; cyano; ═O; ═S; and ═NR′, where R′ is H, alkyl, aryl, or heterocyclyl. In certain embodiments, alkyl groups may be optionally substituted with halo, amino, hydroxy, methoxy, nitro, cyano, etc. Each of the substituents may itself be unsubstituted or, as valency permits, substituted with unsubstituted substituent(s) defined herein for each respective group.

As used herein, the term “alkylene”, whether as part of another term or used independently, refers to a divalent substituent that is a monovalent alkyl having one hydrogen atom replaced with a valency. Alkylene groups may be unsubstituted or substituted. An optionally substituted alkylene is an alkylene that is optionally substituted as described herein for alkyl.

As used herein, the term “alkenyl”, whether as part of another term or used independently, refers to linear or branched-chain hydrocarbon radical having at least one carbon-carbon double bond, which may be optionally substituted (i.e., unsubstituted or substituted) independently with one or more substituents described herein, and includes radicals having “cis” and “trans” orientations, or alternatively, “E” and “Z” orientations. In certain embodiments, alkenyl groups contain 2 to 12 carbon atoms. In certain embodiments, alkenyl groups contain 2 to 11 carbon atoms. In certain embodiments, alkenyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms. In certain embodiments, alkenyl groups contain 2 carbon atoms. Non-limiting examples of alkenyl groups include ethylenyl (or vinyl), propenyl, butenyl, pentenyl, 1-methyl-2-buten-1-yl, 5-hexenyl, and the like. An optionally substituted alkenyl is an alkenyl that is optionally substituted as described herein for alkyl.

As used herein, the term “alkenylene”, whether as part of another term or used independently, a divalent substituent that is a monovalent alkenyl having one hydrogen atom replaced with a valency. Alkenylene groups may be unsubstituted or substituted. An optionally substituted alkenylene is an alkenylene that is optionally substituted as described herein for alkyl.

As used herein, the term “alkynyl”, whether as part of another term or used independently, refers to a linear or branched hydrocarbon radical having at least one carbon-carbon triple bond, which may be optionally substituted (i.e., unsubstituted or substituted) independently with one or more substituents described herein. In certain embodiments, alkynyl groups contain 2 to 12 carbon atoms. In certain embodiments, alkynyl groups contain 2 to 11 carbon atoms. In certain embodiments, alkynyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms. In certain embodiments, alkynyl groups contain 2 carbon atoms. Non-limiting examples of alkynyl group include ethynyl, 1-propynyl, 2-propynyl, and the like. An optionally substituted alkynyl is an alkynyl that is optionally substituted as described herein for alkyl.

As used herein, the term “alkynylene”, whether as part of another term or used independently, refers to a divalent substituent that is a monovalent alkynyl having one hydrogen atom replaced with a valency. Alkynylene groups may be unsubstituted or substituted. An optionally substituted alkynylene is an alkynylene that is optionally substituted as described herein for alkyl.

As used herein, the term “cycloalkyl”, whether as part of another term or used independently, refers to a monovalent non-aromatic, saturated or partially unsaturated monocyclic and polycyclic ring system, in which all the ring atoms are carbon and which contains at least three ring forming carbon atoms. In certain embodiments, the cycloalkyl may contain 3 to 12 ring forming carbon atoms, 3 to 10 ring forming carbon atoms, 3 to 9 ring forming carbon atoms, 3 to 8 ring forming carbon atoms, 3 to 7 ring forming carbon atoms, 3 to 6 ring forming carbon atoms, 3 to 5 ring forming carbon atoms, 4 to 12 ring forming carbon atoms, 4 to 10 ring forming carbon atoms, 4 to 9 ring forming carbon atoms, 4 to 8 ring forming carbon atoms, 4 to 7 ring forming carbon atoms, 4 to 6 ring forming carbon atoms, 4 to 5 ring forming carbon atoms. Particularly, cycloalkyl groups may 3 to 10 ring forming carbon atoms (i.e., a C₃₋₁₀ cycloalkyl). Particularly, cycloalkyl groups may be monocyclic or bicyclic. Bicyclic cycloalkyl groups may be of bicyclo[p.q.0]alkyl type, in which each of p and q is, independently, 1, 2, 3, 4, 5, 6, or 7, provided that the sum of p and q is 2, 3, 4, 5, 6, 7, or 8.

Alternatively, bicyclic cycloalkyl groups may include bridged cycloalkyl structures, e.g., bicyclo[p.q.r]alkyl, in which r is 1, 2, or 3, each of p and q is, independently, 1, 2, 3, 4, 5, or 6, provided that the sum of p, q, and r is 3, 4, 5, 6, 7, or 8. The cycloalkyl group may be a spirocyclic group, e.g., spiro[p.q]alkyl, in which each of p and q is, independently, 2, 3, 4, 5, 6, or 7, provided that the sum of p and q is 4, 5, 6, 7, 8, or 9. Non-limiting examples of cycloalkyl include cyclopropyl, cyclobutyl, cyclopentyl, cyclohexyl, cycloheptyl, 1-bicyclo[2.2.1.]heptyl, 2-bicyclo[2.2.1.]heptyl, 5-bicyclo[2.2.1.]heptyl, 7-bicyclo[2.2.1.]heptyl, and decalinyl. The cycloalkyl group may be optionally substituted (i.e., unsubstituted or substituted) with one, two, three, four, or five substituents independently selected from the group consisting of: alkyl; alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thiol; silyl; cyano; ═O; ═S; ═NR′, where R′ is H, alkyl, aryl, or heterocyclyl. Each of the substituents may itself be unsubstituted or substituted with unsubstituted substituent(s) defined herein for each respective group.

As used herein, the term “cycloalkylene”, whether as part of another term or used independently, refers to a divalent substituent that is a cycloalkyl having one hydrogen atom replaced with a valency. Cycloalkylene groups may be unsubstituted or substituted. An optionally substituted cycloalkylene is a cycloalkylene that is optionally substituted as described herein for cycloalkyl.

As used herein, the term “cycloalkoxy”, whether as part of another term or used independently, refers to a group —OR, where R is cycloalkyl. Cycloalkoxy groups may be unsubstituted or substituted. An optionally substituted cycloalkoxy is cycloalkoxy that is optionally substituted as described herein for cycloalkyl.

As used herein, the term “aryl”, whether as part of another term or used independently, refers to a mono-, bicyclic, or multicyclic carbocyclic ring system having at least one aromatic rings. Aryl groups may be 6- to 12-membered, for example, 8- to 12-membered, 6- to 10-membered, 6-membered. All atoms within an unsubstituted carbocyclic aryl group are carbon atoms. Non-limiting examples of carbocyclic aryl groups include phenyl, naphthyl, 1,2-dihydronaphthyl, 1,2,3,4-tetrahydronaphthyl, fluorenyl, indanyl, indenyl, etc. The aryl group may be optionally substituted (i.e., unsubstituted or substituted) with one, two, three, four, or five substituents independently selected from the group consisting of: alkyl; alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thiol; silyl; and cyano. Each of the substituents may itself be unsubstituted or substituted with unsubstituted substituent(s) defined herein for each respective group.

As used herein, the term “arylene”, whether as part of another term or used independently, refers to a divalent substituent that is an aryl having one hydrogen atom replaced with a valency. Arylene groups may be unsubstituted or substituted. An optionally substituted arylene is an arylene that is optionally substituted as described herein for aryl.

As used herein, the term “acyl”, whether as part of another term or used independently, refers to a chemical substituent of formula —C(O)—R, where R is alkyl, aryl, arylalkyl, cycloalkyl, heterocyclyl, heterocyclylalkyl, heteroaryl, or heteroarylalkyl. An optionally substituted acyl is an acyl that is optionally substituted as described herein for each group R.

As used herein, the term “acyloxy”, whether as part of another term or used independently, refers to a chemical substituent of formula —OR, where R is acyl. An optionally substituted acyloxy is an acyloxy that is optionally substituted as described herein for acyl.

As used herein, the term “alkoxy”, whether as part of another term or used independently, refers to a chemical substituent of formula —OR, where R is an alkyl group, particularly C₁₋₁₂ alkyl, C₁₋₁₀ alkyl, C₁₋₆ alkyl, etc. Alkoxy groups may be unsubstituted or substituted. An optionally substituted alkoxy is an alkoxy group that is optionally substituted as defined herein for alkyl.

As used herein, the term “heteroalkyl”, whether as part of another term or used independently, refers to an alkyl group (e.g., an alkyl group defined herein) interrupted one or more times by one or two heteroatoms each time. Each heteroatom is independently O, N, or S.

None of the heteroalkyl groups includes two contiguous oxygen atoms. The heteroalkyl group may be unsubstituted or substituted (e g., optionally substituted heteroalkyl). When heteroalkyl is substituted and the substituent is bonded to the heteroatom, the substituent is selected according to the nature and valency of the heteroatom. Thus, the substituent bonded to the heteroatom, valency permitting, is selected from the group consisting of ═O, —N(R^{N2})₂, —SO₂OR^{N3}, —SO₂R^{N2}, —SOR^{N3}, —COOR^{N3}, an N protecting group, alkyl, aryl, cycloalkyl, heterocyclyl, or cyano, where each R^{N2} is independently H, alkyl, cycloalkyl, aryl, or heterocyclyl, and each R^{N3} is independently alkyl, cycloalkyl, aryl, or heterocyclyl. Each of these substituents may itself be unsubstituted or substituted with unsubstituted substituent(s) defined herein for each respective group. When heteroalkyl is substituted and the substituent is bonded to carbon, the substituent is selected from those described for alkyl, provided that the substituent on the carbon atom bonded to the heteroatom is not Cl, Br, or L In certain embodiments, carbon atoms are found at the termini of a heteroalkyl group. In certain embodiments, heteroalkyl is PEG.

As used herein, the term “heteroalkylene”, whether as part of another term or used independently, refers to a divalent substituent that is a heteroalkyl having one hydrogen atom replaced with a valency. Heteroalkylene groups may be unsubstituted or substituted. An optionally substituted heteroalkylene is a heteroalkylene that is optionally substituted as described herein for heteroalkyl.

As used herein, the term “heteroaryl”, whether as part of another term or used independently, refers to a monocyclic ring system, or a fused or bridged bicyclic, tricyclic, or tetracyclic ring system; the ring system contains one, two, three, or four heteroatoms independently selected from the group consisting of nitrogen, oxygen, and sulfur; and at least one of the rings is an aromatic ring. Heteroaryl groups may be 5- to 12-membered, for example, 8- to 12-membered, 5- to 10-membered, 5- to 6-membered. Heteroaryl groups have a carbon count of 1 to 16 carbon atoms unless otherwise specified. Certain heteroaryl groups may have a carbon count up to 9 carbon atoms. Non-limiting examples of heteroaryl groups include benzimidazolyl, benzofuryl, benzothiazolyl, benzothienyl, benzoxazolyl, furyl, imidazolyl, indolyl, isoindazolyl, isoquinolinyl, isothiazolyl, isothiazolyl, isoxazolyl, oxadiazolyl, oxazolyl, purinyl, pyrrolyl, pyridinyl, pyrazinyl, pyrimidinyl, qunazolinyl, quinolinyl, thiadiazolyl (e.g., 1,3,4-thiadiazole), thiazolyl, thienyl, triazolyl (e.g., 1H-1,2,3-triazolyl), tetrazolyl, dihydroindolyl, tetrahydroquinolyl, tetrahydroisoquinolyl, etc. The term bicyclic, tricyclic, and tetracyclic heteroaryl groups include at least one ring having at least one heteroatom as described above and at least one aromatic ring. For example, a ring having at least one heteroatom may be fused to one, two, or three carbocyclic rings, e.g., an aryl ring, a cyclohexane ring, a cyclohexene ring, a cyclopentane ring, a cyclopentene ring, or another monocyclic heterocyclic ring. Non-limiting examples of fused heteroaryl groups include 1,2,3,5,8,8a-hexahydroindolizine; 2,3-dihydrobenzofuran; 2,3-dihydroindole; and 2,3-dihydrobenzothiophene. Heteroaryl groups may be optionally substituted with one, two, three, four, or five substituents independently selected from the group consisting of: alkyl; alkoxy; acyloxy; aryloxy; amino; arylalkoxy; cycloalkyl; cycloalkoxy; halogen; heterocyclyl; heterocyclylalkyl; heteroaryl; heteroarylalkyl; lheterocyclyloxy; heteroaryloxy; hydroxyl; nitro; thiol; cyano; ═O; —NR₂, where each R is independently hydrogen, alkyl, acyl, aryl, arylalkyl, cycloalkyl, heterocyclyl, or heteroaryl; —COOR^{A}, where R^{A} is hydrogen, alkyl, aryl, arylalkyl, cycloalkyl, heterocyclyl, or heteroaryl; and —CON(R^{B})₂, where each R^{B} is independently hydrogen, alkyl, aryl, arylalkyl, cycloalkyl, heterocyclyl, or heteroaryl. Each of the substituents may itself be unsubstituted or substituted with unsubstituted substituent(s) defined herein for each respective group.

As used herein, the term “heteroarylene”, whether as part of another term or used independently, refers to a divalent substituent that is a heteroaryl having one hydrogen atom replaced with a valency. Heteroarylene groups may be substituted or unsubstituted. An optionally substituted heteroarylene is a heteroarylene that is optionally substituted as described herein for heteroaryl.

As used herein, the term “heteroaryloxy”, whether as part of another term or used independently, refers to a structure —OR, in which R is heteroaryl. Heteroarylene groups may be substituted or unsubstituted. An optionally substituted heteroaryloxy can be a heteroaryloxy optionally substituted as defined for heteroaryl.

As used herein, the term “heterocyclyl”, whether as part of another term or used independently, refers to a monocyclic, bicyclic, tricyclic, or tetracyclic ring system having fused or bridged 4-, 5-, 6-, 7-, or 8-membered rings, unless otherwise specified, the ring system containing one, two, three, or four heteroatoms independently selected from the group consisting of nitrogen, oxygen, and sulfur. Heterocyclyl groups may be 3- to 12-membered, for example, 4- to 12-membered, 4- to 10-membered, 5- to 12-membered, 5- to 10-membered, 5- to 8-membered. Heterocyclyl may be aromatic or non-aromatic. An aromatic heterocyclyl is heteroaryl as described herein. Non-aromatic 5-membered heterocyclyl has zero or one double bonds, non-aromatic 6- and 7-membered heterocyclyl groups have zero to two double bonds, and non-aromatic 8-membered heterocyclyl groups have zero to two double bonds and/or zero or one carbon-carbon triple bond. Heterocyclyl groups have a carbon count of 1 to 16 carbon atoms unless otherwise specified. Certain heterocyclyl groups may have a carbon count up to 9 carbon atoms. Non-aromatic heterocyclyl groups include pyrrolinyl, pyrrolidinyl, pyrazolinyl, pyrazolidinyl, imidazolinyl, imidazolidinyl, piperidinyl, homopiperidinyl, piperazinyl, pyridazinyl, oxazolidinyl, morpholinyl, thiomorpholinyl, thiazolidinyl, isothiazolidinyl, thiazolidinyl, tetrahydrofuranyl, dihydrofuranyl, tetrahydrothienyl, dihydrothienyl, pyranyl, dihydropyranyl, dithiazolyl, etc. The term “heterocyclyl” also represents a heterocyclic compound having a bridged multicyclic structure in which one or more carbons and/or heteroatoms bridges two non-adjacent members of a monocyclic ring, e.g., quinuclidine, tropanes, or diaza-bicyclo[2.2.2]octane. The term “heterocyclyl” includes bicyclic, tricyclic, and tetracyclic groups in which any of the above heterocyclic rings is fused to one, two, or three carbocyclic rings, e.g., a cyclohexane ring, a cyclohexene ring, a cyclopentane ring, a cyclopentene ring, or another heterocyclic ring. Non-limiting examples of fused heterocyclyls include 1,2,3,5,8,8a-hexahydroindolizine; 2,3-dihydrobenzofuran; 2,3-dihydroindole; and 2,3-dihydrobenzothiophene. The heterocyclyl group may be unsubstituted or substituted with one, two, three, four or five substituents independently selected from the group consisting of: alkyl; alkoxy; acyloxy; aryloxy; amino; arylalkoxy; cycloalkyl; cycloalkoxy; halogen; heterocyclyl; heterocyclyl alkyl; heteroaryl; heteroaryl alkyl; heterocyclyloxy; heteroaryloxy; hydroxyl; nitro; thiol; cyano; ═O; ═S; —NR₂, where each R is independently hydrogen, alkyl, acyl, aryl, arylalkyl, cycloalkyl, heterocyclyl, or heteroaryl; —COOR^{A}, where R^{A} is hydrogen, alkyl, aryl, arylalkyl, cycloalkyl, heterocyclyl, or heteroaryl; and —CON(R^{B})₂, where each R^{B} is independently hydrogen, alkyl, aryl, arylalkyl, cycloalkyl, heterocyclyl, or heteroaryl.

As used herein, the term “heterocyclylalkyl”, whether as part of another term or used independently, refers to an alkyl group substituted with a heterocyclyl group. Heterocyclylalkyl groups may be unsubstituted or substituted. The heterocyclyl and alkyl portions of an optionally substituted heterocyclylalkyl are optionally substituted as described for heterocyclyl and alkyl, respectively.

As used herein, the term “heterocyclylene”, whether as part of another term or used independently, refers to a divalent substituent that is a heterocyclyl having one hydrogen atom replaced with a valency. Heterocyclylene groups may be unsubstituted or substituted. An optionally substituted heterocyclylene is a heterocyclylene that is optionally substituted as described herein for heterocyclyl.

As used herein, the term “thioheterocyclylene” refers to a divalent group —S—R′—, where R′ is a heterocyclylene as defined herein.

As used herein, the term “thiol” refers to an —SH group.

As used herein, the term “triazolocycloalkenylene” refers to the cycloalkenylene containing a 1,2,3-triazole ring fused to an 8-membered ring, all of the endocyclic atoms of which are carbon atoms, and bridgehead atoms are sp2-hybridized carbon atoms. Triazolocycloalkenylene groups may be unsubstituted or substituted. An optionally substituted triazolocycloalkenylene is a triazolocycloalkenylene that optionally substituted in a manner described for cycloalkenyl.

As used herein, the term “triazoloheterocyclylene” refers to the heterocyclylene containing a 1,2,3-triazole ring fused to an 8-membered ring containing at least one heteroatom. The bridgehead atoms in triazoloheterocyclylene are carbon atoms. Triazoloheterocyclylene groups may be unsubstituted or substituted. An optionally substituted triazoloheterocyclylene is a triazoloheterocyclylene optionally substituted in a manner described for heterocyclyl.

As used herein, the term “oxo” refers to a divalent oxygen atom, e.g., the structure of oxo may be shown as ═O.

As used herein, the term “halogen” or “halo” refers to fluoride, chloride, bromide and iodide, particularly fluoride and chloride, and more particularly fluoride.

As used herein, the term “substituted”, when refers to a chemical group, means the chemical group has one or more hydrogen atoms that is/are removed and replaced by substituents. The term “substituent”, as used herein, has the ordinary meaning known in the art and refers to a chemical moiety that is covalently attached to, or if appropriate, fused to, a parent group. It is to be understood that substitution at a given atom is limited by valency. Examples of substituents include, but not limited to, halo, alkyl, alkenyl, alkynyl, aryl, heterocyclyl, thiol, alkylthio, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic group, and aliphatic group. It is understood that the substituent can be further substituted.

When a moiety is noted as being “optionally substituted” in Formula (I) or any embodiment thereof, it means that Formula (I) or the embodiment thereof encompasses both compounds that are substituted with the noted substituent (or substituents) on the moiety and compounds that do not contain the noted substituent (or substituents) on the moiety (i.e., wherein the moiety is unsubstituted).

As used herein, the term “protecting group” is well known in the art and include those described in detail in Greene's Protective Groups in Organic Synthesis, P. G. M. Wuts and T. W. Greene, 4^{th} Edition, Wiley-Inter science, 2006, the entirety of which is incorporated herein by reference.

In certain embodiments, the substituent present on an oxygen atom is an oxygen protecting group, also referred to herein as an “hydroxyl protecting group”, which refers to a labile chemical moiety which protects a hydroxyl group against undesired reactions during synthetic procedure(s). After the synthetic procedure(s), the hydroxy protecting group may be selectively removed. Suitable hydroxyl protecting groups are well known in the art and include those described in detail in Protecting Groups in Organic Synthesis, T. W. Greene and P. G. M. Wuts, 3^{rd} Edition, John Wiley & Sons, 1999, incorporated herein by reference in its entirety. Non-limiting examples of hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, tosylate (Ts), 4,4′-dimethoxytriphenylmethyl (DMTr), etc. In certain embodiments, non-limiting examples of hydroxyl protecting groups include acetyl, benzyl, benzoyl, trimethylsilyl, 4,4′-dimethoxytriphenylmethyl (DMTr), etc.

In certain embodiments, the substituent present on a sulfur atom is a sulfur protecting group, also referred to as a “thiol protecting group”, which refers to a labile chemical moiety which protects a thiol group against undesired reactions during synthetic procedure(s). After the synthetic procedure(s), the thiol protecting group may be selectively removed. Suitable sulfur protecting groups are well known in the art and include those described in detail in Protecting Groups in Organic Synthesis, T. W. Greene and P. G. M. Wuts, 3^{rd} Edition, John Wiley & Sons, 1999, incorporated herein by reference. Non-limiting examples of thiol protecting groups include p-methoxybenzyl (Mob), trityl (Trt), acetamidomethyl (Acm), etc.

In certain embodiments, the substituent present on the nitrogen atom is a nitrogen protecting group, also referred to as an “amino protecting group”, which refers to a labile chemical moiety which protects an amino group against undesired reactions during synthetic procedure(s). After the synthetic procedure(s), the amino protecting group may be selectively removed. Suitable amino protecting groups are well known in the art and include those described in detail in Protecting Groups in Organic Synthesis, T. W. Greene and P. G. M. Wuts, 3^{rd} Edition, John Wiley & Sons, 1999, incorporated herein by reference. Non-limiting examples of amino protecting groups may include acetyl, tert-butoxycarbonyl (BOC), trityl (Tr), benzyloxycarbonyl (Cbz), 9-fluorenylmethoxycarbonyl (FMOC), trimethylsilyl (TMS), tert-butyldimethylsilyl (TBS), etc.

In the foregoing definitions, hydroxyl, thiol, and amino protecting groups are not exhaustively defined. The function of such groups is to protect the reactive functional groups during the preparative steps and then to be removed at some later point in time without disrupting the remainder of the molecule. Many protecting groups are known in the art, and the use of other protecting groups not specifically referred to hereinabove are equally applicable.

As used herein, the term “carbohydrate” refers to compounds consisting of carbon (C), hydrogen (H) and oxygen (O) and having the general formula Cₓ(H₂O)_{y} where x and y may be the same or different. Carbohydrates may include monosaccharides, disaccharides, trisaccharides, oligosaccharides and polysaccharides. The term “monosaccharide” as used herein refers to a carbohydrate possessing a single carbon chain, which may be straight, branched or in cyclic form; “disaccharide” and “trisaccharide” refer to molecules containing two or three such monosaccharide units joined together by glycosidic bonds; “oligosaccharide” and “polysaccharide” refer to larger such aggregates, with about 4 to 9 monosaccharide units and even more monosaccharide units, respectively. A monosaccharide or a monosaccharide unit may be D- or L-configuration, and include 3 or more carbon atoms, particularly 4 or more carbon atoms, preferably 4 to 8 carbon atoms, e.g., 4 carbon atoms (terose), 5 carbon atoms (pentose), 6 carbon atoms (hexose), 7 carbon atoms (heptose), or 8 carbon atoms (octose). In certain embodiments, a monosaccharide or a monosaccharide unit may include 5 or 6 carbon atoms. In a disaccharide, trisaccharide, oligosaccharide and polysaccharide, each monosaccharide unit may be the same or different. Non-limiting examples of monosaccharides include, e.g., glucose, fructose, and galactose. Non-limiting examples of disaccharides include, e.g., gentiobiose, isomaltose, melibiose, trehalose, sucrose, lactose, maltose, and cellobiose. Non-limiting examples of trisaccharides include, e.g., lactosucrose, raffinose, etc. Non-limiting examples of polysaccharides include, e.g., starch, cellulose, glycogen, etc. The term “carbohydrate” may be used herein interchangeably with “sugar” and “saccharide”.

As used herein, the term “carbohydrate mimetic” refers to any carbohydrate derivative or other compound that has multiple hydroxy groups and thus looks somewhat like a sugar or saccharide with a certain modification in structure. For example, in connection with monosaccharide, the carbohydrate mimetic may be a deoxy sugar (alcoholic hydroxy group replaced by hydrogen), amino sugar (alcoholic hydroxy group replaced by amino group), a thio sugar (alcoholic hydroxy group replaced by thiol, or C═O replaced by C═S, or a ring oxygen of cyclic form replaced by sulfur), a seleno sugar, a telluro sugar, an aza sugar (ring carbon replaced by nitrogen), an imino sugar (ring oxygen replaced by nitrogen), a phosphano sugar (ring oxygen replaced with phosphorus), a phospha sugar (ring carbon replaced with phosphorus), a C-substituted monosaccharide (hydrogen at a non-terminal carbon atom replaced with carbon), an unsaturated monosaccharide, an alditol (carbonyl group replaced with CHOH group), aldonic acid (aldehydic group replaced by carboxy group), a carbasugar (ring oxygen replaced with carbon) a ketoaldonic acid, a uronic acid, an aldaric acid, a C-glycoside (anomeric oxygen replaced by carbon), carboxylated sugars, amidated sugars, fused cyclic sugars, and so forth. The mimetics may include one or more of such structural modifications. Particularly, non-limiting examples of monosaccharide mimetics may include one or more modifications on the carbohydrate structure selected from the group consisting of deoxysugars, aminosugars, N-glycosides, iminosugars, unsaturated sugars, carboxylated sugars, amidated sugars, fused cyclic sugars and carbasugars of a monosaccharide. It is to be understood that carbohydrates may be further substituted. Also included are amino sugars having a cyclized amino group, e.g., triazolyl. In certain embodiments, the monosaccharide mimetic may be 2-((1H-1,2,3-triazol-1-yl)methyl)tetrahydro-2H-pyran-3,4,5-triol which is optionally further substituted. In connection with a disaccharide, trisaccharide, oligosaccharide or polysaccharide, the carbohydrate mimetic refers to carbohydrates which have one or more monosaccharide units that are replaced by a mimetic of monosaccharide as described above.

In certain embodiments, non-limiting examples of carbohydrate mimetics may have the structure:

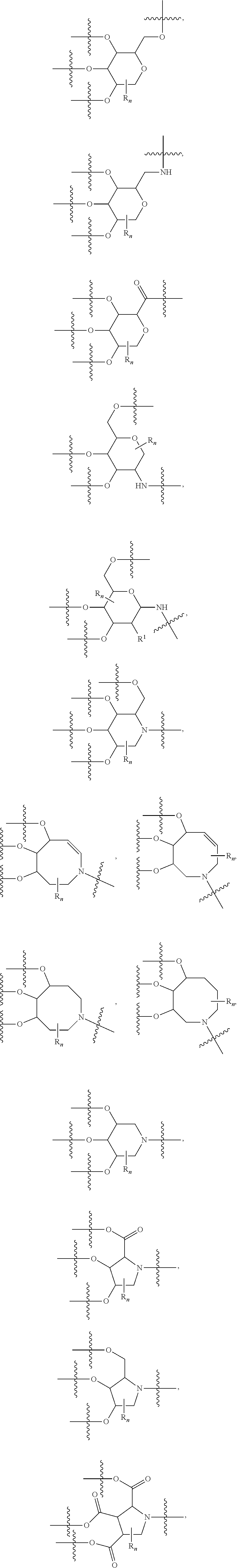

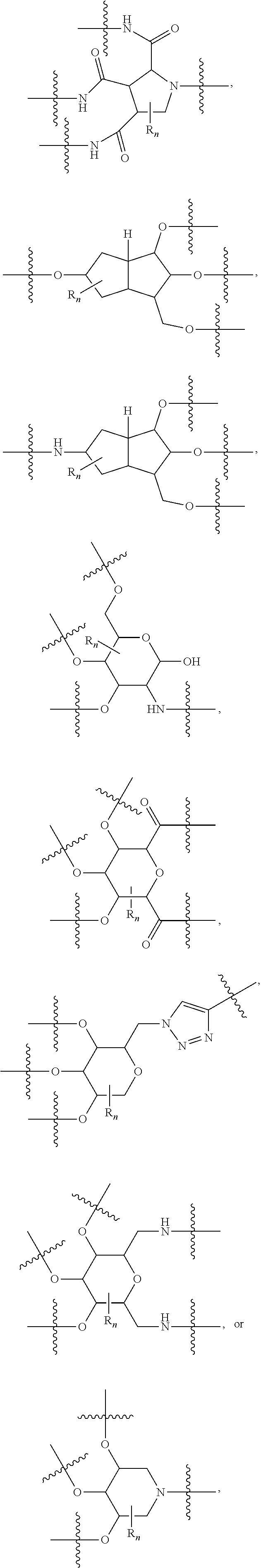

wherein
R¹ is H, C₁₋₆ alkyl, halogen or —NH(R²), wherein R² is H or acetyl;
each R is independently H, halogen, —CN, —C≡CH, —NH₂, —OC₁₋₆ alkyl, or C₁₋₆ alkyl, wherein said alkyl of —C₁₋₆alkyl and —OC₁₋₆alkyl is substituted with 0 to 5 halogen atoms; or two R together with the carbon to which they are attached form a C₃₋₆cycloalkyl or 3- to 6-membered heterocycloalkyl group, wherein said cycloalkyl of —C₃₋₆cycloalkyl and heterocycloalkyl of 3- to 6-membered heterocycloalkyl is substituted with 0 to 5 halogen atoms; and
n is 0, 1, 2 or 3, as valency permits; particularly n is 0.

In certain embodiments, non-limiting examples of carbohydrate mimetics may have the structure:

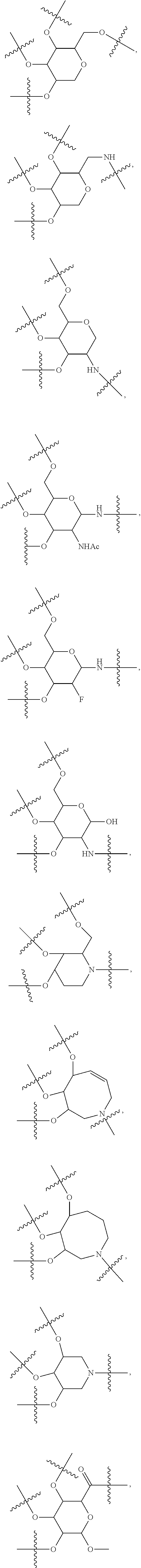

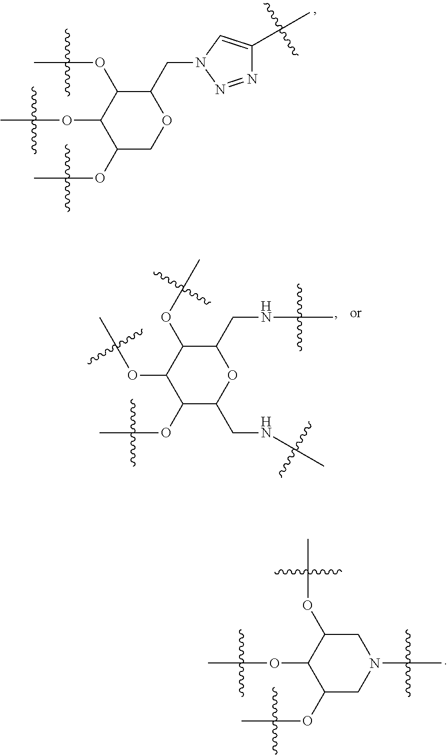 [SMILES: *OC1COC(Cn2cc(*)nn2)C(O*)C1O* ; *NCC1OC(CN*)C(O*)C(O*)C1O* ; *OC1CN(*)CC(O*)C1O*]

In certain embodiments, the non-limiting examples of carbohydrate mimetics may have the structure:

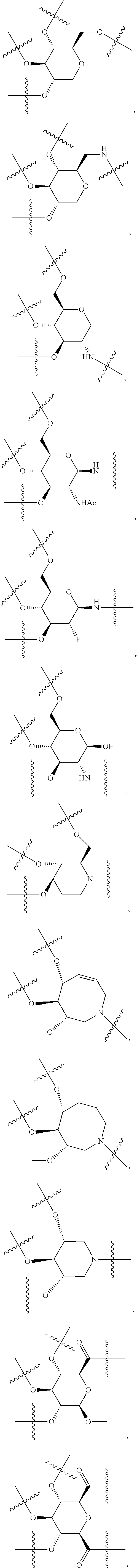

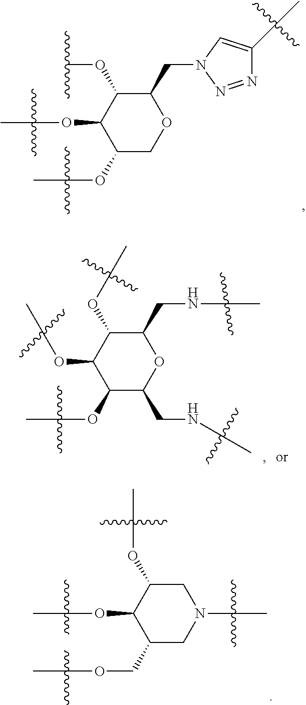 [SMILES: *O[C@H]1[C@H](O*)[C@@H](Cn2cc(*)nn2)OC[C@@H]1O* ; *NC[C@@H]1O[C@H](CN*)[C@@H](O*)[C@H](O*)[C@@H]1O* ; *OC[C@H]1CN(*)C[C@@H](O*)[C@@H]1O*]

In certain embodiments, non-limiting examples of carbohydrate mimetics joined with ligands may have the structure:

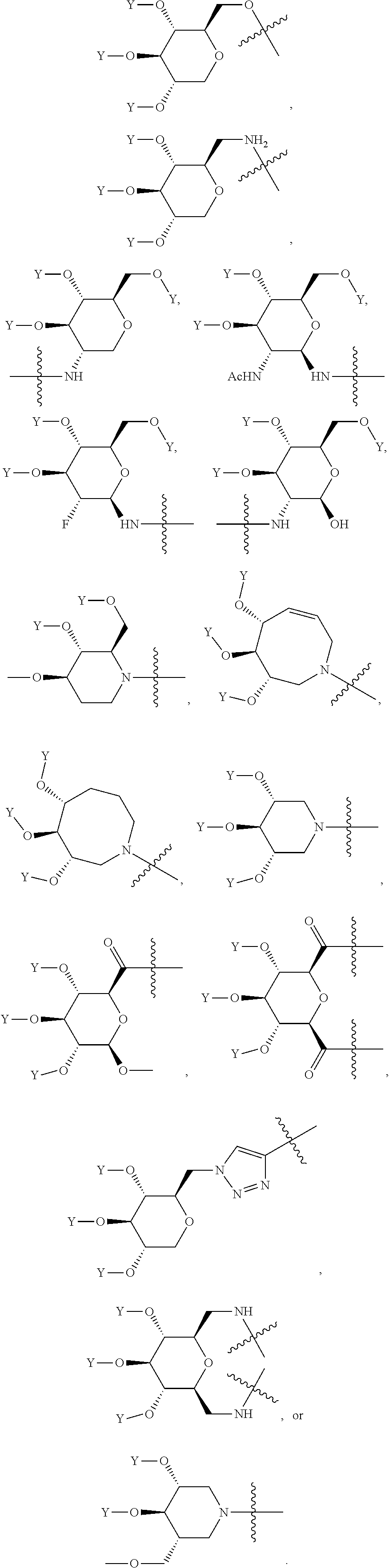

wherein Y is a ligand-linker moiety.

In certain embodiments, the non-limiting examples of carbohydrate mimetics may have the structure:

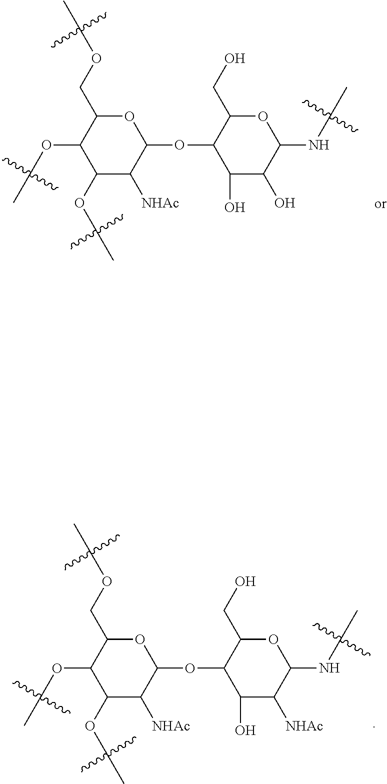 [SMILES: *NC1OC(CO)C(OC2OC(CO*)C(O*)C(O*)C2NC(C)=O)C(O)C1O ; *NC1OC(CO)C(OC2OC(CO*)C(O*)C(O*)C2NC(C)=O)C(O)C1NC(C)=O]

In certain embodiments, the non-limiting examples of carbohydrate mimetics may have the structure:

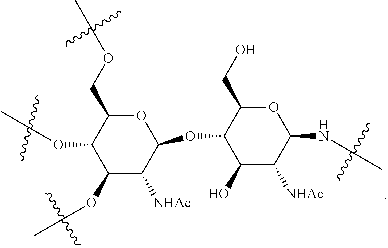 [SMILES: *N[C@@H]1O[C@H](CO)[C@@H](O[C@@H]2O[C@H](CO*)[C@@H](O*)[C@H](O*)[C@H]2NC(C)=O)[C@H](O)[C@H]1NC(C)=O]

As used herein, the wavy line, “ [symbol] ” denotes a point of attachment of a moiety to another moiety.

In terms of ligands (i.e., T in Formula (I)), both carbohydrates and carbohydrate mimetics may be used. Therefore, as used herein, the term “carbohydrate ligand” means to include carbohydrates, carbohydrate mimetics, or a combination thereof. In certain embodiments, the carbohydrate ligand is selected from the group consisting of N-acetyl-galactosamine (GalNAc), allose, altrose, arabinose, cladinose, erythrose, erythrulose, fructose, D-fucitol, L-fucitol, fucosamine, fucose, fuculose, galactosamine, D-galactosaminitol, galactose, glucosamine, N-acetyl-glucosamine, glucosaminitol, glucose, glucose-6-phosphate, gulose glyceraldehyde, L-glycero-D-mannos-heptose, glycerol, glycerone, gulose, idose, lyxose, mannosamine, mannose, mannose-6-phosphate, psicose, quinovose, quinovosamine, rhamnitol, rhamnosamine, rhamnose, ribose, ribulose, sedoheptulose, sorbose, tagatose, talose, tartaric acid, threose, xylose and xylulose, in unprotected or protected form. In certain embodiments, the carbohydrate ligand is N-acetyl-galactosamine (GalNAc) or N-acetyl-galactosamine triacetate. In certain embodiments, the carbohydrate ligand is N-acetyl-galactosamine (GalNAc).

The compounds of Formula I may have one or more chiral (asymmetric) centers. The present invention encompasses all stereoisomeric forms of the compounds of Formula I. Centers of asymmetry that are present in the compounds of Formula I can all independently of one another have (R) or (S) configuration. When bonds to a chiral carbon are depicted as straight lines in the structural Formulas of the invention, or when a compound name is recited without an (R) or (S) chiral designation for a chiral carbon, it is understood that both the (R) and (S) configurations of each such chiral carbon, and hence each enantiomer or diastereomer and mixtures thereof, are embraced within the Formula or by the name. The production of specific stereoisomers or mixtures thereof may be identified in the Examples where such stereoisomers or mixtures were obtained, but this in no way limits the inclusion of all stereoisomers and mixtures thereof from being within the scope of this invention.

The invention includes all possible enantiomers and diastereomers and mixtures of two or more stereoisomers, for example mixtures of enantiomers and/or diastereomers, in all ratios. Thus, enantiomers are a subject of the invention in enantiomerically pure form, both as levorotatory and as dextrorotatory antipodes, in the form of racemates and in the form of mixtures of the two enantiomers in all ratios. In the case of a cis/trans isomerism the invention includes both the cis form and the trans form as well as mixtures of these forms in all ratios. The preparation of individual stereoisomers can be carried out, if desired, by separation of a mixture by customary methods, for example by chromatography or crystallization, by the use of stereochemically uniform starting materials for the synthesis or by stereoselective synthesis. Optionally a derivatization can be carried out before a separation of stereoisomers. The separation of a mixture of stereoisomers can be carried out at an intermediate step during the synthesis of a compound of Formula I or it can be done on a final racemic product. Absolute stereochemistry may be determined by X-ray crystallography of crystalline products or crystalline intermediates which are derivatized, if necessary, with a reagent containing a stereogenic center of known configuration. Alternatively, absolute stereochemistry may be determined by Vibrational Circular Dichroism (VCD) spectroscopy analysis.

As used herein, the term “therapeutically active agent” refers to compounds and compound classes known as being therapeutically active. For example, a therapeutically active agent may be therapeutically active oligonucleotide. Non-limiting examples of therapeutically active agents may include an antisense oligonucleotide (ASO), a small interfering RNA (siRNA), a microRNA (miRNA), a microRNA mimic, an anti-miRNA oligonucleotide (AMO), a long non-coding RNA, a peptide nucleic acid (PNA), a helper lipid, and a phosphorodiamidate morpholino oligomer (PMO), wherein the nucleic acid is unmodified or modified. In certain embodiments, the therapeutically active agent may be an iRNA agent.

As used herein, the term “targeting moiety” refers to a moiety (e.g., N-acetylgalactosamine cluster) that specifically binds or reactively associates or complexes with a receptor or other receptive moiety associated with a given target cell population.

As used herein, the term “linker” refers to an organic moiety that connects two parts of a compound. Linkers may typically comprise a direct bond or an atom such as oxygen or sulfur, a unit such as NR⁸, C(O), C(O)NH, NHC(O), OC(O), C(O)O, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO2, N(R⁸), C(O), substituted or unsubstituted aryl, substituted or unsubstituted heteroaryl, substituted or unsubstituted heterocyclyl; where R⁸ is hydrogen, acyl, aliphatic or substituted aliphatic group. In certain embodiments, the linker is between 1-24 atoms, preferably 4-24 atoms, preferably 6-18 atoms, more preferably 8-18 atoms, and most preferably 8-16 atoms. Other examples of linkers are described in International Publication No. WO 2009/082607 and U.S. Patent Publication Nos. 2009/0239814, 2012/0136042, 2013/0158824, or 2009/0247608.

As used herein, the term “nucleoside” refers to sugar-nucleobase compounds and groups known in the art (e.g., modified or unmodified ribofuranose-nucleobase and 2′-deoxyribofuranose-nucleobase compounds and groups known in the art). The sugar may be ribofuranose. The sugar may be modified or unmodified. An unmodified sugar nucleoside is ribofuranose or 2′-deoxyribofuranose having an anomeric carbon bonded to a nucleobase. An unmodified nucleoside is ribofuranose or 2′-deoxyribofuranose having an anomeric carbon bonded to an unmodified nucleobase. Non-limiting examples of unmodified nucleosides include adenosine, cytidine, guanosine, uridine, 2′-deoxyadenosine, 2′-deoxycytidine, 2′-deoxyguanosine, and thymidine. The modified compounds and groups include one or more modifications selected from the group consisting of nucleobase modifications and sugar modifications described herein. A nucleobase modification is a replacement of an unmodified nucleobase with a modified nucleobase. A sugar modification may be, e.g., a 2′-substitution, locking, carbocyclization, or unlocking. A 2′-substitution is a replacement of 2′-hydroxyl in ribofuranose with 2′-fluoro, 2-methoxy, or 2′-(2-methoxy)ethoxy. A locking modification is an incorporation of a bridge between 4′-carbon atom and 2′-carbon atom of ribofuranose. Nucleosides having a locking modification are known in the art as bridged nucleic acids, e.g., locked nucleic acids (LNA), ethylene-bridged nucleic acids (ENA), and cEt nucleic acids. The bridged nucleic acids are typically used as affinity enhancing nucleosides.

As used herein, the term “nucleotide” refers to a nucleoside bonded to an internucleoside linkage or a monovalent group of the following structure —X¹—P(X²)(R¹)₂, where X¹ is O, S, or NH, and XV is absent, ═O, or ═S, and each R¹ is independently —OH, —N(R²)₂, or —O—CH₂CH₂CN, where each R² is independently an optionally substituted alkyl, or both R² groups, together with the nitrogen atom to which they are attached, combine to form an optionally substituted heterocyclyl.

As used herein, the term “oligonucleotide” refers to a structure containing 10 or more (e.g., 10 to 50) contiguous nucleosides covalently bound together by internucleoside linkages. An oligonucleotide includes a 5′ end and a 3′ end. The 5′ end of an oligonucleotide may be, e.g., hydroxyl, a targeting moiety, a hydrophobic moiety, 5′ cap, phosphate, diphosphate, triphosphate, phosphorothioate, diphosphorothioate, triphosphorothioate, phosphorodithioate, diphosphorodithioate, triphosphorodithioate, phosphonate, phosphoramidate, a cell penetrating peptide, an endosomal escape moiety, or a neutral organic polymer. The 3′ end of an oligonucleotide may be, e.g., hydroxyl, a targeting moiety, a hydrophobic moiety, phosphate, diphosphate, triphosphate, phosphorothioate, diphosphorothioate, triphosphorothioate, phosphorodithioate, disphosphorodithioate, triphosphorodithioate, phosphonate, phosphoramidate, a cell penetrating peptide, an endosomal escape moiety, or a neutral organic polymer (e.g., polyethylene glycol). An oligonucleotide having a 5′-hydroxyl or 5′-phosphate has an unmodified 5′ terminus. An oligonucleotide having a 5′ terminus other than 5′-hydroxyl or 5′-phosphate has a modified 5′ terminus. An oligonucleotide having a 3′-hydroxyl or 3′-phosphate has an unmodified 3′ terminus. An oligonucleotide having a 3′ terminus other than 3′-hydroxyl or 3′-phosphate has a modified 3′ terminus.

As used herein, “ [symbol] ” refers to an oligonucleotide (e.g., iRNA agents), unless otherwise depicted according to the context.

Unless otherwise stated, structures depicted herein are also meant to include compounds that differ only in the presence of one or more isotopically enriched atoms. For example, compounds having the depicted structures that differ only in the replacement of hydrogen by deuterium or tritium, or the replacement of a carbon by ¹³C or ¹⁴C are within the scope of this invention. Such compounds may be useful, for example, as analytical tools, as probes in biological assays, or as therapeutically active agents in accordance with the present invention.

### Ligands

The ligand can be any ligand described herein, e.g., those selected from the group consisting of carbohydrate ligands, polypeptide ligands and lipophile ligands. Ligands may be protected or unprotected. Preferred exemplary examples of ligands include N-acetylgalactosamine (GalNAc), e.g., N-acetyl-D-galactosylamine, and N-acetylgalactosamine triacetate, etc.

Other suitable ligands are described in U.S. Patent Publication Nos. 2009/073809, 2012/0136042, 2013/0158824, or 2009/0247608, U.S. Pat. No. 8,106,022 or WO2009/073809, each of which is hereby incorporated by reference.

The ligand moiety (e.g., a carbohydrate moiety) facilitates delivery of the oligonucleotide to the target site. One way a ligand moiety can improve delivery is by receptor mediated endocytotic activity. Without being bound by any particular theory, it is believed that this mechanism of uptake involves the movement of the oligonucleotide bound to membrane receptors into the interior of an area that is enveloped by the membrane via invagination of the membrane structure or by fusion of the delivery system with the cell membrane. This process is initiated via activation of a cell-surface or membrane receptor following binding of a specific ligand to the receptor. Receptor-mediated endocytotic systems include those that recognize sugars such as galactose. The ligand moiety therefore may include one or more monosaccharides, disaccharides, trisaccharides, tetrasaccharides, oligosaccharides, or polysaccharides, such as those described above. In preferred embodiments, the ligand moiety may be a moiety which is recognized by a human asialoglycoprotein receptor (ASGPR), such as human asialoglycoprotein receptor 2 (ASGPR2). Such a carbohydrate moiety may, for instance, comprise a sugar (e.g., galactose or N-acetyl-D-galactosylamine).

### Oligonucleotide

The oligonucleotide may be a chemically modified or unmodified nucleic acid molecule (RNA or DNA) having a length of less than about 100 nucleotides, for example, less than about 50 nucleotides. The nucleic acid may, for example, be (i) single stranded DNA or RNA, (ii) double stranded DNA or RNA, including double stranded DNA or RNA having a hairpin loop, or (iii) DNA/RNA hybrids. Non-limiting examples of double stranded RNA include siRNA (small interfering RNA). Single stranded nucleic acids include, e.g., antisense oligonucleotides, ribozymes, microRNA, and triplex forming oligonucleotides. In certain embodiments, the oligonucleotide has a length ranging from about 5 to about 50 nucleotides, e.g., from about 10 to about 50 nucleotides. In certain embodiments, the oligonucleotide has a length ranging from about 6 to about 30 nucleotides, e.g., from about 15 to about 30 nucleotides, e.g., from about 18 to about 23 nucleotides.

The oligonucleotide described herein can be an siRNA, microRNA, antimicroRNA, microRNA mimics, antimiR, antagomir, dsRNA, ssRNA, aptamer, immune stimulatory, decoy oligonucleotides, splice altering oligonucleotides, triplex forming oligonucleotides, G-quadruplexes or antisense. In certain embodiments, the oligonucleotide is an iRNA agent.

As used herein, the term “iRNA agent” refers to an RNA agent (or an agent that can be cleaved into an RNA agent) which can down regulate the expression of a target gene (e.g., an siRNA), preferably an endogenous or pathogen target RNA. While not wishing to be bound by theory, an iRNA agent may act by one or more of a number of mechanisms, including post-transcriptional cleavage of a target mRNA (referred to in the art as RNAi), or pre-transcriptional or pre-translational mechanisms. An iRNA agent can include a single strand or can include more than one strands, e.g., it can be a double stranded iRNA agent. If the iRNA agent is a single strand, it can include a 5′ modification which includes one or more phosphate groups or one or more analogs of a phosphate group. In certain embodiments, the iRNA agent is double stranded.

The iRNA agent typically includes a region of sufficient homology to the target gene, and is of sufficient length in terms of nucleotides, such that the iRNA agent, or a fragment thereof, can mediate down regulation of the target gene. The iRNA agent is or includes a region which is at least partially, and in certain embodiments fully, complementary to the target RNA. It is not necessary that there be perfect complementarity between the iRNA agent and the target, but the correspondence is preferably sufficient to enable the iRNA agent, or a cleavage product thereof, to direct sequence specific silencing, e.g., by RNAi cleavage of the target RNA, e.g., mRNA.

The nucleotides in the iRNA agent may be modified (e.g., one or more nucleotides may include a 2′-F or 2′-OCH₃ group), or be nucleotide surrogates. The single stranded regions of an iRNA agent may be modified or include nucleoside surrogates, e.g., the unpaired region or regions of a hairpin structure, e.g., a region which links two complementary regions, can have modifications or nucleoside surrogates. Modification to stabilize one or more 3′- or 5′-terminus of an iRNA agent, e.g., against exonucleases. Modifications can include C3 (or C6, C7, C12) amino linkers, thiol linkers, carboxyl linkers, non-nucleotidic spacers (C3, C6, C9, C12, abasic, triethylene glycol, hexaethylene glycol), special biotin or fluorescein reagents that come as phosphoramidites and that have another DMT-protected hydroxyl group, allowing multiple couplings during RNA synthesis. Modifications can also include, e.g., the use of modifications at the 2′—OH group of the ribose sugar, e.g., the use of deoxyribonucleotides, e.g., deoxythymidine, instead of ribonucleotides, and modifications in the phosphate group, e.g., phosphothioate modifications.

In certain embodiments, the different strands will include different modifications. In certain embodiments, strands may be chosen such that the iRNA agent includes a single strand or unpaired region at one or both ends of the molecule. A double stranded iRNA agent preferably has its strands paired with an overhang, e.g., one or two 5′ or 3′ overhangs (preferably at least a 3′ overhang of 2-3 nucleotides). For example, iRNA gents may have single-stranded overhangs, such as 3′ overhangs, of one, two or three nucleotides in length at each end. The overhangs can be the result of one strand being longer than the other, or the result of two strands of the same length being staggered.

Preferred lengths for the duplexed regions between the strands of the iRNA agent are between 6 and 30 nucleotides in length. The preferred duplexed regions are between 15 and 30, most preferably 18, 19, 20, 21, 22, and 23 nucleotides in length. Other preferred duplexed regions are between 6 and 20 nucleotides, most preferably 6, 7, 8, 9, 10, 11 and 12 nucleotides in length.

The oligonucleotide may be that described in U.S. Patent Publication Nos. 2009/0239814, 2012/0136042, 2013/0158824, or 2009/0247608, each of which is hereby incorporated by reference.

As used herein, the term “single strand siRNA” refers to an siRNA compound which is made up of a single molecule. It may include a duplexed region, formed by intra-strand pairing, e.g., it may be, or include, a hairpin or pan-handle structure. Single strand siRNA compounds may be antisense with regard to the target molecule.

A single strand siRNA may be sufficiently long that it can enter the RISC and participate in RISC mediated cleavage of a target mRNA. A single strand siRNA compound is at least 14, and in other embodiments at least 15, 20, 25, 29, 35, 40, or 50 nucleotides in length. In certain embodiments, it is less than 200, 100, or 60 nucleotides in length.

Hairpin siRNA compounds will have a duplex region equal to or at least 17, 18, 19, 29, 21, 22, 23, 24, or 25 nucleotide pairs. The duplex region will may be equal to or less than 200, 100, or 50, in length. In certain embodiments, ranges for the duplex region are 15-30, 17 to 23, 19 to 23, and 19 to 21 nucleotides pairs in length. The hairpin may have a single strand overhang or terminal unpaired region. In certain embodiments, the overhangs are 2-3 nucleotides in length. In certain embodiments, the overhang is at the sense side of the hairpin and in certain embodiments on the antisense side of the hairpin.

As used herein, the term “double stranded siRNA compound” refers to an siRNA compound which includes more than one, and in some cases two, strands in which interchain hybridization can form a region of duplex structure.

The antisense strand of a double stranded siRNA compound may be equal to or at least, 14, 15, 16 17, 18, 19, 25, 29, 40, or 60 nucleotides in length. It may be equal to or less than 200, 100, or 50, nucleotides in length. Ranges may be 17 to 25, 19 to 23, and 19 to 21 nucleotides in length. As used herein, the term “antisense strand” refers to the strand of an siRNA compound that is sufficiently complementary to a target molecule, e.g., a target RNA. The sense strand of a double stranded siRNA compound may be equal to or at least 14, 15, 16 17, 18, 19, 25, 29, 40, or 60 nucleotides in length. It may be equal to or less than 200, 100, or 50 nucleotides in length. Ranges may be 17 to 25, 19 to 23, and 19 to 21 nucleotides in length.

The double strand portion of a double stranded siRNA compound may be equal to or at least, 14, 15, 16 17, 18, 19, 20, 21, 22, 23, 24, 25, 29, 40, or 60 nucleotide pairs in length. It may be equal to or less than 200, 100, or 50, nucleotides pairs in length. Ranges may be 15-30, 17 to 23, 19 to 23, and 19 to 21 nucleotides pairs in length. In many embodiments, the siRNA compound is sufficiently large that it can be cleaved by an endogenous molecule, e.g., by Dicer, to produce smaller siRNA compounds, e.g., siRNAs agents.

The sense and antisense strands may be chosen such that the double-stranded siRNA compound includes a single strand or unpaired region at one or both ends of the molecule. Thus, a double-stranded siRNA compound may contain sense and antisense strands, paired to contain an overhang, e.g., one or two 5′ or 3′ overhangs, or a 3′ overhang of one to three nucleotides. The overhangs can be the result of one strand being longer than the other, or the result of two strands of the same length being staggered. Some embodiments will have at least one 3′ overhang. In certain embodiments, both ends of an siRNA molecule will have a 3′ overhang. In certain embodiments, the overhang is 2 nucleotides.

In certain embodiments, the length for the duplexed region is between 15 and 30, or 18, 19, 20, 21, 22, and 23 nucleotides in length, e.g., in the ssiRNA compound range described above. ssiRNA compounds can resemble in length and structure the natural Dicer processed products from long dsiRNAs. Embodiments in which the two strands of the ssiRNA compound are linked, e.g., covalently linked are also included. Hairpin, or other single strand structures which provide the required double stranded region, and a 3′ overhang are also contemplated.

The siRNA compounds described herein, including double-stranded siRNA compounds and single-stranded siRNA compounds can mediate silencing of a target RNA, e.g., mRNA, e.g., a transcript of a gene that encodes a protein. For convenience, such mRNA is also referred to herein as mRNA to be silenced. Such a gene is also referred to as a target gene. In general, the RNA to be silenced is an endogenous gene or a pathogen gene. In addition, RNAs other than mRNA, e.g., tRNAs, and viral RNAs, can also be targeted.

As used herein, the term “mediates RNAi” refers to the ability to silence, in a sequence specific manner, a target RNA. While not wishing to be bound by theory, it is believed that silencing uses the RNAi machinery or process and a guide RNA, e.g., an ssiRNA compound of 21 to 23 nucleotides.

In certain embodiments, an siRNA compound is “sufficiently complementary” to a target RNA, e.g., a target mRNA, such that the siRNA compound silences production of protein encoded by the target mRNA. In certain embodiments, the siRNA compound is “exactly complementary” to a target RNA, e.g., the target RNA and the siRNA compound anneal, for example to form a hybrid made exclusively of Watson-Crick base pairs in the region of exact complementarity. A “sufficiently complementary” target RNA can include an internal region (e.g., of at least 10 nucleotides) that is exactly complementary to a target RNA. Moreover, in certain embodiments, the siRNA compound specifically discriminates a single-nucleotide difference. In this case, the siRNA compound only mediates RNAi if exact complementary is found in the region (e.g., within 7 nucleotides of) the single-nucleotide difference.

Micro RNAs (miRNAs) are a highly conserved class of small RNA molecules that are transcribed from DNA in the genomes of plants and animals, but are not translated into protein. Processed miRNAs are single-stranded ˜17-25 nucleotide (nt) RNA molecules that become incorporated into the RNA-induced silencing complex (RISC) and have been identified as key regulators of development, cell proliferation, apoptosis and differentiation. They are believed to play a role in regulation of gene expression by binding to the 3′-untranslated region of specific mRNAs. RISC mediates down-regulation of gene expression through translational inhibition, transcript cleavage, or both. RISC is also implicated in transcriptional silencing in the nucleus of a wide range of eukaryotes.

The number of miRNA sequences identified to date is large and growing, illustrative examples of which can be found, e.g., in: “miRBase: microRNA sequences, targets and gene nomenclature” Griffiths-Jones S, Grocock RJ, van Dongen S, Bateman A, Enright AJ. NAR, 2006, 34, Database Issue, D140-D144; “The microRNA Registry” Griffiths-Jones S. NAR, 2004, 32, Database Issue, D109-D111; and also, at http://microrna.sanger.ac.uk/sequences/.

In certain embodiments, a nucleic acid is an antisense oligonucleotide directed to a target polynucleotide. As used herein, the term “antisense oligonucleotide” or simply “antisense” is meant to include oligonucleotides that are complementary to a targeted polynucleotide sequence. Antisense oligonucleotides are single strands of DNA or RNA that are complementary to a chosen sequence, e.g., a target gene mRNA. Antisense oligonucleotides are thought to inhibit gene expression by binding to a complementary mRNA. Binding to the target mRNA can lead to inhibition of gene expression either by preventing translation of complementary mRNA strands by binding to it, or by leading to degradation of the target mRNA. Antisense DNA can be used to target a specific, complementary (coding or non-coding) RNA. If binding takes places this DNA/RNA hybrid can be degraded by the enzyme RNase H. In certain embodiments, antisense oligonucleotides contain from about 10 to about 50 nucleotides, more particularly, about 15 to about 30 nucleotides. The term also encompasses antisense oligonucleotides that may not be exactly complementary to the desired target gene. Thus, instances where non-target specific-activities are found with antisense, or where an antisense sequence containing one or more mismatches with the target sequence is the most preferred for a particular use, are contemplated.

Antisense oligonucleotides have been demonstrated to be effective and targeted inhibitors of protein synthesis, and, consequently, can be used to specifically inhibit protein synthesis by a targeted gene. The efficacy of antisense oligonucleotides for inhibiting protein synthesis is well established. Methods of producing antisense oligonucleotides are known in the art and can be readily adapted to produce an antisense oligonucleotide that targets any polynucleotide sequence. Selection of antisense oligonucleotide sequences specific for a given target sequence is based upon analysis of the chosen target sequence and determination of secondary structure, Tm, binding energy, and relative stability. Antisense oligonucleotides may be selected based upon their relative inability to form dimers, hairpins, or other secondary structures that would reduce or prohibit specific binding to the target mRNA in a host cell. Highly preferred target regions of the mRNA include those regions at or near the AUG translation initiation codon and those sequences that are substantially complementary to 5′ regions of the mRNA.

Antagomirs are RNA-like oligonucleotides that harbor various modifications for RNAse protection and pharmacologic properties, such as enhanced tissue and cellular uptake. They differ from normal RNA by, e.g., complete 2′-O-methylation of sugar, phosphorothioate backbone and, for example, a cholesterol-moiety at 3′-end. Antagomirs may be used to efficiently silence endogenous miRNAs by forming duplexes comprising the antagomir and endogenous miRNA, thereby preventing miRNA-induced gene silencing. An example of antagomir-mediated miRNA silencing is the silencing of miR-122, described in Krutzfeldt et al., Nature, 2005, 438: 685-689, which is expressly incorporated by reference herein in its entirety. Antagomir RNAs may be synthesized using standard solid phase oligonucleotide synthesis protocols. See U.S. Patent Application Publication Nos. 2007/0123482 and 2007/0213292, each of which is incorporated herein by reference in its entirety.

An antagomir can include ligand-conjugated monomer subunits and monomers for oligonucleotide synthesis. Non-limiting examples of monomers are described in U.S. Patent Application Publication No. 2005/0107325, which is incorporated herein by reference in its entirety. An antagomir can have a ZXY structure, such as is described in WO 2004/080406, which is incorporated herein by reference in its entirety. An antagomir can be complexed with an amphipathic moiety. Non-limiting examples of amphipathic moieties for use with oligonucleotide agents are described in WO 2004/080406, which is incorporated herein by reference in its entirety.

Aptamers are nucleic acid or peptide molecules that bind to a particular molecule of interest with high affinity and specificity (Tuerk and Gold, Science 249:505 (1990); Ellington and Szostak, Nature 346:818 (1990)). DNA or RNA aptamers have been successfully produced which bind many different entities from large proteins to small organic molecules. See Eaton, Curr. Opin. Chem. Biol. 1:10-16 (1997), Famulok, Curr. Opin. Struct. Biol. 9:324-9 (1999), and Hermann and Patel, Science 287:820-5 (2000). Aptamers may be RNA or DNA based, and may include a riboswitch. A riboswitch is a part of an mRNA molecule that can directly bind a small target molecule, and whose binding of the target affects the gene's activity. Thus, an mRNA that contains a riboswitch is directly involved in regulating its own activity, depending on the presence or absence of its target molecule. Generally, aptamers are engineered through repeated rounds of in vitro selection or equivalently, SELEX (systematic evolution of ligands by exponential enrichment) to bind to various molecular targets such as small molecules, proteins, nucleic acids, and even cells, tissues and organisms. The aptamer may be prepared by any known method, including synthetic, recombinant, and purification methods, and may be used alone or in combination with other aptamers specific for the same target. Further, the term “aptamer” specifically includes “secondary aptamers” containing a consensus sequence derived from comparing two or more known aptamers to a given target.

According to another embodiment, nucleic acid-lipid particles are associated with ribozymes. Ribozymes are RNA molecules complexes having specific catalytic domains that possess endonuclease activity (Kim and Cech, Proc Natl Acad Sci USA. 1987 December, 84(24): 8788-92; Forster and Symons, Cell. 1987 Apr. 24, 49(2): 211-20). For example, a large number of ribozymes accelerate phosphoester transfer reactions with a high degree of specificity, often cleaving only one of several phosphoesters in an oligonucleotide substrate (Cech et al, Cell. 1981 December, 27 (3 Pt 2): 487-96; Michel and Westhof, J Mol Biol. 1990 Dec. 5, 216(3): 585-610; Reinhold-Hurek and Shub, Nature. 1992 May 14; 357(6374): 173-6). This specificity has been attributed to the requirement that the substrate bind via specific base-pairing interactions to the internal guide sequence (“IGS”) of the ribozyme prior to chemical reaction.

At least six basic varieties of naturally-occurring enzymatic RNAs are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nucleic acid molecule may be formed in a hammerhead, hairpin, a hepatitis δ virus, group I intron or RNaseP RNA (in association with an RNA guide sequence) or Neurospora VS RNA motif, for example. Specific examples of hammerhead motifs are described by Rossi et al. Nucleic Acids Res. 1992 Sep. 11, 20(17):4559-65. Examples of hairpin motifs are described by Hampel et al. (Eur. Pat. Appl. Publ. No. EP 0360257), Hampel and Tritz, Biochemistry 1989 Jun. 13, 28(12): 4929-33; Hampel et al., Nucleic Acids Res. 1990 Jan. 25, 18(2): 299-304 and U.S. Pat. No. 5,631,359. An example of the hepatitis δ virus motif is described in Perrotta and Been, Biochemistry, 1992 Dec. 1, 31(47): 11843-52; an example of the RNaseP motif is described in Guerrier-Takada et al., Cell. 1983 December, 35(3 Pt 2): 849-57; Neurospora VS RNA ribozyme motif is described in Saville and Collins, Cell, 1990 May 18, 61(4): 685-96; Saville and Collins, Proc Natl Acad Sci USA. 1991 Oct. 1, 88(19): 8826-30; Collins and Olive, Biochemistry, 1993 Mar. 23, 32(11): 2795-9); and an example of the Group I intron is described in U.S. Pat. No. 4,987,071. Important characteristics of enzymatic nucleic acid molecules used are that they have a specific substrate binding site which is complementary to one or more of the target gene DNA or RNA regions, and that they have nucleotide sequences within or surrounding that substrate binding site which impart an RNA cleaving activity to the molecule. Thus the ribozyme constructs need not be limited to specific motifs mentioned herein.

Methods of producing a ribozyme targeted to any polynucleotide sequence are known in the art. Ribozymes may be designed as described in Int. Pat. Appl. Publ. Nos. WO 93/23569 and WO 94/02595, and synthesized to be tested in vitro and in vivo, as described therein.

Ribozyme activity can be optimized by altering the length of the ribozyme binding arms or chemically synthesizing ribozymes with modifications that prevent their degradation by serum ribonucleases (see e.g., Int. Pat. Appl. Publ. Nos. WO 92/07065, WO 93/15187, and WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem II bases to shorten RNA synthesis times and reduce chemical requirements.

Nucleic acids associated with lipid particles may be immunostimulatory, including immunostimulatory oligonucleotides (ISS; single- or double-stranded) capable of inducing an immune response when administered to a subject, which may be a mammal or other patient. ISS include, e.g., certain palindromes leading to hairpin secondary structures (see Yamamoto S., et al., (1992) J. Immunol. 148: 4072-4076), or CpG motifs, as well as other known ISS features (such as multi-G domains, see WO 96/11266).

The immune response may be an innate or an adaptive immune response. The immune system is divided into a more innate immune system, and acquired adaptive immune system of vertebrates, the latter of which is further divided into humoral cellular components. In certain embodiments, the immune response may be mucosal. In certain embodiments, an immunostimulatory nucleic acid is only immunostimulatory when administered in combination with a lipid particle, and is not immunostimulatory when administered in its “free form”. Such an oligonucleotide is considered to be immunostimulatory. Immunostimulatory nucleic acids are considered to be non-sequence specific when it is not required that they specifically bind to and reduce the expression of a target polynucleotide in order to provoke an immune response. Thus, certain immunostimulatory nucleic acids may comprise a sequence corresponding to a region of a naturally occurring gene or mRNA, but they may still be considered non-sequence specific immunostimulatory nucleic acids.

In certain embodiments, the immunostimulatory nucleic acid or oligonucleotide comprises at least one CpG dinucleotide. The oligonucleotide or CpG dinucleotide may be unmethylated or methylated. In certain embodiments, the immunostimulatory nucleic acid comprises at least one CpG dinucleotide having a methylated cytosine. In certain embodiments, the nucleic acid comprises a single CpG dinucleotide, wherein the cytosine in said CpG dinucleotide is methylated. In an alternative embodiment, the nucleic acid comprises at least two CpG dinucleotides, wherein at least one cytosine in the CpG dinucleotides is methylated. In a further embodiment, each cytosine in the CpG dinucleotides present in the sequence is methylated. In certain embodiments, the nucleic acid comprises a plurality of CpG dinucleotides, wherein at least one of said CpG dinucleotides comprises a methylated cytosine.

In nucleotide sequences, “G”, “C”, “A”, “T” and “U” each generally stand for a nucleotide that contains guanine, cytosine, adenine, thymidine and uracil as a base, respectively. However, it will be understood that the term “ribonucleotide” or “nucleotide” can also refer to a modified nucleotide, or a surrogate replacement moiety. The skilled person is well aware that guanine, cytosine, adenine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of dsRNA featured in the invention by a nucleotide containing, for example, inosine. Sequences containing such replacement moieties may be suitable for the compositions and methods described herein.

As used herein, the term “solid support” refers to in particular any particle, bead, or surface upon which synthesis can occur. Solid supports which can be used in the different embodiments of the processes described herein can be selected for example from inorganic supports and organic supports. Non-limiting examples of inorganic supports include silica gel and controlled pore glass (CPG). Non-limiting examples of organic supports include highly crosslinked polystyrene, Tentagel (grafted copolymers consisting of a low crosslinked polystyrene matrix on which polyethylene glycol (PEG or POE) is grafted), polyvinylacetate (PVA), Poros—a copolymer of polystyrene/divinyl benzene, aminopolyethyleneglycol, cellulose, etc. In certain embodiments, solid supports may include those that are hydrophobic. Some embodiments of the invention may utilize polystyrene based solid supports. Many other solid supports are commercially available and suitable for the present invention.

### Usage

The present invention relates to ligand (e.g., carbohydrate ligand) conjugates of oligonucleotides (e.g., an iRNA agent) or other therapeutically active agents, which have one or more advantageous properties, such as improved in vivo and/or in vitro delivery of the oligonucleotide or other therapeutically active agents, lower manufacturing costs or fewer manufacturing issues, or better chemical stability. These conjugates provide effective delivery of oligonucleotides or other therapeutically active agents. In certain embodiments, ligand conjugates of the invention can be prepared and used to deliver therapeutically active agents to cells, tissues, and organs. Non-limiting examples of therapeutically active agents that can be delivered include an antisense oligonucleotide (ASO), a small interfering RNA (siRNA), a microRNA (miRNA), a microRNA mimic, an anti-miRNA oligonucleotide (AMO), a long non-coding RNA, a peptide nucleic acid (PNA), a helper lipid, and a phosphorodiamidate morpholino oligomer (PMO), wherein the nucleic acid is unmodified or modified.

In certain embodiments, the ligand conjugate of the invention is delivered to and contacted with a cell. In certain embodiments of the invention a contacted cell is in culture and in other embodiments a contacted cell is in a subject. Non-limiting examples of cells that may be contacted with the ligand conjugate of the invention include liver cells, muscle cells, cardiac cells, circulatory cells, neuronal cells, glial cells, fat cells, skin cells, hematopoietic cells, epithelial cells, immune system cells, endocrine cells, exocrine cells, endothelial cells, sperm, oocytes, muscle cells, adipocytes, kidney cells, hepatocytes, or pancreas cells. In certain embodiments, the cell contacted with the ligand conjugate of the invention is a liver cell.

The ligand conjugate of the invention may be useful for targeting a gene for which expression is undesired in a subject. For example, TTR for polyneuropathy of hereditary transthyretin-mediated amyloidosis, ALAS1 for acute hepatic porphyria (AHP), GO for primary hyperoxaluria type 1 (PH1), PCSK9 for hypercholesterolemia, AGT for hypertension, LPA for atherosclerotic cardiovascular diseases (CVDs), or ANGPTL3 for dyslipidemia.

The ligand conjugate of the invention may also be used to treat disorders in a subject, including disorders characterized by unwanted cell proliferation, hematological disorders, metabolic disorders, liver disorders, completement mediated disorders, genetic rare disorders and disorders characterized by inflammation or chronic virus infection. For example, nonalcoholic fatty liver disease (NAFLD), nonalcoholic steatohepatitis (NASH). In certain embodiments, a disorder in a subject is treated by administering one or more ligand conjugates of the invention that have a sequence that is substantially identical to a sequence in a gene involved in the disorder.

The ligand conjugate of the invention may be useful for targeting a gene for which expression is undesired in the liver. For example, the ligand conjugate of the invention can target a nucleic acid expressed by a hepatitis virus (e.g., hepatitis C, hepatitis B, hepatitis A, hepatitis D, hepatitis E, hepatitis F, hepatitis G, or hepatitis H).

The ligand conjugate of the invention may also be used to treat other liver disorders, including disorders characterized by unwanted cell proliferation, hematological disorders, metabolic disorders, and disorders characterized by inflammation. A proliferation disorder of the liver can be, for example, a benign or malignant disorder, e.g., a cancer, e.g., a hepatocellular carcinoma (HCC), hepatic metastasis, or hepatoblastoma. A hepatic hematology or inflammation disorder can be a disorder involving clotting factors, a complement-mediated inflammation or a fibrosis, for example. Metabolic diseases of the liver include dyslipidemias and irregularities in glucose regulation. In certain embodiments, a liver disorder is treated by administering one or more ligand conjugates of the invention that have a sequence that is substantially identical to a sequence in a gene involved in the liver disorder.

In certain embodiments, a ligand conjugate of the invention targets a nucleic acid expressed in a subject, such as angiopoietin-like protein 3 RNA, apolipoprotein C3 RNA, proprotein convertase subtilisin/kexin type 9 (PCSK9) RNA, LAP RNA or Angiotensinogen (AGT) RNA c-jun RNA, beta-catenin RNA, or glucose-6-phosphatase RNA.

In certain embodiments, a ligand conjugate of the invention targets a nucleic acid expressed in the liver, such as ApoB RNA, c-jun RNA, beta-catenin RNA, or glucose-6-phosphatase mRNA.

Therefore, in some aspect, the present invention is directed to a method of modulating the expression of a target gene in a cell, comprising delivering to said cell a ligand conjugate as described herein. In certain embodiments, the target gene is relevant to the metabolic disease. In certain embodiments, the metabolic disease is dyslipidemia. In certain embodiments, the target gene is relevant to the liver disease. In certain embodiments, the liver disease is nonalcoholic fatty liver disease (NAFLD), nonalcoholic steatohepatitis (NASH), HBV, liver fibrosis, and liver cirrhosis.

In certain embodiments, a biological sample may be obtained and assessed for delivery of a therapeutically active agent such as a nucleic acid using a ligand conjugate as described herein. As used herein, the term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

In some aspect, the present invention is directed to use of the ligand conjugate of the invention in the manufacture of a medicament for modulating the expression of a target gene in a cell. In certain embodiments, the target gene is relevant to the metabolic disease. In certain embodiments, the metabolic disease is hypercholesterolemia, hypertriglyceridemia or atherosclerosis. In certain embodiments, the target gene is relevant to the liver disease. In certain embodiments, the liver disease is nonalcoholic fatty liver disease (NAFLD), nonalcoholic steatohepatitis (NASH), HBV, liver fibrosis, and liver cirrhosis.

In some aspect, the present invention is directed to the ligand conjugate of the invention for use in a method of modulating the expression of a target gene in a cell, wherein the ligand conjugate of the invention is delivered to said cell. In certain embodiments, the target gene is relevant to the metabolic disease. In certain embodiments, the metabolic disease is hypercholesterolemia, hypertriglyceridemia or atherosclerosis. In certain embodiments, the target gene is relevant to the liver disease. In certain embodiments, the liver disease is nonalcoholic fatty liver disease (NAFLD), nonalcoholic steatohepatitis (NASH), HBV, liver fibrosis, and liver cirrhosis.

### Administration

In certain embodiments, the ligand conjugate of the invention can be administered to a subject. The ligand conjugate described herein may be used to deliver the therapeutically active agent to a cell in the subject. In certain embodiments, the therapeutically active agent is an oligonucleotide. In certain embodiments the oligonucleotide comprises an inhibitor RNA, or siRNA molecule selected to reduce expression of the siRNA's target gene upon delivery. In certain embodiments, the present invention relates to methods of treating a disease or condition associated with expression of a gene in a cell or cells of a subject, wherein the administration of the iRNA agent reduces expression of the gene and treats the disease or condition in the subject. Administration of the ligand conjugate of the invention may be done using routine methods.

As used herein, the term “subject” refers to a human or vertebrate mammal including but not limited to a dog, cat, horse, goat, cow, sheep, rodent, and primate, e.g., monkey. Thus, the invention can be used to treat diseases or conditions in human and non-human subjects. For example, conjugates, compositions and methods of the invention can be used in veterinary applications as well as in human prevention and treatment regimens. In certain embodiments, the subject is a domesticated animal. In certain embodiments, the subject is a mammal. In certain embodiments, the subject is a human (e.g., a man, a woman, or a child). The human may be of either sex and may be at any stage of development. In certain embodiments, the subject has been diagnosed with a condition or disease to be treated. In other embodiments, the subject is at risk of developing a condition or disease. In certain embodiments, the subject is an experimental animal (e.g., mouse, rat, rabbit, dog, pig, or primate).

As used herein, the terms “administration” and “administer” refer to implanting, absorbing, ingesting, injecting, inhaling, or otherwise introducing the compound of the invention, or a pharmaceutical composition thereof. The terms “treatment” and “treat” refer to reversing, alleviating, delaying the onset of, or inhibiting the progress of a “pathological condition” (e.g., a disease, disorder, or condition, or one or more signs or symptoms thereof) described herein. In certain embodiments, treatment may be administered after one or more signs or symptoms of a disease or condition have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. As used herein, the terms “disease”, “disorder”, “condition”, and “pathological condition” are used interchangeably.

Dosage levels for administration can be determined by those skilled in the art by routine experimentation. In certain embodiments, a unit dose may contain between about 0.01 mg/kg and about 100 mg/kg body weight of siRNA. Alternatively, the dose can be from 10 mg/kg to 25 mg/kg body weight, or 1 mg/kg to 10 mg/kg body weight, or 0.05 mg/kg to 5 mg/kg body weight, or 0.1 mg/kg to 5 mg/kg body weight, or 0.1 mg/kg to 1 mg/kg body weight, or 0.1 mg/kg to 0.5 mg/kg body weight, or 0.5 mg/kg to 1 mg/kg body weight. Clinical trials are routinely used to assess dosage levels for therapeutically active agents.

The ligand conjugate of the invention may be formulated as pharmaceutically acceptable salts. When used in medicine, the salts should be pharmaceutically acceptable, but non-pharmaceutically acceptable salts may conveniently be used to prepare pharmaceutically-acceptable salts thereof and are not excluded from the scope of the invention.

As used herein, the term “pharmaceutically acceptable salt” refers to those salts which are, within the scope of sound medical judgment, suitable for use in contact with the tissues of humans and other animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19.

Pharmaceutically acceptable salts of the compounds of this invention include those derived from suitable inorganic and organic acids and bases. The salts can be prepared during the final isolation and purification of the compounds or separately by reacting the appropriate compound in the form of the free base with a suitable acid. Pharmacologically and pharmaceutically acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically acceptable salts can be prepared as alkaline metal or alkaline earth salts, such as sodium, potassium or calcium salts.

Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxy ethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quatemized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable salts include inorganic acids such as hydrochloric acid, hydrobromic acid, sulfuric acid, and phosphoric acid; and organic acids such as oxalic acid, maleic acid, succinic acid, and citric acid.

Representative base salts are formed from bases which form non-toxic salts. Examples may include, but not limited to, the aluminium, arginine, benzathine, calcium, choline, diethylamine, bis(2-hydroxyethyl)amine (diolamine), glycine, lysine, magnesium, meglumine, 2-aminoethanol (olamine), potassium, sodium, 2-Amino-2-(hydroxymethyl)propane-1,3-diol (tris or tromethamine) and zinc salts. Hemisalts of acids and bases may also be formed, for example, hemisulfate and hemicalcium salts. For a review on suitable salts, see, Stahl and Wermuth, Handbook of Pharmaceutical Salts: Properties, Selection, and Use (Wiley-VCH, 2002).

The ligand conjugate of the invention may be formulated as being associated with a solvent, usually by a solvolysis reaction. This physical association may include hydrogen bonding. Conventional solvents include water, methanol, ethanol, acetic acid, DMSO, THF, diethyl ether, and the like. The compounds of the invention may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. The term “solvate” encompasses both solution-phase and isolable solvates. Representative solvates include hydrates, ethanolates, and methanolates. The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·xH₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R≠6H₂O)).

A variety of administration routes for the ligand conjugate of the invention are available. The particular delivery mode selected will depend upon the particular condition being treated and the dosage required for therapeutic efficacy. Methods of this invention, generally speaking, may be practiced using any mode of administration that is medically acceptable, meaning any mode that produces effective levels of treatment without causing clinically unacceptable adverse effects. In certain embodiments, the ligand conjugate of the invention may be administered via an oral, enteral, mucosal, percutaneous, and/or parenteral route. The term “parenteral” includes subcutaneous, intrathecal, intravenous, intramuscular, intraperitoneal, and intrastemal injection, or infusion techniques. Other routes include but are not limited to nasal, dermal, vaginal, rectal, and sublingual. Delivery routes of the invention may include intrathecal, intraventricular, or intracranial. In certain embodiments, the ligand conjugate of the invention may be administered via parenteral route. In certain embodiments, the ligand conjugate of the invention may be administered via subcutaneous injection route.

In certain embodiments, the ligand conjugate of the invention may be administered directly to a tissue. Direct tissue administration may be achieved by direct injection, or other art-known means. The ligand conjugate of the invention may be administered once, or alternatively may be administered in a plurality of administrations. If administered multiple times, the ligand conjugate of the invention may be administered via different routes. For example, the first (or the first few) administrations may be made directly into an affected tissue or organ while later administrations may be systemic.

The ligand conjugate of the invention, when it is desirable to have it administered systemically, may be formulated for parenteral administration by injection, e.g., by bolus injection or continuous infusion. Formulations for injection may be presented in unit dosage form, e.g., in ampoules or in multi-dose containers, with or without an added preservative.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day may be used as needed to achieve appropriate systemic or local levels of one or more ligand conjugates of the invention, to result in a desired level of the therapeutically active agent, for example, a desired level of siRNA.

In certain embodiments of the invention, the ligand conjugate of the invention may be delivered using the bioerodible implant by way of diffusion, or by degradation of the polymeric matrix. Non-limiting examples of synthetic polymers for such use are well known in the art. Biodegradable polymers and non-biodegradable polymers can be used for delivery of one or more of the ligand conjugates of the invention using art-known methods. Such methods may also be used to deliver one or more ligand conjugates of the invention for treatment. Additional suitable delivery systems can include time-release, delayed release or sustained-release delivery systems. Such systems can avoid repeated administrations of the ligand conjugate of the invention, increasing convenience to the subject and the health-care provider. Many types of release delivery systems are available and known to those of ordinary skill in the art. (See for example: U.S. Pat. Nos. 5,075,109; 4,452,775; 4,675,189; 5,736,152; 3,854,480; 5,133,974; and 5,407,686). In addition, pump-based hardware delivery systems can be used, some of which are adapted for implantation.

Use of a long-term sustained release implant may also be suitable for prophylactic treatment of subjects and for subjects at risk of developing a recurrent disease or condition to be prevented and/or treated with a therapeutically active agent, e.g., an siRNA, delivered using the ligand conjugate as described herein. Long-term release, as used herein, means that the implant is constructed and arranged to delivery therapeutic levels of the active ingredient for at least 30 days, 60 days, 90 days or longer. Long-term sustained release implants are well-known to those of ordinary skill in the art and include some of the release systems described above.

In certain embodiments, the compound of the present invention may be administered in combination with an additional therapeutically active agent. Non-limiting examples of the additional therapeutically active agents include an agent for the treatment of liver disease. The additional therapeutically active agents can be administered before, after, or at the same time that the ligand conjugate of the invention is administered.

### Pharmaceutical Compositions

In some aspect, the present invention is directed to a pharmaceutical composition comprising the ligand conjugate as described herein, and a pharmaceutically acceptable carrier or excipient.

As used herein, the term “pharmaceutically acceptable carrier or excipient” refers to a carrier or excipient that is useful in preparing a pharmaceutical composition that is generally safe, non-toxic and neither biologically nor otherwise undesirable, and includes carrier or excipient that is acceptable for veterinary use as well as human pharmaceutical use. A “pharmaceutically acceptable carrier or excipient” as used herein includes both one and more than one such carrier or excipient. The particular excipient, carrier, or diluent or used will depend upon the means and purpose for which the compounds of the present invention is being applied. Suitable carriers and excipients are well known to those skilled in the art and are described in detail in, e.g., Ansel, Howard C, et al., Ansel's Pharmaceutical Dosage Forms and Drug Delivery Systems. Philadelphia: Lippincott, Williams & Wilkins, 2004; Gennaro, Alfonso R., et al., Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., the compound or pharmaceutical composition as described herein) or aid in the manufacturing of the pharmaceutical product (i.e., medicament).

The compositions of the present invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form depends on the intended mode of administration and therapeutic application.

Pharmaceutical compositions of the present invention may be prepared by any of the well-known techniques of pharmacy, such as effective formulation and administration procedures.

The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pennsylvania, 1975; Liberman et al., Eds., Pharmaceutical Dosage Forms, Marcel Decker, New York, N.Y., 1980; and Kibbe et al., Eds., Handbook of Pharmaceutical Excipients (3^{rd} Ed.), American Pharmaceutical Association, Washington, 1999.

### Synthesis

The compounds of the present invention may be prepared by the general and specific methods described below, using the common general knowledge of one skilled in the art of synthetic organic chemistry. Such common general knowledge can be found in standard reference books such as Comprehensive Organic Chemistry, Ed. Barton and Ollis, Elsevier; Comprehensive Organic Transformations: A Guide to Functional Group Preparations, Larock, John Wiley and Sons; and Compendium of Organic Synthetic Methods, Vol. I-XII (published by Wiley-Interscience). The starting materials used herein are commercially available or may be prepared by routine methods known in the art.

In the preparation of the compounds of the present invention, it is noted that some of the preparation methods described herein may require protection of remote functionality. The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see T. W. Greene, Protective Groups in Organic Synthesis, John Wiley & Sons, New York, 1991.

The Schemes described below are intended to provide a general description of the methodology employed in the preparation of the compounds of the present invention. Some of the compounds of the present invention may contain single or multiple chiral centers with the stereochemical designation (R) or (S). It will be apparent to one skilled in the art that all of the synthetic transformations can be conducted in a similar manner whether the materials are enantioenriched or racemic. Moreover, the resolution to the desired optically active material may take place at any desired point in the sequence using well known methods such as described herein and in the chemistry literature.

### EXAMPLES

In order that the invention described herein may be more fully understood, the following examples are set forth. The examples described in this application are offered to illustrate the methods and compositions provided herein and are not to be construed in any way as limiting their scope.

All reagents and materials are purchased from commercial vendors or may be readily prepared by those skilled in the art. A list of abbreviations for reagents used may be found in Table 1, below.

TABLE 1
Abbreviations of reagents or organic Moieties
Reagents or Organic Moieties

| Abbreviation | Full Name |
|---|---|
| AC₂O | 1,1′-(chlorophenylmethylene)bis[4-methoxybenzene] |
| AIBN | azodiisobutyronitrile |
| Bn | benzyl |
| Boc₂O | di-tert-butyl dicarbonate |
| DCM | dichloromethane |
| DEAD | diethyl azodicarboxylate |
| DIAD | diisopropyl azodicarbo |
| DIBAL-H | diisobutylaluminum hydride |
| DIEA | n,n-diisopropylethylamine |
| DMAP | 4-(dimethylamino)pyridine |
| DMF | dimethylformamide |
| DMSO | dimethyl sulfoxide |
| DMTr | 4,4′-dimethoxytriphenylmethyl |
| DMTrCl | 4,4′-dimethoxytriphenylmethyl chloride |
| EDC/EDCI | 1-(3-dimethylaminopropyl)-3-ethylcarbodiimide |
| Et | ethyl |
| HATU | 1-[bis(dimethylamino)methylene]-1h-1,2,3-triazolo[4,5- |
| | b]pyridinium 3-oxide hexafluorophosphate |
| HOBT | 1-hydroxybenzotriazole |
| i-Bu | iso-butyl |
| LDA | lithium diisopropylamide |
| NMP | 1-methyl-2-pyrrolidone |
| Ph | phenyl |
| p-TsOH | p-toluenesulfonic acid |
| Py | pyridine |
| TBA | tert-butyl alcohol |
| TBAF | tetrabutylammonium fluoride |
| TBAHS | tetrabutylammonium hydrogen sulfate |
| TBAI | tetrabutylammonium iodide |
| TBDPS | tert-butyldiphenylsilyl |
| TBDPSCl | tert-butyldiphenylchlorosilane |
| TBS | tert-butyldimethylsilyl |
| t-Bu | tert-butyl |
| Tf₂O | trifluoromethanesulfonic anhydride |
| TFA | trifluoroacetic acid |
| THF | tetrahydrofuran |
| TMSOTf | trimethylsilyl trifluoromethylsulfonate |
| TsCl | 4-Methylbenzenesulfonyl chloride |

### Example 1

### (3R,5S)-5-((bis(4-methoxyphenyl)(phenyl)methoxy)methyl)pyrrolidin-3-ol (Intermediate I-A)

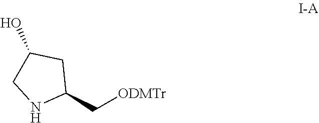

The title compound may be synthesized according to the synthetic route below.

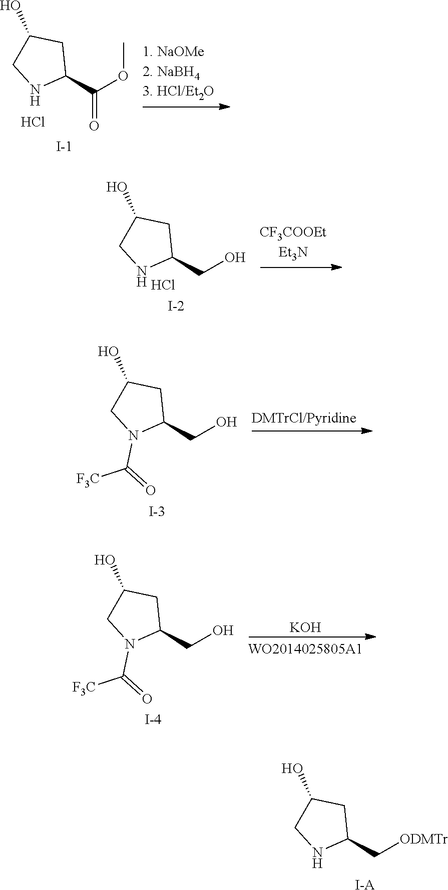

### 12-((2S,4R)-2-((bis(4-methoxyphenyl)(phenyl)methoxy)methyl)-4-hydroxypyrrolidin-1-yl)-12-oxododecanoic acid (Intermediate I-B)

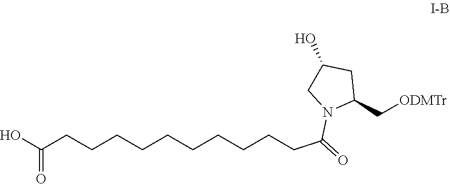

The title compound may be synthesized according to the synthetic route below.

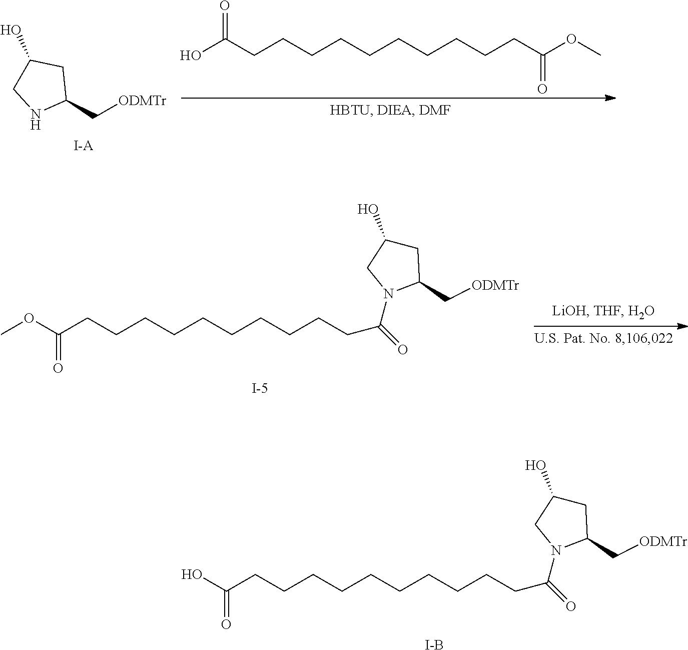

### Example 2

### 5-(((2R,3R,4R,5R,6R)-3-acetamido-4,5-diacetoxy-6-(acetoxymethyl)tetrahydro-2H-pyran-2-yl)oxy)pentanoic acid (Intermediate II-A)

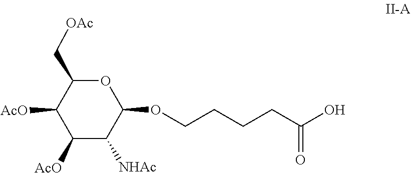 [SMILES: CC(=O)N[C@H]1[C@H](OCCCCC(=O)O)O[C@H](COC(C)=O)[C@H](OC(C)=O)[C@@H]1OC(C)=O]

The title compound may be synthesized according to the synthetic route below.

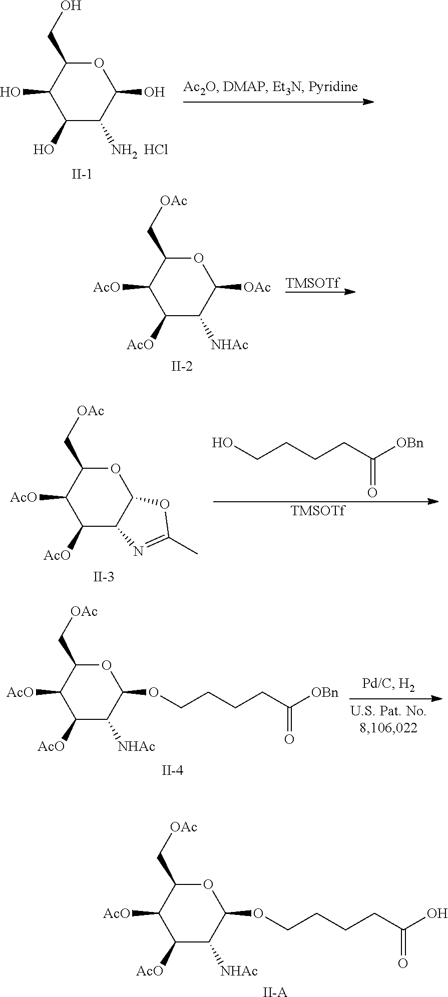

### 5-(((2R,3R,4R,5R,6R)-3-acetamido-4,5-bis(benzoyloxy)-6-((benzoyloxy)methyl)tetrahydro-2H-pyran-2-yl)oxy)pentanoic acid (Intermediate I-B)

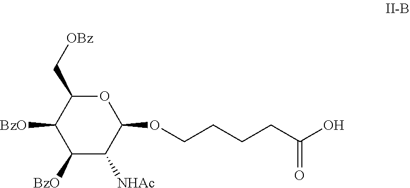

The title compound may be synthesized according to the synthetic route below.

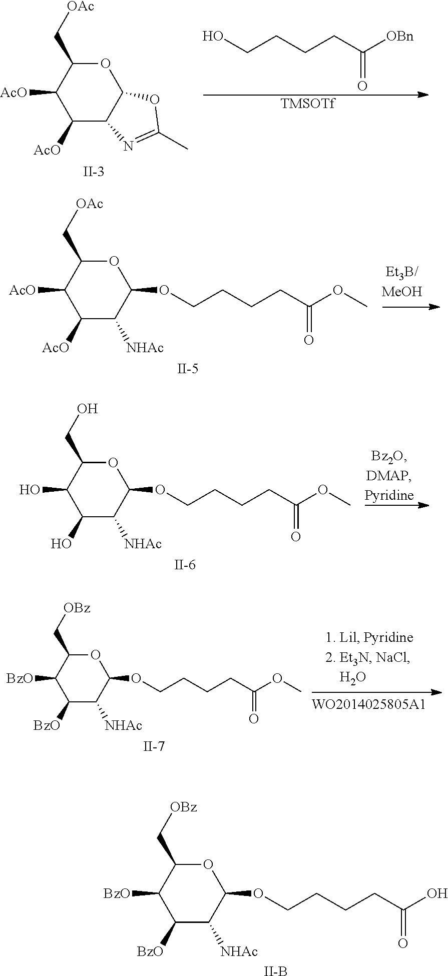

### Example 3

### (2R,3S,4R,5S)-2-(hydroxymethyl)tetrahydro-2H-pyran-3,4,5-triol (Intermediate III)

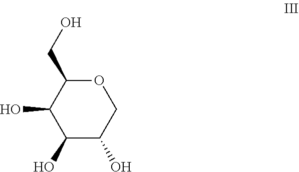 [SMILES: OC[C@H]1OC[C@H](O)[C@@H](O)[C@H]1O]

The title compound may be synthesized according to the synthetic route below.

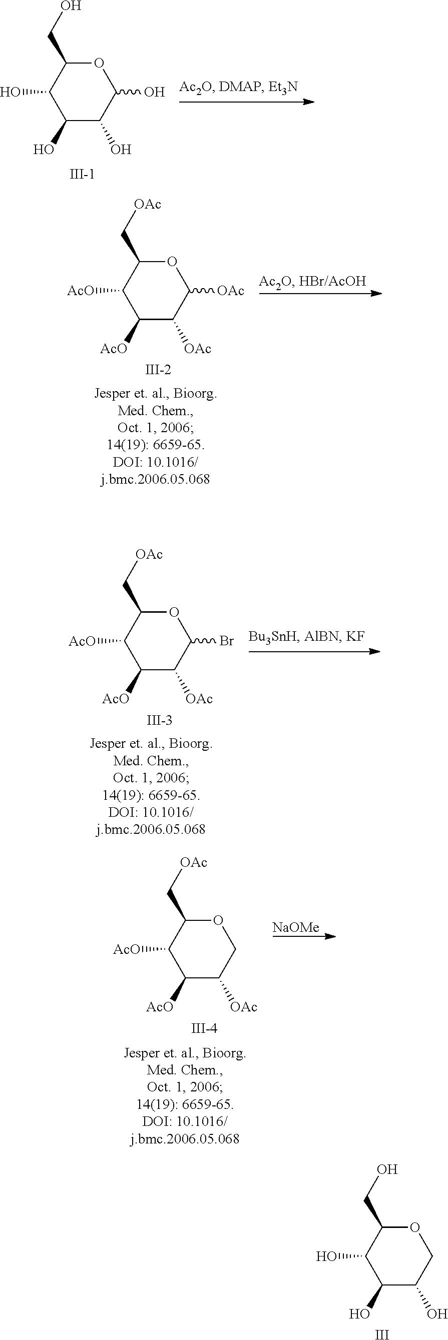

### Example 4

### (2R,3S,4R,5S)-5-amino-2-(hydroxymethyl)tetrahydro-2H-pyran-3,4-diol hydrochloride (Intermediate IV)

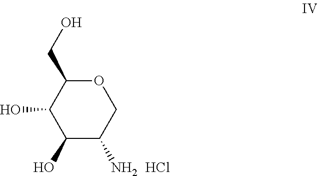 [SMILES: N[C@H]1CO[C@H](CO)[C@@H](O)[C@@H]1O ; Cl]

The title compound was synthesized according to the synthetic route below.

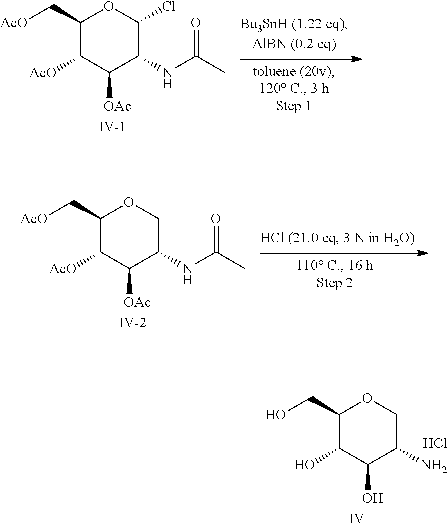

Step 1. (2R,3S,4R,5R,6R)-5-acetamido-2-(acetoxymethyl)-6-chlorotetrahydro-2H-pyran-3,4-diyl diacetate (IV-1) (40.0 g, 109 mmol, 1.00 eq) was added to anhydrous toluene (800 mL), (n-Bu)₃SnH (39.0 g, 134 mmol, 35.4 mL, 1.22 eq) was added, then AIBN (3.59 g, 21.9 mmol, 0.20 eq) was added at 15° C. The resulting mixture was stirred 120° C. for 3 h under N₂. TLC (Petroleum ether/EtOAc=1/2 (PMA)) indicated IV-1 was consumed completely and LCMS indicated desired product MS. After the reaction was completed, the mixture was cooled to 20° C., concentrated under vacuum at 45° C. The crude product was triturated with (i-Pr)₂O (200 mL) and toluene (50 mL) at 15° C. for 2 h, the solid was collected to provide (2R,3S,4R,5S)-5-acetamido-2-(acetoxymethyl)tetrahydro-2H-pyran-3,4-diyl diacetate (IV-2) (35.2 g). LCMS: calcd for [M+H]: 332.1, found: 332.2. ¹H NMR: (400 MHz CDCl₃) δ 5.93 (d, J=7.6 Hz, 1H), 5.06-4.99 (m, 1H), 4.98-4.90 (m, 1H), 4.23-4.05 (m, 4H), 3.59-3.48 (m, 1H), 3.22-3.08 (m, 1H), 2.05 (s, 3H), 2.02 (s, 3H), 2.00 (s, 3H), 1.90 (s, 3H).

Step 2. A mixture of compound IV-2 (39.0 g, 118 mmol, 1.00 eq) in aqueous HCl (3.00 M, 825 mL, 21.0 eq) was stirred at 110° C. for 16 h. TLC (Petroleum ether/EtOAc=0/1 (PMA) indicated compound IV-2 was consumed completely and LCMS indicated desired product MS. The brown solution was extracted with EtOAc (300 mL×3), the aqueous layer was concentrated under reduced pressure to give a residue at 45° C., co-evaporated with ACN (300 mL×3) and toluene (300 mL×3) to remove H₂O/HCl at 50° C. (2R,3S,4R,5S)-5-amino-2-(hydroxymethyl)tetrahydro-2H-pyran-3,4-diol hydrochloride (Intermediate IV) (23.6 g, crude) was obtained as a white solid and used directly in the next step without further purification. LCMS: calcd for [M+H]: 164.08, found: 164.2. ¹H NMR: (400 MHz D₂O) δ 4.19 (dd, J=4.8, 11.2 Hz, 1H), 3.90 (dd, J=2.0, 12.4 Hz, 1H), 3.72 (dd, J=5.2, 12.4 Hz, 1H), 3.65 (dd, J=8.4, 10.0 Hz, 1H), 3.56 (t, J=11.2 Hz, 1H), 3.49-3.38 (m, 2H), 3.35-3.25 (m, 1H).

### Example 5

### (2R,3R,4R)-2-(hydroxymethyl)piperidine-3,4-diol (Intermediate V)

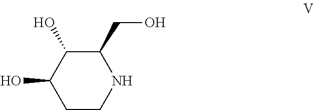

The title compound may be synthesized according to the synthetic route below.

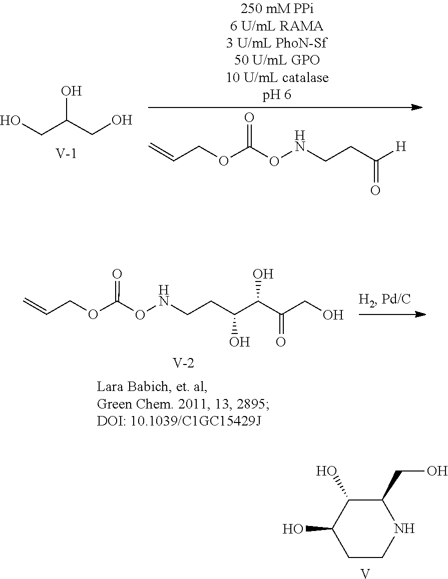

### Example 6

### (3S,4S,5R,Z)-1,2,3,4,5,8-hexahydroazocine-3,4,5-triol (Intermediate VI)

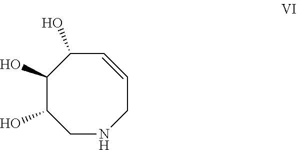 [SMILES: O[C@H]1[C@H](O)/C=C\CNC[C@@H]1O]

The title compound may be synthesized according to the synthetic route below.

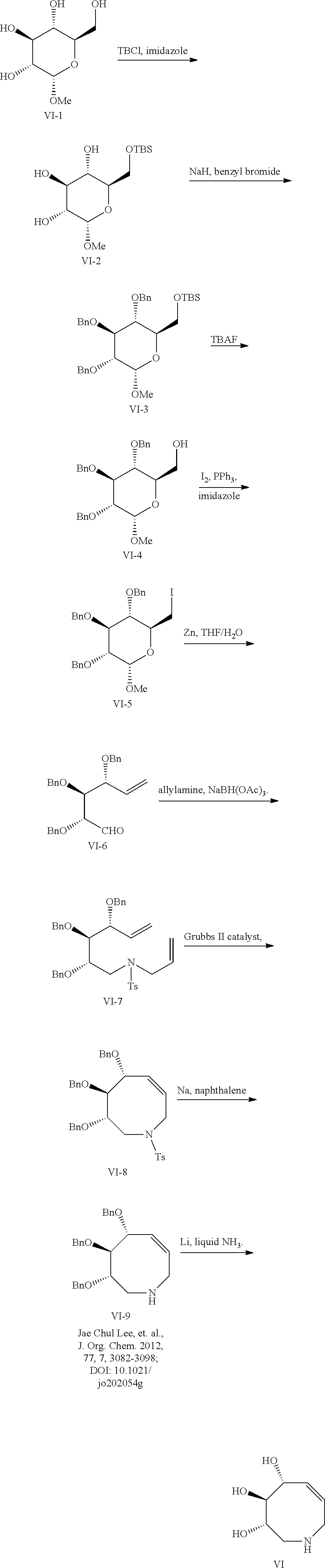

### Example 7

### (3S,4S,5R)-azocane-3,4,5-triol (Intermediate VII)

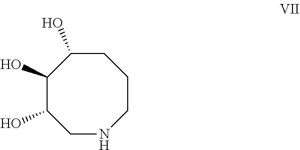 [SMILES: O[C@H]1[C@H](O)CCCNC[C@@H]1O]

The title compound may be synthesized according to the synthetic route below.

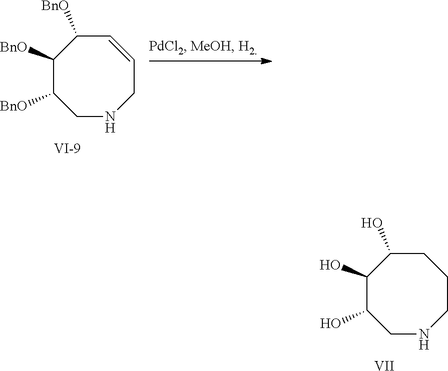

### Example 8

### (3R,4r,5S)-piperidine-3,4,5-triol (Intermediate VIII)

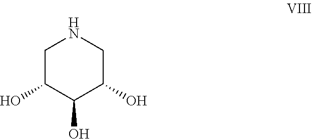

The title compound may be synthesized according to the synthetic route below.

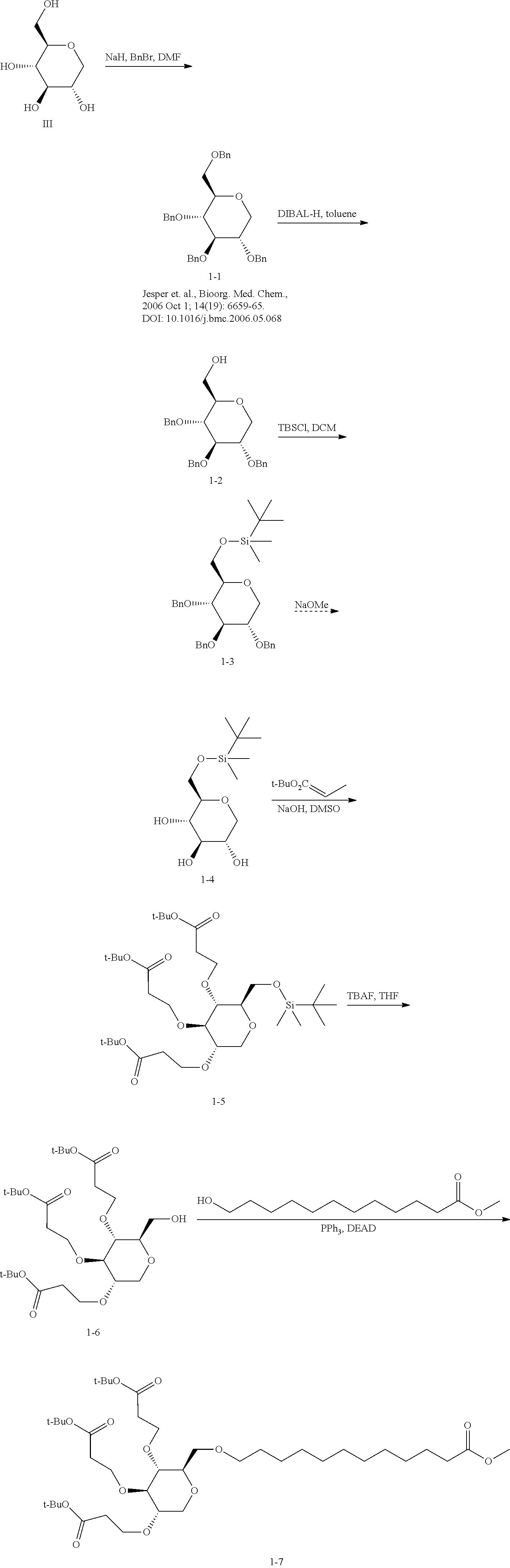

### Example 9

### tri-tert-butyl 3,3′,3″-(((2R,3R,4R,5S)-2-(((12-methoxy-12-oxododecyl)oxy)methyl)tetrahydro-2H-pyran-3,4,5-triyl)tris(oxy))tripropionate (Intermediate 1-7)

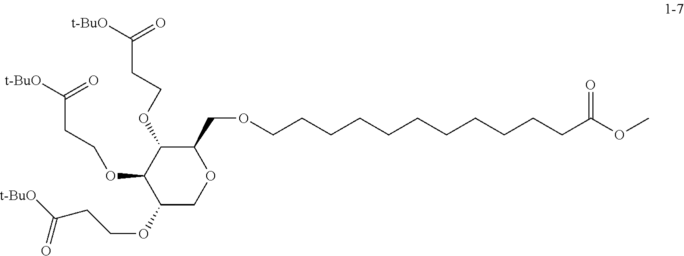 [SMILES: COC(=O)CCCCCCCCCCCOC[C@H]1OC[C@H](OCCC(=O)OC(C)(C)C)[C@@H](OCCC(=O)OC(C)(C)C)[C@@H]1OCCC(=O)OC(C)(C)C]

The title compound may be synthesized according to the synthetic route below.

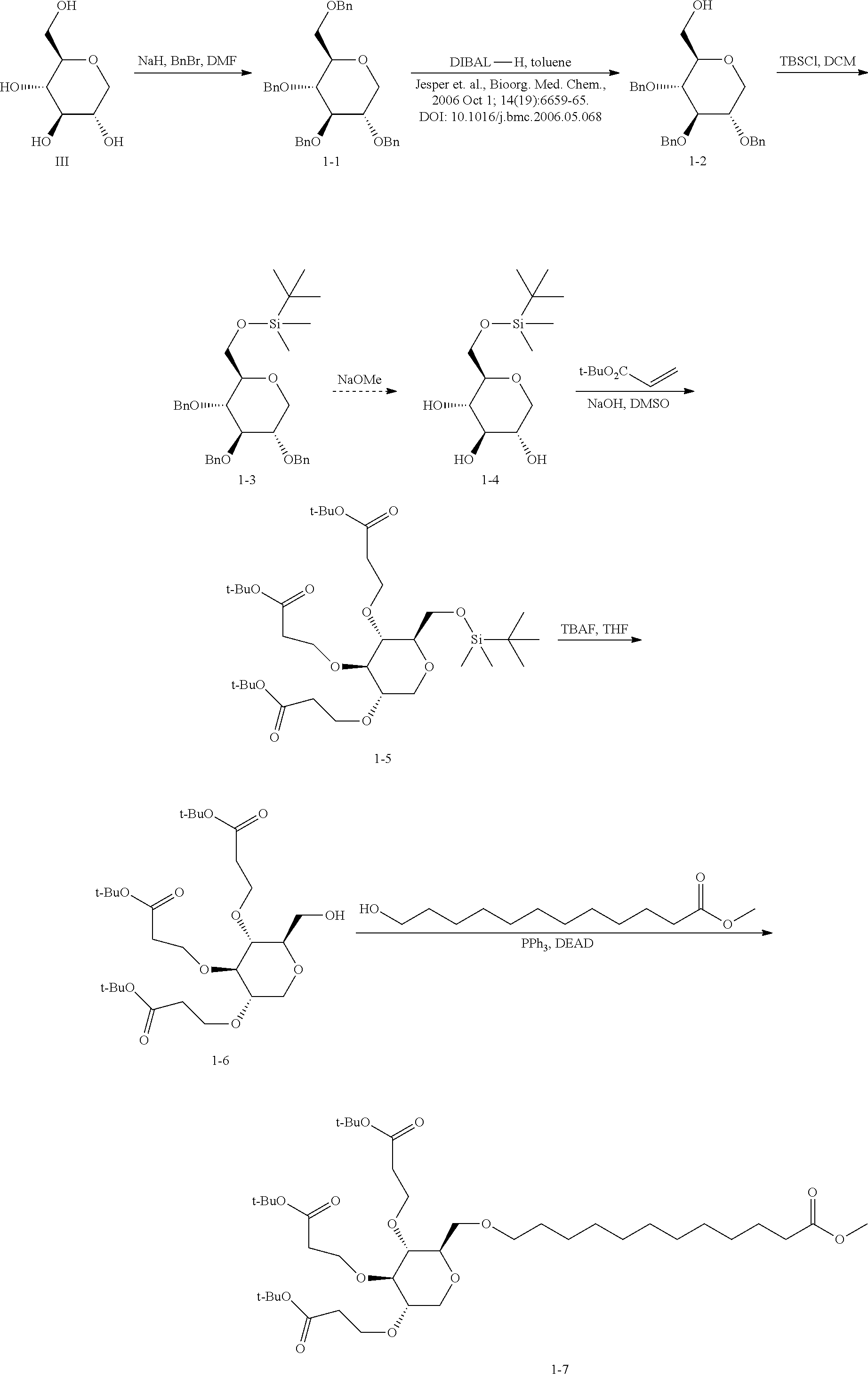

### Example 10

### tri-tert-butyl 3,3′,3″-(((2R,3R,4R,5S)-2-((((benzyloxy)carbonyl)amino)methyl)tetrahydro-2H-pyran-3,4,5-triyl)tris(oxy))tripropionate (Intermediate 2-3)

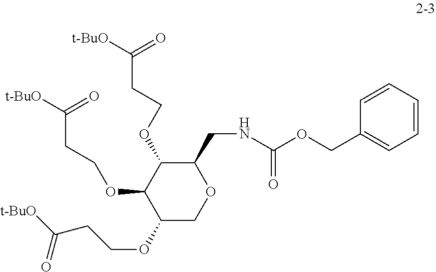 [SMILES: CC(C)(C)OC(=O)CCO[C@H]1[C@H](OCCC(=O)OC(C)(C)C)[C@@H](CNC(=O)OCc2ccccc2)OC[C@@H]1OCCC(=O)OC(C)(C)C]

The title compound may be synthesized according to the synthetic route below.

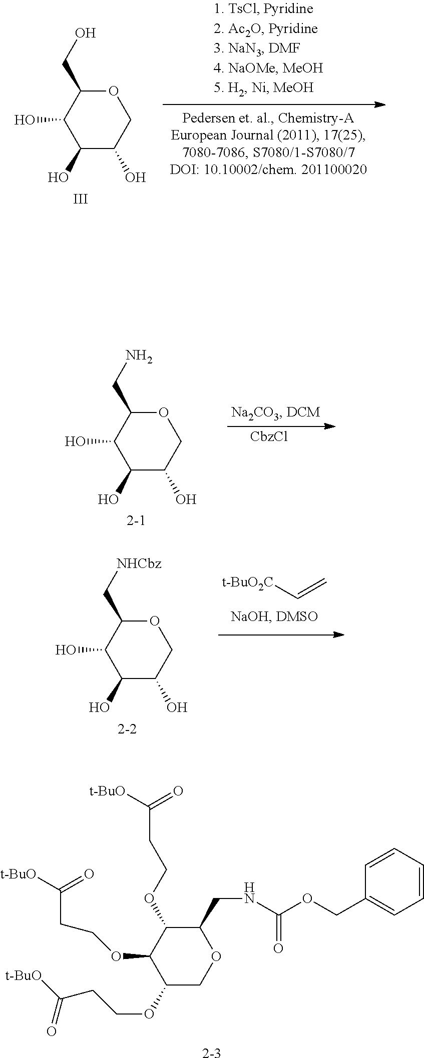

### Example 11

### tri-tert-butyl 3,3′,3″-(((2R,3R,4R,5S)-2-((2-(((benzyloxy)carbonyl)amino)ethoxy)methyl)tetrahydro-2H-pyran-3,4,5-triyl)tris(oxy))tripropionate (Intermediate 3-4)

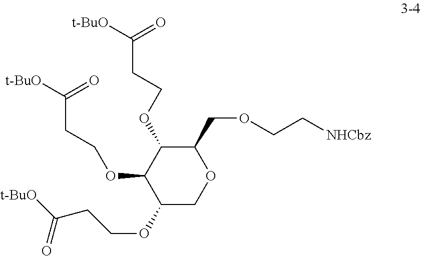 [SMILES: CC(C)(C)OC(=O)CCO[C@H]1[C@H](OCCC(=O)OC(C)(C)C)[C@@H](COCCNC(=O)OCc2ccccc2)OC[C@@H]1OCCC(=O)OC(C)(C)C]

The title compound may be synthesized according to the synthetic route below.

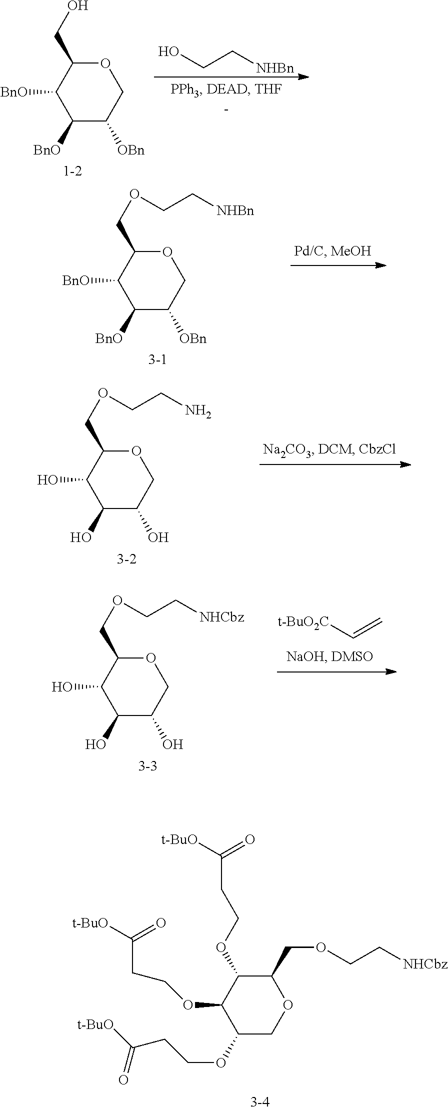

### Example 12

### tri-tert-butyl 3,3′,3″-(((2R,3R,4R,5S)-2-(((2-(((benzyloxy)carbonyl)amino)ethyl)amino)methyl)tetrahydro-2H-pyran-3,4,5-triyl)tris(oxy))tripropionate (Intermediate 4-4)

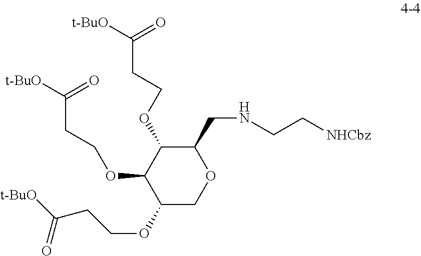 [SMILES: CC(C)(C)OC(=O)CCO[C@H]1[C@H](OCCC(=O)OC(C)(C)C)[C@@H](CNCCNC(=O)OCc2ccccc2)OC[C@@H]1OCCC(=O)OC(C)(C)C]

The title compound may be synthesized according to the synthetic route below.

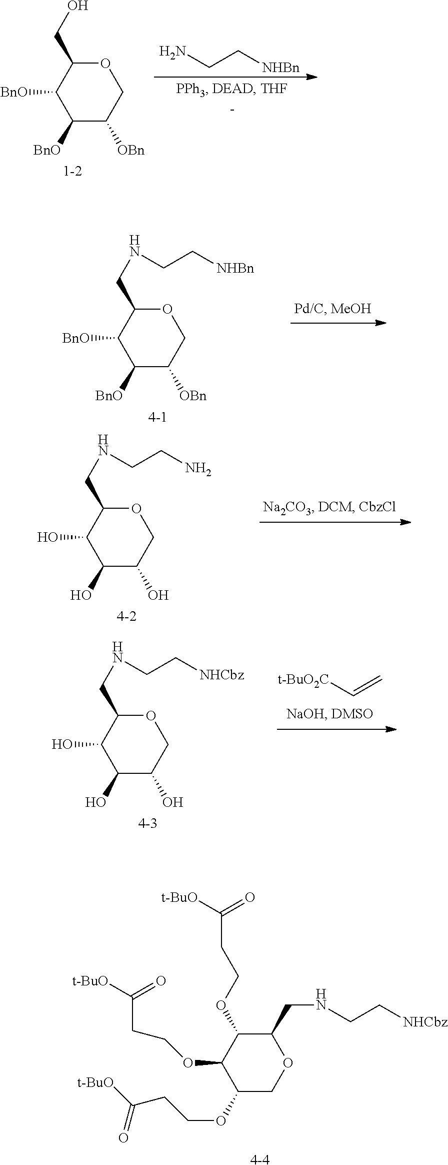

### Example 13

### di-tert-butyl 3,3′-(((2R,3S,4R,5S)-2-((3-(tert-butoxy)-3-oxopropoxy)methyl)-5-(12-methoxy-12-oxododecanamido)tetrahydro-2H-pyran-3,4-diyl)bis(oxy))dipropionate (Intermediate 5-4)

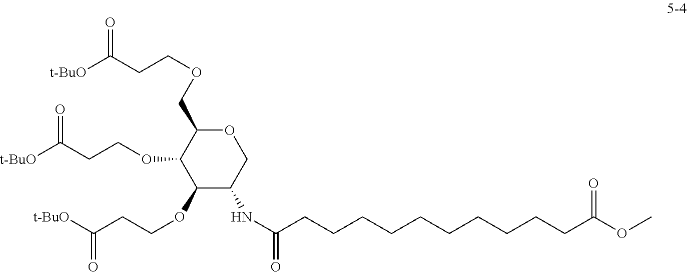 [SMILES: COC(=O)CCCCCCCCCCC(=O)N[C@H]1CO[C@H](COCCC(=O)OC(C)(C)C)[C@@H](OCCC(=O)OC(C)(C)C)[C@@H]1OCCC(=O)OC(C)(C)C]

The title compound may be synthesized according to the synthetic route below.

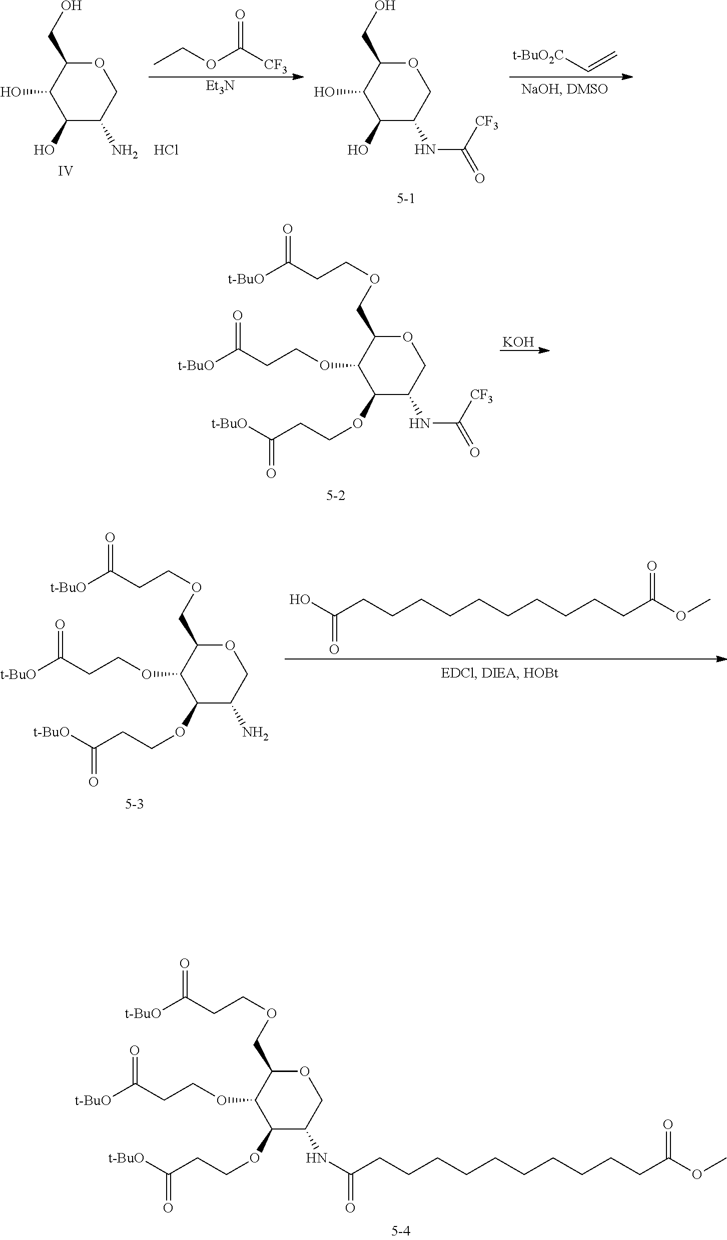

### Example 14

### di-tert-butyl 3,3′-(((2R,3S,4R,5R,6R)-5-acetamido-2-((3-(tert-butoxy)-3-oxopropoxy)methyl)-6-(12-methoxy-12-oxododecanamido)tetrahydro-2H-pyran-3,4-diyl)bis(oxy))dipropionate (Intermediate 6-4)

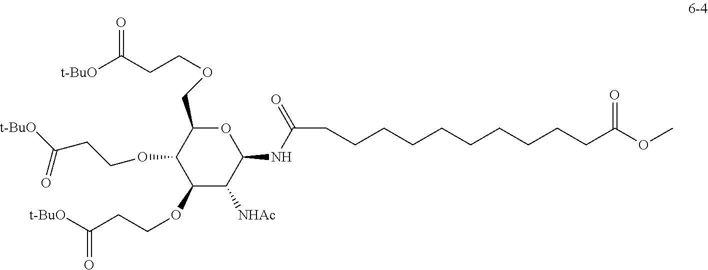 [SMILES: COC(=O)CCCCCCCCCCC(=O)N[C@@H]1O[C@H](COCCC(=O)OC(C)(C)C)[C@@H](OCCC(=O)OC(C)(C)C)[C@H](OCCC(=O)OC(C)(C)C)[C@H]1NC(C)=O]

The title compound may be synthesized according to the synthetic route below.

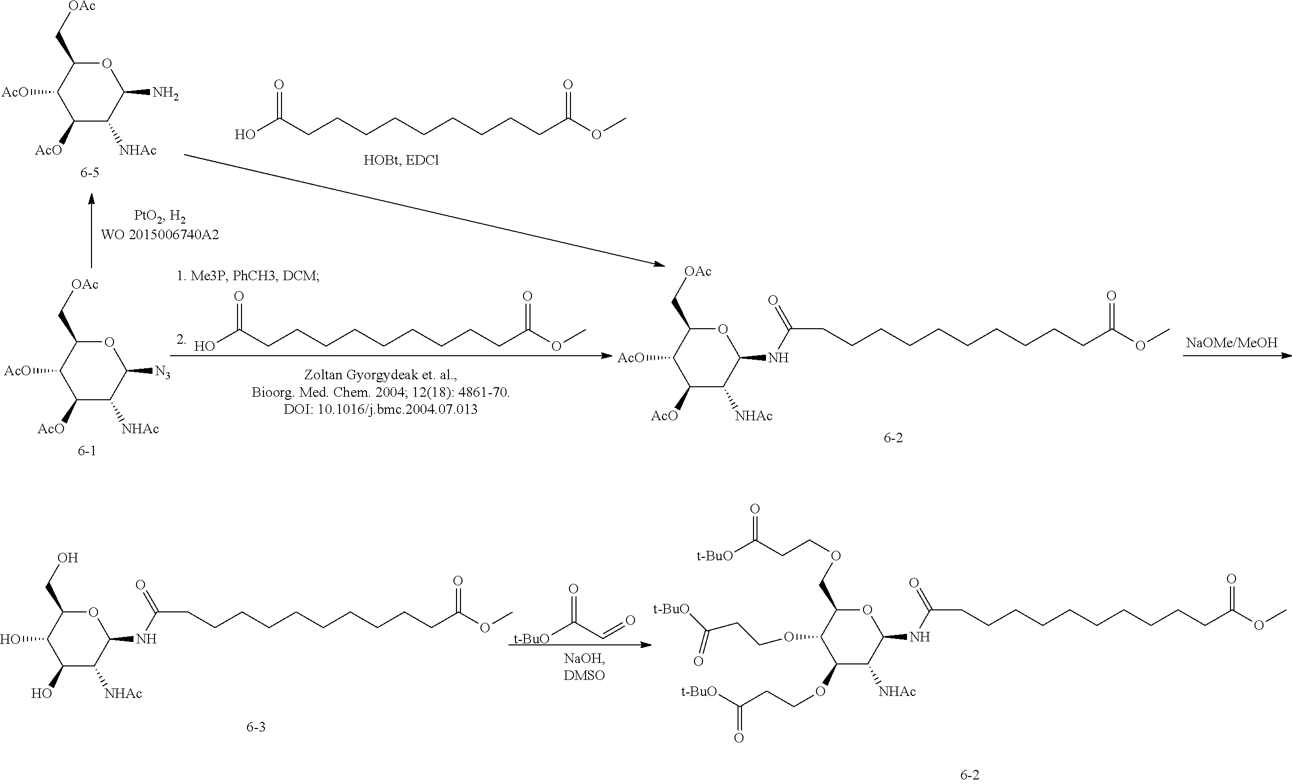

### Example 15

### di-tert-butyl 3,3′-(((2R,3R,4S,5R,6R)-2-((3-(tert-butoxy)-3-oxopropoxy)methyl)-5-fluoro-6-(12-methoxy-12-oxododecanamido)tetrahydro-2H-pyran-3,4-diyl)bis(oxy))dipropionate (Intermediate 7-8)

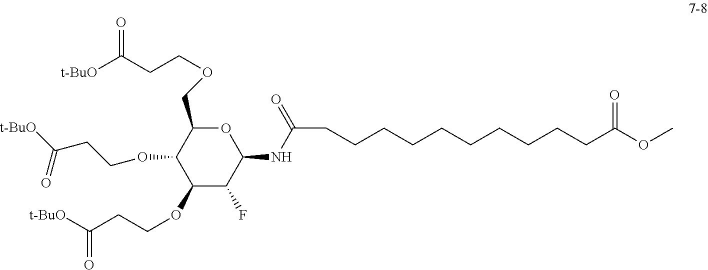 [SMILES: COC(=O)CCCCCCCCCCC(=O)N[C@@H]1O[C@H](COCCC(=O)OC(C)(C)C)[C@@H](OCCC(=O)OC(C)(C)C)[C@H](OCCC(=O)OC(C)(C)C)[C@H]1F]

The title compound may be synthesized according to the synthetic route below.

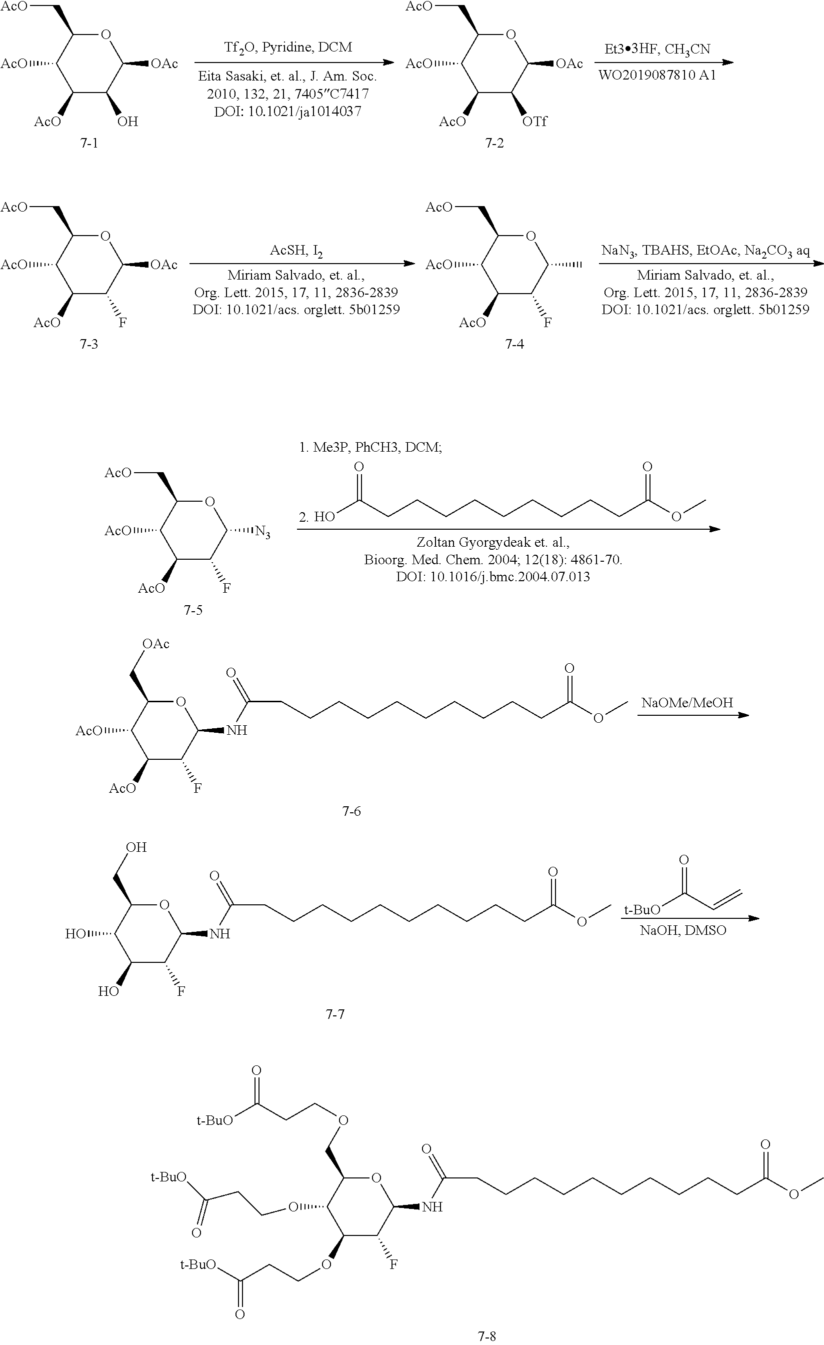

### Example 16

### di-tert-butyl 3,3′-(((2R,3S,4R,5R,6S)-2-((3-(tert-butoxy)-3-oxopropoxy)methyl)-6-((tert-butyldiphenylsilyl)oxy)-5-(12-methoxy-12-oxododecanamido)tetrahydro-2H-pyran-3,4-diyl)bis(oxy))dipropionate (Intermediate 8-10)

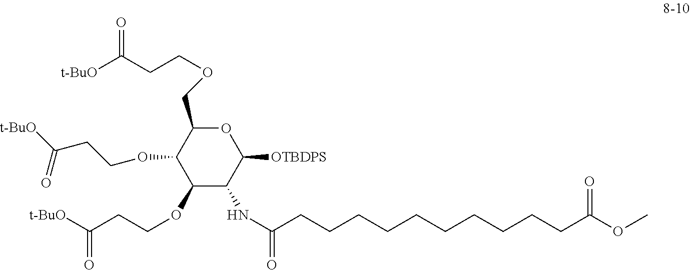

The title compound may be synthesized according to the synthetic route below.

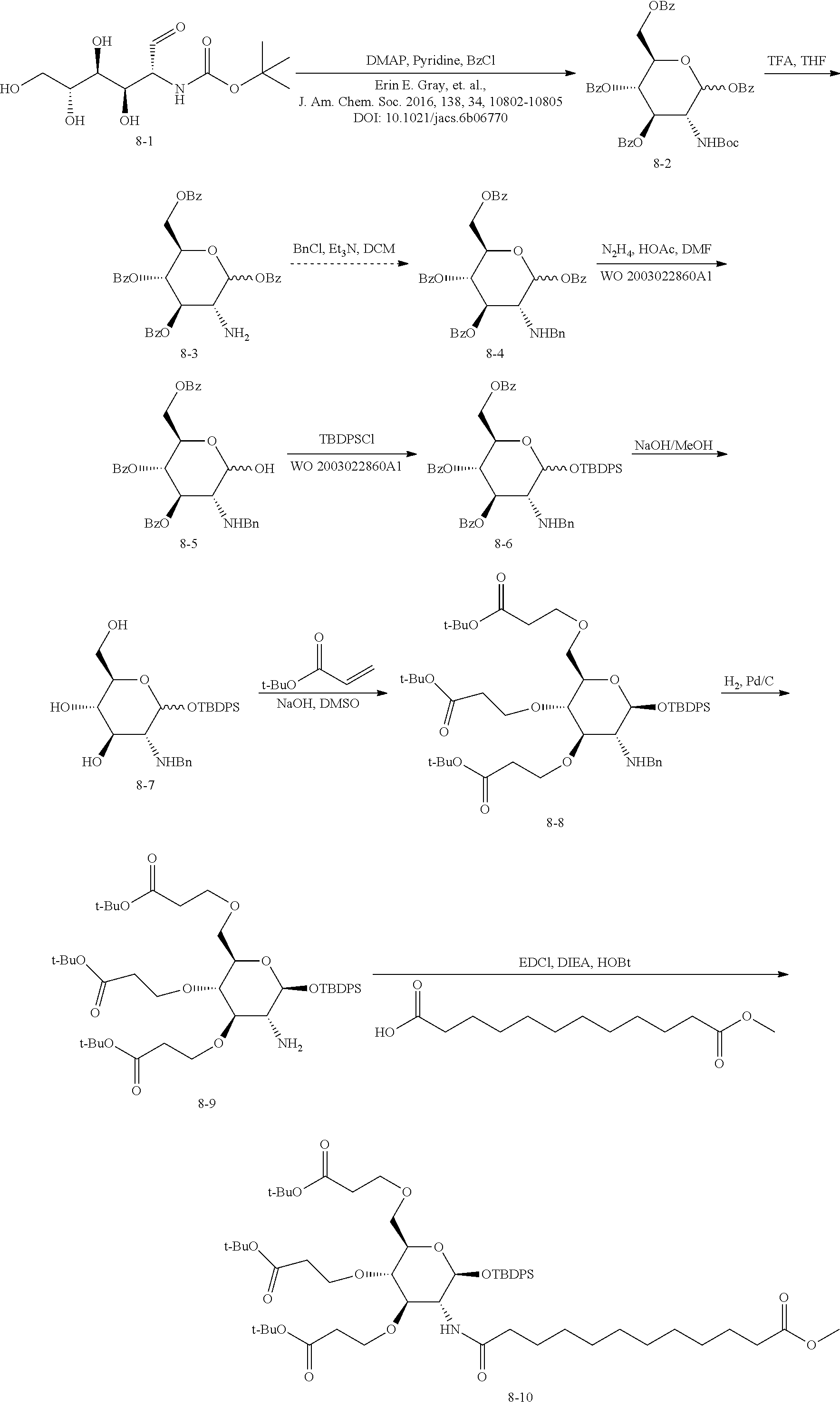

### Example 17

### di-tert-butyl 3,3′-(((2R,3R,4R)-1-((benzyloxy)carbonyl)-2-((3-(tert-butoxy)-3-oxopropoxy)methyl)piperidine-3,4-diyl)bis(oxy))dipropionate (Intermediate 9-2)

The title compound may be synthesized according to the synthetic route below.

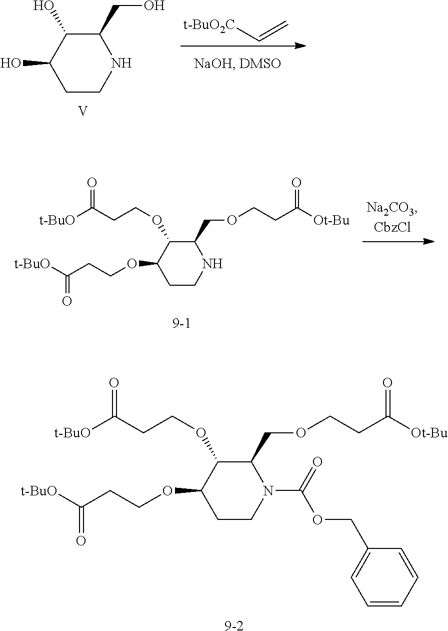

### Example 18

### tri-tert-butyl 3,3′,3″-(((3S,4S,5R,Z)-1-((benzyloxy)carbonyl)-1,2,3,4,5,8-hexahydroazocine-3,4,5-triyl)tris(oxy))tripropionate (Intermediate 10-2)

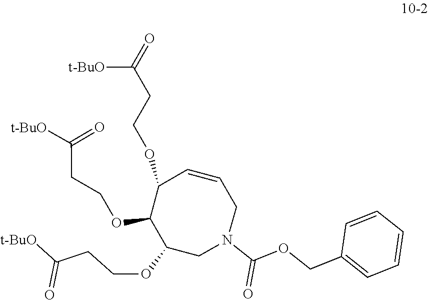 [SMILES: CC(C)(C)OC(=O)CCO[C@@H]1[C@@H](OCCC(=O)OC(C)(C)C)CN(C(=O)OCc2ccccc2)C/C=C\[C@H]1OCCC(=O)OC(C)(C)C]

The title compound may be synthesized according to the synthetic route below.

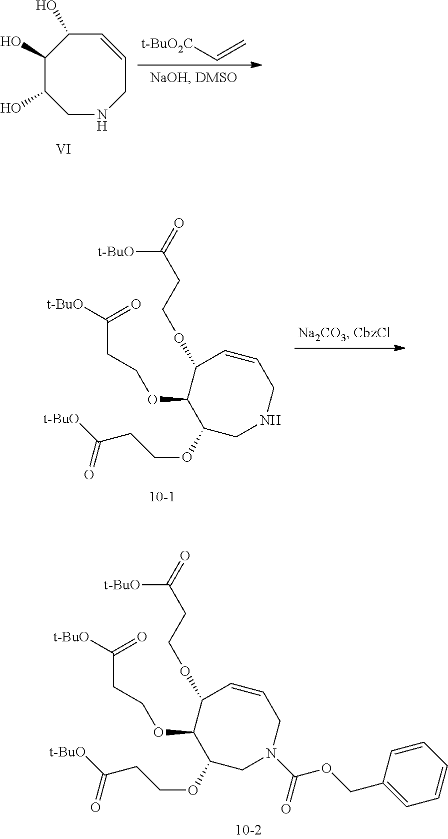

### Example 19

### tri-tert-butyl 3,3′,3″-(((3S,4S,5R)-1-((benzyloxy)carbonyl)azocane-3,4,5-triyl)tris(oxy))tripropionate (Intermediate 11-2)

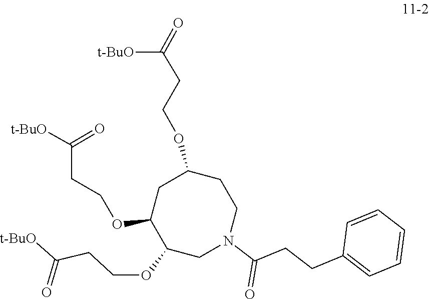 [SMILES: CC(C)(C)OC(=O)CCO[C@@H]1CCN(C(=O)CCc2ccccc2)C[C@H](OCCC(=O)OC(C)(C)C)[C@@H](OCCC(=O)OC(C)(C)C)C1]

The title compound may be synthesized according to the synthetic route below.

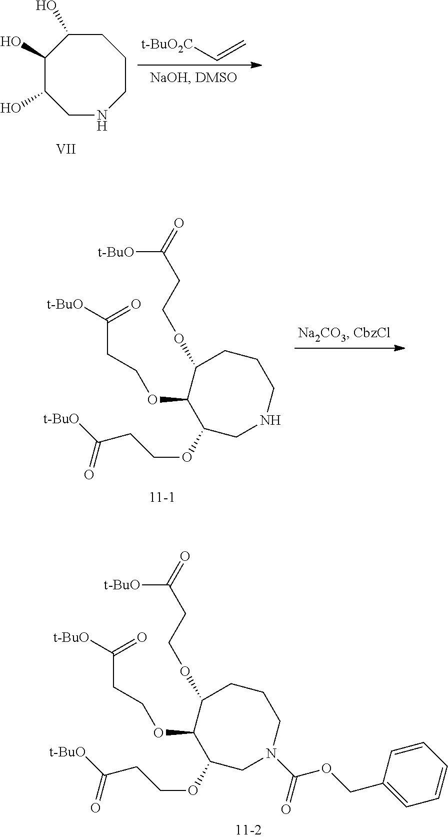

### Example 20

### tri-tert-butyl 3,3′,3″-(((3S,4r,5R)-1-((benzyloxy)carbonyl)piperidine-3,4,5-triyl)tris(oxy))tripropionate (Intermediate 12-2)

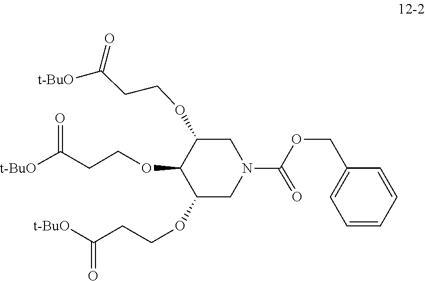 [SMILES: CC(C)(C)OC(=O)CCO[C@@H]1[C@@H](OCCC(=O)OC(C)(C)C)CN(C(=O)OCc2ccccc2)C[C@H]1OCCC(=O)OC(C)(C)C]

The title compound may be synthesized according to the synthetic route below.

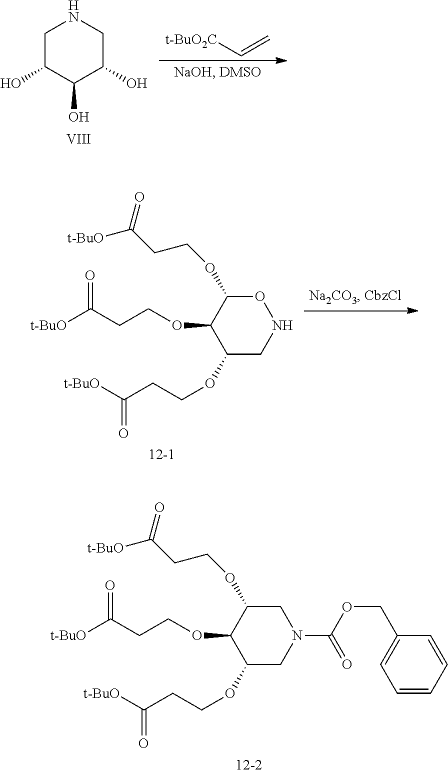

### Example 21

### Synthesis of Compound 1-11

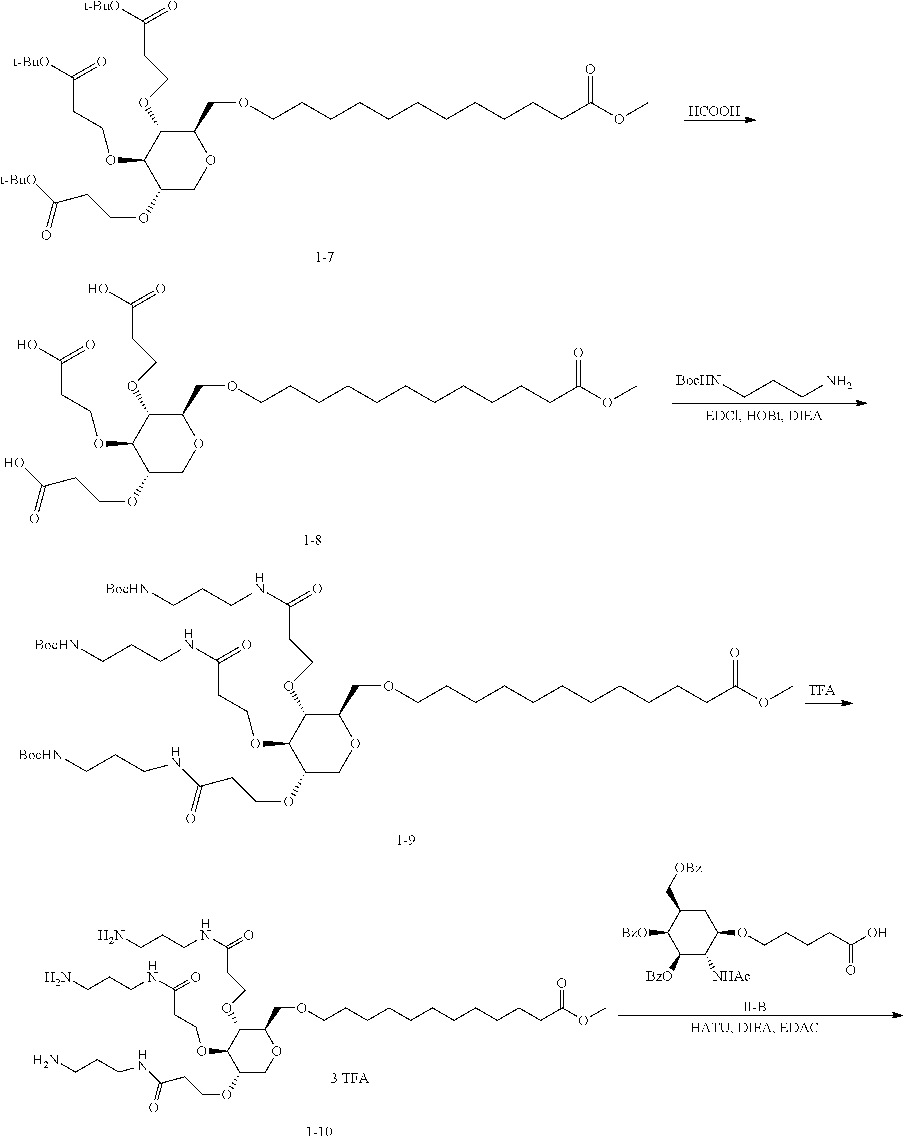

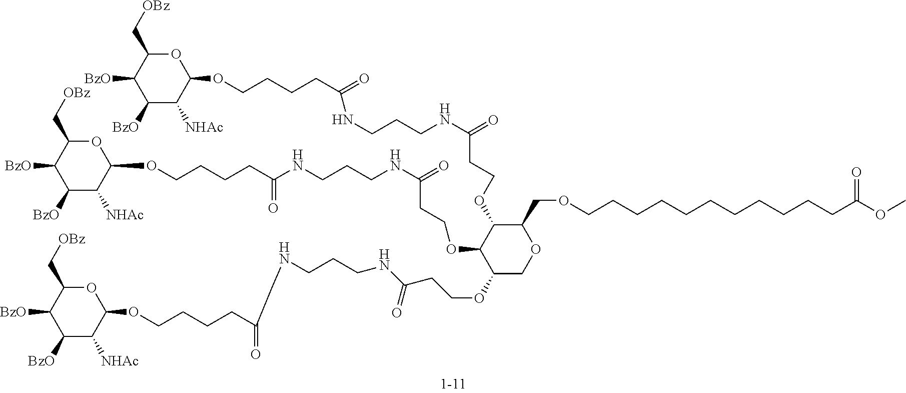

### Example 22

### Synthesis of Compound 1-14

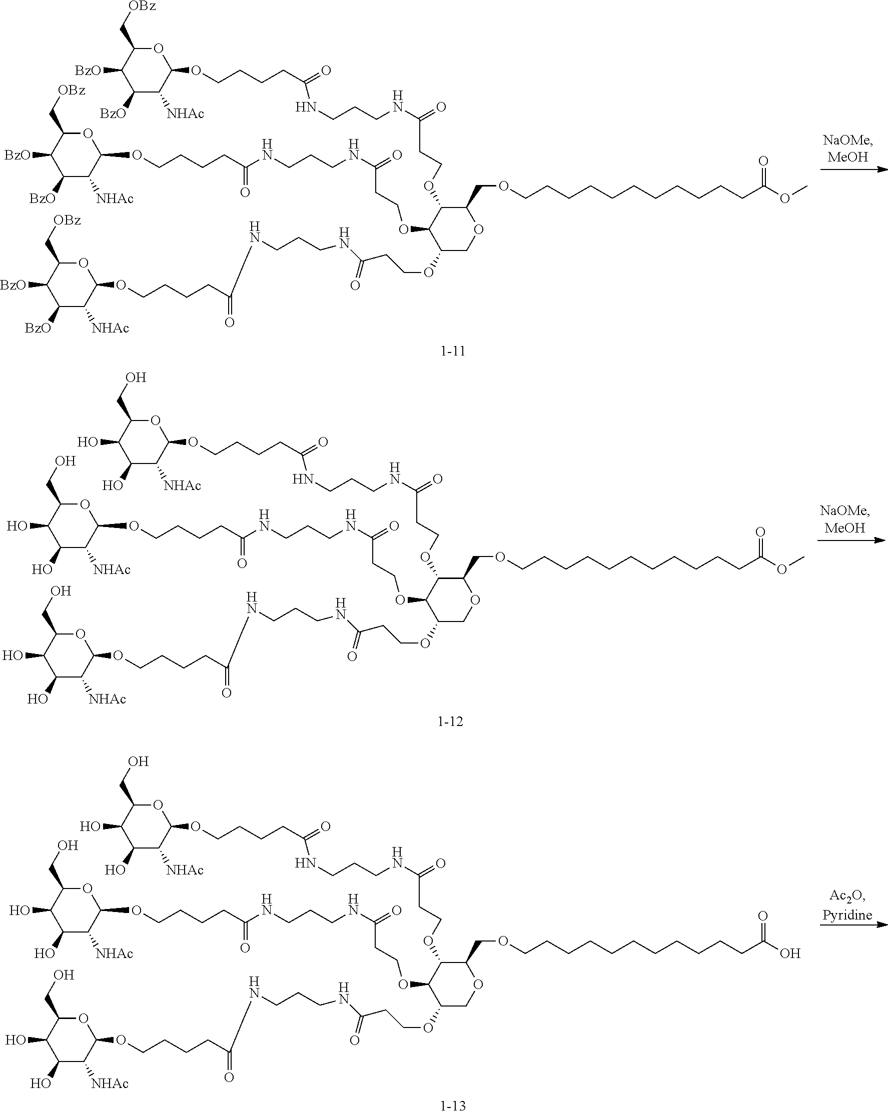

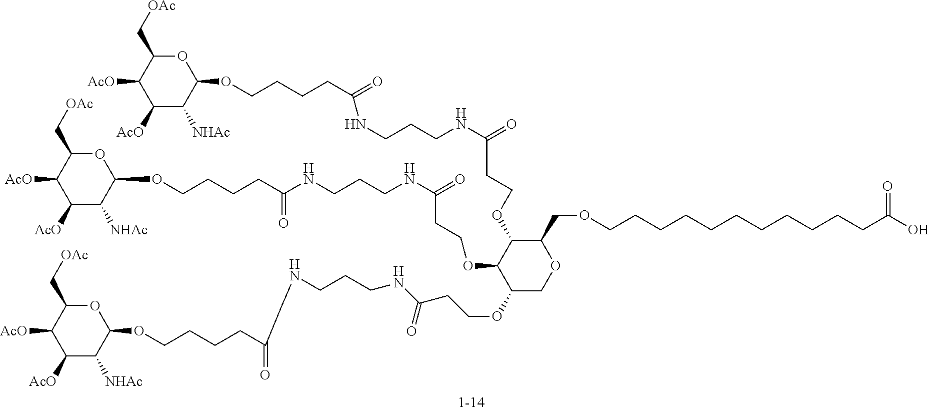 [SMILES: CC(=O)N[C@H]1[C@H](OCCCCC(=O)CNCCCNC(=O)CCO[C@H]2CO[C@H](COCCCCCCCCCCCC(=O)O)[C@@H](OCCC(=O)NCCCNC(=O)CCCCO[C@@H]3O[C@H](COC(C)=O)[C@H](OC(C)=O)[C@H](OC(C)=O)[C@H]3NC(C)=O)[C@@H]2OCCC(=O)NCCCNC(=O)CCCCO[C@@H]2O[C@H](COC(C)=O)[C@H](OC(C)=O)[C@H](OC(C)=O)[C@H]2NC(C)=O)O[C@H](COC(C)=O)[C@H](OC(C)=O)[C@@H]1OC(C)=O]

### Example 23

### Synthesis of Compound 1

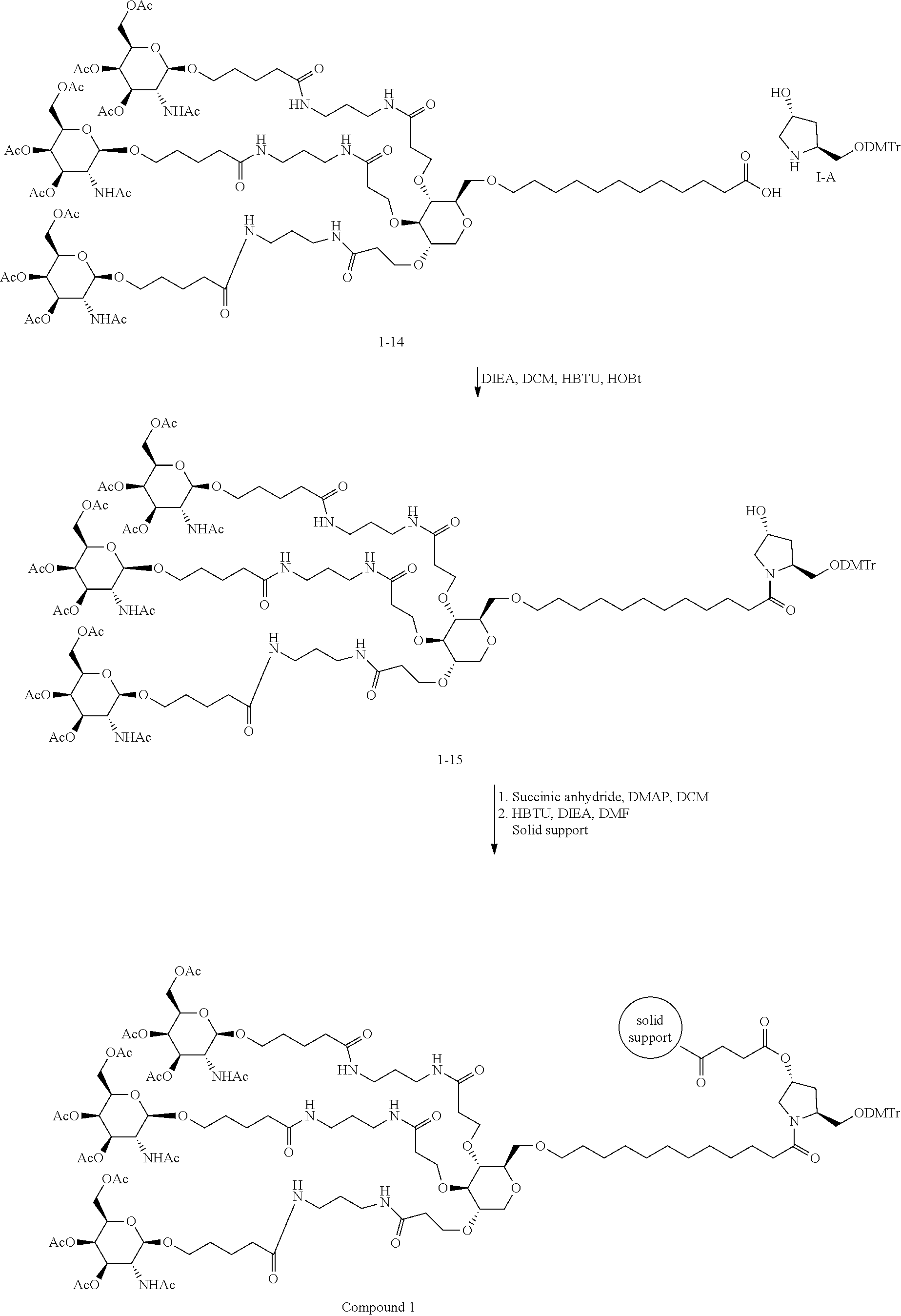

### Example 24

### Synthesis of Compound 2-8

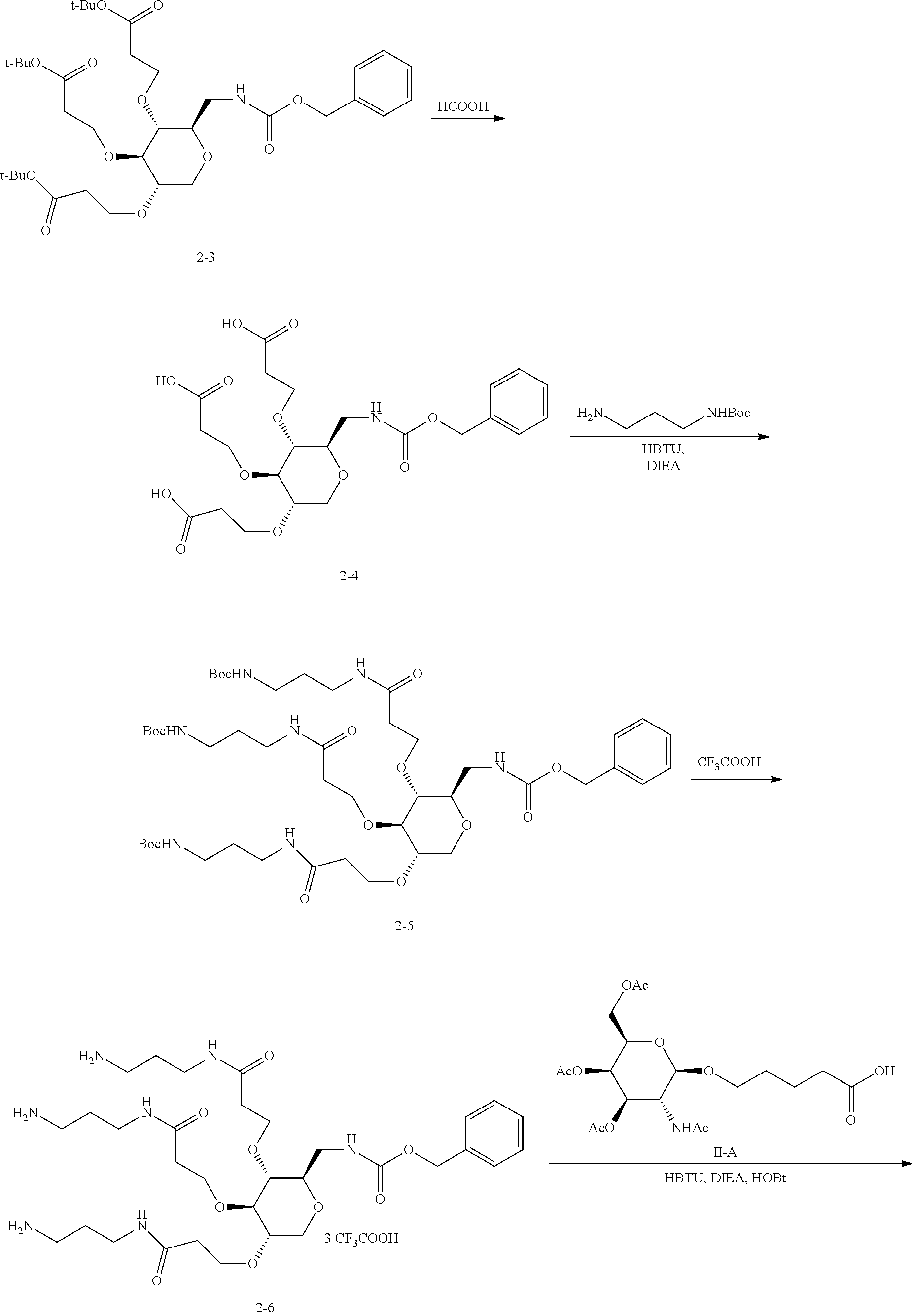

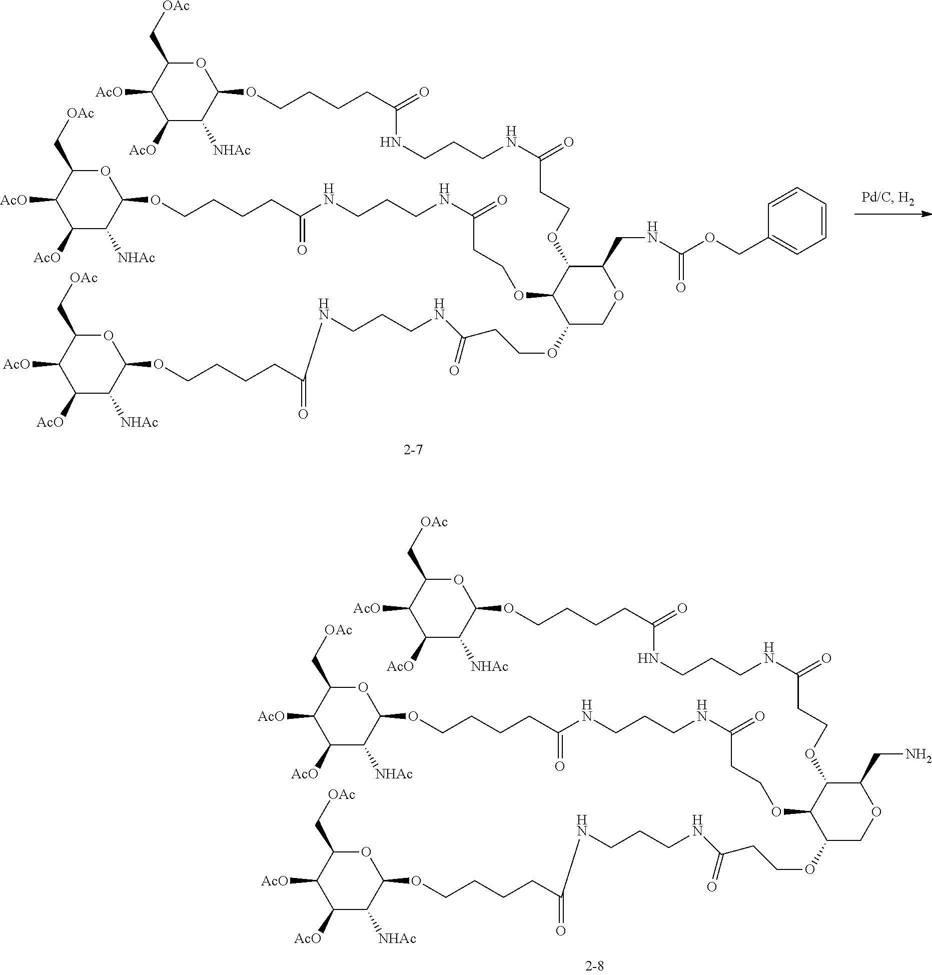

### Example 25

### Synthesis of Compound 2

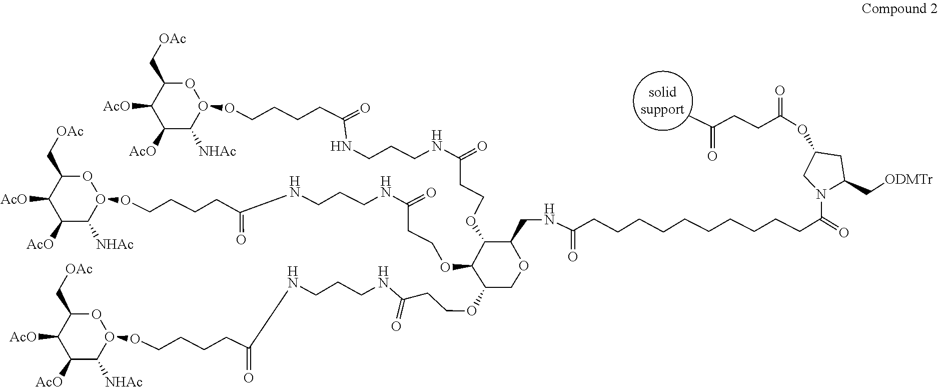

The title compound was synthesized according to the synthetic route below.

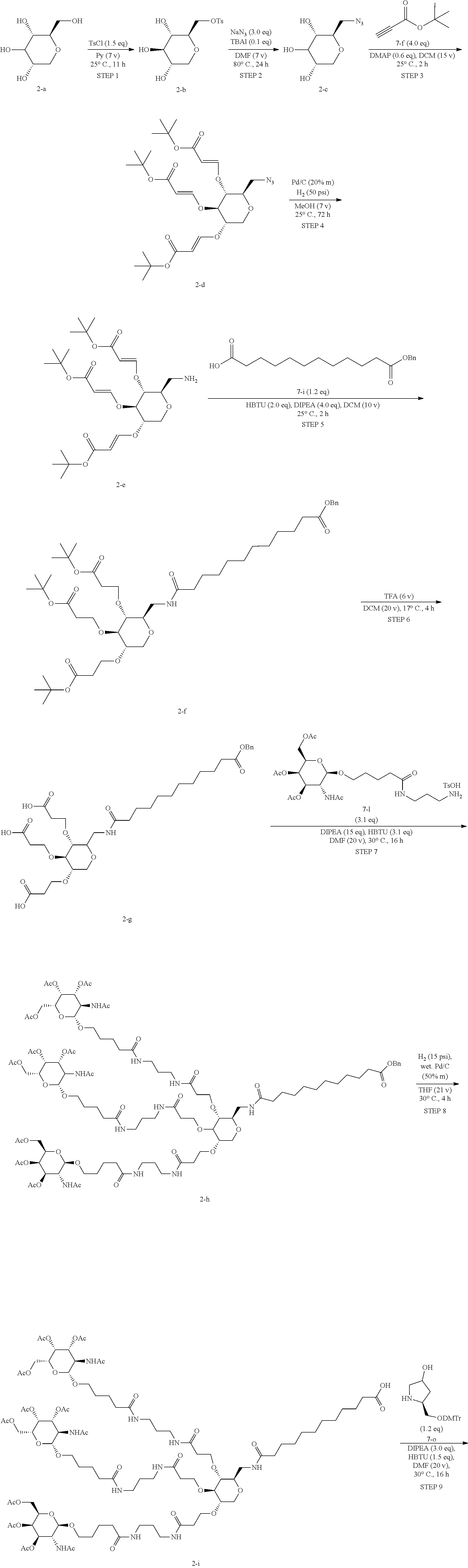

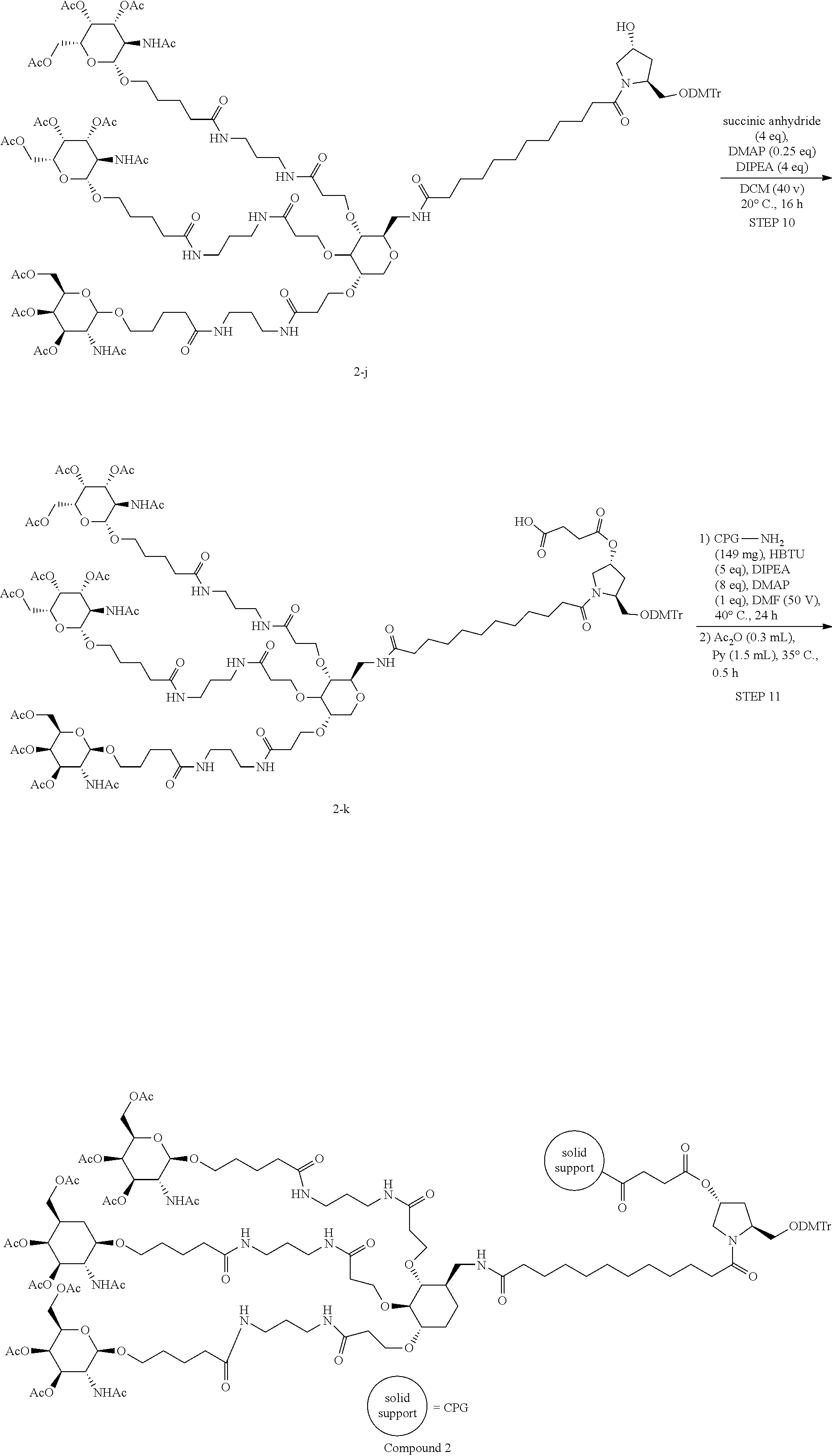

STEP 1: To a solution of Cpd. 2-a (7.50 g, 45.7 mmol, 1.00 eq) in Py (52.5 mL) was added TsCl (13.1 g, 68.5 mmol, 1.5 eq) at 0° C. The mixture was stirred at 25° C. for 11 h. LCMS (RT of Compound 5-1a=1.368 min) showed Cpd. 2-a was consumed and one main peak of desired mass. The reaction mixture was concentrated under reduced pressure to give a residue. The combined residue was purified by reversed-phase HPLC (neutral condition). Cpd. 2-b (13.6 g, 42.7 mmol, 46.7% yield) was obtained as a yellow solid. ¹H NMR (400 MHz CD₃OD) (Product) δ 7.78 (d, J=8.4 Hz, 2H), 7.44 (d, J=8.0 Hz, 2H), 4.29 (dd, J=2.0, 10.8 Hz, 2H), 4.17-4.05 (m, 1H), 3.85-3.75 (m, 1H), 3.42-3.33 (m, 1H), 3.30-3.00 (m, 4H), 2.45 (s, 3H).

STEP 2: To a solution of Cpd. 2-b (20.0 g, 62.8 mmol, 1.00 eq) in DMF (140 mL) was added NaN₃ (12.3 g, 188 mmol, 3.00 eq) and TBAI (2.32 g, 6.28 mmol, 0.1 eq). The mixture was stirred at 80° C. for 24 h. TLC (Petroleum ether/EtOAc=0/1 (KMnO₄), R_{f} of Cpd. 2-c=0.20) showed Cpd. 2-b was consumed and one main spot was detected. The reaction mixture was purified by column chromatography (Al₂O₃, Petroleum ether/EtOAc=1/1 to 0/1). Cpd. 2-c (4.56 g, 24.1 mmol, 38.4% yield) was obtained as yellow oil. ¹H NMR: ET52873-23-P1A1 (400 MHz CD₃OD) (Product) δ 3.95-3.85 (m, 1H), 3.65-3.41 (m, 2H), 3.40-3.32 (m, 1H), 3.28-3.15 (m, 4H).

STEP 3: To a solution of Cpd. 2-c (400 mg, 2.11 mmol, 1.00 eq) in DCM (6.00 mL) was added Cpd. 7-f (1.07 g, 8.46 mmol, 1.16 mL, 4.00 eq) and DMAP (155 mg, 1.27 mmol, 0.06 eq). The mixture was stirred at 25° C. for 2 h. TLC (Petroleum ether/EtOAc=2/1, R_{f} of Cpd. 2-d=0.50) showed Cpd. 2-c was consumed and one main spot detected. The reaction mixture was diluted with H₂O (15 mL) and extracted with EtOAc (6 mL×3). The combined organic layers were dried over Na₂SO₄, filtered and concentrated under reduced pressure to give a residue. The residue was purified by column chromatography (SiO₂, Petroleum ether/EtOAc=1/0 to 2/1). Cpd. 2-d (600 mg, 1.06 mmol, 49.9% yield) was obtained as brown oil. ¹H NMR (400 MHz CDCl₃) (Product) δ 7.40-7.35 (m, 2H), 7.25-7.18 (m, 1H), 5.35-5.25 (m, 3H), 4.25-4.15 (m, 1H), 4.10-3.85 (m, 3H), 3.66-3.60 (m, 1H), 3.51-3.44 (m, 1H), 3.40-3.24 (m, 1H), 1.50-1.44 (m, 27H).

STEP 4: To a suspension of Pd/C (1.00 g, 8.81 mmol, 10% purity) in MeOH (35 mL) was added Cpd. 2-d (5.00 g, 8.81 mmol, 1.00 eq) under Ar. The suspension was degassed under vacuum and purged with H₂ several times. The mixture was stirred under H₂ (50 psi) at 25° C. for 72 h. ¹H NMR showed Cpd. 2-d was consumed and product was detected. The reaction mixture was filtered and concentrated under reduced pressure to give a residue. Cpd. 2-e (4.10 g, 7.49 mmol, 84.9% yield) was obtained as a brown oil. ¹H NMR (400 MHz CDCl₃) (Product) δ 4.85-4.29 (m, 1H), 4.11-3.58 (m, 5H), 3.55-2.66 (m, 8H), 2.60-2.37 (m, 5H), 1.56-1.35 (m, 27H).

STEP 5: To a solution of Cpd. 7-i (2.11 g, 6.57 mmol, 1.20 eq) in DMF (30 mL) was added HBTU (4.15 g, 10.9 mmol, 2.00 eq) and DIPEA (2.83 g, 21.9 mmol, 3.82 mL, 4.00 eq) at 25° C. Then the mixture was added Cpd. 2-e (3.00 g, 5.48 mmol, 1.00 eq) and stirred at 25° C. for 2 h. LCMS (RT of Cpd. 2-f=3.162 min) showed Cpd. 2-e was consumed, many peaks formed and one new peak of desired mass detected. The reaction mixture was diluted with H₂O (60.0 mL) and extracted with DCM (20 mL×3). The combined organic layers were dried over Na₂SO₄, filtered and concentrated under reduced pressure to give a residue. The residue was purified by prep-HPLC (column: Welch Xtimate C18 250×70 mm #10 um; mobile phase: [water (NH₄HCO₃)-ACN]; B %: 65%-98%, 20 min). Cpd. 2-f (1.44 g, 1.69 mmol, 30.9% yield) was obtained as a brown oil. ¹H NMR (400 MHz CDCl₃) (Product) δ 7.52-7.28 (m, 5H), 5.12 (s, 2H), 4.16-3.50 (m, 8H), 3.41-2.95 (m, 6H), 2.61-2.19 (m, 10H), 1.64-1.55 (m, 4H), 1.50-1.38 (m, 27H), 1.36-1.21 (m, 12H).

STEP 6: Cpd. 2-f (650 mg, 765 umol, 1.00 eq) was dissolved in anhydrous DCM (13.0 mL) at 17° C., then TFA (6.01 g, 52.7 mmol, 3.90 mL, 68.9 eq) was added and stirred at 17° C. for 4 h under N₂. TLC (Petroleum ether/EtOAc=1/1 (PMA), R_{f} of Cpd. 2-f=0.60) indicated the reaction was completed and LCMS (RT of Cpd. 2-g=0.610 min) indicated desired MS. The reaction was concentrated under reduced pressure to give a residue at 35° C., evaporation with anhydrous toluene/THF (30 mL/30 mL)×5 at 40° C. and used into the next step without further purification. Cpd. 2-g (600 mg, crude) was obtained as a brown gum and LCMS (RT of Cpd. 2-g=0.609 min) indicated ˜80.6% purity. LCMS: [M+H]=682.3 (Product). ¹H NMR (400 MHz CDCl₃) (Product) δ 7.40-7.29 (m, 5H), 5.11 (s, 2H), 4.16-3.51 (m, 8H), 3.49-2.99 (m, 6H), 2.75-2.49 (m, 6H), 2.48-2.26 (m, 5H), 1.69-1.50 (m, 4H), 1.39-1.19 (m, 14H).

STEP 7: Cpd. 2-g (540 mg, 792 umol, 1.00 eq) was dissolved in DMF (10.8 mL), then added DIPEA (1.54 g, 11.9 mmol, 2.07 mL, 15.0 eq) and stirred for 10 min, then added HBTU (931 mg, 2.46 mmol, 3.10 eq), Cpd. 7-1 (1.66 g, 2.46 mmol, 3.10 eq, TsOH salt) was added at 17° C., the mixture was stirred at 30° C. (oil bath) for 16 h under N₂. TLC (DCM/MeOH=10/1, Id AcOH, (PMA), R_{f} of R1=0.30) indicated the reaction was completed and LCMS (RT of Cpd. 2-h=0.768 min) indicated desired MS and de-1Ac (1049). The reaction was poured into a stirred water (100 mL) and DCM (80 mL), stirred, separated, the aqueous layer was extracted with DCM (80 mL×3), the combined organic layers were washed with brine (100 mL), dried over Na₂SO₄, filtered and concentrated under reduced pressure to give a residue at 35° C. Three batches (54 mg&216 mg&60 mg scale) were performed as above and were combined to purification. The residue was purified by column chromatography (SiO₂, DCM/MeOH=20/1, 15/1, 10/1, 5/1, 0/1). The residue was purified by prep-HPLC (column: Waters Xbridge BEH C18 100×25 mm×5 um; mobile phase: [water (NH₄HCO₃)-ACN]; B %: 15%-45%, 5 min). The solution was extracted with DCM (300 mL, 200 mL, 200 mL). The combined organic layers were washed with brine (200 mL), dried over Na₂SO₄, filtered and concentrated under reduced pressure to give a residue at 35° C. Cpd. 2-h (940 mg, crude) was obtained as a light brown solid. LCMS: ESI+, calcd for [M+2H]/2=1069.5, found 1069.9 (Product).

STEP 8: Cpd. 2-h (940 mg, 439 umol, 1.00 eq) was dissolved in THE (20 mL), then added wet. Pd/C (500 mg, 10% purity) and stirred at 30° C. (oil bath) for 4 h under H₂ (15 psi, balloon). TLC (DCM/MeOH=5/1, (PMA), R_{f} of Cpd. 2-h=0.30, R_{f} of Cpd. 2-i=0.10) indicated the reaction was completed and LCMS (RT of Cpd. 2-i=0.637 min) indicated desired MS. The mixture was filtered through diatomite, filtrated was concentrated at 35° C. Cpd. 2-i (840 mg, crude) was obtained as a white solid. LCMS: ESI+, calcd for [M+2H]/2=1024.5, found 1025.0 (Product).

STEP 9: Cpd. 2-i (200 mg, 97.6 umol, 1.00 eq) was dissolved in DMF (4 mL), DIPEA (37.9 mg, 293 umol, 51.0 uL, 3.00 eq) was added, HBTU (55.6 mg, 146 umol, 1.50 eq) was added, then Cpd. 7-o (49.2 mg, 117 umol, 1.20 eq) was added and stirred at 30° C. (oil bath) for 16 h under N₂. LCMS indicated no Cpd. 2-i and LCMS (RT of Cpd. 2-j=0.804 min) indicated desired MS. The reaction was poured into a stirred water (40 mL) and DCM (40 mL), stirred, separated, the aqueous layer was extracted with DCM (40 mL×3), the combined organic layers were washed with brine (50 mL), dried over Na₂SO₄, filtered and concentrated under reduced pressure to give a residue at 35° C. Combined with another batch (200 mg scale) for purification. The residue was purified by prep-HPLC (column: Phenomenex C18 75×30 mm×3 um; mobile phase: [water (NH₄HCO₃)-ACN]; B %: 35%-55%, 8 min). Cpd. 2-j (210 mg) was obtained as a light yellow solid. LCMS: ESI⁻, calcd for [M−2H]/2=1223.1, found 1223.6 (Product).

STEP 10: Cpd. 2-j (100 mg, 40.8 umol, 1.00 eq) was dissolved in anhydrous DCM (4 mL), then DMAP (1.25 mg, 10.2 umol, 0.25 eq) and DIPEA (21.1 mg, 163 umol, 28.4 uL, 4.00 eq) and succinic anhydride (16.3 mg, 163 umol, 4.00 eq) were added, and stirred at 20° C. (oil bath) for 16 h under N₂. LCMS (RT of Cpd. 2-k=0.788 min) indicated ˜33.3% desired MS, ˜27.4% Cpd. 2-j. The reaction was poured into a stirred saturated NaHCO₃ (15 mL) and DCM (15 mL), stirred, separated, the aqueous layer was extracted with DCM (10 mL×4), the combined organic layers were washed with brine (10 mL), dried over Na₂SO₄, filtered and concentrated under reduced pressure to give a residue at 35° C. The residue was purified by prep-HPLC (column: Waters Xbridge BEH C18 100*30 mm*10 um; mobile phase: [water (NH₄HCO₃)-ACN]; B %: 35%-55%, 8 min). Cpd. 2-k (4-(((3R,5S)-5-((bis(4-methoxyphenyl)(phenyl)methoxy)methyl)-1-(12-oxo-12-((((2R,3R,4R,5S)-3,4,5-tris(3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)tetrahydro-2H-pyran-2-yl)methyl)amino)dodecanoyl)pyrrolidin-3-yl)oxy)-4-oxobutanoic acid) (19.0 mg, 7.40 umol, 18.1% yield, NH₄ salt) was obtained as a white solid. LCMS: ESI⁻, calcd for [M−2H]/2=1273.1, found 1273.2 (Product).

STEP 11: To a solution of Cpd. 2-k (19.4 mg, 7.56 umol, 1.00 eq, NH₄ salt) in anhydrous MF (0.97 mL) was added HBTU (14.3 mg, 37.8 umol, 5.00 eq), DIPEA (7.81 mg, 60.4 umol, 10.5 uL, 8.00 eq), DMAP (923 ug, 7.56 umol, 1.00 eq) in one portion at 17° C. Then Resin CPG-NH₂ (149 mg, 7.56 umol; Chemical Name: Aminoalkyl-CPG, 500A from Hebei DNAchem Biotechnology Co., Ltd.) was added to the mixture. The mixture was stirred at 40° C. for 24 h. LCMS indicated the reaction was completed. After that, it was filtered and the filter cake was washed with MeOH (5.00 mL×4) and DCM (5.00 mL×4). The filter cake was dried under N₂ stream and obtained as a pale yellow solid (222 mg). Ac₂O (327 mg, 3.20 mmol, 300 uL, 36.8 eq) was added to Py (1.5 mL) and mixed, added to pale yellow solid above (222 mg) and stirred at 35° C. for 0.5 h. After that, it was filtered and the filter cake was washed with DCM (5.00 mL×4) and MeOH (5.00 mL×4). The resulted pale yellow solid was dried under vacuum for 12 h to give 132 mg supported product (Loading: 32.3 umol/g).

### Example 26

### Synthesis of Compound 3

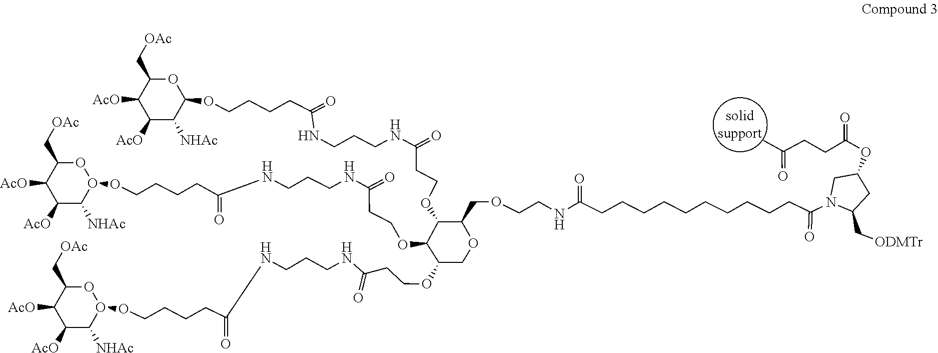

Compound 3 may be synthesized following the synthetic route described above for compound 2 by using Intermediate 3-4 as the starting material.

### Example 27

### Synthesis of Compound 4

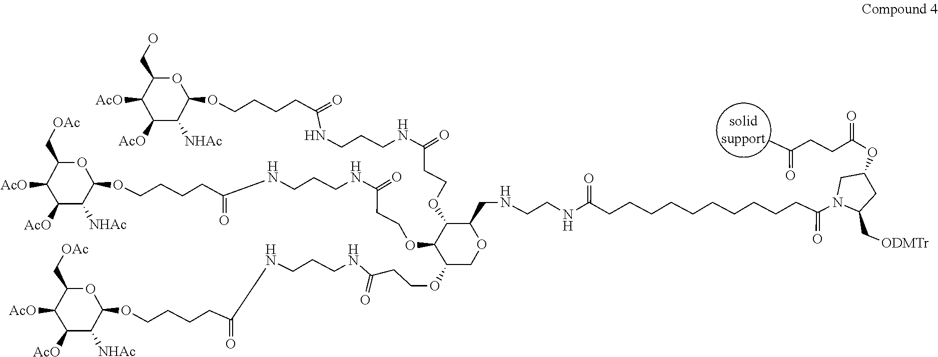

Compound 4 may be synthesized following the synthetic route described above for compound 2 by using Intermediate 4-4 as the starting material.

### Example 28

### Synthesis of Compound 5

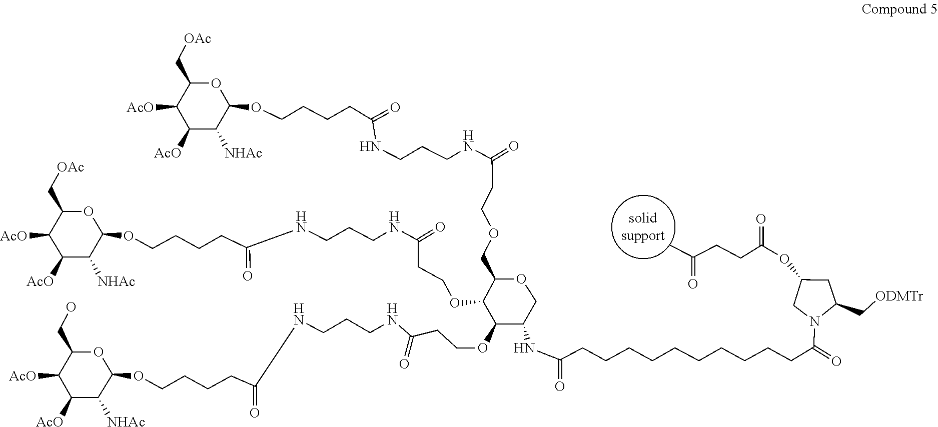

Compound 5 was synthesized following the procedure of compound 6 in example 29 by using 5-j as the starting material of which the synthesis was shown below. Loading: 27.0 μmol/g.

### 4-(((3R,5S)-1-(12-(((3S,4R,5S,6R)-4,5-bis(3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)-6-((3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)methyl)tetrahydro-2H-pyran-3-yl)amino)-12-oxododecanoyl)-5-((bis(4-methoxyphenyl)(phenyl)methoxy)methyl)pyrrolidin-3-yl)oxy)-4-oxobutanoic acid (5-j)

The title compound (5-j) was synthesized following the procedure of compound 6-j in example 29 by using 5-f as the starting material of which the synthesis was shown below. LCMS: calcd for [M−2H]/2: 1273.1, found: 1273.7.

### The synthesis of 3,3′-(((2R,3S,4R,5S)-5-(12-(benzyloxy)-12-oxododecanamido)-2-((2-carboxyethoxy)methyl)tetrahydro-2H-pyran-3,4-diyl)bis(oxy))dipropionic acid (5-f)

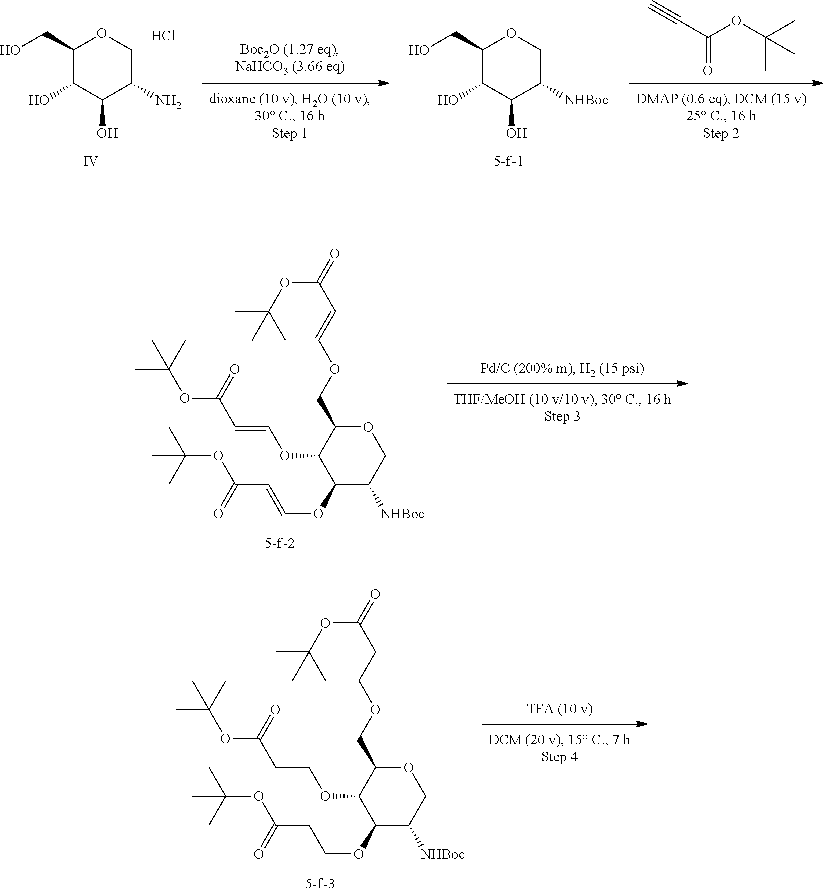

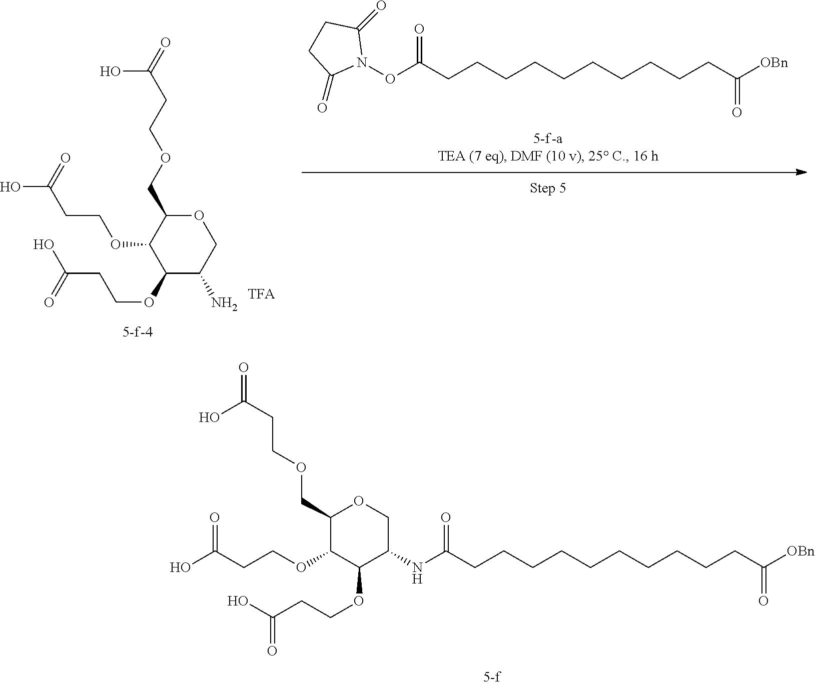 [SMILES: N[C@H]1CO[C@H](COCCC(=O)O)[C@@H](OCCC(=O)O)[C@@H]1OCCC(=O)O ; O=C(CCCCCCCCCCC(=O)ON1C(=O)CCC1=O)OCc1ccccc1 ; O=C(O)CCOC[C@H]1OC[C@H](NC(=O)CCCCCCCCCCC(=O)OCc2ccccc2)[C@@H](OCCC(=O)O)[C@@H]1OCCC(=O)O]

Step 1. Compound IV (27.2 g, 136 mmol, 1.00 eq, HCl salt) was dissolved in dioxane (270 mL) and H₂O (270 mL), NaHCO₃ (41.9 g, 499 mmol, 19.4 mL, 3.66 eq) was added, Boc₂O (37.7 g, 173 mmol, 39.6 mL, 1.27 eq) was added at 15° C. and stirred at 30° C. for 16 h under N₂. TLC (i-PrOH/H₂O/NH₃·H₂O=6/3/1) indicated compound IV (example 4) was consumed completely and LCMS indicated desired product MS. The mixture was filtered and filtrate was concentrated under reduced pressure to give a residue at 45° C. The residue was purified by column chromatography (SiO₂, DCM/MeOH=15/1, 10/1, 8/1, 4/1, 0/1) to provide tert-butyl ((3S,4R,5S,6R)-4,5-dihydroxy-6-(hydroxymethyl)tetrahydro-2H-pyran-3-yl)carbamate (5-f-1) (33.7 g; yield: 93.9% yield). LCMS: calcd for [M−Boc+H]: 164.1, found: 164.1. ¹H NMR: (400 MHz CD₃OD) δ 3.91 (dd, J=5.2, 11.2 Hz, 1H), 3.83 (dd, J=2.0, 11.6 Hz, 1H), 3.61 (dd, J=6.0, 12.0 Hz, 1H), 3.54-3.40 (m, 1H), 3.36-3.23 (m, 2H), 3.19-3.03 (m, 2H), 1.44 (s, 9H).

Step 2. Compound 5-f-1 (4.55 g, 17.3 mmol, 1.00 eq) was dissolved in anhydrous DCM (68 mL), DMAP (1.27 g, 10.4 mmol, 0.60 eq) was added, then tert-butyl propiolate (8.72 g, 69.1 mmol, 9.49 mL, 4.00 eq) was added to the white mixture and stirred at 25° C. (oil bath) for 16 h under N₂. TLC (DCM/MeOH=5/1(PMA) indicated compound 5-f-1 was consumed completely and LCMS indicated ˜31.6% desired product MS. The black solution was concentrated at 40° C. The residue was purified by column chromatography (SiO₂, Petroleum ether/EtOAc=20/1, 10/1, 8/1, 5/1, 4/1, 1/1, 0/1) to provide di-tert-butyl 3,3′-(((2R,3S,4R,5S)-2-((((E)-3-(tert-butoxy)-3-oxoprop-1-en-1-yl)oxy)methyl)-5-((tert-butoxycarbonyl)amino)tetrahydro-2H-pyran-3,4-diyl)bis(oxy))(2E,2′E)-diacrylate (5-f-2) (3.42 g; yield: 30.8% yield). LCMS: calcd for [M+NH₄]: 659.3, found: 659.4. ¹H NMR: (400 MHz CDCl₃) δ 7.45 (d, J=12.4 Hz, 1H), 7.35-7.17 (m, 2H), 5.36-5.21 (m, 2H), 5.15 (d, J=12.4 Hz, 1H), 4.69 (d, J=8.0 Hz, 1H), 4.25-3.83 (m, 6H), 3.77-3.37 (m, 3H), 1.54-1.33 (m, 27H).

Step 3. Compound 5-f-2 (2.42 g, 3.77 mmol, 1.00 eq) was dissolved in THE (24 mL) and MeOH (24 mL), wet. Pd/C (4.84 g, 156 umol, 10% purity) was added, then stirred at 30° C. for 48 h under H₂ (15 psi, balloon). LCMS indicated the complete consumption of compound 5-f-2 and desired product MS. The mixture was filtered through diatomite, the cake was washed with MeOH, filtrated was concentrated at 40° C. to provide di-tert-butyl 3,3′-(((2R,3S,4R,5S)-2-((3-(tert-butoxy)-3-oxopropoxy)methyl)-5-((tert-butoxycarbonyl)amino)tetrahydro-2H-pyran-3,4-diyl)bis(oxy))dipropionate (5-f-3) (2.40 g; yield: 98.3%; yellow syrup). ¹H NMR: (400 MHz CDCl₃)

δ 5.18-4.91 (m, 1H), 4.24-4.08 (m, 1H), 4.04-3.94 (m, 1H), 3.93-3.59 (m, 7H), 3.55-3.41 (m, 1H), 3.35-3.18 (m, 3H), 3.10 (t, J=10.8 Hz, 1H), 2.62-2.38 (m, 6H), 1.54-1.33 (m, 36H).

Step 4. Compound 5-f-3 (2.40 g, 3.70 mmol, 1.00 eq) was dissolved in anhydrous DCM (48.0 mL) at 15° C., then TFA (37.0 g, 324 mmol, 24.0 mL, 87.5 eq) was added and stirred at 15° C. for 7 h under N₂. LCMS indicated the complete consumption of compound 5-f-3 and desired product MS. The solution was concentrated under reduced pressure to give a residue at 40° C., co-evaporated with ACN (50 mL×4) and toluene (50 mL×4) to remove TFA at 45° C. to provide 3,3′-(((2R,3S,4R,5S)-5-amino-2-((2-carboxyethoxy)methyl)tetrahydro-2H-pyran-3,4-diyl)bis(oxy))dipropionic acid (5-f-4) (2.00 g, TFA salt, yellow gum). LCMS: calcd for [M+H]: 380.1, found: 380.1. ¹H NMR: (400 MHz D₂O) δ 4.25-4.08 (m, 2H), 4.05-3.71 (m, 7H), 3.68-3.41 (m, 4H), 3.39-3.24 (m, 1H), 2.83-2.61 (m, 6H), 2.06 (s, 2H).

Step 5. Compound 5-f-4 (2.00 g, 4.05 mmol, 1.00 eq, TFA salt) was dissolved in anhydrous DMF (20 mL), TEA (2.87 g, 28.4 mmol, 3.95 mL, 7.00 eq) was added, solid was detected, 5-f-a (1.86 g, 4.46 mmol, 1.10 eq) of which the synthesis was shown below was added and stirred at 25° C. (oil bath) for 16 h under N₂, light yellow solution. LCMS indicated ˜59.3% desired product MS. The reaction was concentrated under reduced pressure to give a residue at 45° C. The residue was roughly purified by column chromatography to provide compound 5-f (1.83 g, white solid) which was used in next step without further purification. LCMS: calcd for [M+H]: 682.3, found: 682.4.

### The synthesis of 1-benzyl 12-(2,5-dioxopyrrolidin-1-yl) dodecanedioate (5-f-a)

To a mixture of 12-(benzyloxy)-12-oxododecanoic acid (5.00 g, 15.6 mmol, 1.00 eq) in DCM (35.0 mL) was added N-hydroxysuccinimide (2.16 g, 18.7 mmol, 1.20 eq) and DCC (4.19 g, 20.3 mmol, 4.10 mL, 1.30 eq) in one portion at −5° C. under N₂. The mixture was stirred at −5° C. for 2 hrs, then heated to 30° C. and stirred for 10 hrs. TLC (petroleum ether:ethyl acetate=1:1, Rf=0.7) indicated 12-(benzyloxy)-12-oxododecanoic acid was completely consumed, and one major new spot with lower polarity was detected. Filtered and concentrated in vacuum. The crude product was triturated with i-prOH:heptane=1:1 at 25° C. for 60 min. Then the mixture was filtered and the liquid was concentrated in vacuum to provide compound 5-f-a (6.00 g; yield: 92.1% yield). LCMS: calcd for [M+NH4]: 435.2, found: 435.3.

### Example 29

### Synthesis of Compound 6

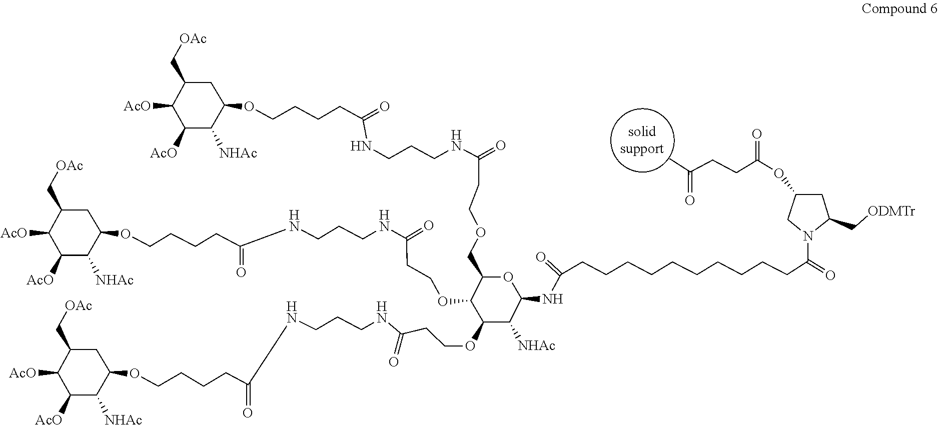

The title compound was synthesized according to the synthetic route below.

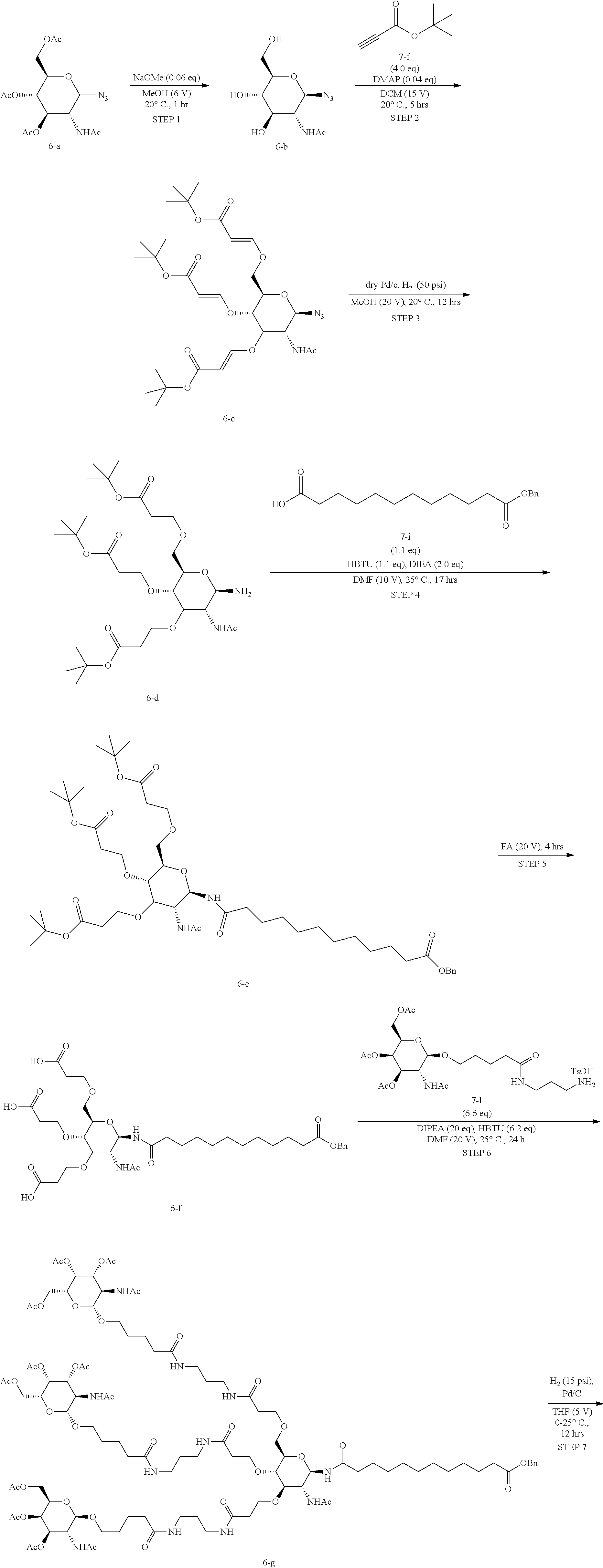

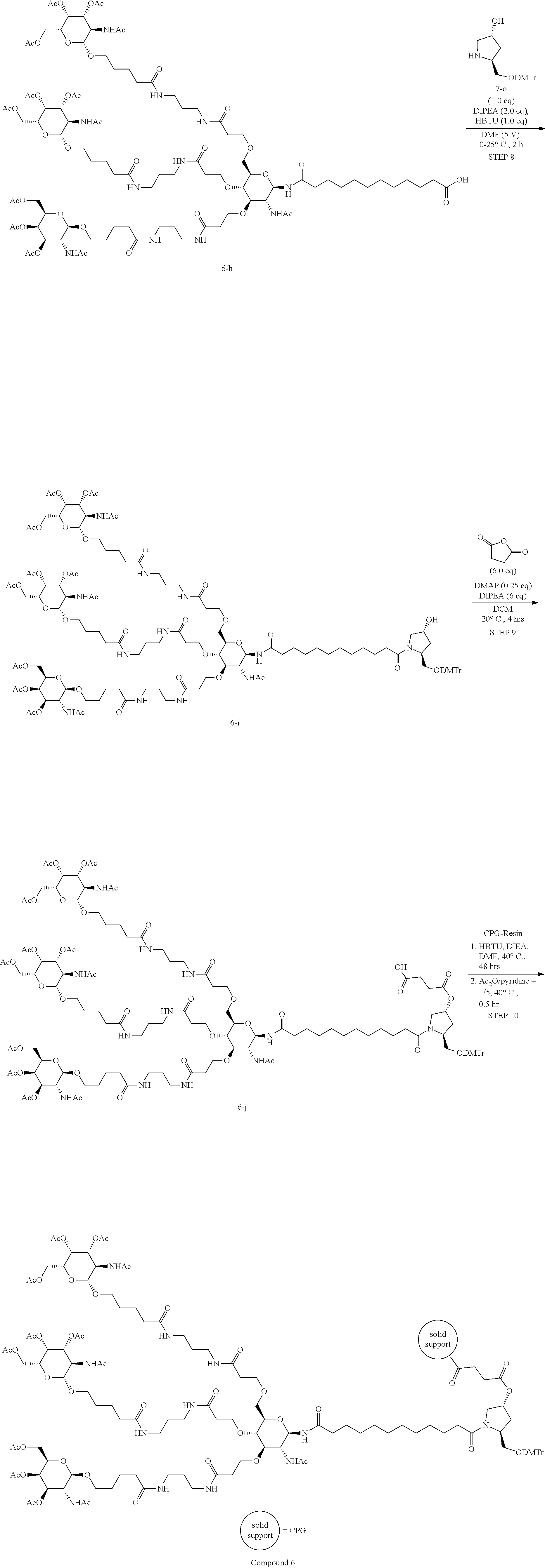

STEP 1: To a solution of Cpd. 6-a (5.00 g, 13.4 mmol) in MeOH (30.0 mL) was added NaOMe (5.40 M, 149 uL) in one portion at 20° C. under N₂. The mixture was stirred at 20° C. for 1 hr. LCMS (product: RT=0.220 min) showed one main peak with desired mass was detected. The mixture was adjusted to pH 6 by Amberlite IR120, Na resin (CAS: 78922-04-0), then filtered and concentrated in vacuum to afford Cpd. 6-b (3.20 g, 96.7% yield) as a white solid. ¹H NMR (400 MHz, CD₃OD) δ 4.50 (d, J=9.2 Hz, 1H), 3.86-3.94 (m, 1H), 3.63-3.75 (m, 2H), 3.42-3.50 (m, 1H), 3.35 (s, 10H), 1.99 (s, 3H).

STEP 2: To a solution of Cpd. 6-b (3.34 g, 13.5 mmol) in DCM (50.0 mL) was added DMAP (662 mg, 5.43 mmol). To this mixture was added Cpd. 7-f (6.85 g, 54.2 mmol, 7.5 mL) and the mixture was stirred at 20° C. for 5 hrs. TLC (Petroleum ether/Ethyl acetate, product: R_{f}=0.43) showed all the starting material was consumed, and LCMS (product: RT=0.220 min) showed one main peak with desired mass was detected. The mixture was concentrated in vacuum. The residue was purified by silica gel chromatography (Petroleum ether/Ethyl acetate=8/1 to 0/1) to give Cpd. 6-c (2.10 g, 33.0% yield) as a yellow solid. ¹H NMR (400 MHz, CD₃OD) δ 4.50 (d, J=6.2 Hz, 3H), 3.80-3.90 (m, 2H), 3.74 (br t, J=6.3 Hz, 2H), 3.61-3.68 (m, 2H), 3.53-3.61 (m, 2H), 3.10-3.27 (m, 4H), 1.82-1.93 (m, 3H), 1.27-1.42 ppm (m, 28H)

STEP 3: To a solution of Cpd. 6-c (100 mg, 160 umol) in MeOH (2.00 mL) was added Pd/C (0.02 g, 10% purity) under Ar. The mixture was stirred under H₂ (50 psi) at 25° C. for 48 hrs. LCMS (product: RT=0.884 min) showed one main peak with desired mass was detected. The mixture was filtered and concentrated in vacuum to give Cpd. 6-d (125 mg, 53.41% yield, 41.36% purity) was obtained as a brown oil. ¹H NMR (400 MHz, CD₃OD) δ 3.80-3.90 (m, 2H), 3.74 (br t, J=6.3 Hz, 2H), 3.61-3.68 (m, 2H), 3.53-3.61 (m, 2H), 3.10-3.27 (m, 4H), 2.27-2.49 (m, 6H) 1.82-1.93 (m, 3H), 1.27-1.42 ppm (m, 27H)

STEP 4: To a solution of Cpd. 7-i (244 mg, 763 umol) in DMF (3.00 mL) was added HBTU (289 mg, 763 umol) and DIPEA (179 mg, 1.39 mmol). To this mixture was added Cpd. 6-d (420 mg, 694 umol) and the mixture was stirred at 25° C. for 17 hrs. LCMS (product: RT=1.07 min) showed one main peak with desired mass was detected. The mixture was poured into sat. NaHCO₃ (aq) (10 mL) with ice and extracted with ethyl acetate (10 mL×4). The combined organic phase was washed with water (10 mL×2) and sat. NaHCO₃ (aq) (10 mL×2). Then the combined organic phase was dried with anhydrous Na₂SO₄, filtered and concentrated in vacuum. The mixture was concentrated in vacuum. The residue was purified by silica gel chromatography (Petroleum ether/Ethyl acetate=20/1, 0/1) to give Cpd. 6-e (450 mg, 496 umol, 71.4% yield) as a white solid. LCMS: [M−H]=905.6.

STEP 5: To a mixture of Cpd. 6-e (1.50 g, 1.65 mmol) was added formic acid (30.0 mL) at 25° C. under N₂. The mixture was stirred at 20° C. for 2 hrs. LCMS (product: RT=0.759 min) showed the starting material was consumed completely. The reaction mixture filtered and concentrated under reduced pressure to give a residue. To the residue was added toluene (4 mL) and concentrated to give a residue and the operation was repeated for 3 times. Finally, Cpd. 6-f (1.15 g, 1.56 mmol, 94.1% yield) was obtained as a yellow solid. ¹H NMR: (400 MHz, D₆-DMSO) (Product) δ=8.13 (d, J=9.3 Hz, 1H), 7.86 (br d, J=9.3 Hz, 1H), 7.28-7.43 (m, 5H), 5.08 (s, 2H), 4.83 (s, 1H), 3.68-3.88 (m, 4H), 3.48-3.67 (m, 5H), 3.27-3.37 (m, 1H), 3.18-3.25 (m, 1H), 3.09 (s, 1H), 2.25-2.56 (m, 10H), 1.92-2.13 (m, 2H), 1.77 (s, 3H), 1.52 (br d, J=6.8 Hz, 5H), 1.21 ppm (br d, J=7.8 Hz, 12H).

STEP 6: To a solution of Cpd. 7-1 (3.41 g, 5.05 mmol, TsOH salt) in DCM (8.00 mL) was added HBTU (1.80 g, 4.74 mmol) and DIPEA (1.98 g, 15.29 mmol, 2.66 mL) in one portion at 25° C. under N₂, and then the mixture was stirred at 25° C. for under N₂ 10 min. To this mixture was added Cpd. 6-f (1.13 g, 1.53 mmol) and the mixture was stirred at 25° C. for 20 hrs. LCMS (product: RT=2.755 min) showed the starting material was consumed completely. The reaction mixture suspended in ethyl acetate (35 mL) and washed with saturated NaHCO₃ (35 mL). The organic phase was collected, washed with saturated NaCl (35 mL×2). The organic phase (a suspension) was collected and Cpd. 6-g (1.35 g, 0.614 mmol, 40.2% yield) was obtained by centrifugation as white solid. LCMS: ESI⁻, calcd for [M−2H]/2=1096.0, found 1096.6 (product).

STEP 7: To a solution of Cpd. 6-g (500 mg, 227 umol) in THE (2.5 mL) was added Pd/C (600 mg, 10% purity) under Ar. The mixture was stirred under H₂ (15 psi) at 25° C. for 48 hours. LCMS (product: RT=2.1 min) showed one main peak with desired mass was detected. The mixture was filtered and concentrated in vacuum to give Cpd. 6-h (430 mg, crude) was obtained as white solid and used into the next step without further purification. LCMS: ESI⁻, calcd for [M−2H]/2=1051.01, found 1051.1 (product).

STEP 8: To a mixture of Cpd. 6-h (170 mg, 80.7 umol) in DMF (2.00 mL) was added HBTU (45.9 mg, 121 umol) and DIPEA (20.8 mg, 161 umol) in one portion at 25° C. under N₂, and then the mixture was stirred at 25° C. for 10 min under N₂ atmosphere. To this mixture was added Cpd. 7-o (50.8 mg, 121 umol) and the mixture was stirred at 17° C. for 17 hrs. LCMS (product: RT=2.86 min) showed the starting material was consumed completely. The residue was purified by precipitation with ACN (3.0 mL). Finally, Cpd. 6-i (150 mg, 59.8 umol, 74.1% yield) was obtained as a white solid. LCMS: ESI⁻, calcd for [M−2H]/2=1251.61, found 1252.1 (product).

STEP 9: To a mixture of succinic anhydride (35.9 mg, 359 umol) in DCM (3.00 mL) was added DIPEA (46.4 mg, 359 umol) and DMAP (1.83 mg, 14.9 umol) in one portion at 25° C. under N₂. While stirring, Cpd. 6-i (150 mg, 59.8 umol) was added and the mixture was stirred at 20° C. for 4 hrs. LCMS (product: RT=2.19 min) showed the starting material was consumed completely. The mixture was poured into DCM (20 mL). The combined organic phase was washed with TEBA (aq) (20 mL×2). Then the combined organic phase was dried with anhydrous Na₂SO₄, filtered and concentrated in vacuum. The crude product was purified by reversed-phase prep-HPLC (0.1M TEAB-ACN; B %: 20%-45%, 20 min) to give Cpd. 6-j (4-(((3R,5S)-1-(12-(((2R,3R,4R,5S,6R)-3-acetamido-4,5-bis(3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)-6-((3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)methyl)tetrahydro-2H-pyran-2-yl)amino)-12-oxododecanoyl)-5-((bis(4-methoxyphenyl)(phenyl)methoxy)methyl)pyrrolidin-3-yl)oxy)-4-oxobutanoic acid) (35.0 mg, 13.4 umol, 22.4% yield) was obtained as a white solid. LCMS: ESI⁻, calcd for [M−2H]/2=1301.62, found 1301.1 (product).

STEP 10: To a solution of Cpd. 6-j (20 mg, 7.67 umol) in DMF (1.50 mL) was added HBTU (14.5 mg, 38.3 umol), DIEA (7.93 mg, 61.3 umol, 10.6 uL), DMAP (937 ug, 7.67 umol) in one portion at 25° C. Then CPG (130 mg; Chemical Name: Aminoalkyl-CPG, 500A from Hebei DNAchem Biotechnology Co., Ltd.) was added to the mixture. The mixture was stirred at 40° C. for 19 hrs. After that, it was filtered and the filter cake was washed with MeOH (5.00 mL×4) and DCM (5.00 mL×4). The filter cake was dried under N₂ stream to give a pale yellow solid. The pale yellow solid above was added into a mixture of dry pyridine (1.50 mL) and Ac₂O (0.30 mL). The mixture was then stirred at 40° C. for 0.5 hr. After that, it was filtered and the filter cake was washed with DCM (5.00 mL×4) and MeOH (5.00 mL×4). The resulted pale yellow solid was dried under vacuum for 12 hrs to give 130 mg solid supported product (loading: 32 umol/g).

### Example 30

### Synthesis of Compound 7

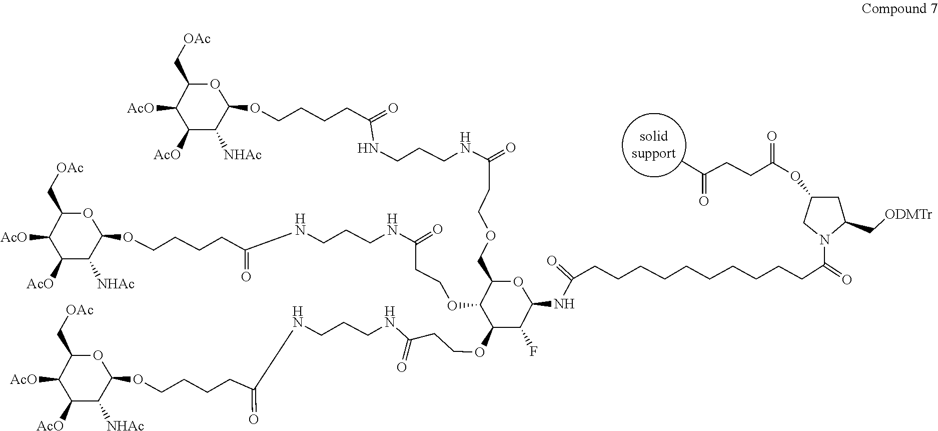

The title compound was synthesized according to the synthetic route below.

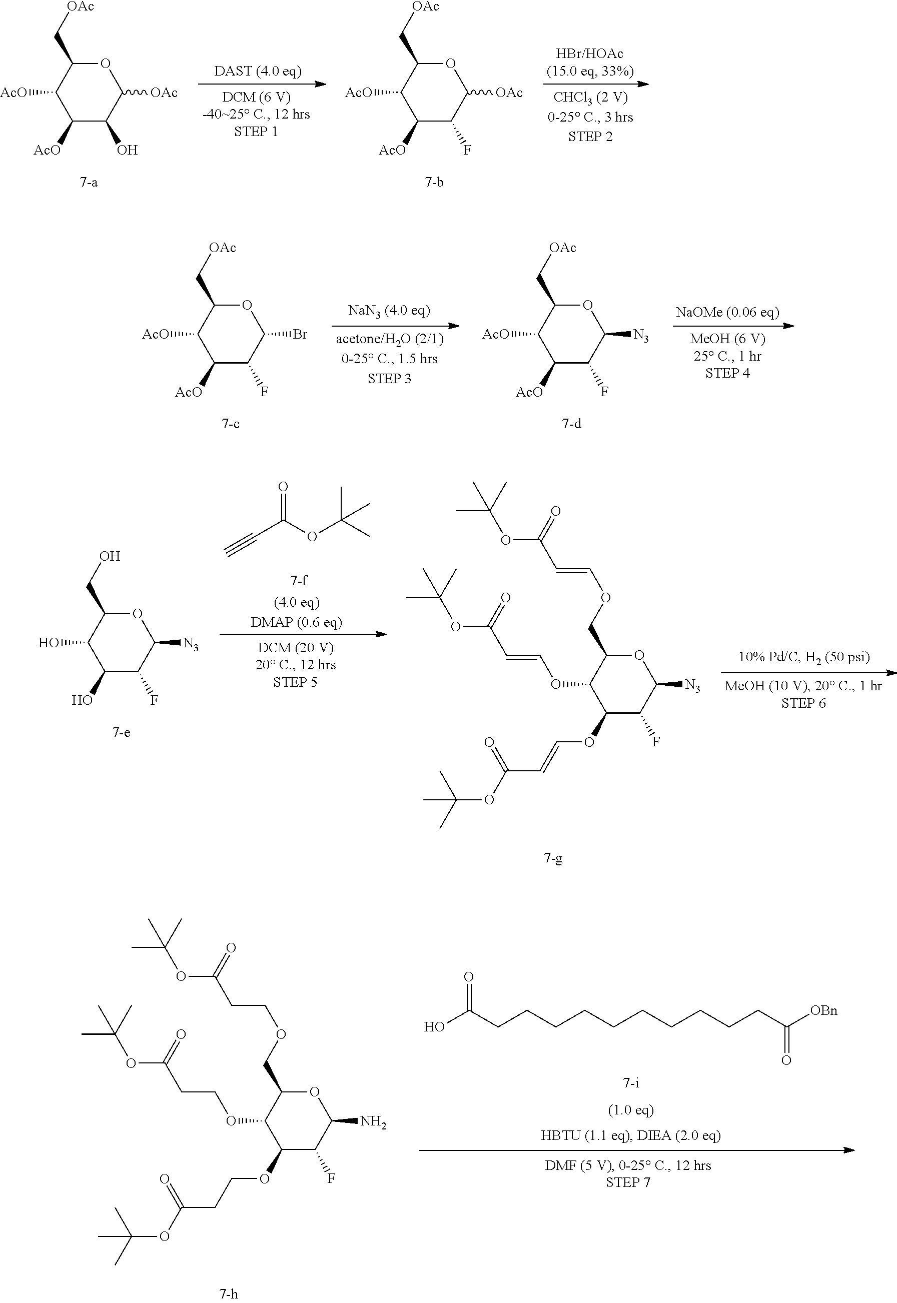

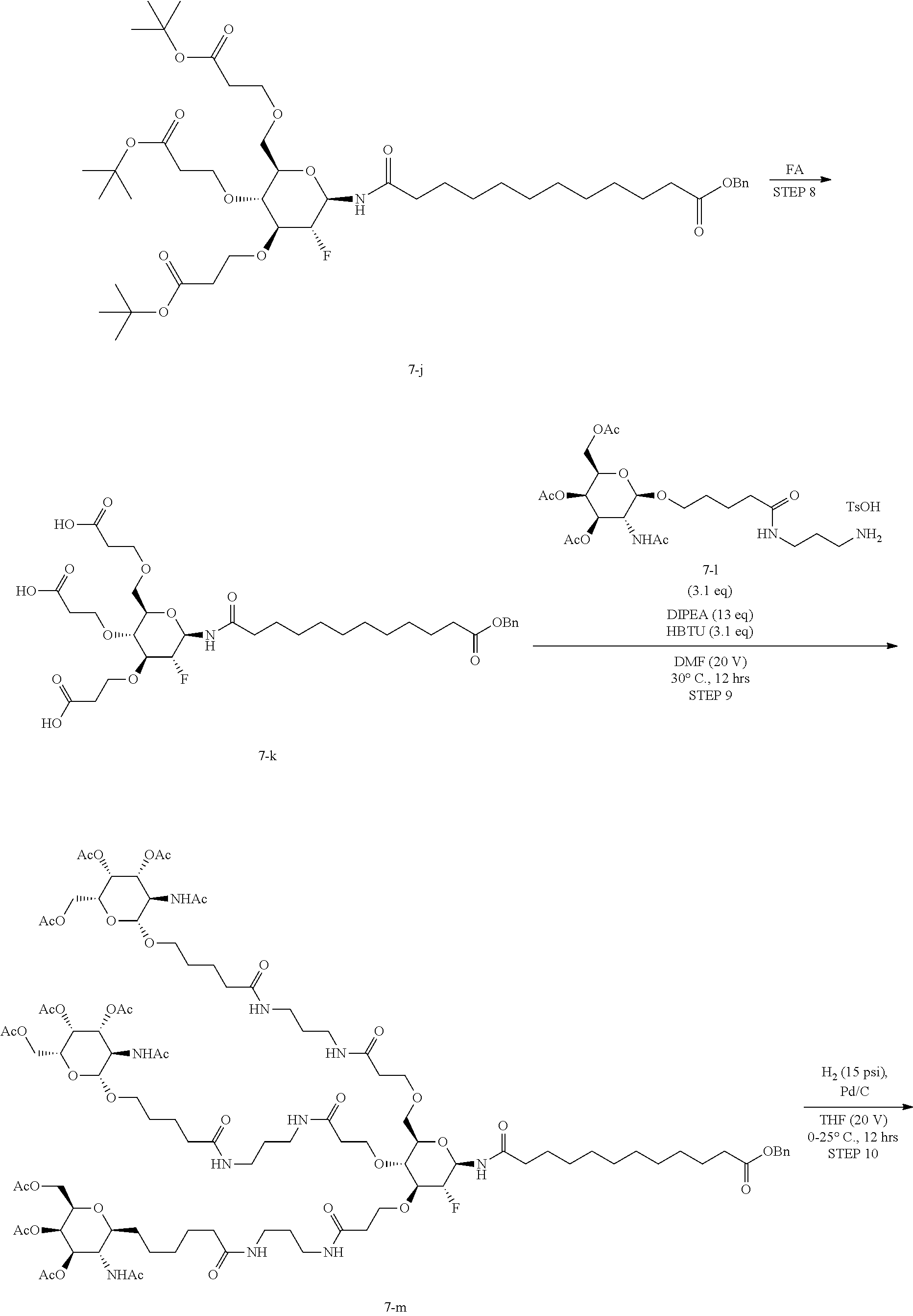

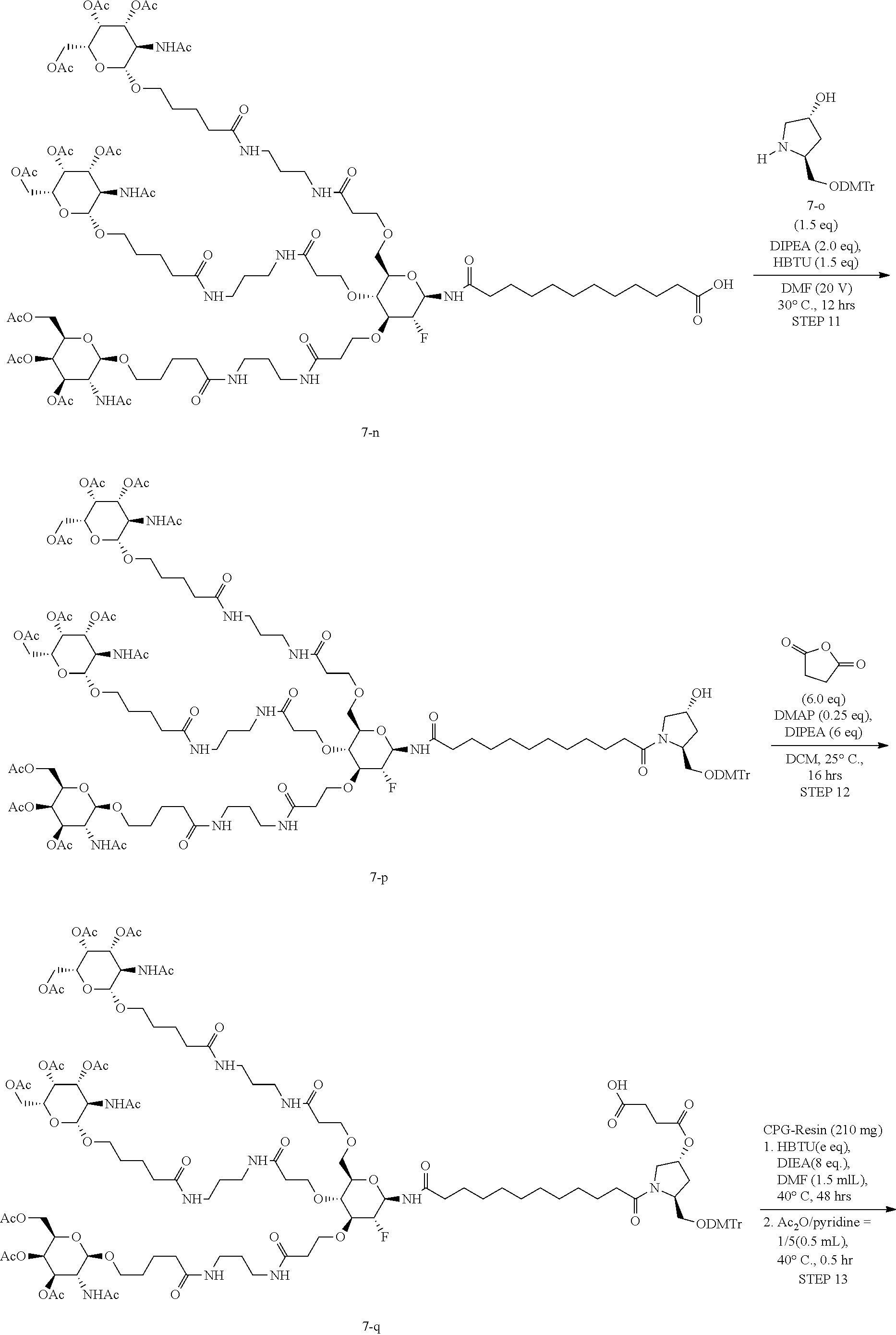

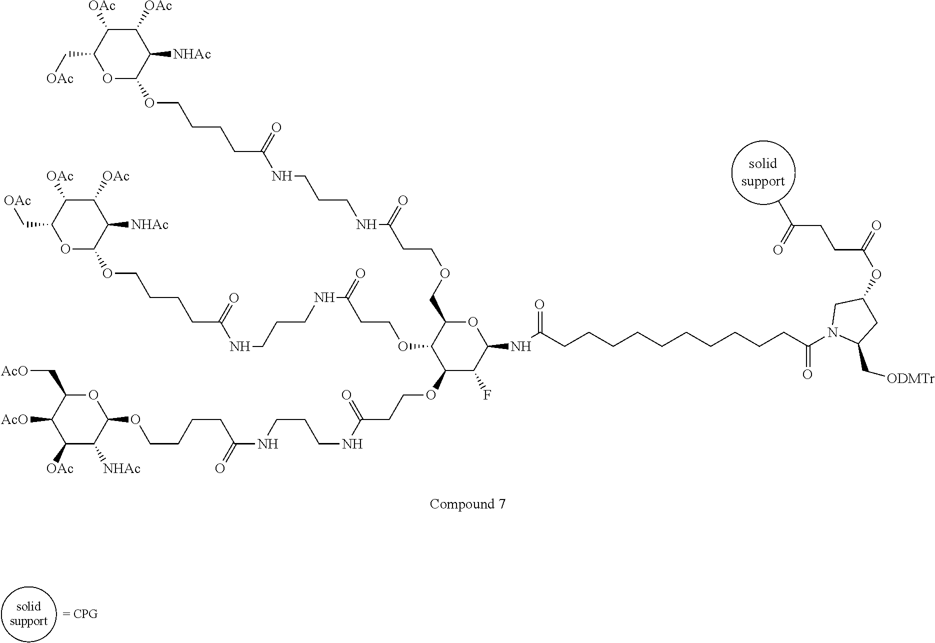

STEP 1: A solution of Cpd. 7-a (5.00 g, 14.4 mmol, 1.00 eq) in DCM (28.0 mL) was added in DAST (9.26 g, 57.4 mmol, 7.59 mL, 4.00 eq) at −40° C., the reaction was stirred at 25° C. for 12 hrs. TLC (petroleum ether:ethyl acetate=2:1, R_{f}=0.4) showed the reaction was complete. The mixture was quenched by addition aq. NH₄Cl (50.0 mL), and then extracted by EtOAc (50.0 mL×3), the combined organic layers concentrated under reduced pressure to give a residue. Four batches were performed in parallel as above and were combined to purification. The residue was purified by column chromatography (SiO₂, Petroleum ether:Ethyl acetate=20:1 to 3:1). Cpd. 7-b (4.65 g, 13.3 mmol, 23.3% yield) was obtained as a yellow oil. ¹H NMR (400 MHz, CDCl₃) (Product) δ 5.77-5.80 (m, 1H), 5.33-5.42 (d, 1H), 5.07 (t, J=8 Hz, 1H), 4.36-4.53 (m, 1H), 4.28-4.32 (m, 1H), 4.08-4.12 (m, 1H), 3.84-3.88 (m, 1H), 2.18 (s, 3H), 2.04-2.09 (m, 9H).

STEP 2: To a solution of Cpd. 7-b (9.50 g, 27.1 mmol, 1.00 eq) in CHCl₃ (20.0 mL) was added hydrogen bromide (99.7 g, 407 mmol, 66.9 mL, 33% purity, 15.0 eq) at 0° C., the mixture was stirred at 25° C. for 3 hrs. TLC (petroleum ether:ethyl acetate=2:1, R_{f}=0.6) showed the reaction was complete. The mixture was poured into, and stirred briefly with, ice water (100 mL) which was then extracted with CHCl₃, (100 mL, in 3 portions). The yellow extract was washed with several portions of ice-cold, saturated NaHCO₃, solution until first the CO₂, evolution ceased, and subsequently the organic layer became colorless. The layer was then washed once with water, dried with Na₂SO₄, and concentrate to dryness. Cpd. 7-c (9.2 g, crude) was obtained as brown oil. ¹H NMR (400 MHz, CDCl₃) (Product) δ 6.54 (d, J=8 Hz, 1H), 5.59-5.67 (m, 1H), 5.09-5.14 (m, 1H), 4.46-4.62 (m, 1H), 4.32-4.53 (m, 2H), 4.11-4.15 (m, 1H), 2.09 (d, J=4 Hz, 6H), 2.06 (s, 3H).

STEP 3: A solution of Cpd. 7-c (9.20 g, 24.8 mmol, 1.00 eq) in acetone (108 mL), then NaN₃ (6.50 g, 100 mmol, 4.03 eq) dissolved in H₂O (90.0 mL) was added dropwise in the mixture at 0° C., then the mixture was stirred at 20° C. for 3 hrs. TLC (petroleum ether: ethyl acetate=2:1, R_{f}=0.5) showed the starting materials was consumed and one new spot formed. The white precipitate was filtered, washed thoroughly with H₂O (150 mL) and dried under vacuum to give the product. Cpd. 7-d (6.60 g, 19.8 mmol, 79.9% yield) was obtained as a white solid. ¹H NMR (400 MHz, CDCl₃) (Product) δ 5.31-5.37 (m, 1H), 5.05 (t, J=8 Hz, 1H), 4.81-4.84 (m, 1H), 4.14-4.34 (m, 3H), 3.80-3.87 (m, 1H), 2.09 (d, J=4 Hz, 6H), 2.04 (s, 3H).

STEP 4: To a solution of Cpd. 7-d (6.50 g, 19.5 mmol, 1.00 eq) in MeOH (39.0 mL) was added NaOMe (211 mg, 1.17 mmol, 30% purity, 0.06 eq), the mixture was stirred at 20° C. for 1 hr. TLC (petroleum ether: ethyl acetate=1:1, R_{f}=0.1) showed the starting materials was consumed and one new spot formed. The reaction was adjusted pH-7 by Amberlite IR120, Na resin (CAS: 78922-04-0), filtered and concentrated under reduced pressure to give a residue. The Cpd. 7-e (4.00 g, crude) was obtained as a yellow oil. ¹H NMR (400 MHz, D₆-DMSO) (Product) δ 5.58 (s, 1H), 5.32 (s, 1H), 4.97-4.99 (m, 1H), 4.70 (s, 1H), 3.81-3.99 (m, 1H), 3.68-3.71 (m, 1H), 3.34-3.53 (m, 2H), 3.32-3.36 (m, 1H), 3.15 (t, J=8 Hz, 1H).

STEP 5: To a solution of Cpd. 7-e (4.00 g, 19.3 mmol, 1.00 eq) in DCM (40.0 mL) was added Cpd. 7-f (9.74 g, 77.2 mmol, 10.6 mL, 4.00 eq) and DMAP (1.42 g, 11.6 mmol, 0.60 eq), the mixture was stirred at 20° C. for 12 hrs. TLC (petroleum ether: ethyl acetate=1:1, R_{f}=0.9) showed the starting materials was consumed and one new spot formed. The solvent was removed in vacuum, water (50.0 ml) was added to the residue. The aqueous layer was extracted with dichloromethane (3×50.0 ml), and the organic layer was dried with Na₂SO₄. The residue was purified by column chromatography (SiO₂, petroleum ether: ethyl acetate=20:1 to 8:1). Cpd. 7-g (5.20 g, 8.88 mmol, 46.0% yield) was obtained as an orange solid. ¹H NMR (400 MHz, CDCl₃) (Product) δ 7.44 (d, J=12 Hz, 1H), 7.28 (d, J=12 Hz, 1H), 5.27-5.36 (m, 2H), 5.15 (d, J=12 Hz, 1H), 4.81 (m, 1H), 4.04-4.32 (m, 4H), 1.44-1.45 (m, 27H).

STEP 6: To a solution Cpd. 7-e (1.00 g, 1.71 mmol, 1.00 eq) in MeOH (12 mL) was added Pd/C (0.6 g, 10% purity, 1.00 eq), the reaction was stirred at 25° C. under H₂ (800 mg, 1.00 eq) for 14 hrs. ¹HNMR showed the starting material was consumed completely. Two batches were performed in parallel as above and were combined to work-up. The mixture was filtered and concentrated under reduced pressure to give a residue. Cpd. 7-h (1.80 g, 3.18 mmol, 93.2% yield) was obtained as a white solid. ¹H NMR (400 MHz, CDCl₃) (Product) (3.47-4.15 (m, 9H), 3.14-3.47 (m, 3H), 2.36-2.47 (m, 6H), 1.89 (s, 1H), 1.33-1.38 (m, 27).

STEP 7: To a solution of Cpd. 7-h (2.10 g, 3.71 mmol, 1.00 eq) in DMF (20.0 mL) was added cpd.7-i (1.31 g, 4.08 mmol, 1.10 eq) and HBTU (2.82 g, 7.42 mmol, 2.00 eq), DIEA (1.44 g, 11.1 mmol, 1.94 mL, 3.00 eq) into the mixture was stirred at 25° C. for 12 hrs. LCMS showed the starting material was consumed completely. Added DCM (150 ml), washed with water (120 ml×3), dried with anhydrous Na₂SO₄, filtered and concentrated in vacuum. Two batches were performed in parallel as above and were combined to purification. The residue was purified by column chromatography (SiO₂, petroleum ether/ethyl acetate=20/1 to 5/1). The Cpd. 7-j (4.1 g, 4.72 mmol, 63.6% yield) was obtained as a yellow solid. ¹H NMR (400 MHz, CDCl₃) (Product) δ 7.35 (s, 5H), 5.96 (d, J=9.2 Hz, 1H), 5.20 (t, J=8 Hz, 1H), 5.11 (s, 2H), 3.65-4.13 (m, 13H), 3.39-3.52 (m, 5H), 2.41-2.52 (m, 8H), 2.33-2.45 (m, 3H), 2.20 (t, J=8 Hz, 2H), 1.62-1.64 (m, 6H), 1.45 (s, 27H), 1.26 (s, 12H).

STEP 8: Add Cpd. 7-j (4.10 g, 4.72 mmol, 1.00 eq) into FA (80.0 mL) and stir at 25° C. for 2 hrs. LCMS showed the starting material was consumed completely. Then the reaction solution was concentrated in vacuum. Add DCM (20.0 ml) to dissolve the residue, after that add toluene (60.0 ml) and THE (60.0 mL) and then concentrated to dryness in vacuum (this operation was repeated for three times). The Cpd. 7-k (2.90 g, crude) was obtained as a yellow solid. ¹H NMR (400 MHz, D₆-DMSO) (Product) δ 12.1 (s, 3H), 8.62 (d, J=8 Hz, 1H), 7.32-7.39 (m, 5H), 5.04-5.08 (m, 3H), 3.98-4.16 (m, 1H), 3.870-3.91 (m, 5H), 3.48-3.66 (m, 6H), 3.40 (d, J=12 Hz, 1H), 3.13 (t, J=8 Hz, 1H), 2.69 (s, 1H), 2.41-2.46 (m, 6H), 2.34 (t, J=8 Hz, 2H), 2.10 (t, J=8 Hz, 2H), 1.48-1.54 (m, 4H), 1.22 (s, 12H).

STEP 9: To a solution of Cpd. 7-k (1.50 g, 2.14 mmol, 1.00 eq) in DMF (30.0 mL) was added HBTU (2.60 g, 6.86 mmol, 3.2 eq) and DIPEA (3.60 g, 27.9 mmol, 4.85 mL, 13.0 eq) into the mixture. Then Add Cpd. 7-1 (4.49 g, 6.65 mmol, 3.10 eq, TsOH salt) (the synthesis of Cpd. 7-1 was shown below) into solution and stirred at 25° C. for 12 hrs. LCMS showed the starting material was consumed completely. The aqueous phase was diluted with DCM (100 mL). The combined organic phase was washed with half saturated brine (100 mL×3), dried with anhydrous Na₂SO₄, filtered and concentrated in vacuum. The residue was purified by column chromatography (SiO₂, DCM/MeOH=20/1 to 5/1). The crude product was purified by reversed-phase HPLC. Column: Welch Xtimate C18 250*70 mm #10 um; mobile phase: [water (NH₄HCO₃)-ACN]; B %: 30%-60%, 20 min. The Cpd.7-m (1.50 g, 696 umol, 32.5% yield) was obtained as a white solid.

### The synthesis of Cpd. 7-1

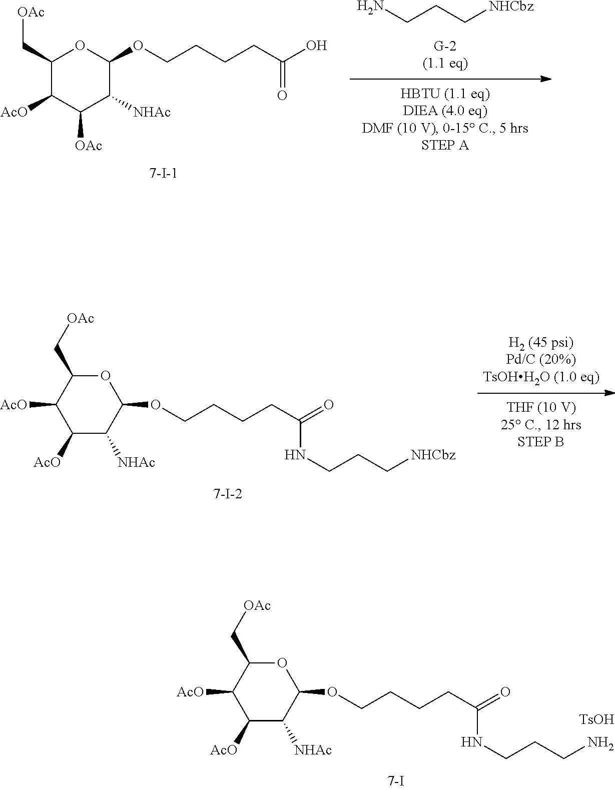 [SMILES: CC(=O)N[C@H]1[C@H](OCCCCC(=O)O)O[C@H](COC(C)=O)[C@H](OC(C)=O)[C@@H]1OC(C)=O ; NCCCNC(=O)OCc1ccccc1 ; CC(=O)N[C@H]1[C@H](OCCCCC(=O)NCCCNC(=O)OCc2ccccc2)O[C@H](COC(C)=O)[C@H](OC(C)=O)[C@@H]1OC(C)=O ; CC(=O)N[C@H]1[C@H](OCCCCC(=O)NCCCN)O[C@H](COC(C)=O)[C@H](OC(C)=O)[C@@H]1OC(C)=O ; "TsOH"]

STEP A: To a solution of Cpd. 7-1-1 (20.0 g, 44.7 mmol, 1.00 eq) in DMF (140 mL) was added DIEA (23.11 g, 179 mmol, 31.1 mL, 4.00 eq), HBTU (18.7 g, 49.2 mmol 1.10 eq) and Cpd. G-2 (10.2 g, 49.2 mmol, 1.10 eq) at 0-5° C. Then the mixture was stirred at 15° C. for 12 hrs. TLC (DCM:MeOH=10:1, R_{f}=0.3) showed Cpd. 7-1-1 was consumed and two main spot detected. The aqueous phase was diluted with DCM (400 mL). The combined organic phase was washed with half saturated brine (200 mL×3), dried with anhydrous Na₂SO₄, filtered and concentrated in vacuum. The crude product (30.0 g) was purified by prep-HPLC. Column: Agela DuraShell C18 250*70 mm*10 um; mobile phase: [water (NH₄HCO₃)-ACN]; B %: 20%-50%, min. Cpd. 7-1-2 (26.0 g, 40.8 mmol, 91.2% yield) was obtained as a yellow oil. ¹H NMR: (400 MHz, DMSO) (Product) δ7.81 (d, J=8 Hz, 1H), 7.73 (t, J=8 Hz, 1H), 7.30-7.38 (m, 5H), 7.20-7.22 (m, 1H), 5.22 (s, 1H), 4.96-5.01 (m, 3H), 4.48-4.50 (d, J=8 Hz, 1H), 4.03 (s, 3H), 3.84-3.92 (m, 1H), 3.70-3.72 (m, 1H), 3.40-3.43 (m, 1H), 2.97-3.06 (m, 4H), 2.10 (s, 2H), 1.99-2.07 (m, 5H), 1.89 (s, 3H), 1.77 (s, 3H), 1.46-1.54 (m, 6H). [M+H]=638.3 (Product).

STEP B: To a solution of Cpd. 7-1-2 (26.0 g, 40.8 mmol, 1.00 eq) in THE (260 mL) was added Pd/C (10.0 g, 10% purity), the reaction was stirred at 25° C. under H2 for 12 hrs. TLC (DCM:MeOH=10:1, R1, R_{f}=0.3, P1, R1=0.2) showed the starting materials was comsumed and one new spot formed. The mixture was filtered and concentrated under reduced pressure to give a residue. Cpd. 7-1 (27.0 g, 40.0 mol, 98% yield, PTSA salt) was obtained as a white solid. ¹H NMR: (400 MHz, DMSO) (Product) δ 7.93 (t, J=4 Hz, 1H), 7.85 (d, J=8 Hz, 1H) 7.69 (s, J=8 Hz, 3H), 7.49 (d, J=8 Hz, 2H), 7.13 (d, J=8 Hz, 2H), 5.22 (s, 1H), 4.96-4.99 (m, 1H), 4.48-4.51 (d, J=12 Hz, 1H), 4.03 (s, 3H), 3.84-3.92 (m, 1H), 3.70-3.72 (m, 1H), 3.38-3.43 (m, 1H), 3.07-3.11 (m, 2H), 2.29 (s, 3H), 2.05-2.10 (m, 5H), 2.00 (s, 3H), 1.89 (s, 3H), 1.76 (s, 1H), 1.64-1.67 (m, 2H), 1.44-1.50 (m, 4H).

STEP 10: To a solution of Cpd. 7-m (850 mg, 394 umol, 1.00 eq) in THE (20 ml) was added Pd/C (850 mg, 10% purity, 1.00 eq), the reaction was stirred at 25° C. under H₂ for 12 hrs. LCMS showed the starting material was consumed completely. The mixture was filtered and concentrated under reduced pressure to give a residue (THF:MeOH=1:1, 100 mL). The Cpd. 7-n (730 mg, crude) was obtained as a white solid. LCMS: ESI⁻, calcd for [M−2H]/2=1031.5, found 1031.9 (Product).

STEP 11: To a solution of Cpd. 7-n (620 mg, 300.07 umol, 1.00 eq) in DMF (6.00 mL) was added HBTU (171 mg, 450 umol, 1.50 eq) and DIPEA (78 mg, 600 umol, 105 uL, 2.00 eq) into the mixture. Then add Cpd. 7-o (189 mg, 450 umol, 1.5 eq) into solution and stirred at 30° C. for 12 hrs. LCMS showed the starting material was consumed completely. The aqueous phase was diluted with DCM (50.0 mL). The combined organic phase was washed with half saturated brine (40.0 mL×3), dried with anhydrous Na₂SO₄, filtered and concentrated in vacuum. Two batches of crude reaction mixture (100 mg and 620 mg scale) were combined for purification. The crude product was purified by reversed-phase HPLC. Column: Waters Xbridge Prep OBD C18 150*40 mm*10 um; mobile phase: [water (NH₄HCO₃)-ACN]; B %: 40%-60%, 8 min. The Cpd. 7-p (500 mg, 203 umol, 55.6% yield) was obtained as a white solid.

STEP 12: To a solution of Cpd. 7-p (100 mg, 40.5 umol, 1.00 eq) in DCM (2.00 mL) was added DMAP (1.24 mg, 10.1 umol, 0.25 eq) and DIPEA (31.4 mg, 243 umol, 42.4 uL, 6.00 eq), into the mixture. Then added succinic anhydride (24.3 mg, 243 umol, 6.00 eq) into solution and stirred at 15° C. for 12 hrs. LCMS showed the starting material was consumed completely. The residue was diluted with DCM (5.00 mL) and extracted with TEAB (15 mL×3). The combined organic layers were washed with H₂O mL (15 mL×2), dried over Na₂SO₄, filtered and concentrated under reduced pressure to give a residue. The crude product was purified by reversed-phase HPLC. Column: C₁₈₋₂ 100×30 mm×5 um; mobile phase: [0.1M TEAB-ACN]; B %: 20%-45%, 20 min. The Cpd. 7-q (4-(((3R,5S)-1-(12-(((2R,3R,4S,5R,6R)-4,5-bis(3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)-6-((3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)methyl)-3-fluorotetrahydro-2H-pyran-2-yl)amino)-12-oxododecanoyl)-5-((bis(4-methoxyphenyl)(phenyl)methoxy)methyl)pyrrolidin-3-yl)oxy)-4-oxobutanoic acid) (55 mg, 21.4 umol, 52.9% yield) was obtained as a white solid. LCMS: ESI⁻, calcd for [M−2H]/2=1282.1, found 1282.6 (Product).

STEP 13: To a solution of Cpd. 7-q (30.0 mg, 11.7 umol, 1.00 eq) in DMF (1.50 mL) was added HBTU (22.1 mg, 58.4 umol, 5.00 eq), DIEA (12.1 mg, 93.5 umol, 16.3 uL, 8.00 eq), DMAP (1.43 mg, 11.7 umol, 1.00 eq) in one portion at 25° C. Then CPG (210 mg; Chemical Name: Aminoalkyl-CPG, 500A from Hebei DNAchem Biotechnology Co., Ltd.) was added to the mixture. The mixture was stirred at 40° C. for 48 hrs. After that, it was filtered and the filter cake was washed with MeOH (5.00 mL×4) and DCM (5.00 mL×4). The filter cake was dried under N₂ stream to give a pale yellow solid. The pale yellow solid above was added into a mixture of dry pyridine (1.50 mL) and Ac₂O (0.30 mL). The mixture was then stirred at 40° C. for 0.5 hr. After that, it was filtered and the filter cake was washed with DCM (5.00 mL×4) and MeOH (5.00 mL×4). The resulted pale yellow solid was dried under vacuum for 12 hrs to give 180 mg solid supported product (loading: 36 umol/g).

### Example 31

### Synthesis of Compound 8

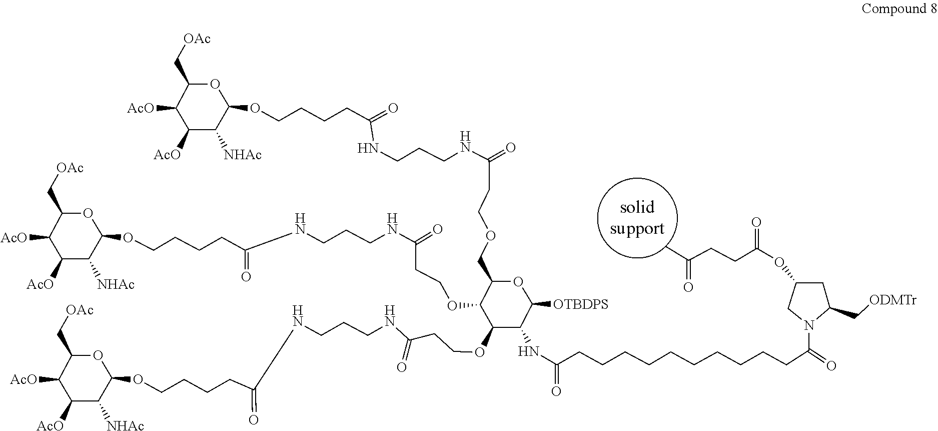

Compound 8 may be synthesized following the synthetic route described above for compound 1 by using Intermediate 8-10 as the starting material.

### Example 32

### Synthesis of Compound 9

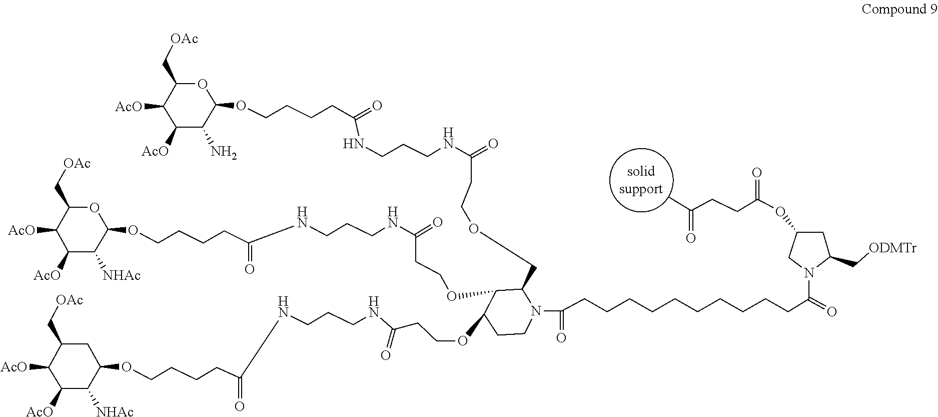

Compound 9 may be synthesized following the synthetic route described above for compound 2 by using intermediate 9-2 as the starting material.

### Example 33

### Synthesis of Compound 10

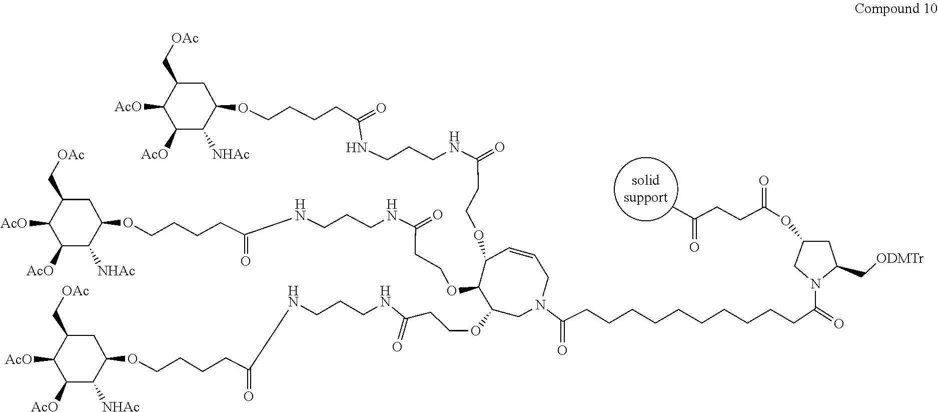

Compound 10 may be synthesized following the synthetic route described above for compound 2 by using Intermediate 10-2 as the starting material.

### Example 34

### Synthesis of Compound 11

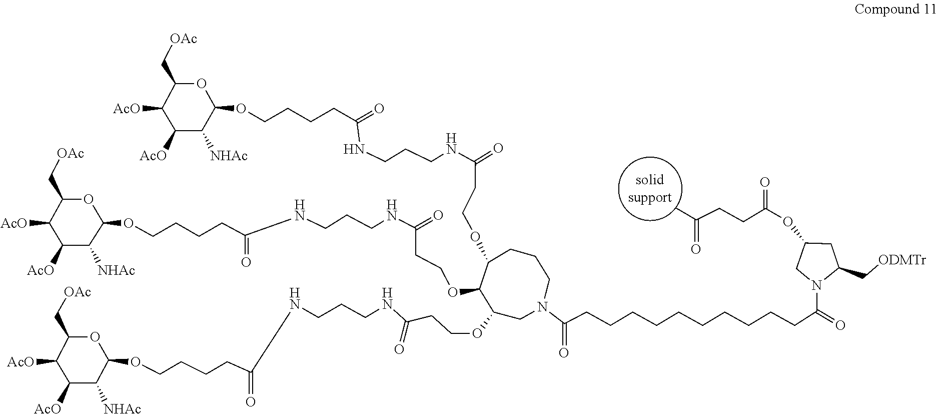

Compound 11 may be synthesized following the synthetic route described above for compound 2 by using Intermediate 11-2 as the starting material.

### Example 35

### Synthesis of Compound 12

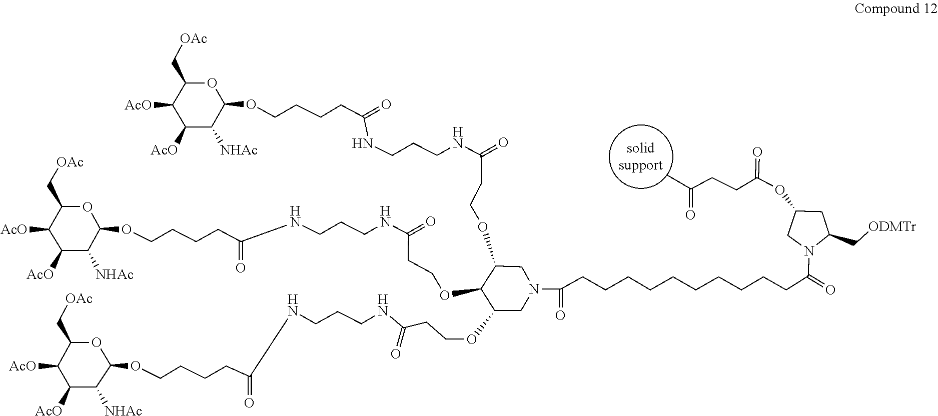

Compound 12 may be synthesized following the synthetic route described above for compound 2 by using Intermediate 12-2 as the starting material.

### Example 36

### Synthesis of Compound 13-7

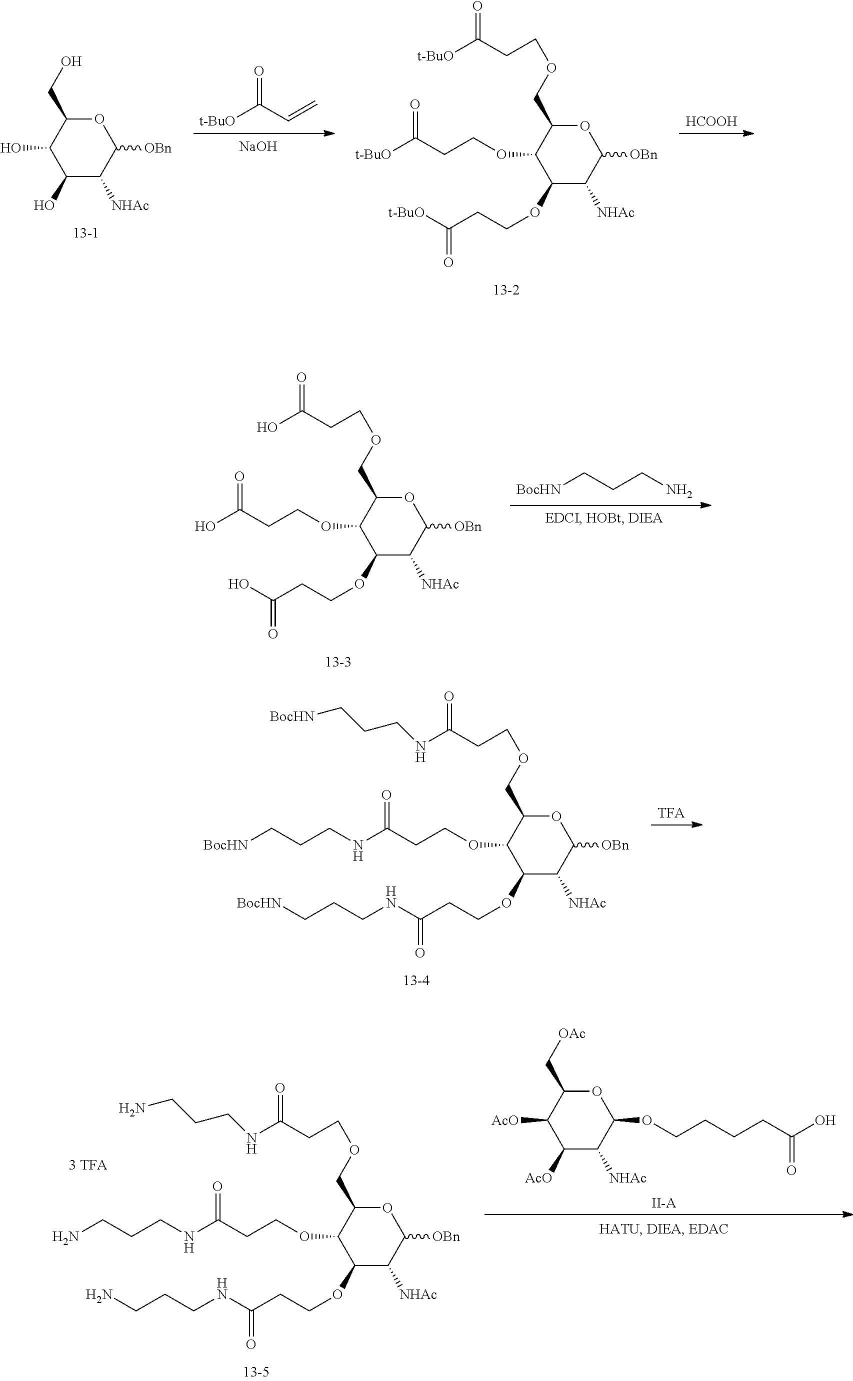

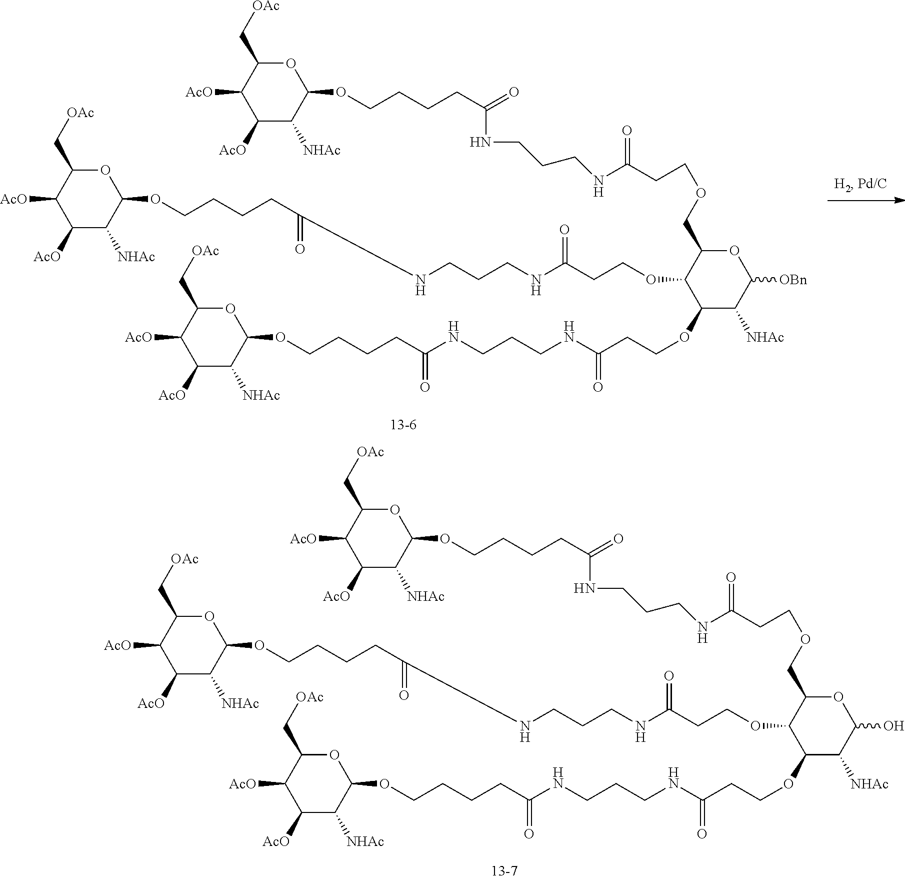

### Example 37

### Synthesis of Compound 13-11

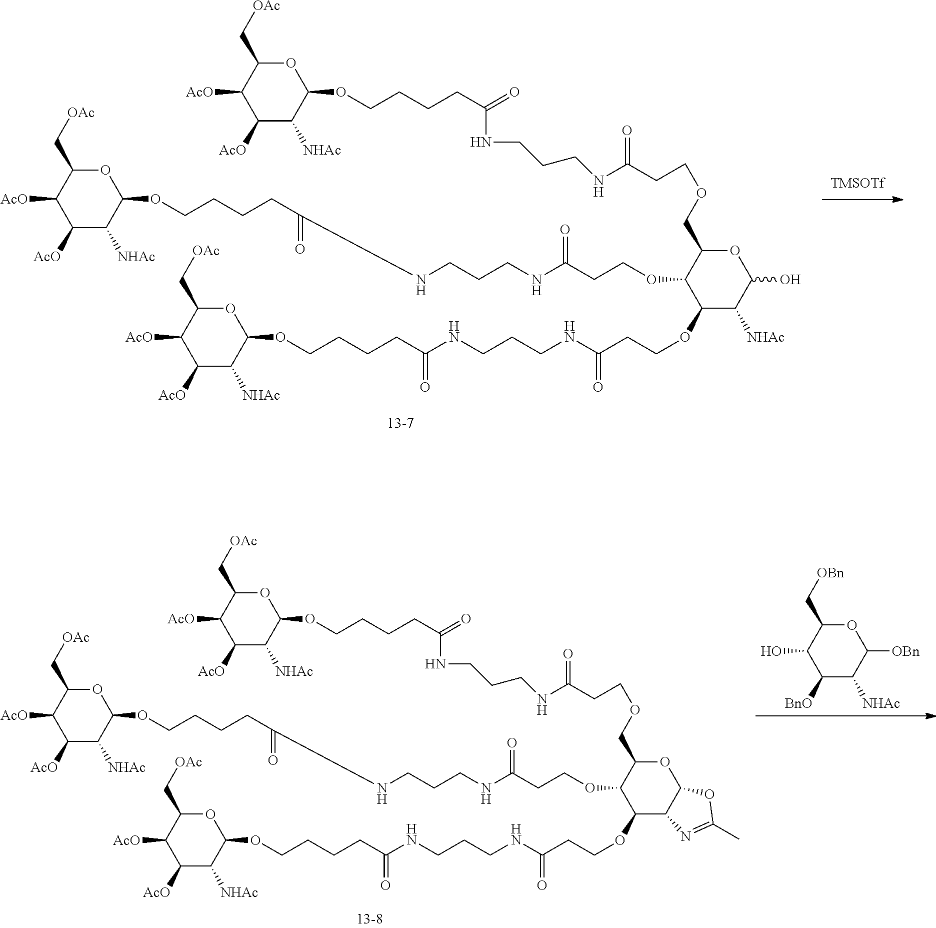

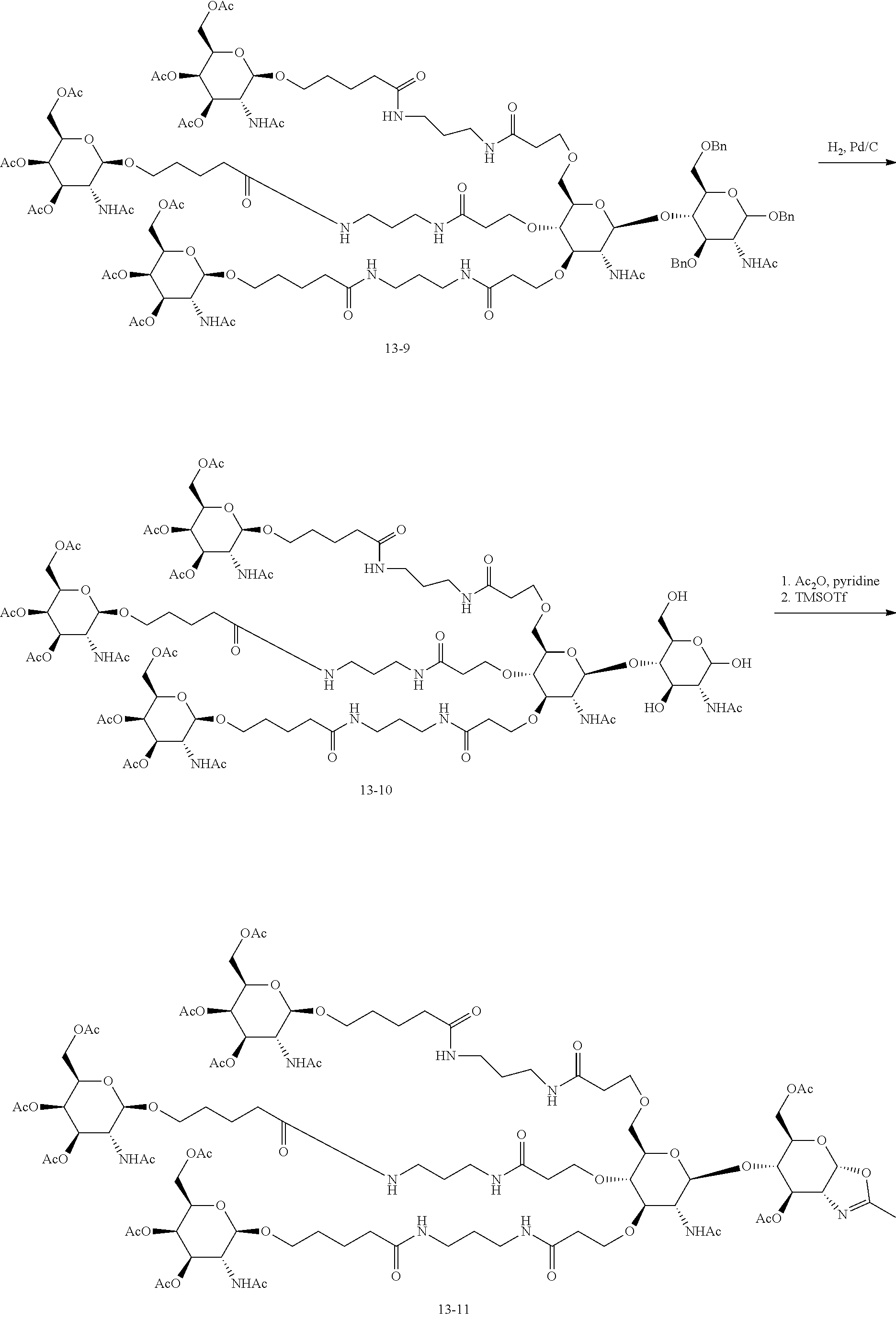

### Example 38

### Synthesis of Compound 13

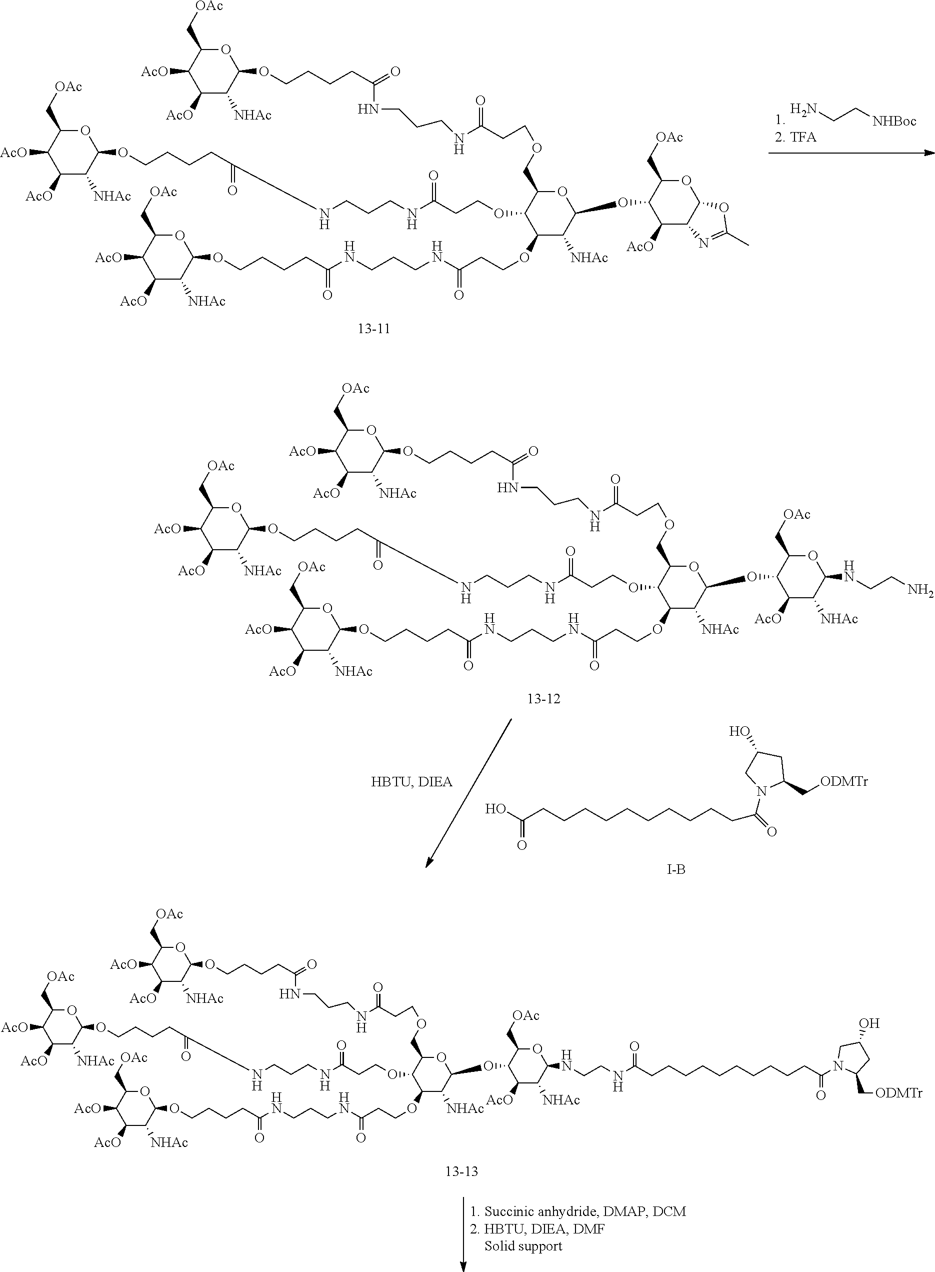

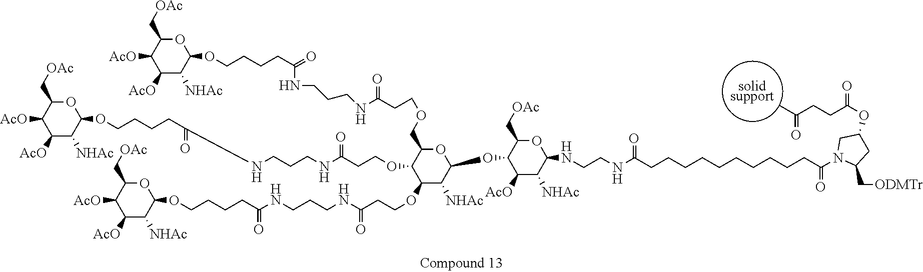

### Example 39

### Synthesis of Compound 14

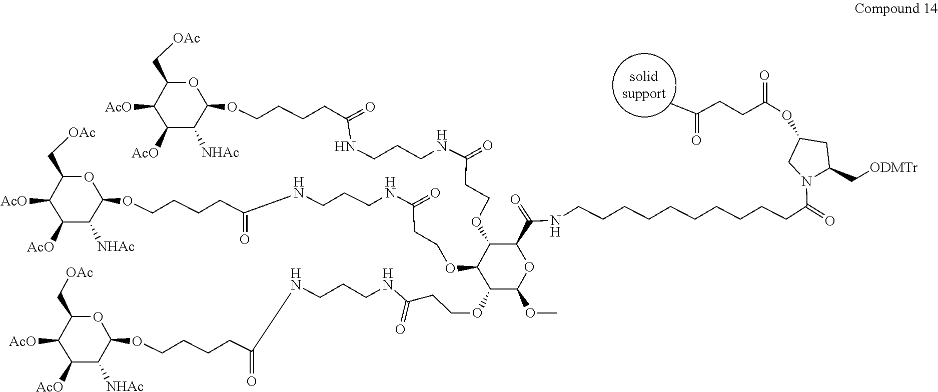

Compound 14 may be synthesized following the synthetic route described above for compound 1 by using Intermediate 14-4 as the starting material of which the synthesis is shown below.

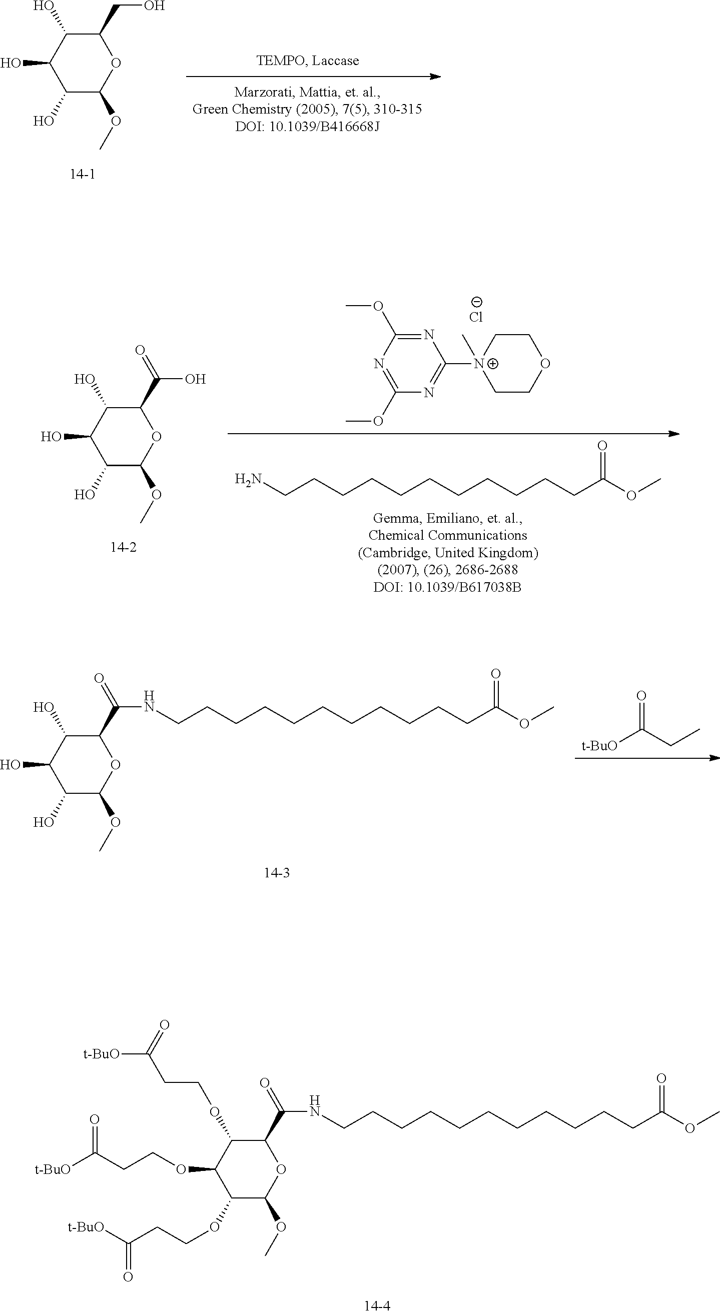

### Example 40

### Synthesis of Compound 15

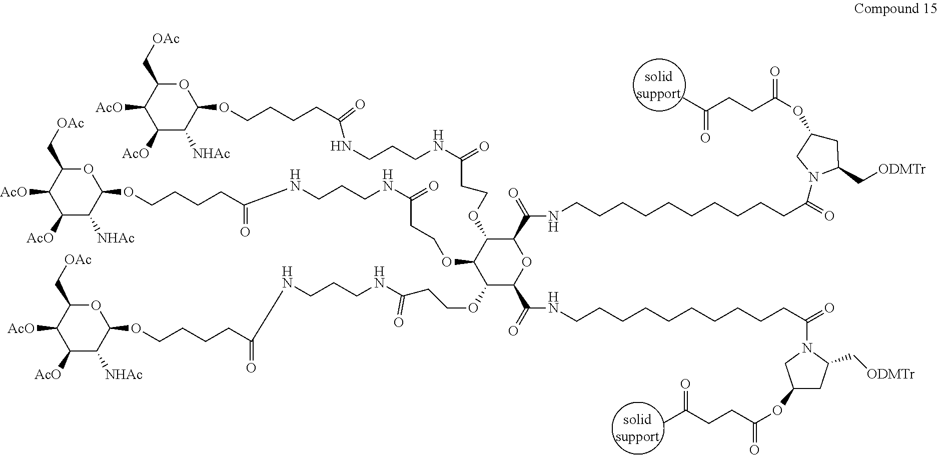

Compound 15 may be synthesized following the synthetic route described above for Compound 1 by using Intermediate 15-3 as the starting material of which the synthesis is shown below.

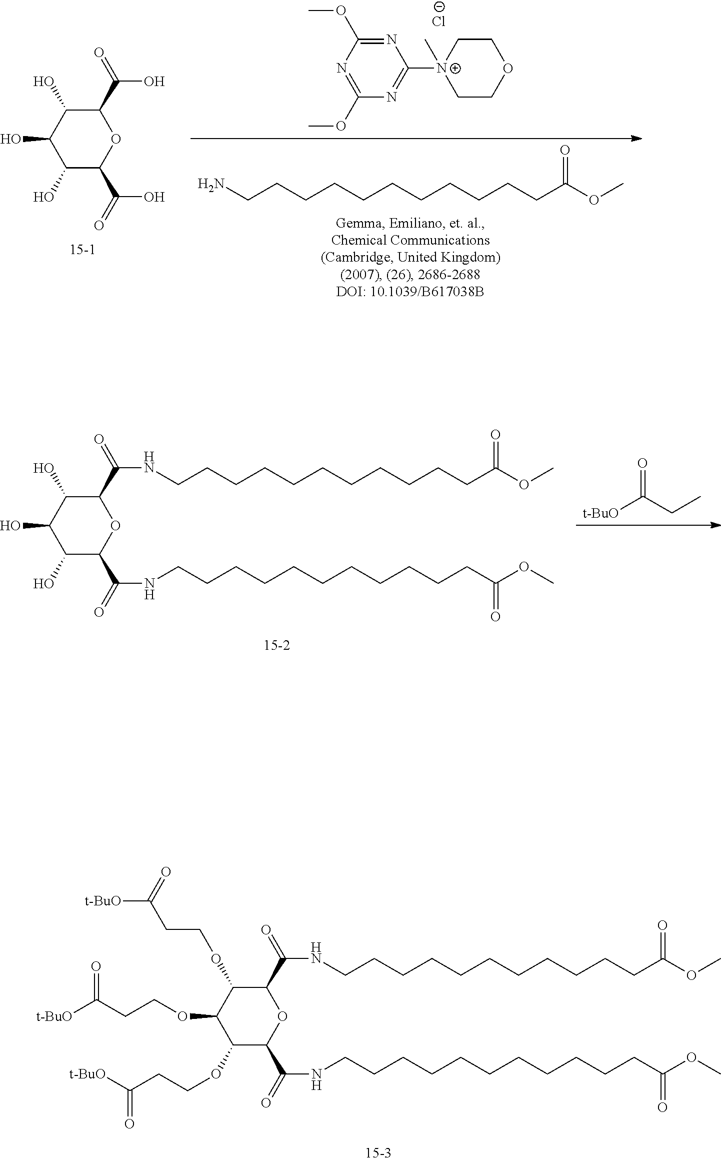

### Example 41

### Synthesis of Compound 16

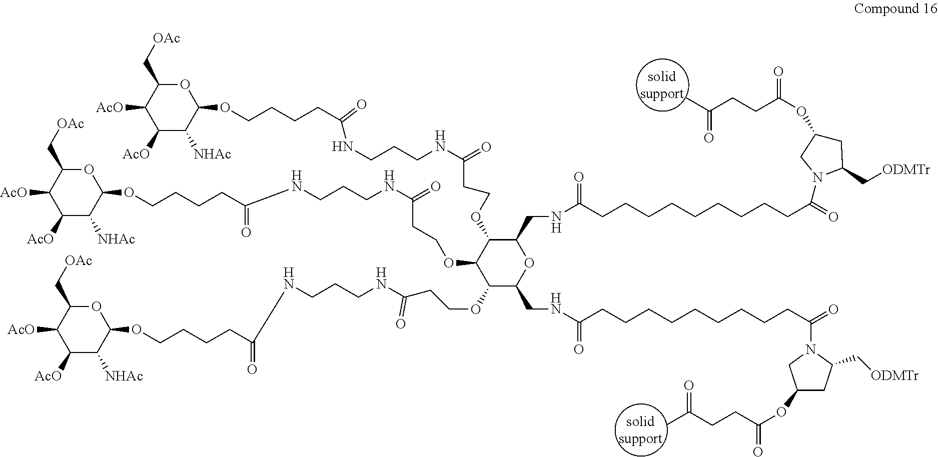

Compound 16 may be synthesized following the synthetic route described above for Compound 2 by using (2R,3S,4s,5R,6S)-2,6-bis(aminomethyl)tetrahydro-2H-pyran-3,4,5-triol as the starting material.

### Examples 42-49

(omitted)

### Example 50

### Synthesis of Compound 25

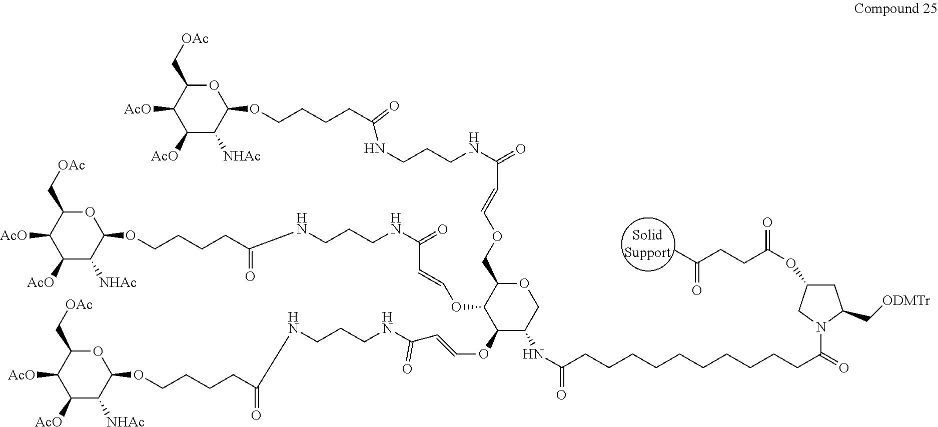

Compound 25 may be synthesized following the synthetic route described above for Compound 2 by using the Intermediate 25-7 as the starting material of which the synthesis is shown below.

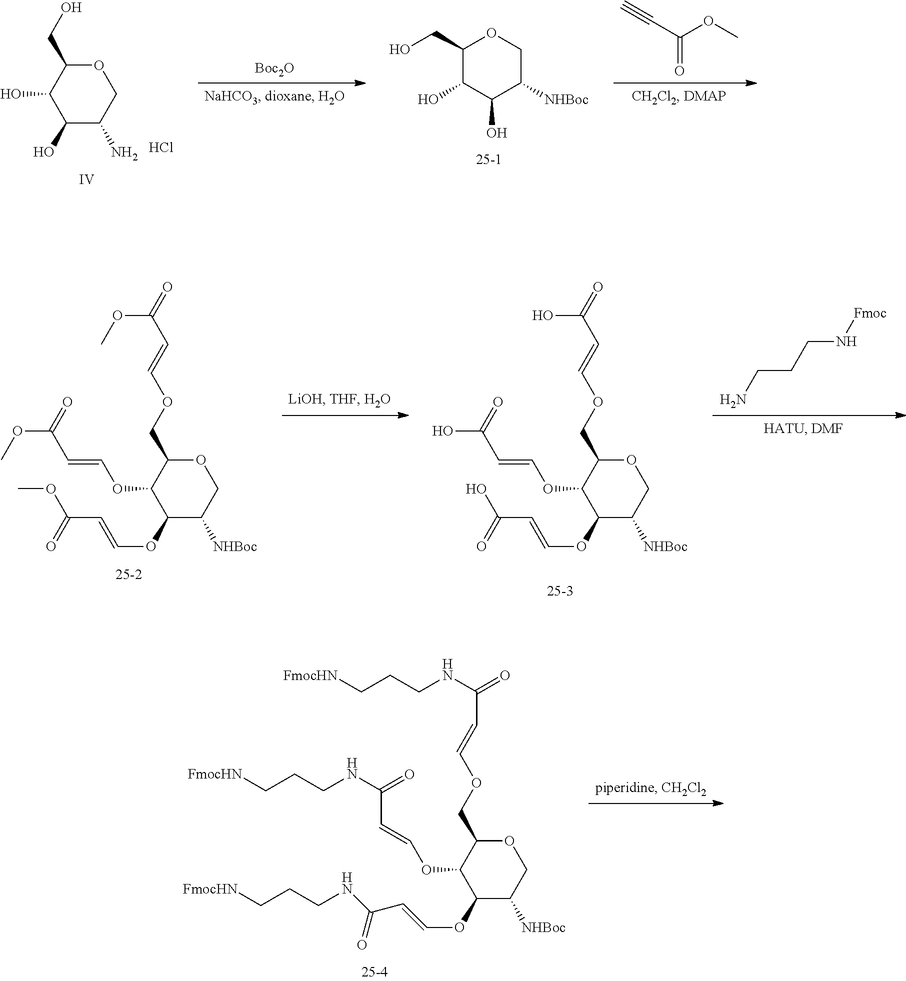

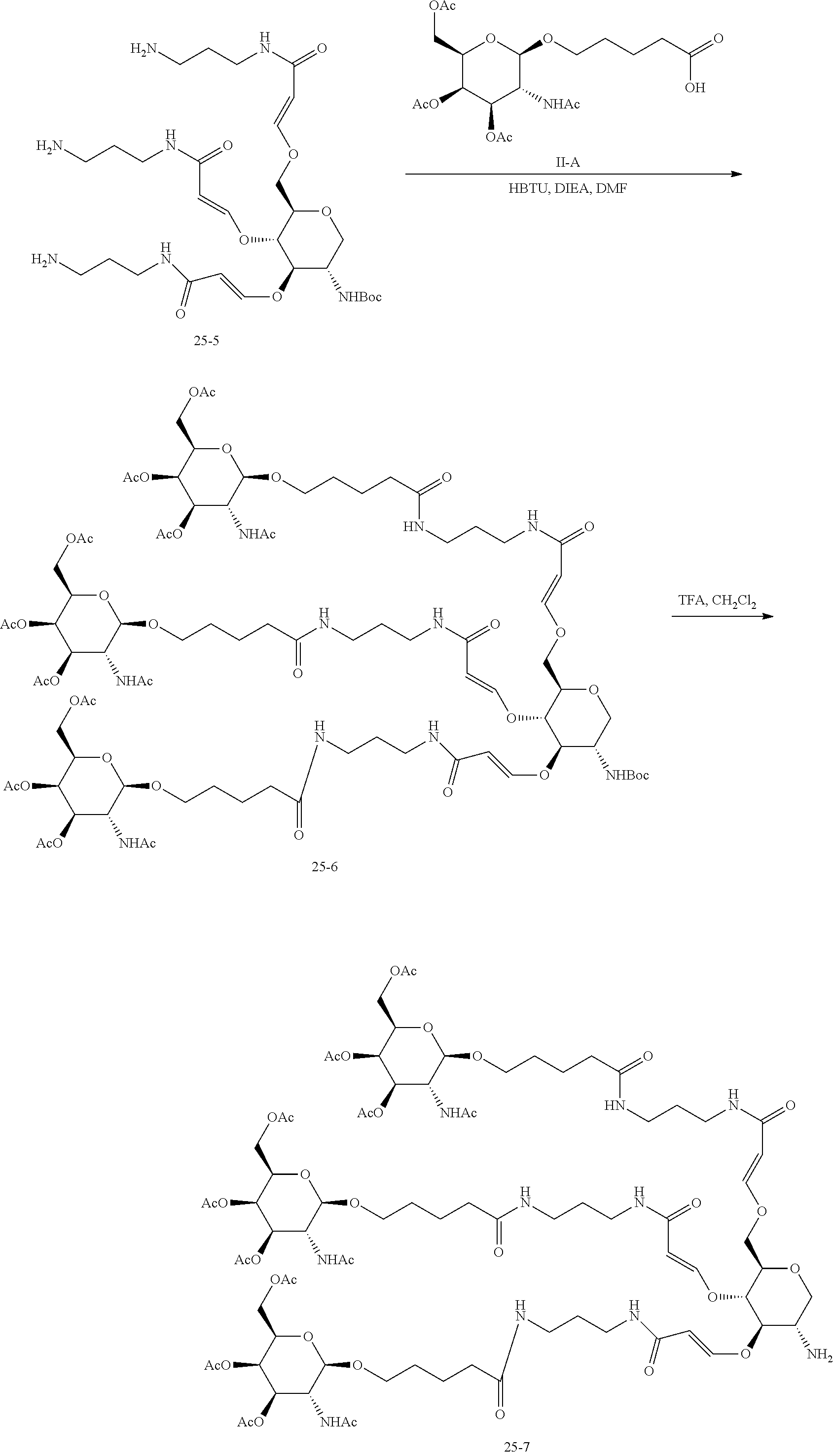

### Example 51

### Synthesis of GalNAc-siRNA conjugate compound 1-F

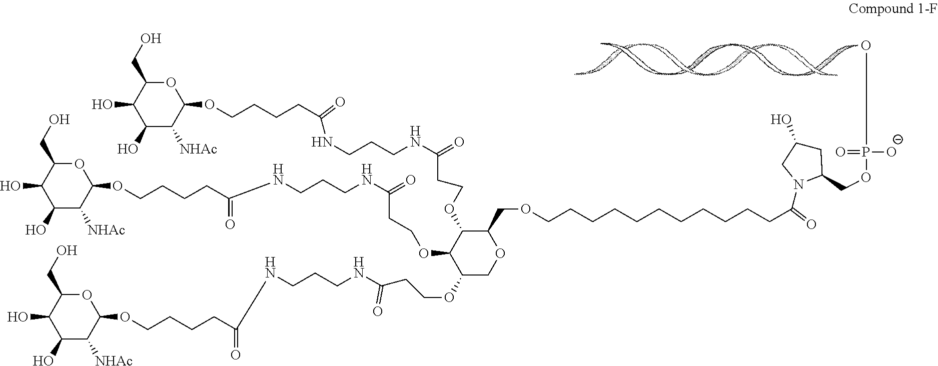 [SMILES: CC(=O)N[C@H]1[C@H](OCCCCC(=O)NCCCNC(=O)CCO[C@H]2[C@H](OCCC(=O)NCCCNC(=O)CCCCO[C@@H]3O[C@H](CO)[C@H](O)[C@H](O)[C@H]3NC(C)=O)[C@@H](COCCCCCCCCCCCC(=O)N3C[C@H](O)C[C@H]3COP(=O)([O-])O)OC[C@@H]2OCCC(=O)NCCCNC(=O)CCCCO[C@@H]2O[C@H](CO)[C@H](O)[C@H](O)[C@H]2NC(C)=O)O[C@H](CO)[C@H](O)[C@@H]1O]

Using the conjugate building block compound 1 described earlier, RNA is synthesized with the ligand attached to 3′-end of the sense strand according to known procedures. This is annealed with an antisense strand. The product is shown above.

### Example 52

### Synthesis of GalNAc-siRNA conjugate compound 2-F

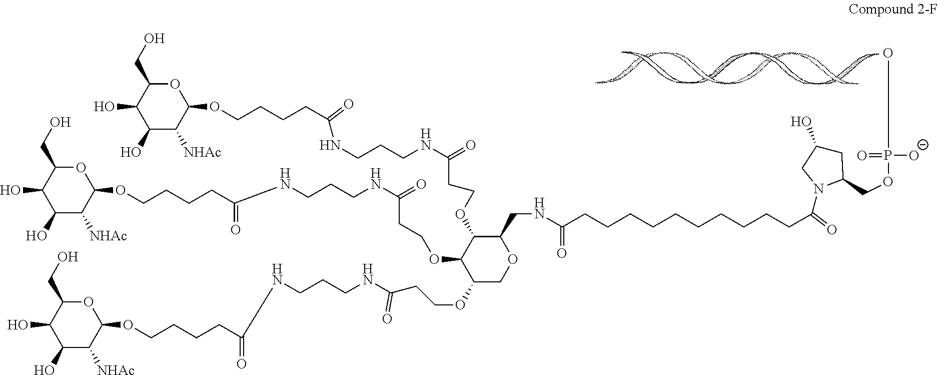 [SMILES: CC(=O)N[C@H]1[C@H](OCCCCC(=O)NCCCNC(=O)CCO[C@H]2[C@H](OCCC(=O)NCCCNC(=O)CCCCO[C@@H]3O[C@H](CO)[C@H](O)[C@H](O)[C@H]3NC(C)=O)[C@@H](CNC(=O)CCCCCCCCCCC(=O)N3C[C@H](O)C[C@H]3COP(=O)([O-])O)OC[C@@H]2OCCC(=O)NCCCNC(=O)CCCCO[C@@H]2O[C@H](CO)[C@H](O)[C@H](O)[C@H]2NC(C)=O)O[C@H](CO)[C@H](O)[C@@H]1O]

Using the conjugate building block compound 2 described earlier (Example 25), RNA is synthesized with the ligand attached to 3′-end of the sense strand according to known procedures. This is annealed with an antisense strand. The product is shown above.

### Example 53

### Synthesis of GalNAc-siRNA conjugate compound 3-F

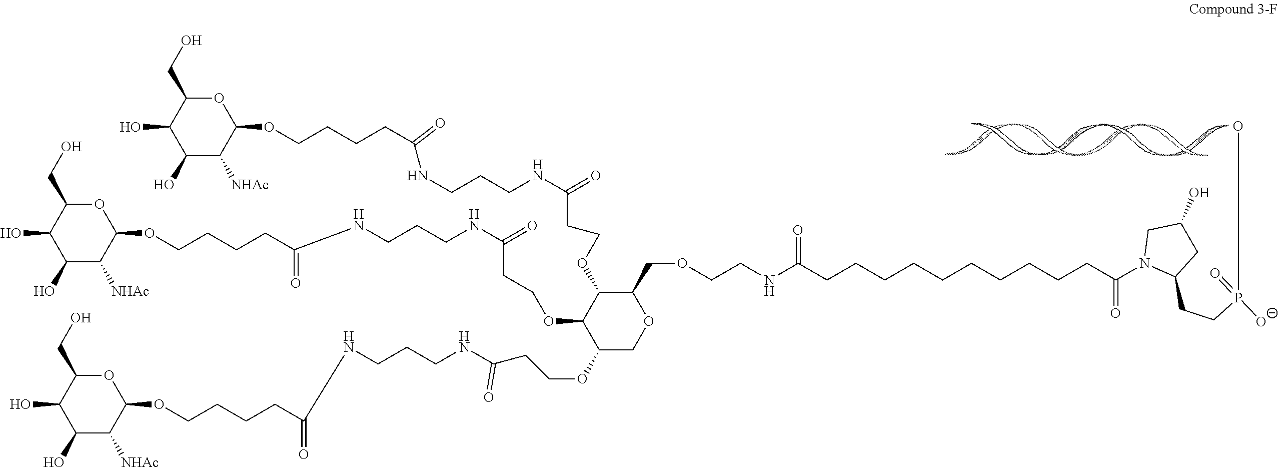 [SMILES: CC(=O)N[C@H]1[C@H](OCCCCC(=O)NCCCNC(=O)CCO[C@H]2[C@H](OCCC(=O)NCCCNC(=O)CCCCO[C@@H]3O[C@H](CO)[C@H](O)[C@H](O)[C@H]3NC(C)=O)[C@@H](COCCNC(=O)CCCCCCCCCCC(=O)N3C[C@H](O)C[C@H]3CCP(=O)([O-])O)OC[C@@H]2OCCC(=O)NCCCNC(=O)CCCCO[C@@H]2O[C@H](CO)[C@H](O)[C@H](O)[C@H]2NC(C)=O)O[C@H](CO)[C@H](O)[C@@H]1O]

Using the conjugate building block compound 3 described earlier, RNA is synthesized with the ligand attached to 3′-end of the sense strand according to known procedures. This is annealed with an antisense strand. The product is shown above.

### Example 54

### Synthesis of GalNAc-siRNA conjugate compound 4-F

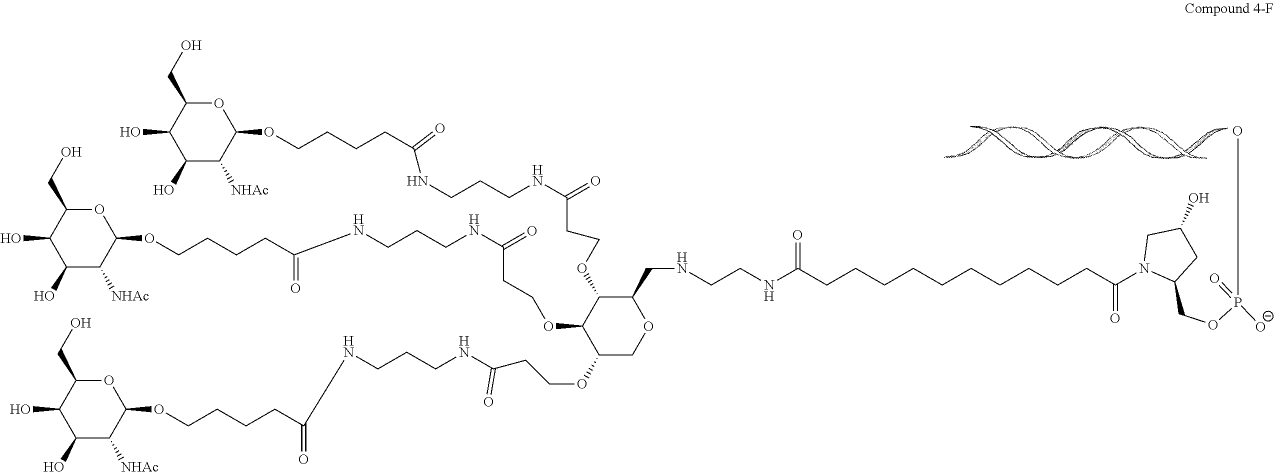 [SMILES: CC(=O)N[C@H]1[C@H](OCCCCC(=O)NCCCNC(=O)CCO[C@H]2[C@H](OCCC(=O)NCCCNC(=O)CCCCO[C@@H]3O[C@H](CO)[C@H](O)[C@H](O)[C@H]3NC(C)=O)[C@@H](CNCCNC(=O)CCCCCCCCCCC(=O)N3C[C@H](O)C[C@H]3COP(=O)([O-])O)OC[C@@H]2OCCC(=O)NCCCNC(=O)CCCCO[C@@H]2O[C@H](CO)[C@H](O)[C@H](O)[C@H]2NC(C)=O)O[C@H](CO)[C@H](O)[C@@H]1O]

Using the conjugate building block compound 4 described earlier, RNA is synthesized with the ligand attached to 3′-end of the sense strand according to known procedures. This is annealed with an antisense strand. The product is shown above.

### Example 55

### Synthesis of GalNAc-siRNA conjugate compound 5-F

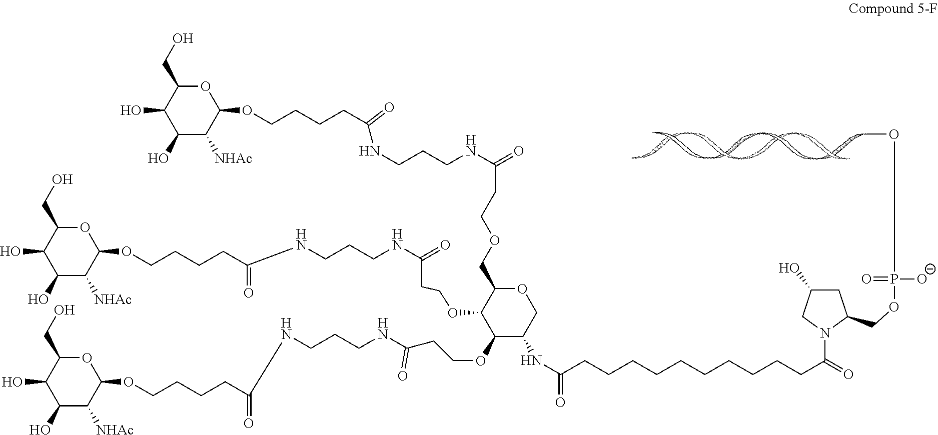 [SMILES: COP(=O)([O-])OC[C@@H]1C[C@@H](O)CN1C(=O)CCCCCCCCCCC(=O)N[C@H]1CO[C@H](COCCC(=O)NCCCNC(=O)CCCCO[C@@H]2O[C@H](CO)[C@H](O)[C@H](O)[C@H]2NC(C)=O)[C@@H](OCCC(=O)NCCCNC(=O)CCCCO[C@@H]2O[C@H](CO)[C@H](O)[C@H](O)[C@H]2NC(C)=O)[C@@H]1OCCC(=O)NCCCNC(=O)CCCCO[C@@H]1O[C@H](CO)[C@H](O)[C@H](O)[C@H]1NC(C)=O]

Using the conjugate building block compound 5 described earlier (example 28), RNA is synthesized with the ligand attached to 3′-end of the sense strand according to known procedures. This is annealed with an antisense strand. The product is shown above.

### Example 56

### Synthesis of GalNAc-siRNA conjugate compound 6-F

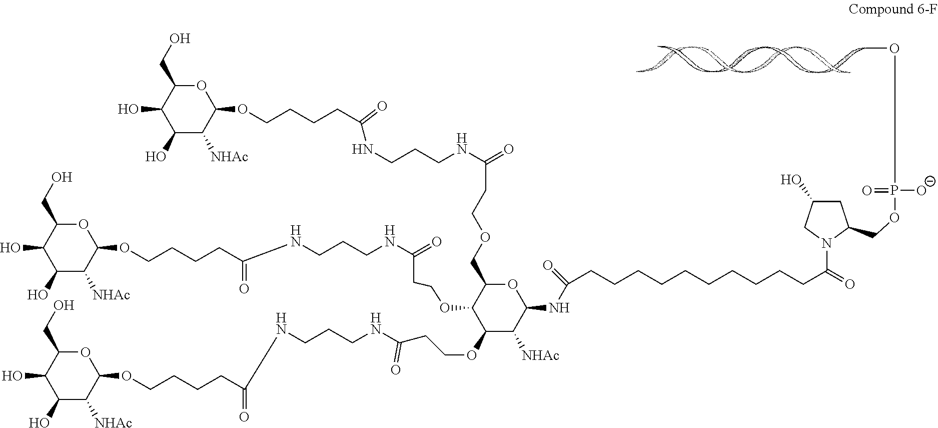 [SMILES: COP(=O)([O-])OC[C@@H]1C[C@@H](O)CN1C(=O)CCCCCCCCCCC(=O)N[C@@H]1O[C@H](COCCC(=O)NCCCNC(=O)CCCCO[C@@H]2O[C@H](CO)[C@H](O)[C@H](O)[C@H]2NC(C)=O)[C@@H](OCCC(=O)NCCCNC(=O)CCCCO[C@@H]2O[C@H](CO)[C@H](O)[C@H](O)[C@H]2NC(C)=O)[C@H](OCCC(=O)NCCCNC(=O)CCCCO[C@@H]2O[C@H](CO)[C@H](O)[C@H](O)[C@H]2NC(C)=O)[C@H]1NC(C)=O]

Using the conjugate building block compound 6 described earlier (Example 29), RNA is synthesized with the ligand attached to 3′-end of the sense strand according to known procedures. This is annealed with an antisense strand. The product is shown above.

### Example 57

### Synthesis of GalNAc-siRNA conjugate compound 7-F

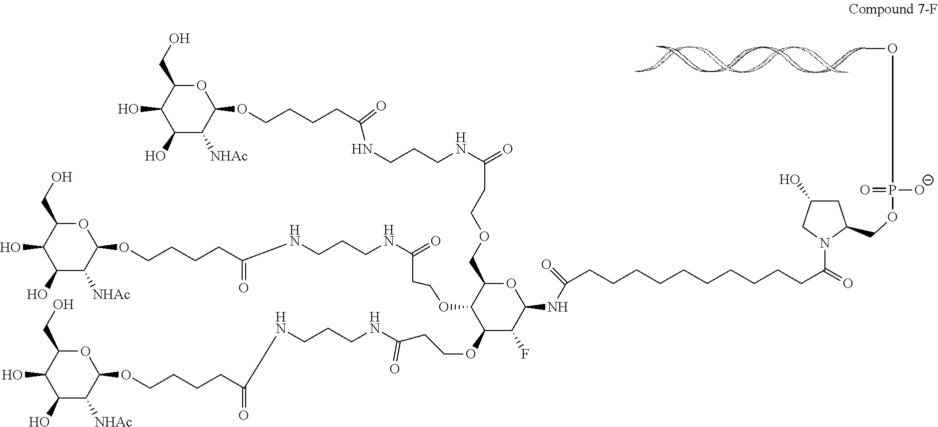 [SMILES: COP(=O)([O-])OC[C@@H]1C[C@@H](O)CN1C(=O)CCCCCCCCCCC(=O)N[C@@H]1O[C@H](COCCC(=O)NCCCNC(=O)CCCCO[C@@H]2O[C@H](CO)[C@H](O)[C@H](O)[C@H]2NC(C)=O)[C@@H](OCCC(=O)NCCCNC(=O)CCCCO[C@@H]2O[C@H](CO)[C@H](O)[C@H](O)[C@H]2NC(C)=O)[C@H](OCCC(=O)NCCCNC(=O)CCCCO[C@@H]2O[C@H](CO)[C@H](O)[C@H](O)[C@H]2NC(C)=O)[C@H]1F]

Using the conjugate building block compound 7 described earlier (Example 30), RNA is synthesized with the ligand attached to 3′-end of the sense strand according to known procedures. This is annealed with an antisense strand. The product is shown above.

### Example 58

### Synthesis of GalNAc-siRNA conjugate compound 8-F

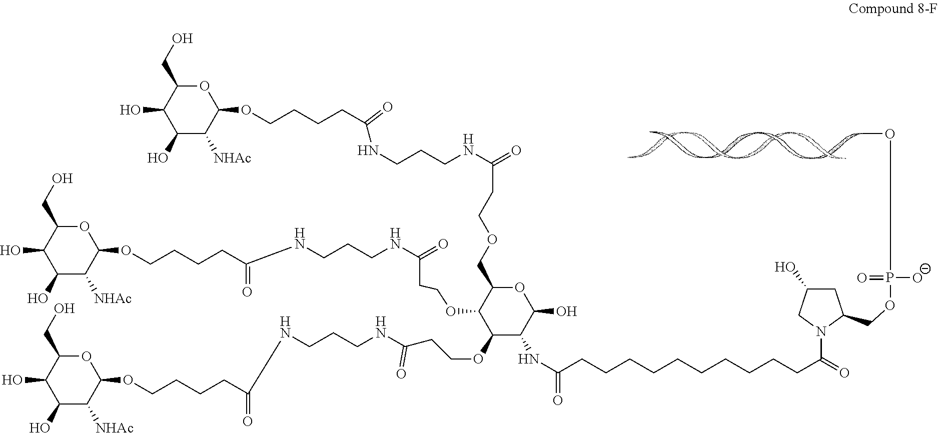 [SMILES: COP(=O)([O-])OC[C@@H]1C[C@@H](O)CN1C(=O)CCCCCCCCCCC(=O)N[C@@H]1[C@@H](OCCC(=O)NCCCNC(=O)CCCCO[C@@H]2O[C@H](CO)[C@H](O)[C@H](O)[C@H]2NC(C)=O)[C@H](OCCC(=O)NCCCNC(=O)CCCCO[C@@H]2O[C@H](CO)[C@H](O)[C@H](O)[C@H]2NC(C)=O)[C@@H](COCCC(=O)NCCCNC(=O)CCCCO[C@@H]2O[C@H](CO)[C@H](O)[C@H](O)[C@H]2NC(C)=O)O[C@H]1O]

Using the conjugate building block compound 8 described earlier, RNA is synthesized with the ligand attached to 3′-end of the sense strand according to known procedures. This is annealed with an antisense strand. The product is shown above.

### Example 59

### Synthesis of GalNAc-siRNA conjugate compound 9-F

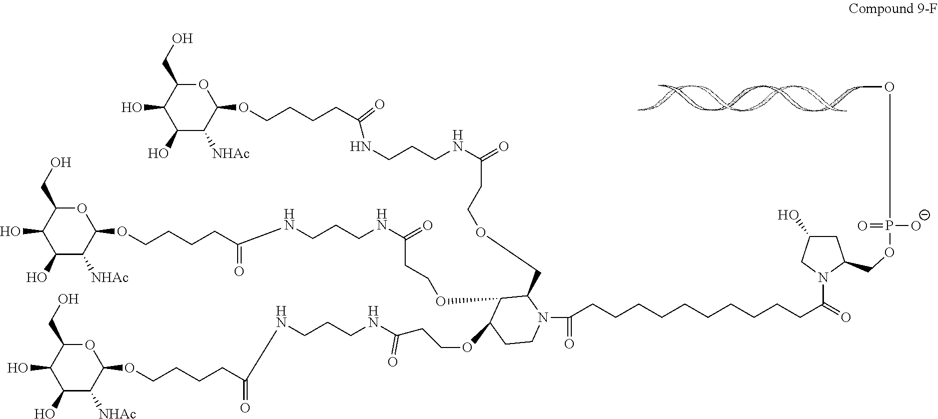 [SMILES: COP(=O)([O-])OC[C@@H]1C[C@@H](O)CN1C(=O)CCCCCCCCCCC(=O)N1CC[C@@H](OCCC(=O)NCCCNC(=O)CCCCO[C@@H]2O[C@H](CO)[C@H](O)[C@H](O)[C@H]2NC(C)=O)[C@H](OCCC(=O)NCCCNC(=O)CCCCO[C@@H]2O[C@H](CO)[C@H](O)[C@H](O)[C@H]2NC(C)=O)[C@H]1COCCC(=O)NCCCNC(=O)CCCCO[C@@H]1O[C@H](CO)[C@H](O)[C@H](O)[C@H]1NC(C)=O]

Using the conjugate building block compound 9 described earlier, RNA is synthesized with the ligand attached to 3′-end of the sense strand according to known procedures. This is annealed with an antisense strand. The product is shown above.

### Example 60

### Synthesis of GalNAc-siRNA conjugate compound 10-F

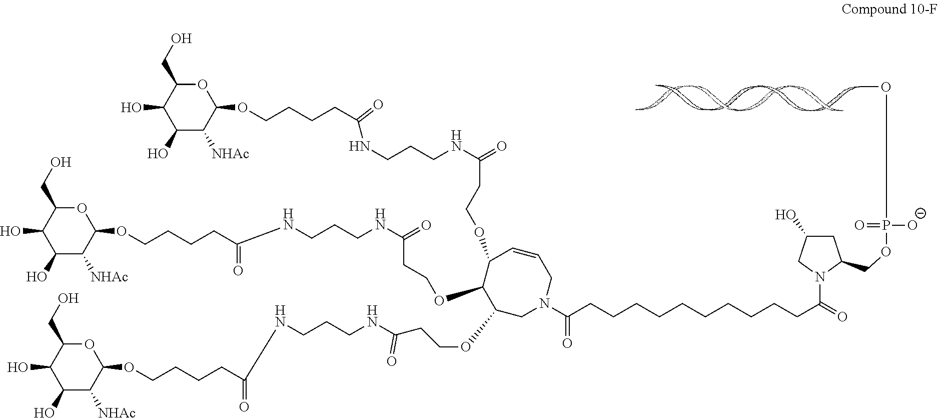 [SMILES: COP(=O)([O-])OC[C@@H]1C[C@@H](O)CN1C(=O)CCCCCCCCCCC(=O)N1C/C=C\[C@@H](OCCC(=O)NCCCNC(=O)CCCCO[C@@H]2O[C@H](CO)[C@H](O)[C@H](O)[C@H]2NC(C)=O)[C@H](OCCC(=O)NCCCNC(=O)CCCCO[C@@H]2O[C@H](CO)[C@H](O)[C@H](O)[C@H]2NC(C)=O)[C@@H](OCCC(=O)NCCCNC(=O)CCCCO[C@@H]2O[C@H](CO)[C@H](O)[C@H](O)[C@H]2NC(C)=O)C1]

Using the conjugate building block compound 10 described earlier, RNA is synthesized with the ligand attached to 3′-end of the sense strand according to known procedures. This is annealed with an antisense strand. The product is shown above.

### Example 61

### Synthesis of GalNAc-siRNA conjugate compound 11-F

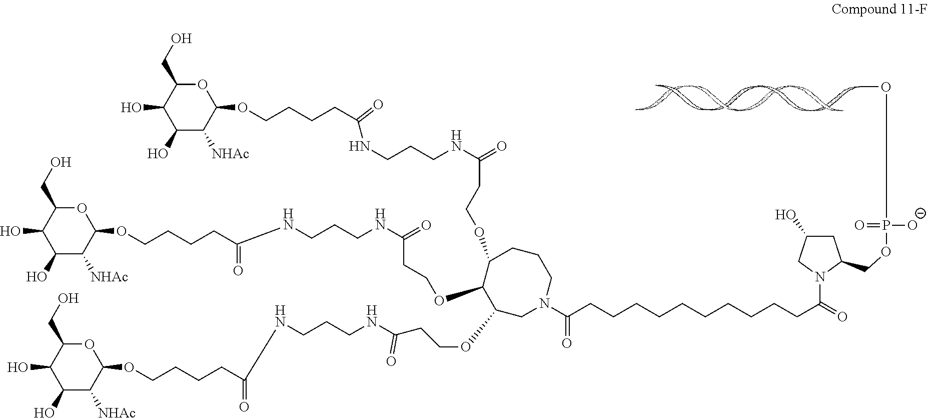 [SMILES: COP(=O)([O-])OC[C@@H]1C[C@@H](O)CN1C(=O)CCCCCCCCCCC(=O)N1CCC[C@@H](OCCC(=O)NCCCNC(=O)CCCCO[C@@H]2O[C@H](CO)[C@H](O)[C@H](O)[C@H]2NC(C)=O)[C@H](OCCC(=O)NCCCNC(=O)CCCCO[C@@H]2O[C@H](CO)[C@H](O)[C@H](O)[C@H]2NC(C)=O)[C@@H](OCCC(=O)NCCCNC(=O)CCCCO[C@@H]2O[C@H](CO)[C@H](O)[C@H](O)[C@H]2NC(C)=O)C1]

Using the conjugate building block compound 11 described earlier, RNA is synthesized with the ligand attached to 3′-end of the sense strand according to known procedures. This is annealed with an antisense strand. The product is shown above.

### Example 62

### Synthesis of GalNAc-siRNA conjugate compound 12-F

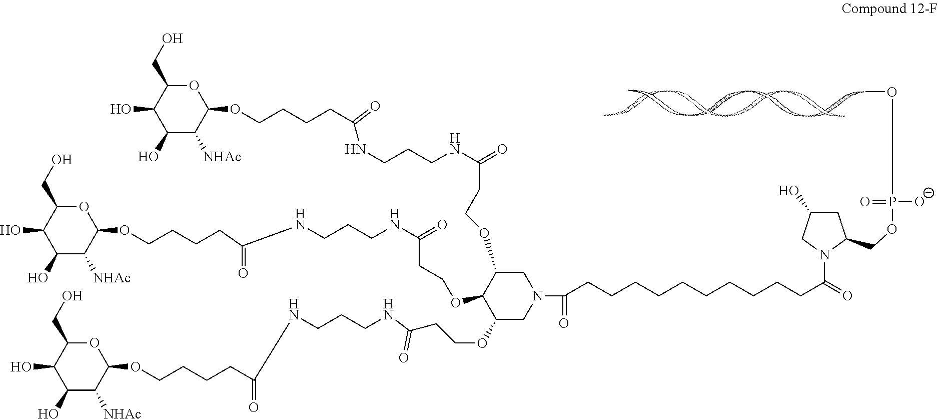 [SMILES: COP(=O)([O-])OC[C@@H]1C[C@@H](O)CN1C(=O)CCCCCCCCCCC(=O)N1C[C@H](OCCC(=O)NCCCNC(=O)CCCCO[C@@H]2O[C@H](CO)[C@H](O)[C@H](O)[C@H]2NC(C)=O)[C@@H](OCCC(=O)NCCCNC(=O)CCCCO[C@@H]2O[C@H](CO)[C@H](O)[C@H](O)[C@H]2NC(C)=O)[C@H](OCCC(=O)NCCCNC(=O)CCCCO[C@@H]2O[C@H](CO)[C@H](O)[C@H](O)[C@H]2NC(C)=O)C1]

Using the conjugate building block compound 12 described earlier, RNA is synthesized with the ligand attached to 3′-end of the sense strand according to known procedures. This is annealed with an antisense strand. The product is shown above.

### Example 63

### Synthesis of GalNAc-siRNA conjugate compound 13-F

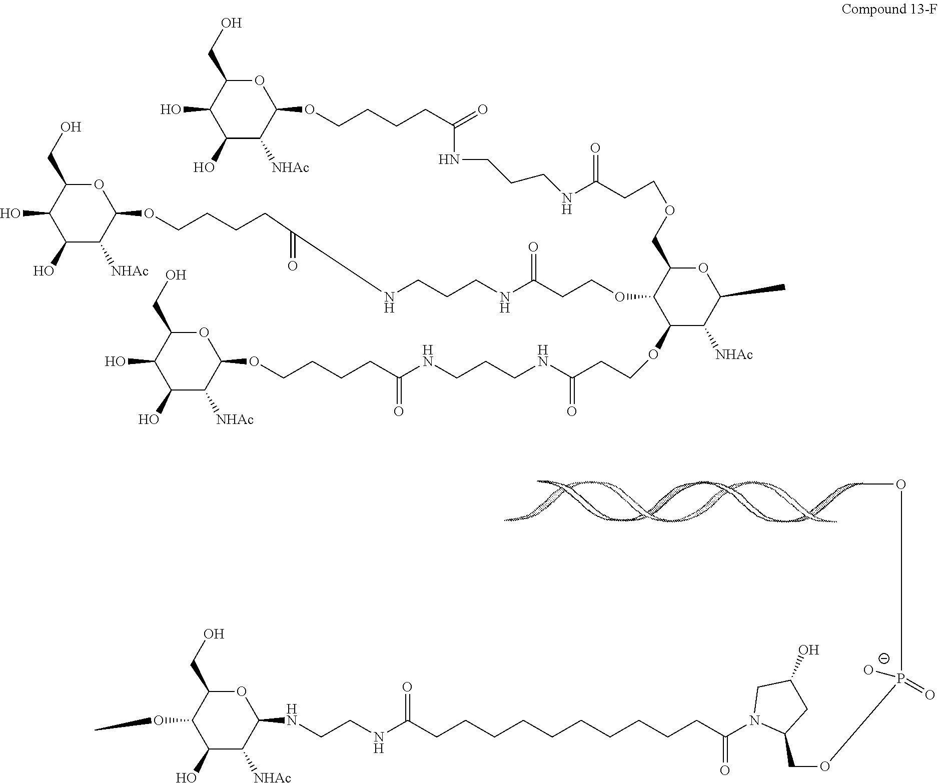

Using the conjugate building block compound 13 described earlier, RNA is synthesized with the ligand attached to 3′-end of the sense strand according to known procedures. This is annealed with an antisense strand. The product is shown above.

### Example 64

### Synthesis of GalNAc-siRNA conjugate compound 14-F

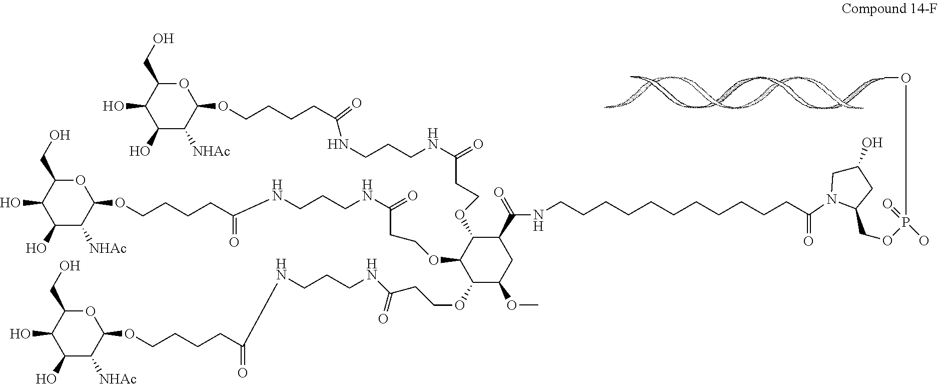 [SMILES: CO[C@@H]1C[C@H](C(=O)NCCCCCCCCCCCC(=O)N2C[C@H](O)C[C@H]2COP(=O)(O)OC)[C@@H](OCCC(=O)NCCCNC(=O)CCCCO[C@@H]2O[C@H](CO)[C@H](O)[C@H](O)[C@H]2NC(C)=O)[C@H](OCCC(=O)NCCCNC(=O)CCCCO[C@@H]2O[C@H](CO)[C@H](O)[C@H](O)[C@H]2NC(C)=O)[C@H]1OCCC(=O)NCCCNC(=O)CCCCO[C@@H]1O[C@H](CO)[C@H](O)[C@H](O)[C@H]1NC(C)=O]

Using the conjugate building block compound 14 described earlier, RNA is synthesized with the ligand attached to 3′-end of the sense strand according to known procedures. This is annealed with an antisense strand. The product is shown above.

### Example 65

### Synthesis of GalNAc-siRNA conjugate compound 15-F

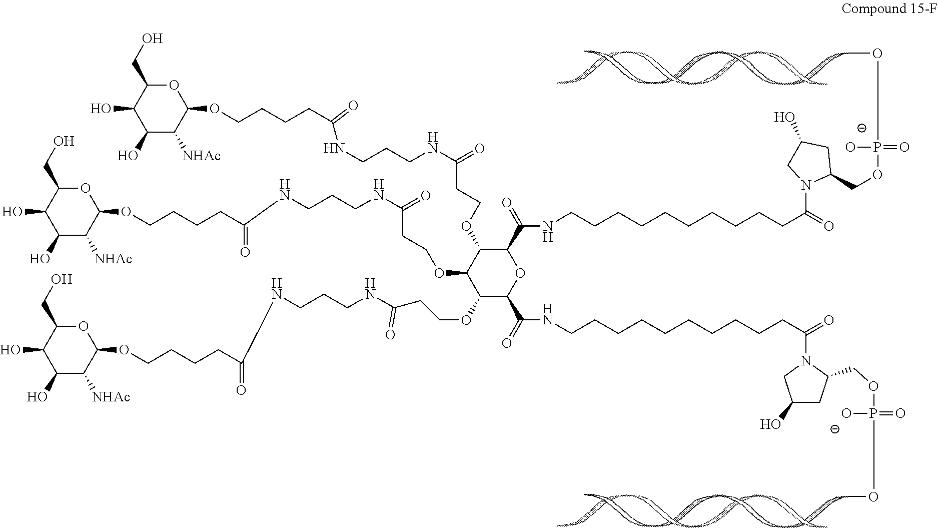 [SMILES: COP(=O)([O-])OC[C@@H]1C[C@@H](O)CN1C(=O)CCCCCCCCCCNC(=O)[C@H]1O[C@@H](C(=O)NCCCCCCCCCCC(=O)N2C[C@H](O)C[C@H]2COP(=O)([O-])OC)[C@H](OCCC(=O)NCCCNC(=O)CCCCO[C@@H]2O[C@H](CO)[C@H](O)[C@H](O)[C@H]2NC(C)=O)[C@@H](OCCC(=O)NCCCNC(=O)CCCCO[C@@H]2O[C@H](CO)[C@H](O)[C@H](O)[C@H]2NC(C)=O)[C@@H]1OCCC(=O)NCCCNC(=O)CCCCO[C@@H]1O[C@H](CO)[C@H](O)[C@H](O)[C@H]1NC(C)=O]

Using the conjugate building block compound 15 described earlier, RNA is synthesized with the ligand attached to 3′-end of the sense strand according to known procedures. This is annealed with an antisense strand. The product is shown above.

### Example 66

### Synthesis of GalNAc-siRNA conjugate compound 16-F

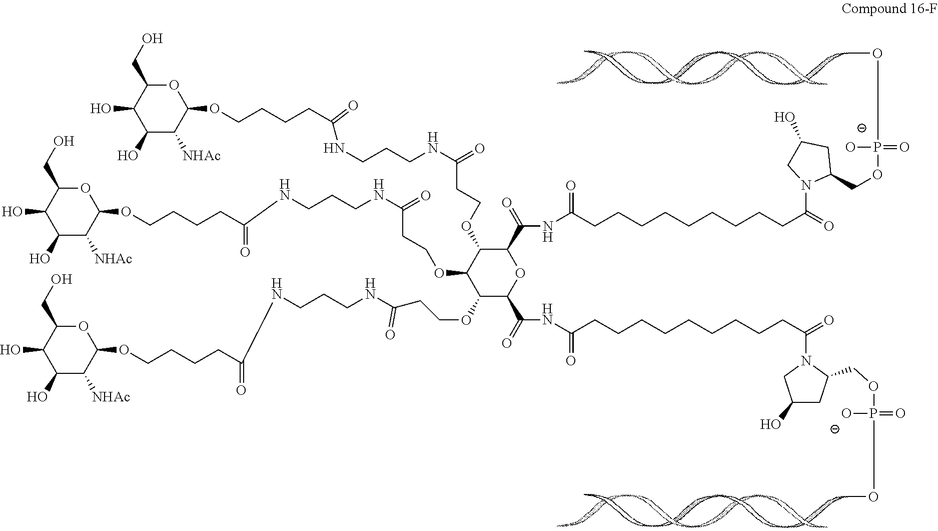 [SMILES: COP(=O)([O-])OC[C@@H]1C[C@@H](O)CN1C(=O)CCCCCCCCCC(=O)NC(=O)[C@H]1O[C@@H](C(=O)NC(=O)CCCCCCCCCC(=O)N2C[C@H](O)C[C@H]2COP(=O)([O-])OC)[C@H](OCCC(=O)NCCCNC(=O)CCCCO[C@@H]2O[C@H](CO)[C@H](O)[C@H](O)[C@H]2NC(C)=O)[C@@H](OCCC(=O)NCCCNC(=O)CCCCO[C@@H]2O[C@H](CO)[C@H](O)[C@H](O)[C@H]2NC(C)=O)[C@@H]1OCCC(=O)NCCCNC(=O)CCCCO[C@@H]1O[C@H](CO)[C@H](O)[C@H](O)[C@H]1NC(C)=O]

Using the conjugate building block compound 16 described earlier, RNA is synthesized with the ligand attached to 3′-end of the sense strand according to known procedures. This is annealed with an antisense strand. The product is shown above.

### Examples 67-74

(omitted)

### Example 75

### Synthesis of GalNAc-siRNA conjugate compound 25-F

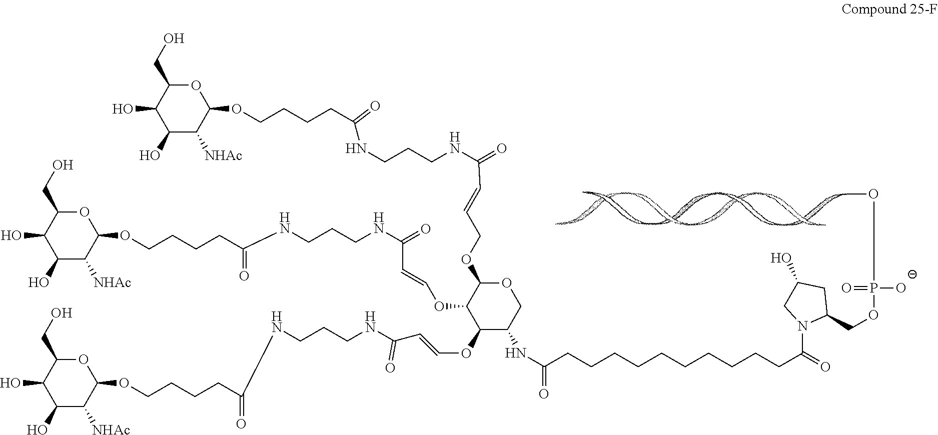 [SMILES: COP(=O)([O-])OC[C@@H]1C[C@@H](O)CN1C(=O)CCCCCCCCCCC(=O)N[C@H]1CO[C@H](OC/C=C/C(=O)NCCCNC(=O)CCCCO[C@@H]2O[C@H](CO)[C@H](O)[C@H](O)[C@H]2NC(C)=O)[C@@H](O/C=C/C(=O)NCCCNC(=O)CCCCO[C@@H]2O[C@H](CO)[C@H](O)[C@H](O)[C@H]2NC(C)=O)[C@@H]1O/C=C/C(=O)NCCCNC(=O)CCCCO[C@@H]1O[C@H](CO)[C@H](O)[C@H](O)[C@H]1NC(C)=O]

Using the conjugate building block compound 25 described earlier, RNA is synthesized with the ligand attached to 3′-end of the sense strand according to known procedures. This is annealed with an antisense strand. The product is shown above.

### Example 76

### Synthesis of Compound 26

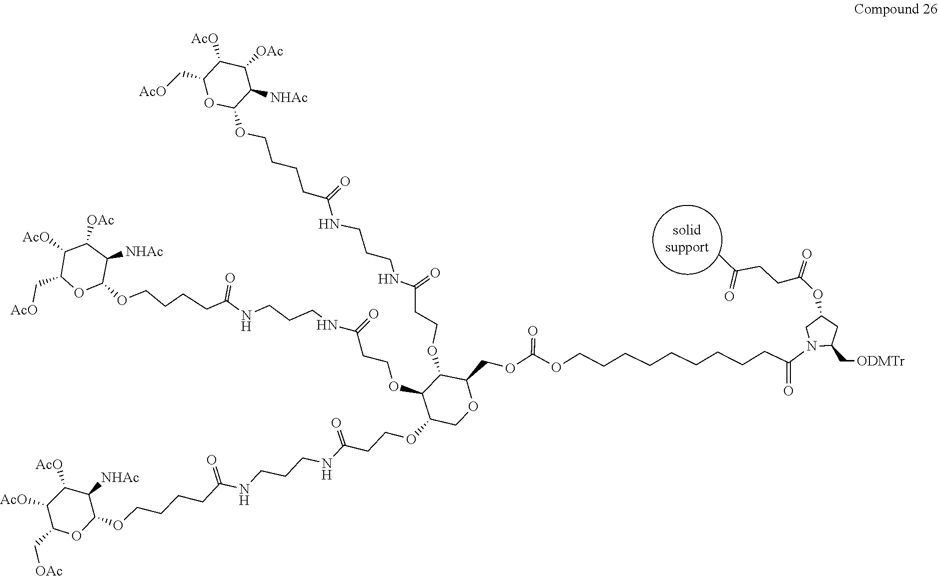

The title compound may be synthesized according to the synthetic route below.

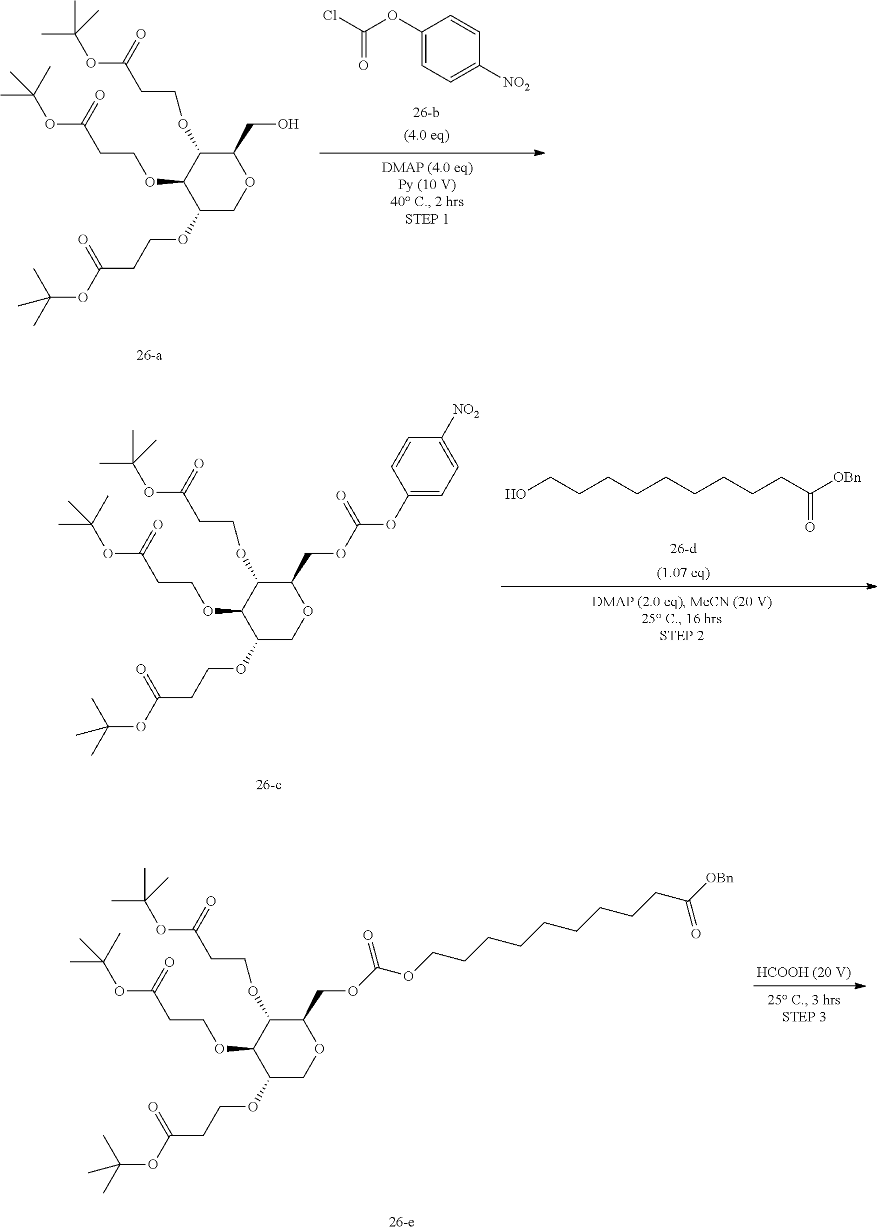 [SMILES: CC(C)(C)OC(=O)CCO[C@H]1[C@H](OCCC(=O)OC(C)(C)C)[C@@H](CO)OC[C@@H]1OCCC(=O)OC(C)(C)C ; O=C(Cl)Oc1ccc([N+](=O)[O-])cc1 ; CC(C)(C)OC(=O)CCO[C@H]1[C@H](OCCC(=O)OC(C)(C)C)[C@@H](COC(=O)Oc2ccc([N+](=O)[O-])cc2)OC[C@@H]1OCCC(=O)OC(C)(C)C ; O=C(CCCCCCCCCO)OCc1ccccc1 ; CC(C)(C)OC(=O)CCO[C@H]1[C@H](OCCC(=O)OC(C)(C)C)[C@@H](COC(=O)OCCCCCCCCCC(=O)OCc2ccccc2)OC[C@@H]1OCCC(=O)OC(C)(C)C]

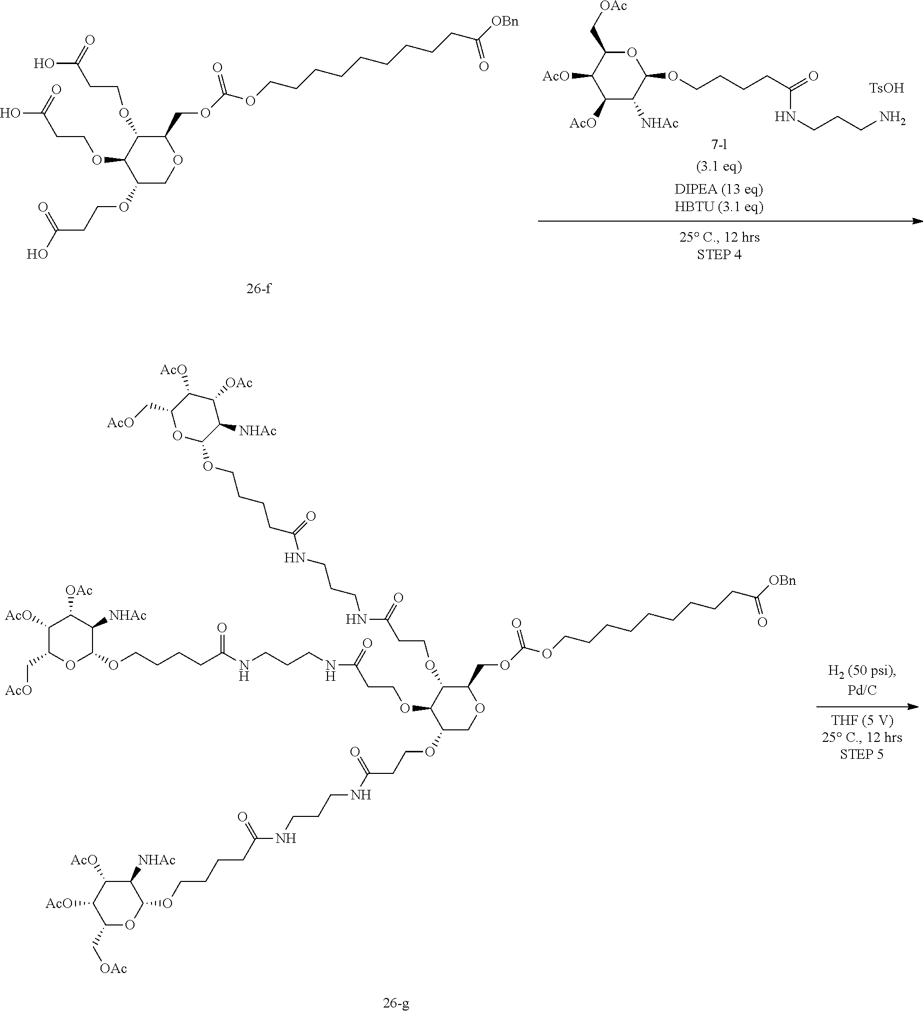

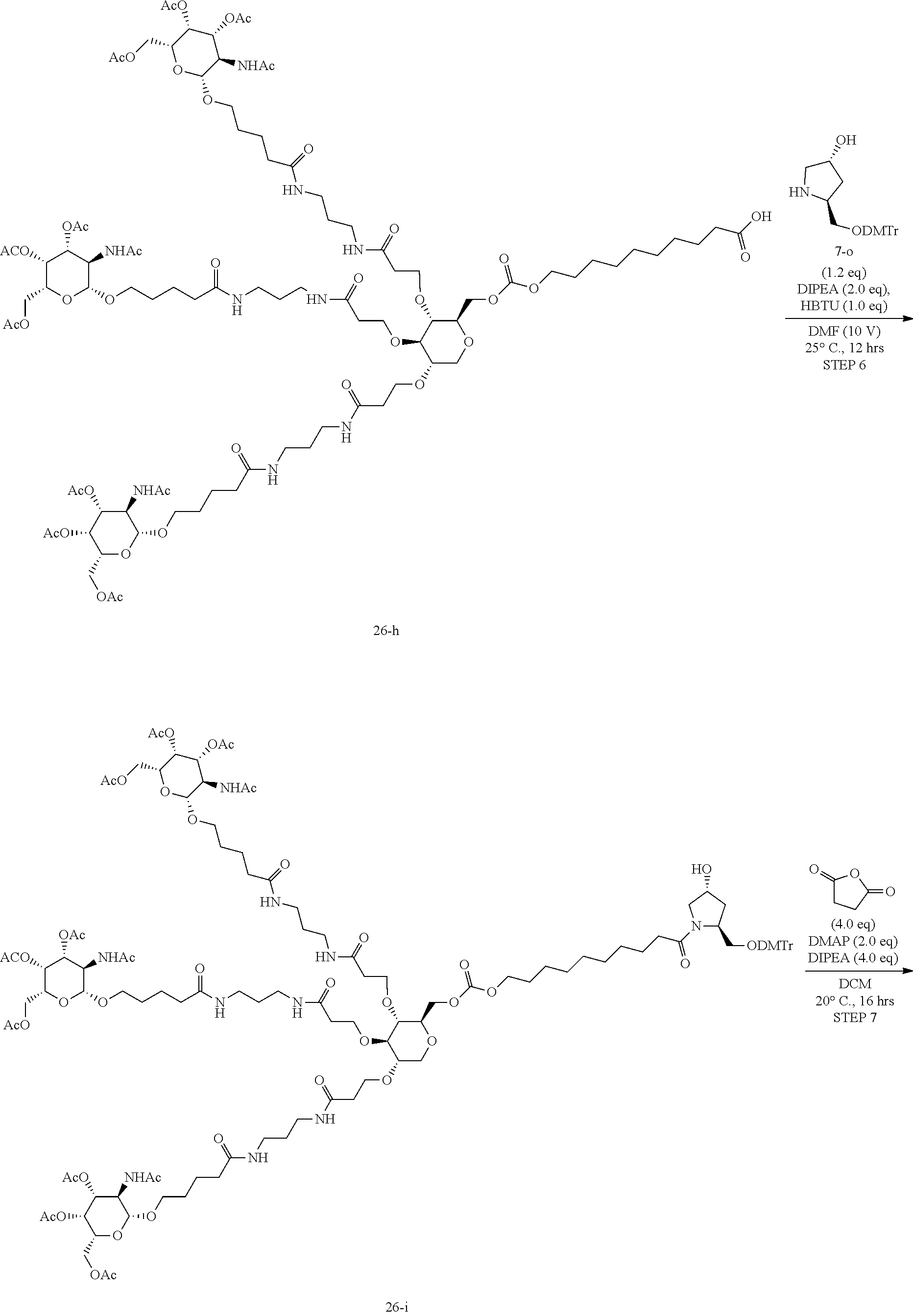

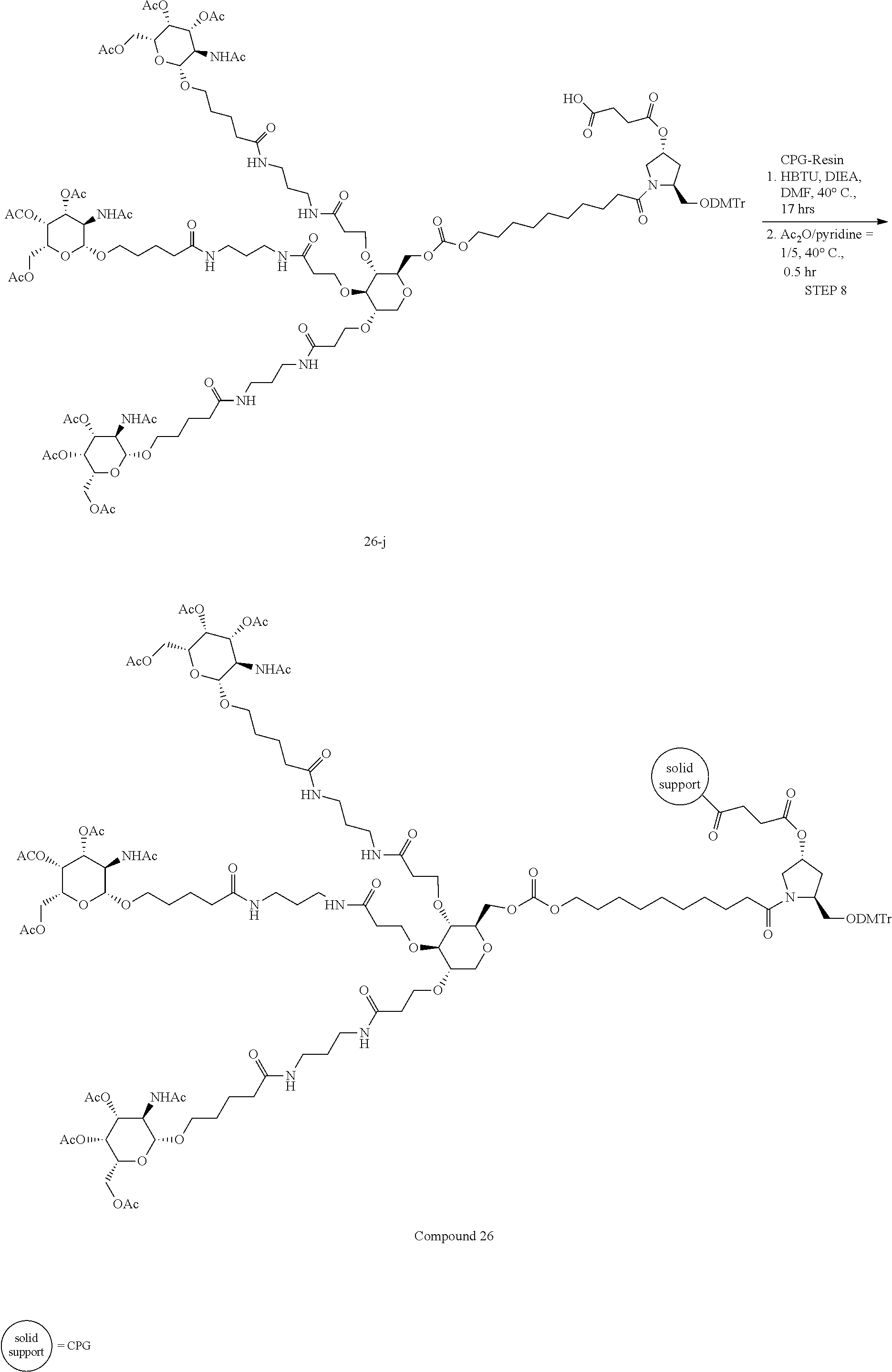

STEP 1: To a solution of Cpd. 26-a (5.00 g, 9.11 mmol) (The synthesis of Cpd. 26-a was shown below) in pyridine (30.0 mL) was added DMAP (4.45 g, 36.4 mmol) and Cpd. 26-b (7.35 g, 36.4 mmol) at 20° C. The reaction was stirred at 20° C. for 2 hrs. LCMS (RT=1.11 min) showed Cpd. 26-c was formed. Two reactions were combined here. The liquid was diluted with H₂O (40.0 mL) and extracted with EtOAc (50.0 mL×3). The combined organic layers were washed with 1N HCl (15.0 ml), NaHCO₃ (15.0 ml), brine (50.0 mL), dried over anhydrous Na₂SO₄, filtered and concentrated under reduced pressure to give Cpd. 26-c (11.5 g, crude) as a yellow oil. ¹H NMR (400 MHz CDCl₃) (Product) 8 8.16 (d, J=2.4 Hz, 2H), 7.43-7.40 (m, 2H), 4.52 (d, J=4.0 Hz, 1H), 4.48-4.31 (m, 1H), 4.07-4.04 (m, 3H), 4.03-4.01 (m, 1H), 3.83-3.81 (m, 3H), 3.27-3.10 (m, 6H), 2.53-2.46 (m, 6H), 1.45 (s, 9H).

The synthesis of Cpd. 26-a

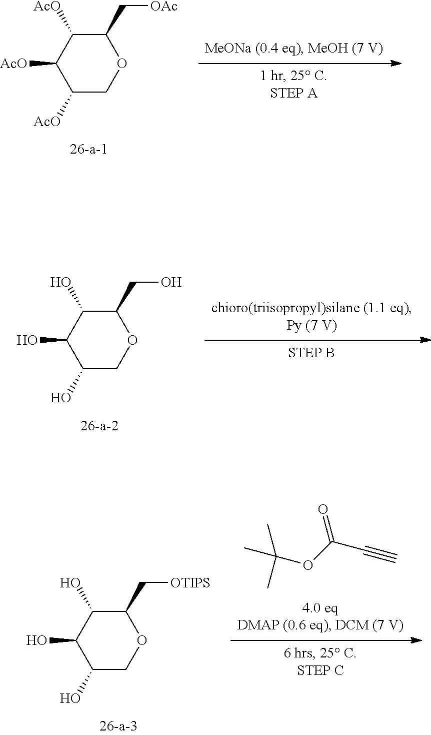

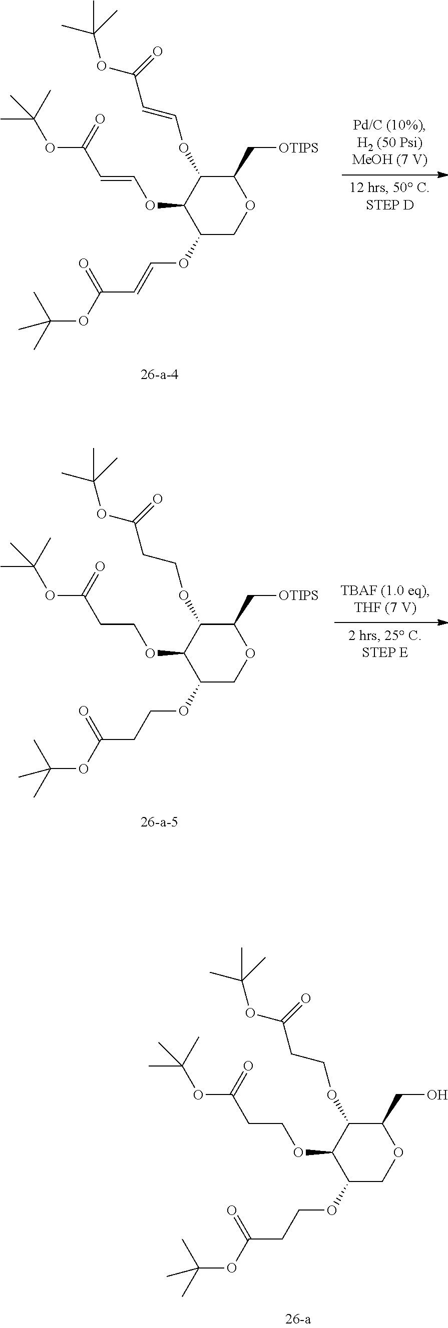

STEP A: To a solution of Cpd. 26-a-1 (142 g, 427.32 mmol) in MeOH (994 mL) was added NaOMe (9.23 g, 170.93 mmol). The mixture was stirred at 25° C. for 1 hr. TLC (Dichloromethane:Methanol=3:1, product: R_{f}=0.40) showed (starting material: R_{f}=0.80) was consumed completely and one new spot formed. Amberlite (H′) was added the above mixture and stirred until the solution showed pH=7. The Amberlite was filtered off and washed with MeOH. Cpd. 26-a-2 (70 g, 426.42 mmol, 99.79% yield) was obtained as colourless oil. ¹H NMR (400 MHz CD₃OD) (Product) δ 3.87-3.79 (m, 2H), 3.29-3.28 (m, 1H), 3.28-3.27 (m, 2H), 3.16-3.13 (m, 2H).

STEP B: To a solution of Cpd. 26-a-2 (7 g, 42.64 mmol) in Py (49 mL) was added chloro(triisopropyl)silane (9.04 g, 46.91 mmol) at 0° C. The mixture was stirred at 25° C. for 12 hrs. TLC (Ethyl acetate, product: R_{f}=0.35) showed (starting material: R_{f}=0.80) was consumed completely and one new main spot formed. The reaction mixture was diluted with H₂O 50 mL and extracted with EtOAC (50 mL×2). The combined organic layers were washed with brine 50 mL, dried over Na₂SO₄, filtered and concentrated under reduced pressure to give a residue. The residue was purified by column chromatography (SiO2, Petroleum ether/Ethyl acetate=1000/1 to 1/1, TLC: Ethyl acetate, R_{f}=0.35). Cpd. 26-a-3 (6.1 g, 19.03 mmol, 44.63% yield) was obtained as a white solid. ¹H NMR (400 MHz CD30D) (Product) δ 4.01-4.00 (d, 1H), 3.87-3.79 (m, 2H), 3.29-3.28 (m, 1H), 3.28-3.27 (m, 2H), 3.16-3.13 (m, 2H), 1.01 (s 21H).

STEP C: To a solution of Cpd. 26-a-3 (14.2 g, 44.31 mmol) in DCM (99.4 mL) was added tert-butyl prop-2-ynoate (22.36 g, 177.24 mmol) and DMAP (3.25 g, 26.59 mmol). The mixture was stirred at 25° C. for 6 hrs. TLC (Petroleum ether:Ethyl acetate=4:1, product: R_{f}=0.40 & 0.50) showed (starting material: R_{f}=0.02) was consumed completely and two new spots formed. The reaction mixture was diluted with H₂O 100 mL and extracted with DCM (100 mL×2). The combined organic layers were dried over Na₂SO₄, filtered and concentrated under reduced pressure to give a residue. The residue was purified by column chromatography (SiO2, Petroleum ether/Ethyl acetate=1000/1 to 1/1, TLC: Petroleum ether:Ethyl acetate=4/1, product: R_{f}=0.40 & 0.50). Cpd. 26-a-4 (30.5 g, 43.64 mmol, 98.48% yield) was obtained as yellow oil. ¹H NMR (400 MHz CD₃OD) (Product) δ 7.47-7.34 (m, 2H), 5.27-5.22 (m, 2H), 4.34-4.23 (m, 2H), 3.95-3.98 (d, J=1.2 Hz, 1H), 3.50-3.42 (m, 2H), 1.45-1.44 (s, 28H), 1.11-1.08 (s, 21H).

STEP D: To a solution of Pd/C (3.00 g, 10% purity) in MeOH (20 mL) was added Cpd. 26-a-4 (30.5 g, 43.64 mmol) in MeOH (190 mL) under Ar atmosphere. The suspension was degassed and purged with H₂ for 3 times. The mixture was stirred under H2 (50 Psi) at 50° C. for 12 hrs. HNMR (ET52646-51-P1A2) showed the starting material was consumed completely. The suspension was filtered through a pad of Celite or silica gel and the pad or filter cake was washed with MeOH 1.00 L. Cpd. 26-a-5 (30.0 g, 42.55 mmol, 97.52% yield) was obtained as colorless oil. ¹H NMR (400 MHz CDCl₃) (Product) δ 3.98-3.96 (m, 4H), 3.95-3.80 (m, 4H), 3.27-3.20 (m, 3H), 3.18-3.05 (m, 2H), 1.46-1.44 (s, 27H), 1.11-1.06 (s, 21H).

STEP E: To a solution of Cpd. 26-a-5 (30 g, 42.55 mmol) in THE (210 mL) was added TBAF (1 M, 42.55 mL). The mixture was stirred at 25° C. for 2 hrs. TLC (Petroleum ether:Ethyl acetate=2:1) showed (starting material: R_{f}=0.85) was consumed completely and one new spot (R_{f}=0.35) formed. The reaction mixture was diluted with H₂O 200 mL and extracted with EtOAc (200 mL×2). The combined organic layers were washed with brine 200 mL, dried over Na₂SO₄, filtered and concentrated under reduced pressure to give a residue. The residue was purified by column chromatography (SiO2, Petroleum ether/Ethyl acetate=1000/1 to 1/1, TLC: Petroleum ether:Ethyl acetate=2/1, product: R_{f}=0.35). Cpd. 26-a (17.9 g, 32.62 mmol, 76.67% yield) was obtained as colorless oil. ¹H NMR (400 MHz CDCl₃) (Product) δ 3.98-3.96 (m, 4H), 3.95-3.80 (m, 4H), 3.27-3.20 (m, 3H), 3.18-3.05 (m, 2H), 1.46-1.44 (s, 27H).

STEP 2: To a solution of Cpd. 26-c (4.00 g, 5.60 mmol) to MeCN (80.0 mL) at 25° C., and then added Cpd. 26-d (2.34 g, 8.41 mmol), DMAP (753 mg, 6.16 mmol) to the mixture. The reaction was stirred at 25° C. for 16 hrs. LCMS (RT=0.861 min) shows Cpd. 26-e was formed. The mixture under reduced pressure to give a residue. The residue was purified firstly by column chromatography (SiO2, Petroleum ether/Ethyl acetate=25/1 to 10/1) and then prep-HPLC column: Phenomenex luna C18 250*150 mm*15 um; mobile phase: [water (TFA)-ACN]; B %: 60%-98%, 25 min. Cpd. 26-e (4 g, 4.69 mmol, 83.7% yield) as a yellow oil. ¹H NMR (400 MHz CDCl₃) (Product) δ 7.37-7.32 (m, 5H), 5.12 (s, 2H), 4.38 (d, J=1.6 Hz, 1H), 4.13-4.10 (m, 1H), 4.03-4.01 (m, 1H), 4.00-3.99 (m, 2H), 3.97-3.88 (m, 5H), 3.80-3.79 (m, 4H), 3.30-3.14 (m, 6H), 2.52-2.45 (m, 8H), 2.37-2.33 (m, 2H), 1.65-1.63 (m, 6H), 1.62 (m, 34H), 1.45-1.28 (m, 11H)

STEP 3: To a solution of Cpd. 26-e (4.00 g, 5.60 mmol) to HCOOH (15.0 g, 312 mmol) at 25° C. The reaction was stirred at 25° C. for 4 hrs. LCMS (RT=0.824 min) shows Cpd. 26-f was formed. The mixture under reduced pressure to give Cpd. 26-f (4.00 g, 4.69 mmol, 83.6% yield) as a colorless oil. ¹H NMR (400 MHz CDCl₃) (Product) δ 7.38-7.27 (m, 4H), 5.11 (s, 2H), 4.38 (d, J=1.6 Hz, 1H), 4.37-3.78 (m, 10H), 3.33-3.08 (m, 5H), 2.50-2.36 (m, 5H), 2.35-2.01 (t, J=1.36, 3H), 1.63 (s, 4H), 1.27 (s, 10H)

STEP 4: To a mixture of Cpd. 7-1 (1.47 g, 2.92 mmol) in DCM (2.50 mL) was added HBTU (858 mg, 2.26 mmol) and DIPEA (943 mg, 7.30 mmol) in one portion at 25° C. under N₂, and then the mixture was stirred at 25° C. for under N₂ atmosphere 10 min. To this mixture was added Cpd. 26-f (500 mg, 730 umol) and the mixture was stirred at 17° C. for 20 hrs. LCMS (product: RT=2.884 min) showed the starting material was consumed completely. The mixture was poured into DCM (20 mL). The combined organic phase was washed with sat. NaHCO₃ (aq) (20.0 mL×2) and saturated brine (20.0 mL×3). Then the combined organic phase was dried with anhydrous Na₂SO₄, filtered and concentrated in vacuum. The crude product was purified by reversed-phase HPLC water (NH₄HCO₃)-ACN; B %: 40%-70%, 10 min to give Cpd. 26-g (350 mg, 163 umol, 22.4% yield) was obtained as a white solid. LCMS: ESI⁻, calcd for [M−2H]/2=1069.0, found 1069.1 (Product).

STEP 5: To a solution of Cpd. 26-g (350 mg, 163 umol) in THE (6 mL) was added Pd/C (600 mg, 10% purity) under Ar. The mixture was stirred under H₂ (15 psi) at 25° C. for 19 hours. LCMS (product: RT=2.149 min) showed one main peak with desired mass was detected. The mixture was filtered and concentrated in vacuum to give Cpd. 26-h (400 mg, crude) was used into the next step without further purification as white solid. LCMS: ESI⁻, calcd for [M−2H]/2=1024.0, found 1024.2 (Product).

STEP 6: To a mixture of Cpd. 26-h (400 mg, 195 umol) in DMF (3.00 mL) was added HBTU (110 mg, 292 umol) and DIPEA (50.41 mg, 390 umol) in one portion at 17° C. under N₂, and then the mixture was stirred at 17° C. for under N₂ 10 min. To this mixture was added Cpd. 7-o (122 mg, 292 umol) and the mixture was stirred at 17° C. for 17 hrs. LCMS (product: RT=2.907 min) showed the starting material was consumed completely. The crude product was purified by reversed-phase HPLC (water (NH₄HCO₃)-ACN; B %: 25%-55%, 20 min) to give Cpd. 26-i (140 mg, 57.0 umol, 29.3% yield) was obtained as a white solid. LCMS: ESI⁻, calcd for [M−2H]/2=1224.6, found 1225.1 (Product).

STEP 7: To a solution of Cpd. 26-i (140 mg, 57.1 umol) in DCM (1.00 mL) was added DIPEA (44.3 mg, 342 umol) and DMAP (1.74 mg, 14.3 umol) in one portion at 25° C. under N₂, and then the mixture was stirred at 20° C. for under N₂ 10 min. To this mixture was added succinic anhydride (34.3 mg, 342 umol) and the mixture was stirred at 20° C. for 4 hrs. LCMS (product: RT=2.611 min) showed the starting material was consumed completely. The mixture was poured into DCM (10 mL). The combined organic phase was washed with TEBA (aq) (5 mL×2). Then the combined organic phase was dried with anhydrous Na₂SO₄, filtered and concentrated in vacuum to give Cpd. 26-j (4-(((3R,5S)-5-((bis(4-methoxyphenyl)(phenyl)methoxy)methyl)-1-(10-(((((2R,3R,4R,5S)-3,4,5-tris(3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)tetrahydro-2H-pyran-2-yl)methoxy)carbonyl)oxy)decanoyl)pyrrolidin-3-yl)oxy)-4-oxobutanoic acid) (90 mg, 35.3 umol, 61.8% yield) was obtained as a white solid. LCMS: ESI⁻, calcd for [M−2H]/2=1274.6, found 1275.1 (Product).

STEP 8: To a solution of Cpd. 26-j (33 mg, 12.9 umol) in DMF (1.50 mL) was added HBTU (24.5 mg, 64.6 umol), DIEA (13.4 mg, 103.4 umol), DMAP (1.58 mg, 12.9 umol) in one portion at 25° C. Then CPG (250 mg; Chemical Name: Aminoalkyl-CPG, 500A from Hebei DNAchem Biotechnology Co., Ltd.) was added to the mixture. The mixture was stirred at 40° C. for 17 hrs. After that, it was filtered and the filter cake was washed with MeOH (5.00 mL×4) and DCM (5.00 mL×4). The filter cake was dried under N₂ stream to give a pale yellow solid. The pale yellow solid above was added into a mixture of dry pyridine (1.50 mL) and Ac₂O (0.30 mL). The mixture was then stirred at 40° C. for 0.5 hr. After that, it was filtered and the filter cake was washed with DCM (5.00 mL×4) and MeOH (5.00 mL×4). The resulted pale yellow solid was dried under vacuum for 12 hrs to give 225 mg solid supported product (loading 32 μmol/g).

### Example 77

### Synthesis of Compound 27

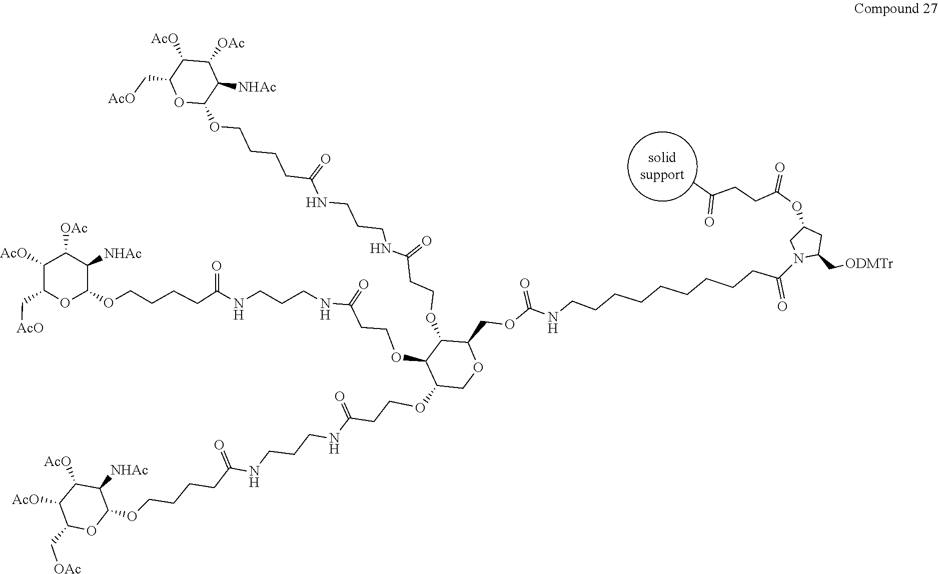

The title compound may be synthesized according to the synthetic route below.

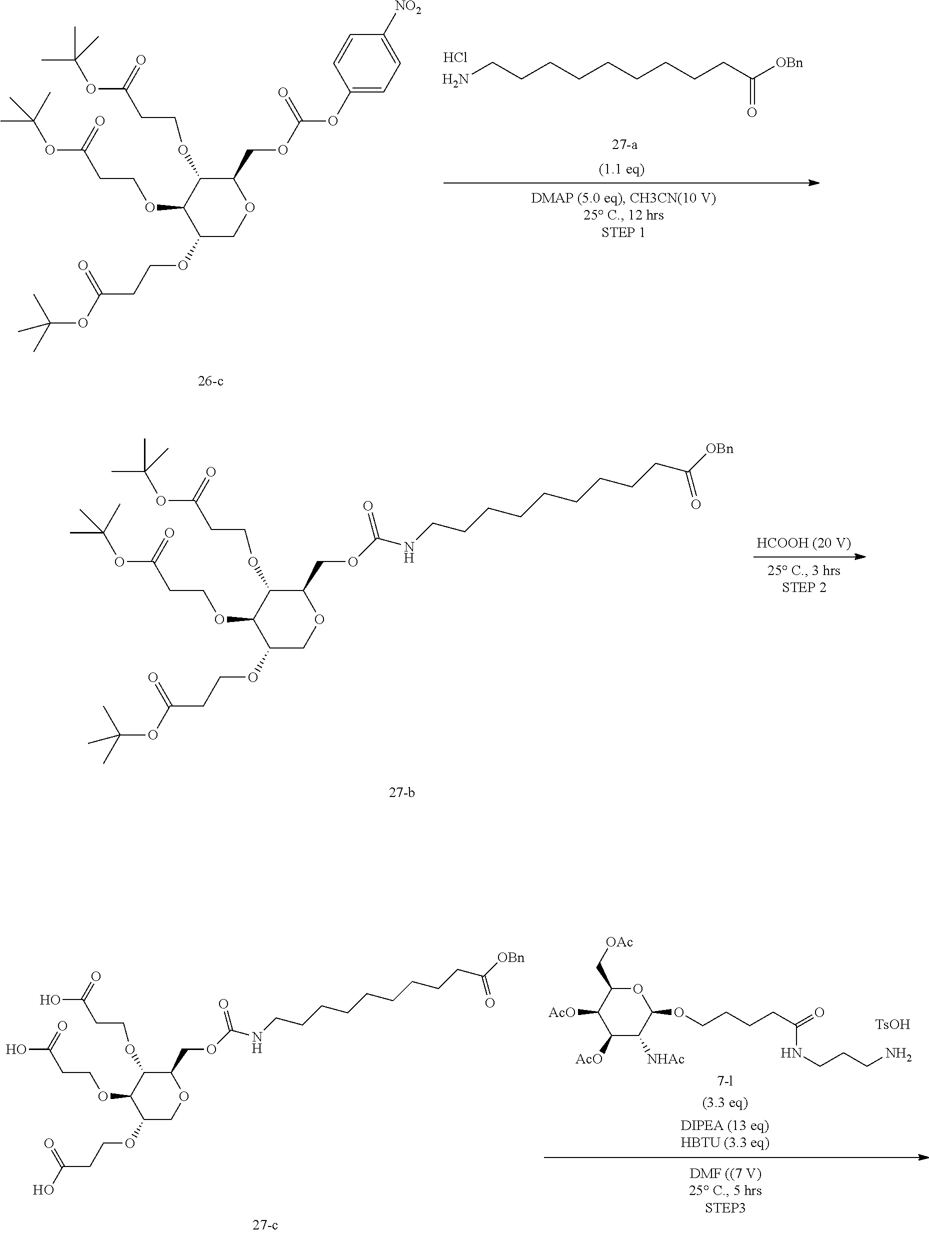

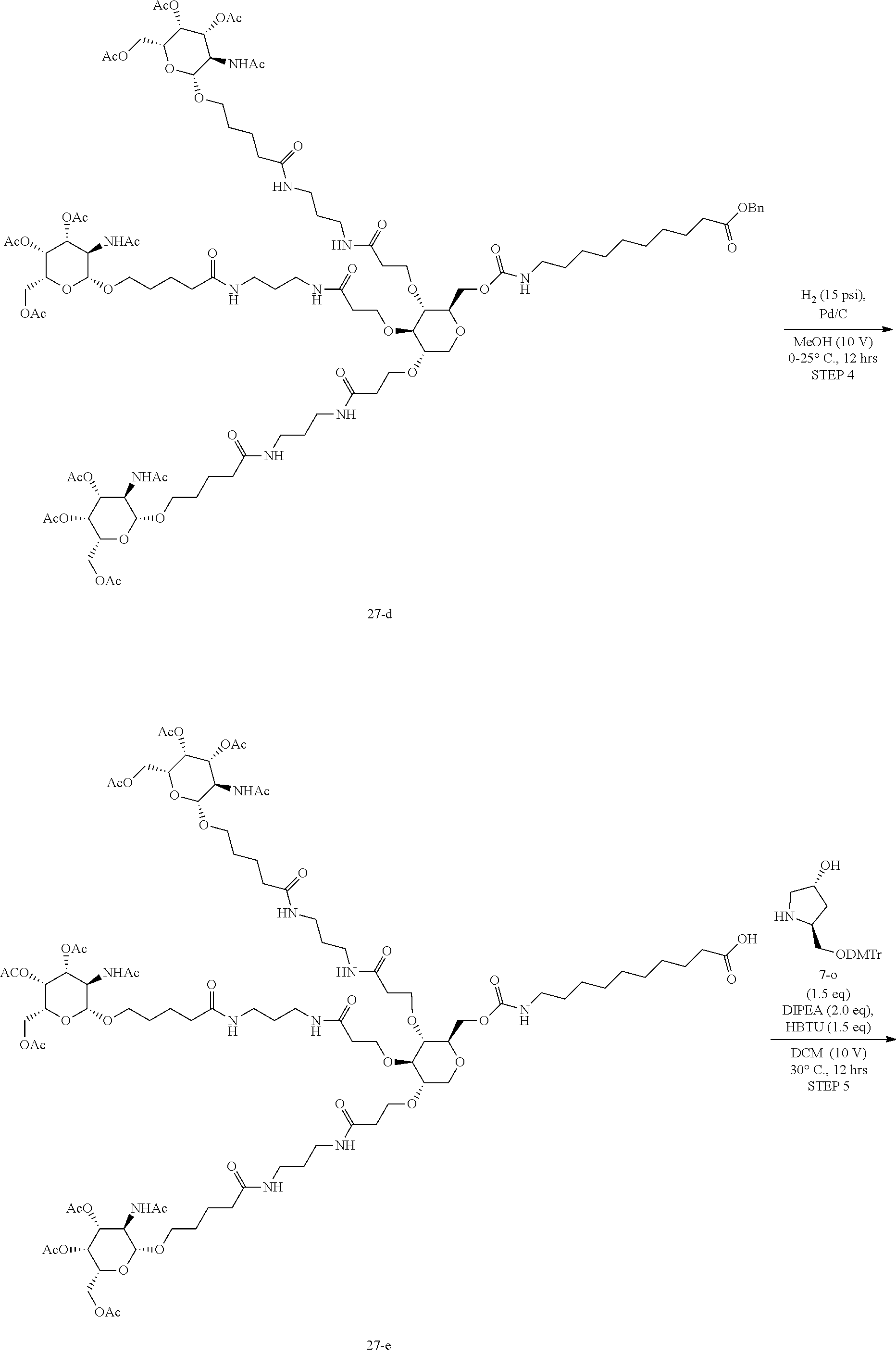

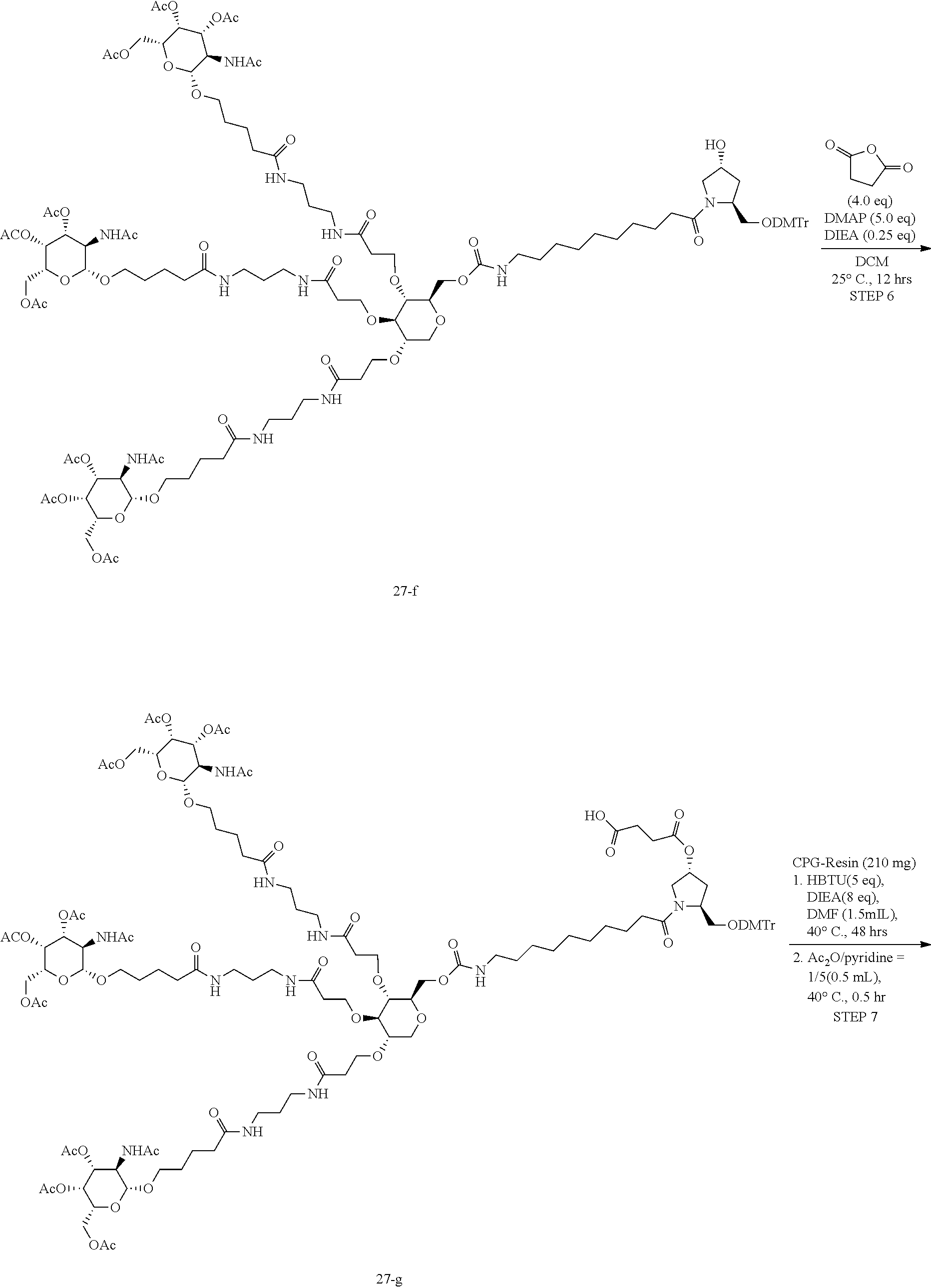

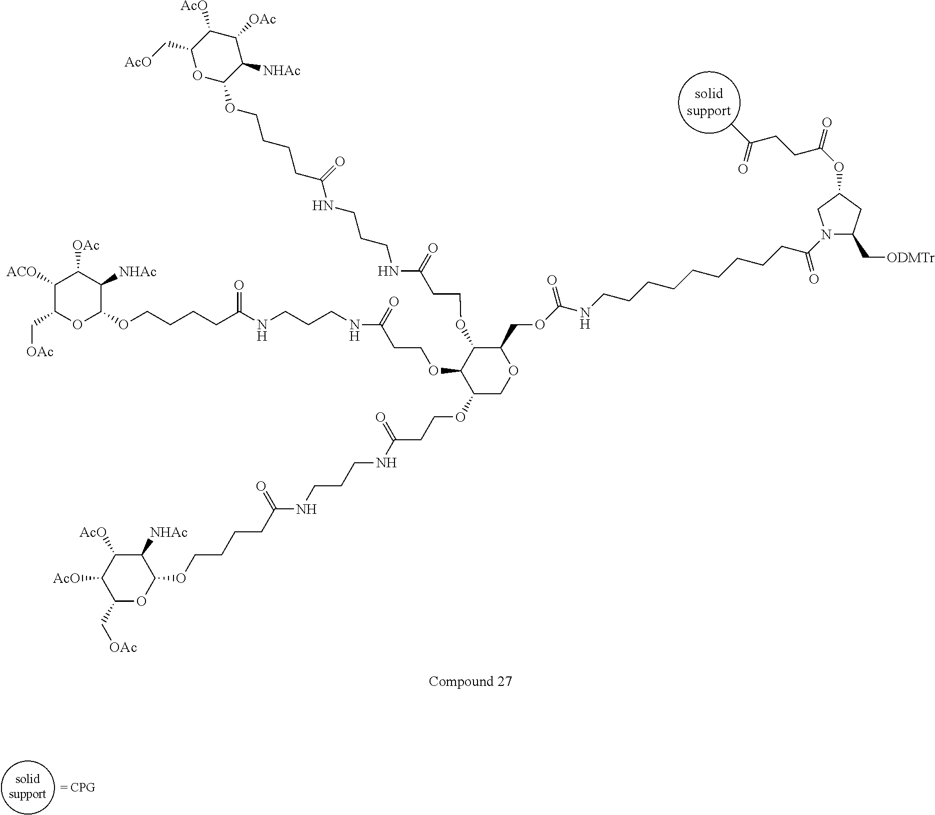

STEP 1: A mixture of Cpd. 26-c (4.00 g, 5.60 mmol, 1.00 eq), Cpd. 27-a (1.71 g, 6.16 mmol, 1.10 eq) and DMAP (3.42 g, 28.0 mmol, 5.00 eq) in CH₃CN (80.0 mL) was degassed and purged with N₂ for 3 times, and then the mixture was stirred at 25° C. for 16 hrs under N₂ atmosphere. LCMS (RT=1.084 min) shows Cpd. 26-c was consumed completely. Concentration of reaction liquid. The residue was purified by column chromatography (SiO2, Dichloromethane:Methanol=1:0 to 0:1). Obtain Cpd. 27-b (4.00 g, 4.47 mmol, 79.8% yield, 95.3% purity) as yellow oil. ¹H NMR (400 MHz CD₃OD) (Product) δ 7.34 (m, 5H), 5.10 (s, 2H), 4.09-4.26 (m, 1H), 3.98-4.01 (m, 6H), 3.85-3.75 (m, 1H), 3.94-3.96 (m, 3H), 3.07-3.31 (m, 8H), 2.37-2.51 (m, 9H), 1.45 (t, J=1.6 Hz, 27H), 1.29 (t, J=1.6 Hz, 12H).

STEP 2: To a solution of Cpd. 27-b (4.00 g, 4.69 mmol, 1.00 eq) in HCOOH (80.0 g, 1.67 mol, 20V). The mixture was stirred at 25° C. for 3 hrs. LCMS (RT=0.789&0.810 min) showed Cpd. 27-b was consumed completely. Concentrate the reaction mixture. Obtain Cpd. 27-c (3.00 g, 4.39 mmol, 93.5% yield) as yellow oil. ¹H NMR (Product) δ 7.19-7.31 (m, 5H), 7.10-14 (m, 4H), 5.04 (s, 2H), 4.12 (d, J=10.8 Hz, 1H), 3.80-3.87 (m, 5H), 3.67-3.69 (m, 3H), 2.90-3.12 (m, 4H), 2.26-2.43 (m, 8H), 1.17-1.50 (m, 12H).

STEP 3: To a solution of Cpd. 27-c (0.60 g, 877 umol, 1.00 eq) in DMF (12.0 mL) was added dropwise HBTU (1.10 g, 2.90 mmol, 3.30 eq) and DIPEA (1.47 g, 11.4 mmol, 1.99 mL, 13.0 eq) 25° C. over 30 min. After addition Cpd. 7-1 (1.46 g, 2.90 mmol, 3.30 eq) the mixture was stirred at this temperature for 12 hrs. LCMS showed Cpd. 27-c was consumed completely. The crude product was purified by reversed-phase HPLC. Column: Phenomenex luna C18 (250*70 mm, 15 um); mobile phase: [Water-ACN]; B %: 30%-60%, 20 min. Obtain Cpd. 27-d (1.5 g, 700.83 umol, 79.86% yield) as yellow oil. LCMS: ESI+, calcd for [M+2H]/2=1070.5, found 1070.5 (Product).

STEP 4: To a solution of Cpd. 27-d (900 mg, 420 umol, 1.00 eq) in MeOH (10.0 ml) was added Pd/C (900 mg, 10% purity, 1.00 eq), the reaction was stirred at 25° C. under H₂ for 12 hrs. LCMS (ET54421-53-P1A1, RT=1.820 min) showed the starting material was consumed completely. The mixture was filtered and concentrated under reduced pressure to give a residue (THF:MeOH=1:1, 50.0 mL). Cpd. 27-e (700 mg, crude) was obtained as a white solid. LCMS: ESI⁻, calcd for [M−2H]/2=1023.5, found 1023.6 (Product).

STEP 5: To a solution of Cpd. 27-e (200 mg, 97.5 umol, 1.00 eq) in DMF (3.00 mL) was added HBTU (171 mg, 450 umol, 1.50 eq) and DIPEA (25.2 mg, 195 umol, 34 uL, 2.00 eq) into the mixture. Then add Cpd. 7-o (61.4 mg, 146 umol, 1.5 eq) into solution and stirred at 30° C. for 12 hrs. LCMS (RT=0.738 min) showed the starting material was consumed completely. The aqueous phase was diluted with DCM (30.0 mL). The combined organic phase was washed with half saturated brine (20.0 mL×3), dried with anhydrous Na₂SO₄, filtered and concentrated in vacuum. The crude product was purified by reversed-phase HPLC. Column: Waters Xbridge BEH C18 100*30 mm*10 um; mobile phase: [water (NH₄HCO₃)-ACN]; B %: 35%-55%, 8 min The Cpd. 27-f (140 mg, 57.1 umol, 31.0 yield) was obtained as a white solid. LCMS: ESI⁻, calcd for [M−2H]/2=1224.1, found 1224.1 (product).

STEP 6: To a solution of Cpd. 27-f (135 mg, 55.0 umol, 1.00 eq) in DCM (1.50 mL) was added DMAP (1.68 mg, 13.7 umol, 0.25 eq) and DIPEA (35.5 mg, 275 umol, 5.00 eq), into the mixture. Then Add tetrahydrofuran-2,5-dione (27.5 mg, 275 umol, 5.00 eq) into solution and stirred at 15° C. for 12 hrs. LCMS showed the starting material was consumed completely. The residue was diluted with DCM (5.00 mL) and extracted with TEAB (15 mL×3). The combined organic layers were washed with H₂O mL (15 mL×2), dried over Na₂SO₄, filtered and concentrated under reduced pressure to give a residue. The crude product was purified by reversed-phase HPLC. Column: C₁₈₋₂ 100*30 mm*5 um; mobile phase: [0.1M TEAB-ACN]; B %: 20%-45%, 20 min. The Cpd. 27-g (4-(((3R,5S)-5-((bis(4-methoxyphenyl)(phenyl)methoxy)methyl)-1-(10-(((((2R,3R,4R,5S)-3,4,5-tris(3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)tetrahydro-2H-pyran-2-yl)methoxy)carbonyl)amino)decanoyl)pyrrolidin-3-yl)oxy)-4-oxobutanoic acid) (50.0 mg, 19.6 umol, 35.7% yield) was obtained as a white solid. LCMS: ESI⁻, calcd for [M−2H]/2=1274.1, found 1274.7 (product).

STEP 7: To a solution of Cpd. 27-g (30.0 mg, 11.7 umol, 1.00 eq) in DMF (1.50 mL) was added HBTU (22.2 mg, 58.7 umol, 5.00 eq), DIEA (12.1 mg, 94.0 umol, 16.3 uL, 8.00 eq), DMAP (1.44 mg, 11.7 umol, 1.00 eq) in one portion at 25° C. Then CPG (220 mg; Chemical Name: Aminoalkyl-CPG, 500A from Hebei DNAchem Biotechnology Co., Ltd.) was added to the mixture. The mixture was stirred at 40° C. for 48 hrs. After that, it was filtered and the filter cake was washed with MeOH (5.00 mL×4) and DCM (5.00 mL×4). The filter cake was dried under N₂ stream to give a pale yellow solid. The pale yellow solid above was added into a mixture of dry pyridine (1.50 mL) and Ac₂O (0.30 mL). The mixture was then stirred at 40° C. for 0.5 hr. After that, it was filtered and the filter cake was washed with DCM (5.00 mL×4) and MeOH (5.00 mL×4). The resulted pale yellow solid was dried under vacuum for 12 hrs to give 195 mg solid supported product (loading 32 μmol/g).

### Example 78

### Synthesis of GalNAc-siRNA conjugate compound 27-F

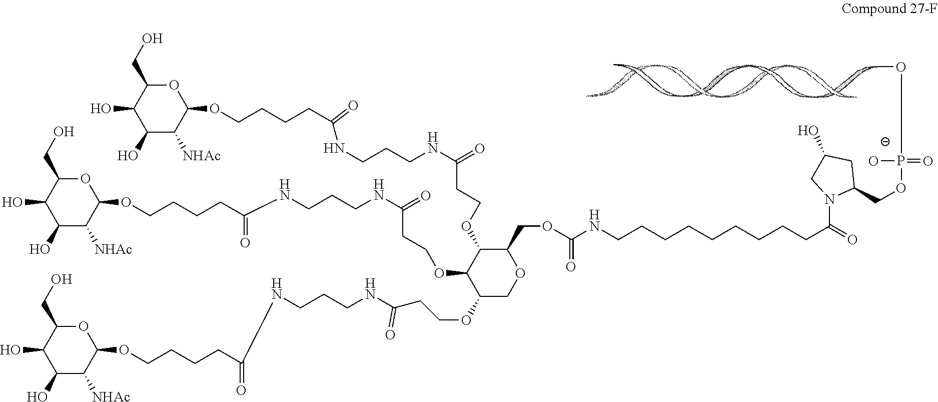 [SMILES: COP(=O)([O-])OC[C@@H]1C[C@@H](O)CN1C(=O)CCCCCCCCCNC(=O)OC[C@H]1OC[C@H](OCCC(=O)NCCCNC(=O)CCCCO[C@@H]2O[C@H](CO)[C@H](O)[C@H](O)[C@H]2NC(C)=O)[C@@H](OCCC(=O)NCCCNC(=O)CCCCO[C@@H]2O[C@H](CO)[C@H](O)[C@H](O)[C@H]2NC(C)=O)[C@@H]1OCCC(=O)NCCCNC(=O)CCCCO[C@@H]1O[C@H](CO)[C@H](O)[C@H](O)[C@H]1NC(C)=O]

Using the conjugate building block compound 27 described earlier (Example 77), RNA is synthesized with the ligand attached to 3′-end of the sense strand according to known procedures. This is annealed with an antisense strand. The product is shown above.

### Example 79

### Synthesis of Compound 28

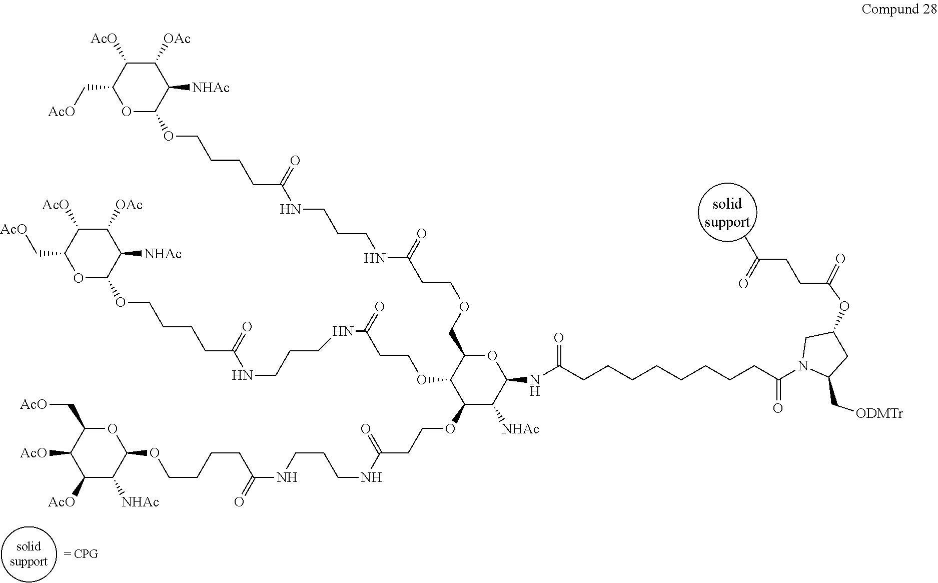

Compound 28 was synthesized following the procedure of compound 6 in example 29 by using 28-j as the starting material of which the synthesis was shown below. Loading: 34.0 μmol/g. 4-(((3R,5S)-1-(10-(((2R,3R,4R,5S,6R)-3-acetamido-4,5-bis(3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)-6-((3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)methyl)tetrahydro-2H-pyran-2-yl)amino)-10-oxodecanoyl)-5-((bis(4-methoxyphenyl)(phenyl)methoxy)methyl)pyrrolidin-3-yl)oxy)-4-oxobutanoic acid (28-j): The title compound was synthesized following the procedure of compound 6-j in example 29. LCMS: ESI⁻, calcd for [M−2H]/2=1287.6, found 1288.2.

### Example 80

### Synthesis of GalNAc-siRNA conjugate compound 28-F

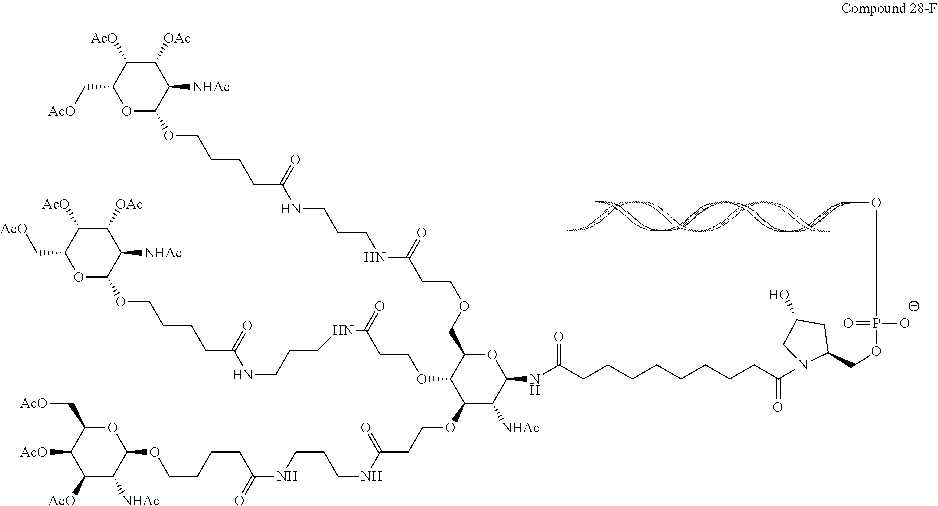 [SMILES: COP(=O)([O-])OC[C@@H]1C[C@@H](O)CN1C(=O)CCCCCCCCC(=O)N[C@@H]1O[C@H](COCCC(=O)NCCCNC(=O)CCCCO[C@@H]2O[C@H](COC(C)=O)[C@H](OC(C)=O)[C@H](OC(C)=O)[C@H]2NC(C)=O)[C@@H](OCCC(=O)NCCCNC(=O)CCCCO[C@@H]2O[C@H](COC(C)=O)[C@H](OC(C)=O)[C@H](OC(C)=O)[C@H]2NC(C)=O)[C@H](OCCC(=O)NCCCNC(=O)CCCCO[C@@H]2O[C@H](COC(C)=O)[C@H](OC(C)=O)[C@H](OC(C)=O)[C@H]2NC(C)=O)[C@H]1NC(C)=O]

Using the conjugate building block compound 28 described earlier (Example 79), RNA is synthesized with the ligand attached to 3′-end of the sense strand according to known procedures. This is annealed with an antisense strand. The product is shown above.

### Example 81

### Synthesis of Compound 29

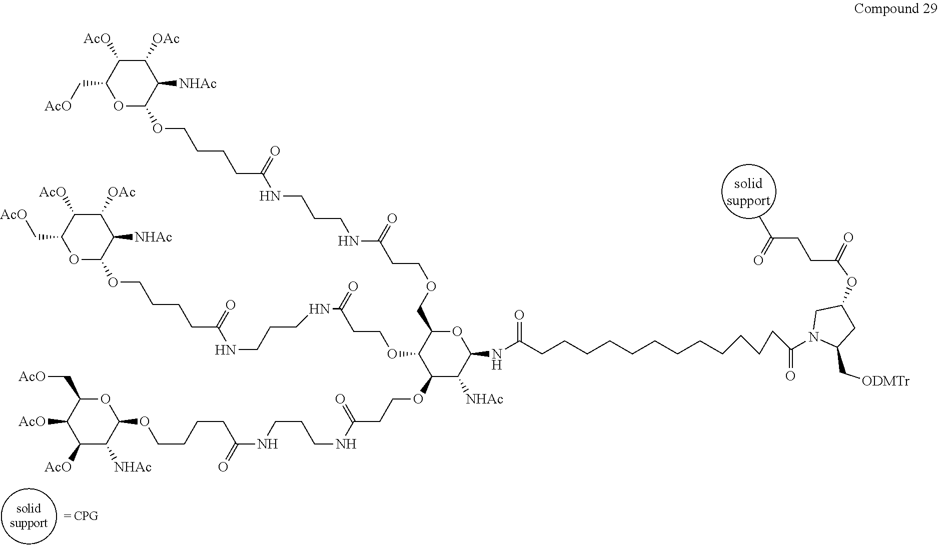

Compound 29 was synthesized following the procedure of compound 6 in example 29 by using 29-j as the starting material of which the synthesis was shown below. Loading: 22.0 μmol/g.

### 4-(((3R,5S)-1-(14-(((2R,3R,4R,5S,6R)-3-acetamido-4,5-bis(3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)-6-((3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)methyl)tetrahydro-2H-pyran-2-yl)amino)-14-oxotetradecanoyl)-5-((bis(4-methoxyphenyl)(phenyl)methoxy)methyl)pyrrolidin-3-yl)oxy)-4-oxobutanoic acid (29-j)

The title compound was synthesized following the procedure of compound 6-j in example 29. LCMS: ESI⁻, calcd for [M−2H]/2=1315.6, found 1316.2.

### Example 82

### Synthesis of GalNAc-siRNA conjugate compound 29-F

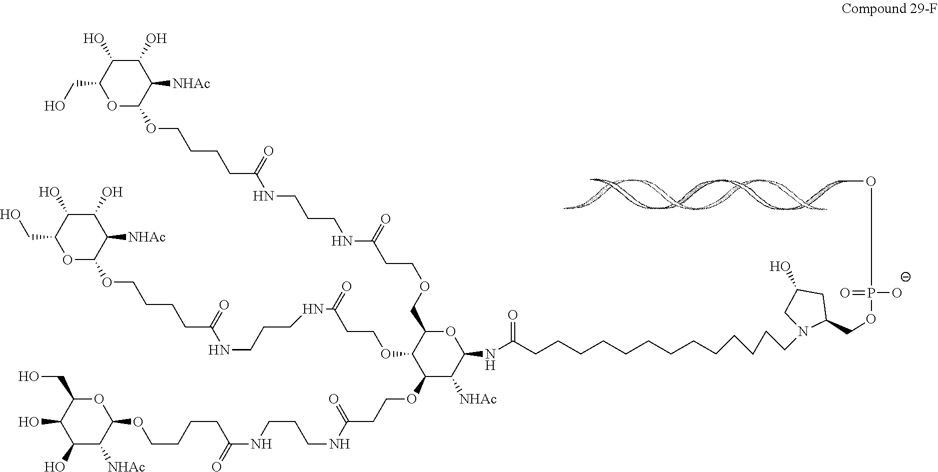 [SMILES: COP(=O)([O-])OC[C@@H]1C[C@@H](O)CN1CCCCCCCCCCCCCC(=O)N[C@@H]1O[C@H](COCCC(=O)NCCCNC(=O)CCCCO[C@@H]2O[C@H](CO)[C@H](O)[C@H](O)[C@H]2NC(C)=O)[C@@H](OCCC(=O)NCCCNC(=O)CCCCO[C@@H]2O[C@H](CO)[C@H](O)[C@H](O)[C@H]2NC(C)=O)[C@H](OCCC(=O)NCCCNC(=O)CCCCO[C@@H]2O[C@H](CO)[C@H](O)[C@H](O)[C@H]2NC(C)=O)[C@H]1NC(C)=O]

Using the conjugate building block compound 29 described earlier (Example 81), RNA is synthesized with the ligand attached to 3′-end of the sense strand according to known procedures. This is annealed with an antisense strand. The product is shown above.

### Example 83

### Synthesis of Compound 30

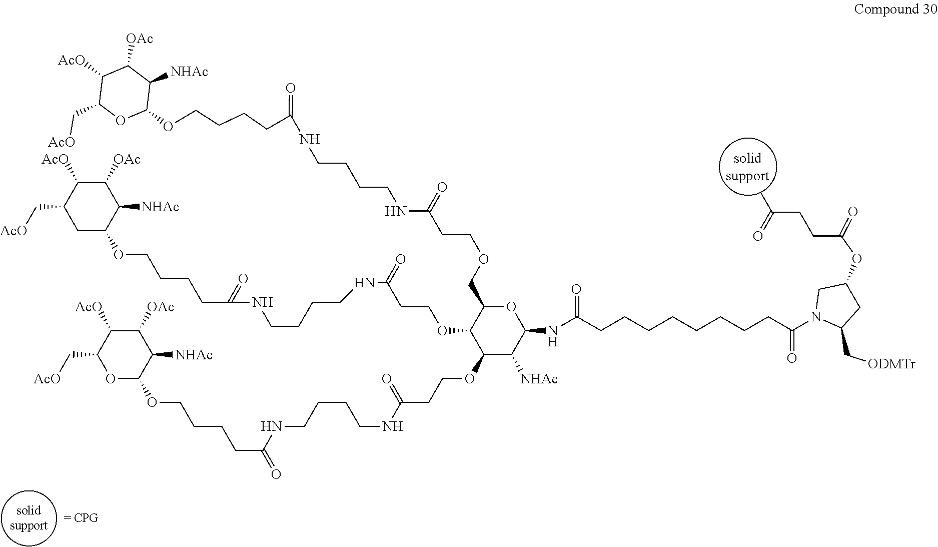

Compound 30 was synthesized following the procedure of compound 6 in example 29 by using 30-j as the starting material of which the synthesis was shown below. Loading: 23.0 μmol/g.

### 4-(((3R,5S)-1-(12-(((2R,3R,4R,5S,6R)-3-acetamido-4,5-bis(3-((4-(5-(((2R,3R,4R,5R,6R)-3-acetamido-4,5-diacetoxy-6-(acetoxymethyl)tetrahydro-2H-pyran-2-yl)oxy)pentanamido)butyl)amino)-3-oxopropoxy)-6-((3-((4-(5-(((2R,3R,4R,5R,6R)-3-acetamido-4,5-diacetoxy-6-(acetoxymethyl)tetrahydro-2H-pyran-2-yl)oxy)pentanamido)butyl)amino)-3-oxopropoxy)methyl)tetrahydro-2H-pyran-2-yl)amino)-12-oxododecanoyl)-5-((bis(4-methoxyphenyl)(phenyl)methoxy)methyl)pyrrolidin-3-yl)oxy)-4-oxobutanoic acid (30-j)

The title compound was synthesized following the procedure of compound 6-j in example 29. LCMS: ESI⁻, calcd for [M−2H]/2=1322.65, found: 1323.2.

### Example 84

### Synthesis of GalNAc-siRNA conjugate compound 30-F

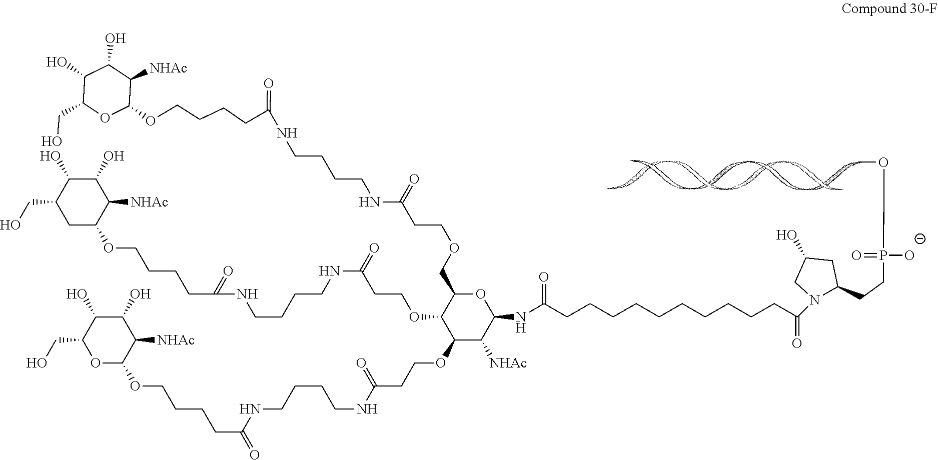

Using the conjugate building block compound 30 described earlier (Example 83), RNA is synthesized with the ligand attached to 3′-end of the sense strand according to known procedures. This is annealed with an antisense strand. The product is shown above.

### Example 85

### Synthesis of Compound 31

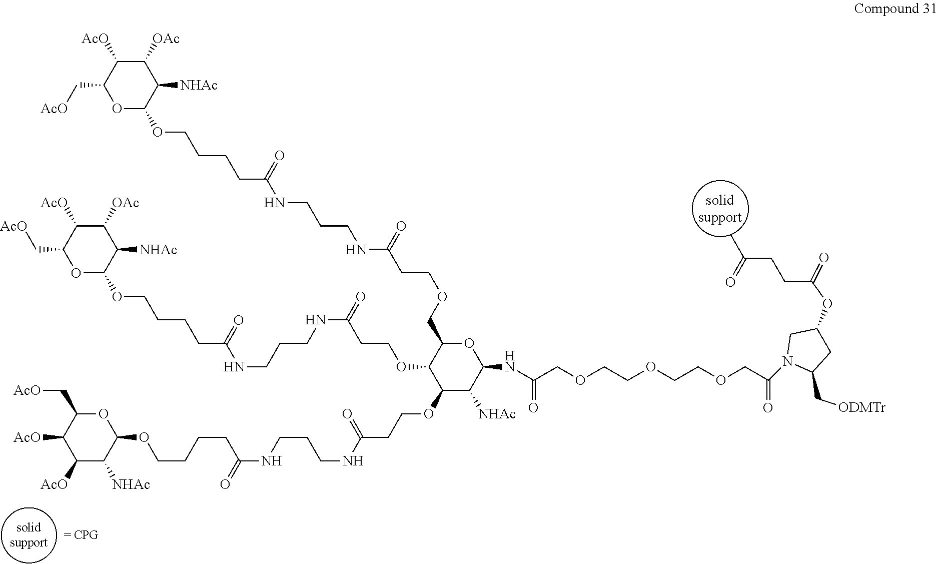

Compound 31 was synthesized following the procedure of compound 6 in example 29 by using 31-j as the starting material of which the synthesis was shown below. Loading: 25.0 μmol/g.

### 4-(((3R,5S)-1-(2-(2-(2-(2-(((2R,3R,4R,5S,6R)-3-acetamido-4,5-bis(3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)-6-((3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)methyl)tetrahydro-2H-pyran-2-yl)amino)-2-oxoethoxy)ethoxy)ethoxy)acetyl)-5-((bis(4-methoxyphenyl)(phenyl)methoxy)methyl)pyrrolidin-3-yl)oxy)-4-oxobutanoic acid (31-j)

The title compound was synthesized following the procedure of compound 6-j in example 29. LCMS: ESI⁻, calcd for [M−2H]/2=1297.58, found: 1298.1.

### Example 86

### Synthesis of GalNAc-siRNA conjugate compound 31-F

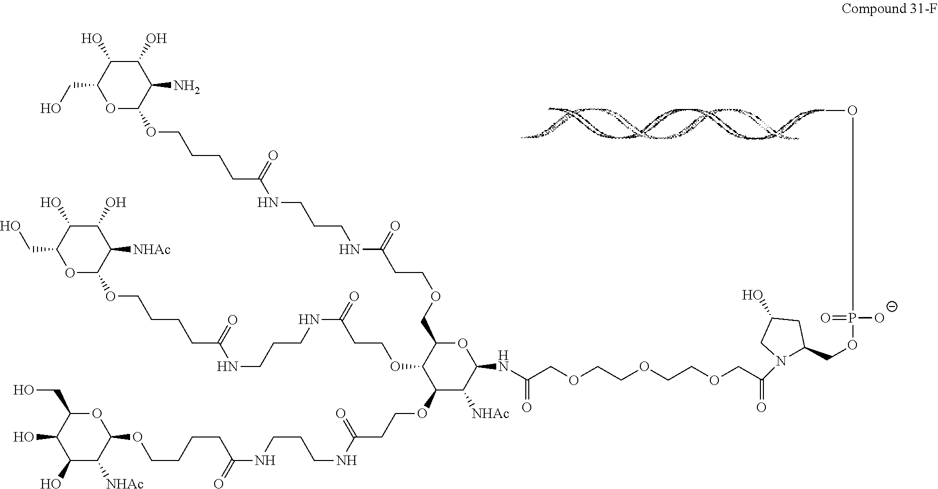 [SMILES: COP(=O)([O-])OC[C@@H]1C[C@@H](O)CN1C(=O)COCCOCCOCC(=O)N[C@@H]1O[C@H](COCCC(=O)NCCCNC(=O)CCCCO[C@@H]2O[C@H](CO)[C@H](O)[C@H](O)[C@H]2N)[C@@H](OCCC(=O)NCCCNC(=O)CCCCO[C@@H]2O[C@H](CO)[C@H](O)[C@H](O)[C@H]2NC(C)=O)[C@H](OCCC(=O)NCCCNC(=O)CCCCO[C@@H]2O[C@H](CO)[C@H](O)[C@H](O)[C@H]2NC(C)=O)[C@H]1NC(C)=O]

Using the conjugate building block compound 31 described earlier (Example 85), RNA is synthesized with the ligand attached to 3′-end of the sense strand according to known procedures. This is annealed with an antisense strand. The product is shown above.

### Example 87

### Synthesis of Compound 32

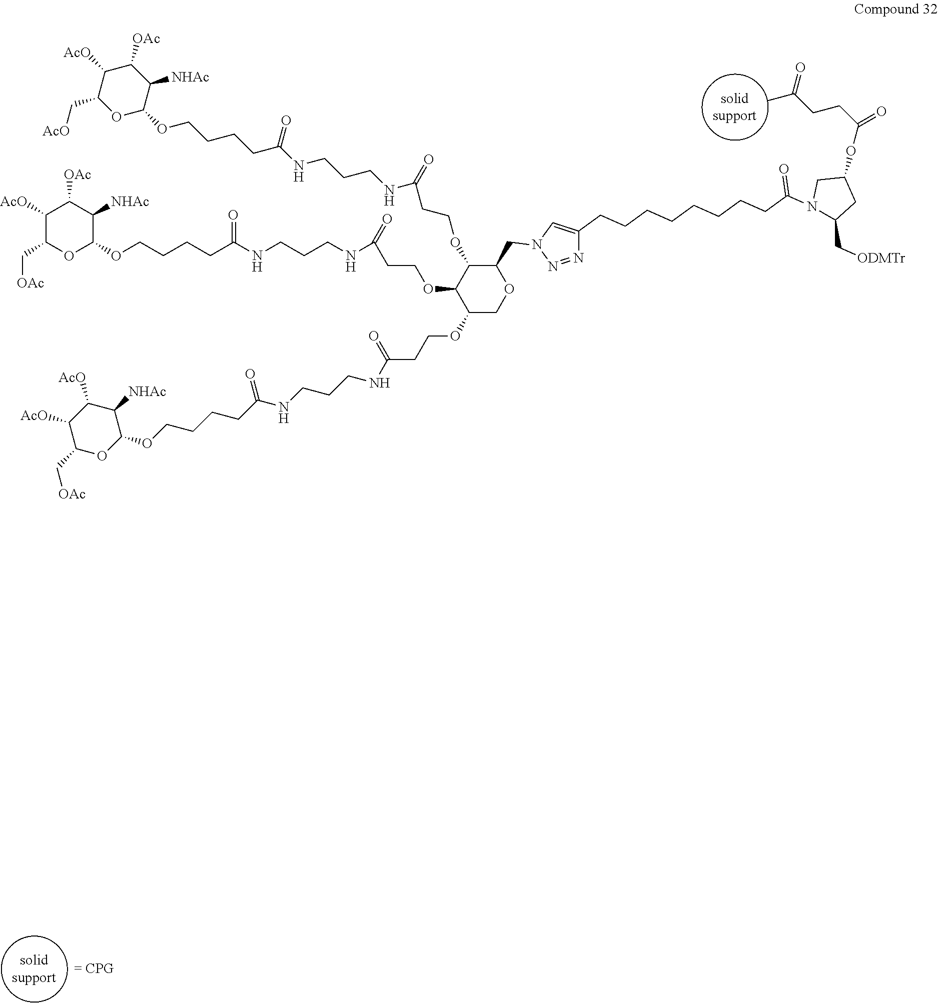

Compound 32 was synthesized following the procedure of compound 6 in example 29 by using 32-j as the starting material of which the synthesis was shown below. Loading: 30.0 μmol/g.

### 4-(((3R,5S)-5-((bis(4-methoxyphenyl)(phenyl)methoxy)methyl)-1-(9-(1-(((2R,3R,4R,5S)-3,4,5-tris(3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)tetrahydro-2H-pyran-2-yl)methyl)-1H-1,2,3-triazol-4-yl)nonanoyl)pyrrolidin-3-yl)oxy)-4-oxobutanoic acid (32-j)

The title compound was synthesized follow the procedure of compound 6-j in example 29 by using 32-e as the starting material of which the synthesis was shown below. LCMS: ESI⁻, calcd for [M−2H]/2=1271.1, found: 1271.7.

The synthesis of compound 32-e

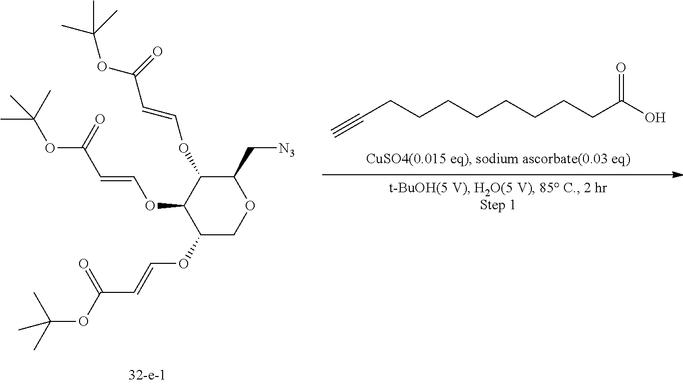

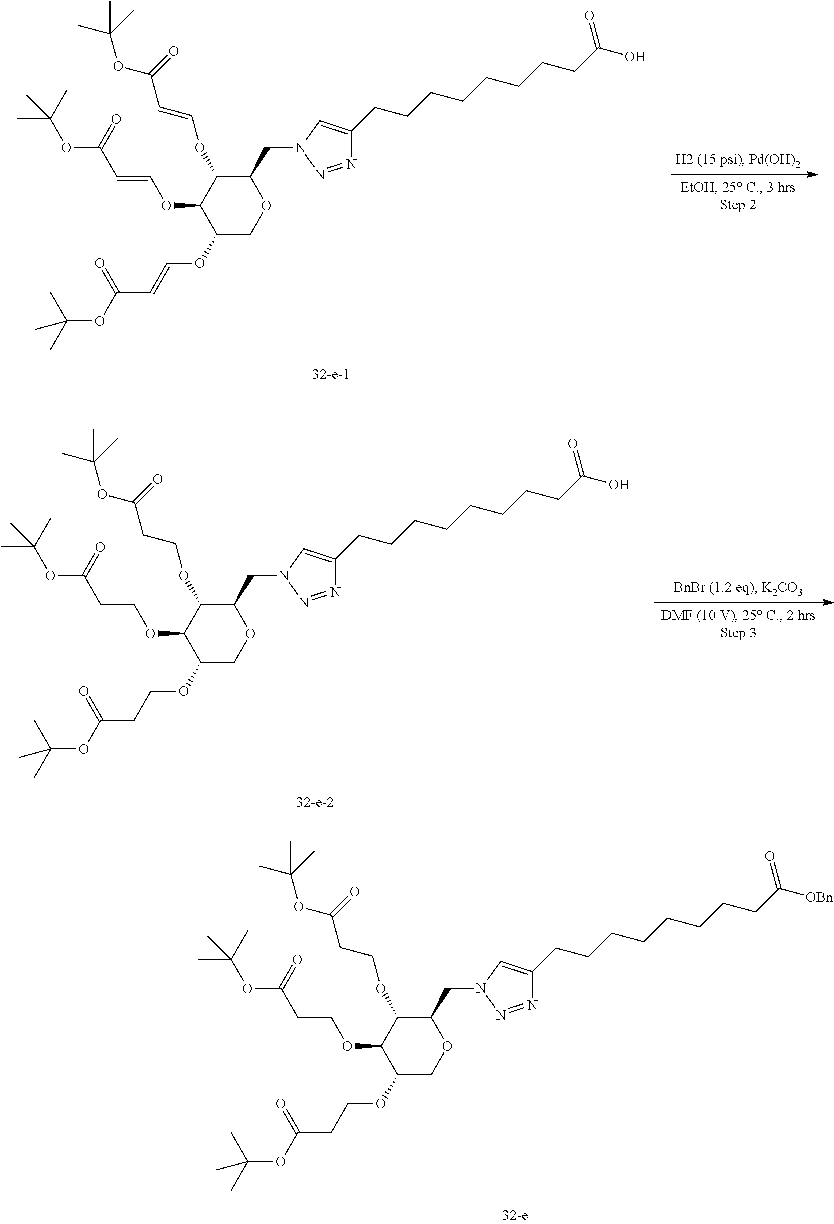

Step 1. To a solution of compound 2-d (4.50 g, 7.93 mmol, 1.00 eq) in t-BuOH (22.5 mL) and H₂O (22.5 mL) was added undec-10-ynoic acid (1.59 g, 8.72 mmol, 1.10 eq) at 25° C. (solution 1). Then, sodium ascorbate (47.1 mg, 238 umol, 0.03 eq) was added into CuSO₄ (19.0 mg, 119 umol, 0.015 eq) in H₂O (1.80 mL)(solution 2). The solution 2 was added into solution 1 at 25° C., the mixture was stirred at 85° C. for 2 hrs. TLC (petroleum ether/ethyl acetate=2/1, R_{f}=0.75) showed the starting materials was consumed. The reaction mixture was poured into water (30.0 mL) and extracted with ethyl acetate (40.0 mL×2). The residue was purified by column chromatography (SiO₂, petroleum ether/ethyl acetate=20/1 to 3/1). The crude product was purified by reversed-phase HPLC. Column: welch xtimate C18×250 70 mm #10 um; mobile phase: [water (NH₄HCO₃)-ACN]; B %: 35%-65%, 20 min to provide 9-(1-(((2R,3R,4R,5S)-3,4,5-tris(((E)-3-(tert-butoxy)-3-oxoprop-1-en-1-yl)oxy)tetrahydro-2H-pyran-2-yl)methyl)-1H-1,2,3-triazol-4-yl)nonanoic acid (32-e-1) (2.20 g; yield: 61.1%). LCMS: calcd for [M+H]=750.4, found 750.5.

Step 2. To a solution of compound 32-e-1 (2.00 g, 2.67 mmol, 1.00 eq) in EtOH (20.0 mL) was added Pd(OH)₂ (3.00 g, 10% purity), the reaction was stirred at 25° C. under H₂ (15 psi) for 2 hrs. LCMS showed the starting material was consumed completely. The mixture was filtered and concentrated under reduced pressure to give 9-(1-(((2R,3R,4R,5S)-3,4,5-tris(3-(tert-butoxy)-3-oxopropoxy)tetrahydro-2H-pyran-2-yl)methyl)-1H-1,2,3-triazol-4-yl)nonanoic acid (32-e-2) (1.98 g; yield: 98%). ¹H NMR: (400 MHz, CDCl₃) δ 7.37 (s, 1H), 4.67 (d, J=12 Hz, 3H), 4.41-4.46 (m, 1H), 3.88-4.05 (m, 5H), 3.70-3.81 (m, 3H), 3.38-3.41 (m, 1H), 3.20-3.27 (m, 2H), 2.86-3.03 (m, 2H), 2.69 (t, J=8 Hz, 2H), 2.44-2.58 (m, 6H), 2.29 (t, J=8 Hz, 2H), 1.60-1.67 (m, 4H), 1.44 (t, J=8 Hz, 27H), 1.31 (s, 8H).

Step 3. To a solution of compound 32-e-2 (1.60 g, 2.12 mmol, 1.00 eq) in DMF (16.0 mL) and H₂O (22.5 mL) was added K₂CO₃ (878 mg, 6.35 mmol, 3.00 eq) and BnBr (398 mg, 2.33 mmol, 277 uL, 1.10 eq), then stirred at 25° C. for 2 hrs. TLC (petroleum ether/ethyl acetate=2/1, R_{f}=0.3) showed the starting materials was consumed. The reaction solution was extracted with EtOAc (200 mL), washed with brine (200 mL), dried with Na₂SO₄, filtered and concentrated in vacuum. The residue was purified by column chromatography (SiO2, petroleum ether/ethyl acetate=20/1 to 2/1) to provide tri-tert-butyl 3,3′,3″-(((2R,3R,4R,5S)-2-((4-(9-(benzyloxy)-9-oxononyl)-1H-1,2,3-triazol-1-yl)methyl)tetrahydro-2H-pyran-3,4,5-triyl)tris(oxy))tripropionate (32-e) (1.30 g; yield: 72.6%). ¹H NMR: (400 MHz, CDCl₃) δ 7.35 (s, 5H), 5.11 (s, 2H), 4.67 (d, J=12 Hz, 1H), 4.41-4.46 (m, 1H), 3.93-4.04 (m, 5H), 3.78-3.79 (m, 2H), 3.40 (s, 1H), 3.24-3.25 (m, 2H), 2.89-3.03 (t, 2H), 2.67-2.69 (m, 2H), 2.46-2.51 (m, 6H), 2.34-2.37 (m, 2H), 1.66 (s, 4H), 1.45-1.46 (m, 27H), 1.31 (s, 8H).

### Example 88

### Synthesis of GalNAc-siRNA Conjugate Compound 32-F

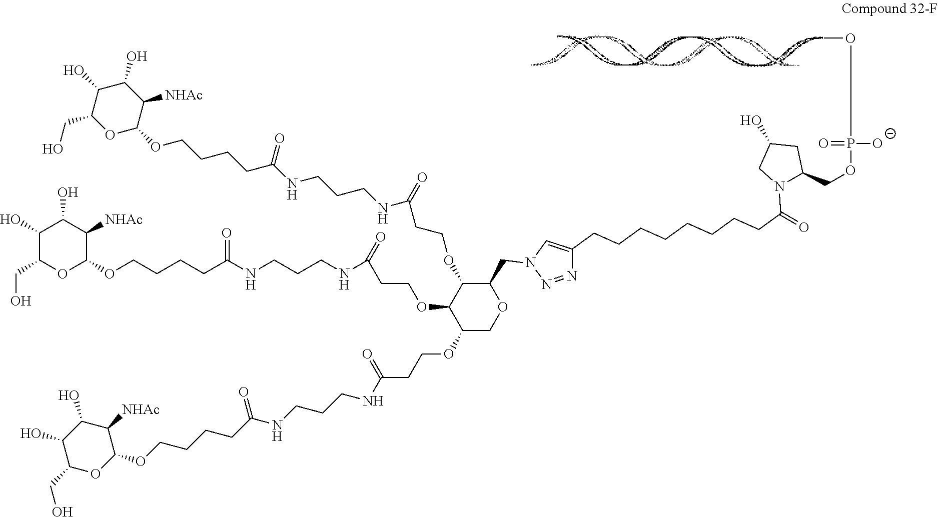 [SMILES: COP(=O)([O-])OC[C@@H]1C[C@@H](O)CN1C(=O)CCCCCCCCc1cn(C[C@H]2OC[C@H](OCCC(=O)NCCCNC(=O)CCCCO[C@@H]3O[C@H](CO)[C@H](O)[C@H](O)[C@H]3NC(C)=O)[C@@H](OCCC(=O)NCCCNC(=O)CCCCO[C@@H]3O[C@H](CO)[C@H](O)[C@H](O)[C@H]3NC(C)=O)[C@@H]2OCCC(=O)NCCCNC(=O)CCCCO[C@@H]2O[C@H](CO)[C@H](O)[C@H](O)[C@H]2NC(C)=O)nn1]

Using the conjugate building block compound 32 described earlier (Example 87), RNA is synthesized with the ligand attached to 3′-end of the sense strand according to known procedures. This is annealed with an antisense strand. The product is shown above.

### Example 89

### Synthesis of Compound 33

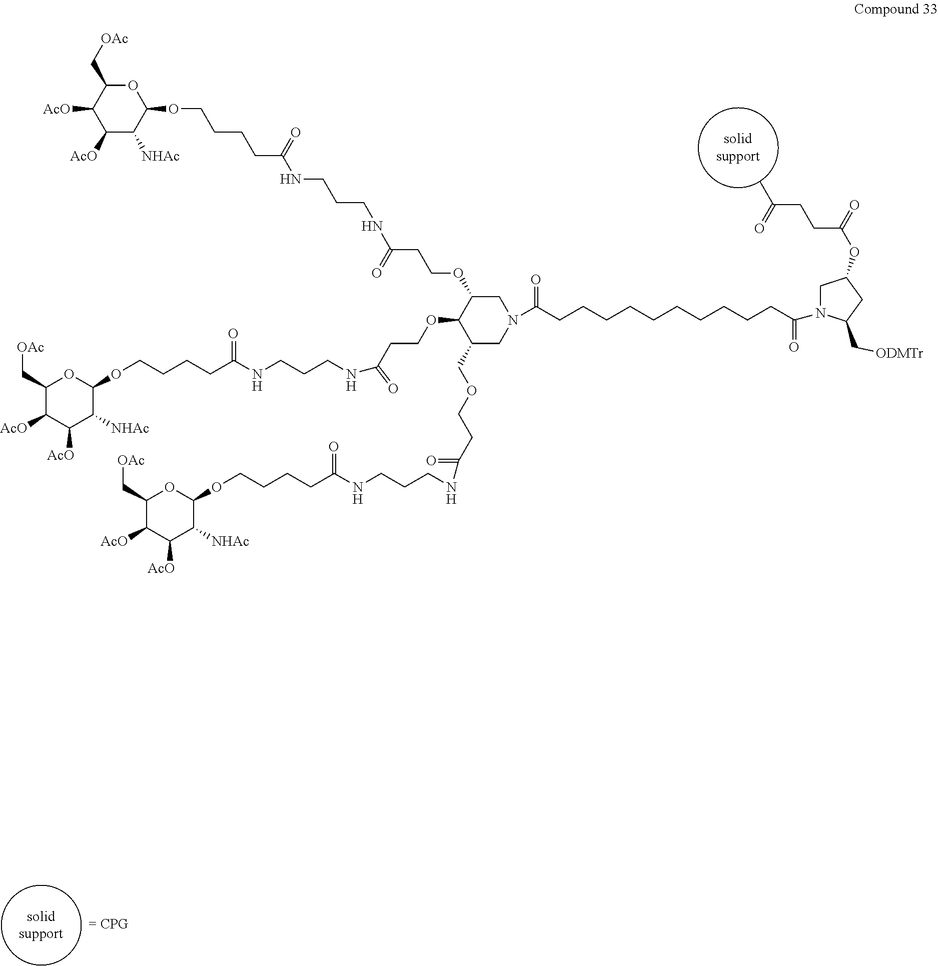

Compound 33 was synthesized following the procedure of compound 6 in example 29 by using 33-j as the starting material of which the synthesis was shown below. Loading: 42.1 μmol/g.

### 4-(((3R,5S)-1-(12-((3R,4R,5R)-3,4-bis(3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)-5-((3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)methyl)piperidin-1-yl)-12-oxododecanoyl)-5-((bis(4-methoxyphenyl)(phenyl)methoxy)methyl)pyrrolidin-3-yl)oxy)-4-oxobutanoic acid (33-j)

The title compound was synthesized following the procedure of compound 6-j in example 29 by using 33-e as the starting material of which the synthesis was shown below. LCMS: ESI⁻, calcd for [M−2H]/2=1265.1, found: 1265.1. ¹H NMR: (400 MHz, CDCl₃) (7.60-7.28 (m, 8H), 7.26-7.15 (m, 4H), 7.12-6.93 (m, 4H), 6.83-6.75 (m, 6H), 5.42-5.31 (m, 3H), 5.25-5.15 (m, 3H), 4.70-4.32 (m, 6H), 4.23-4.05 (m, 12H), 3.96-3.85 (m, 9H), 3.80-3.56 (m, 14H), 3.55-3.40 (m, 7H), 3.35-3.15 (m, 17H), 3.12-3.03 (m, 2H), 2.58-2.50 (m, 8H), 2.45-2.29 (m, 15H), 2.18 (s, 9H), 2.15 (s, 9H), 2.05 (s, 9H), 2.00 (s, 9H), 1.76-1.50 (m, 28H), 1.35-1.17 (m, 18H).

The synthesis of di-tert-butyl 3,3′-(((3R,4R,5R)-1-(12-(benzyloxy)-12-oxododecanoyl)-5-((3-(tert-butoxy)-3-oxopropoxy)methyl)piperidine-3,4-diyl)bis(oxy))dipropionate (33-e):

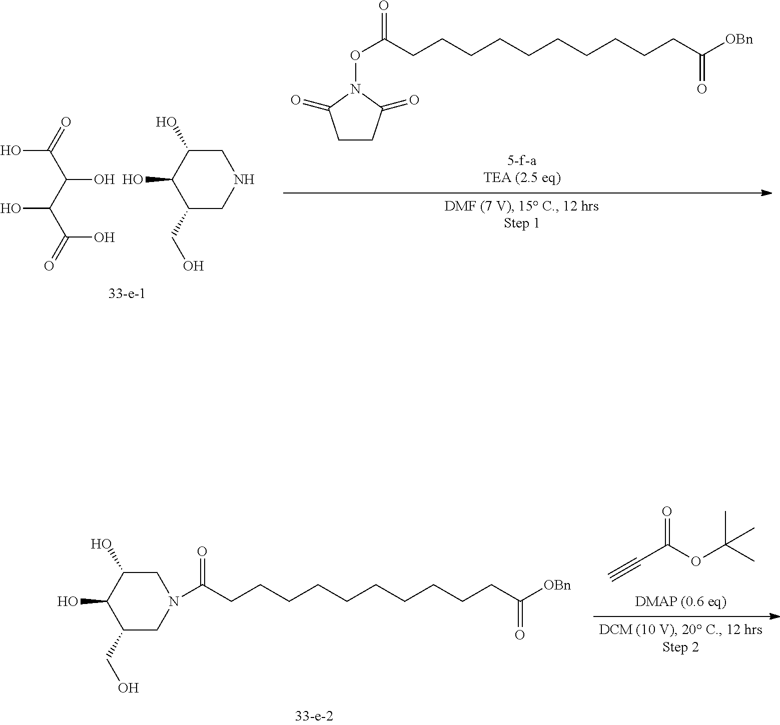

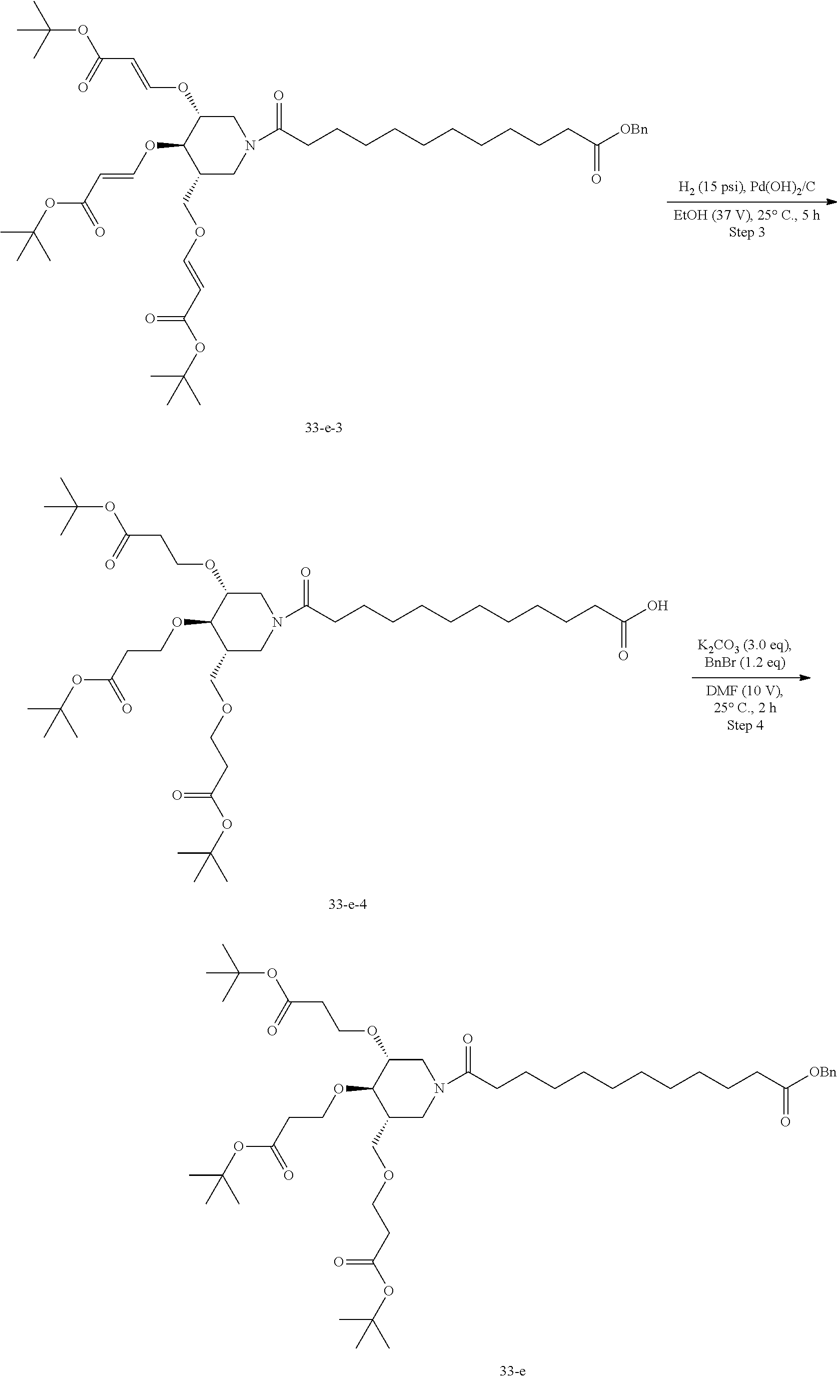

Step 1. A solution of compound 33-e-1 (3.37 g, 11.4 mmol, 1.00 eq) and TEA (2.87 g, 28.4 mmol, 3.95 mL, 2.50 eq) in DMF (33.0 mL) was added 1-benzyl 12-(2,5-dioxopyrrolidin-1-yl) dodecanedioate (5-f-a) (5.21 g, 12.5 mmol, 1.10 eq) in one portion at 15° C. under N₂, the reaction was stirred at 25° C. for 12 hrs. LCMS showed the reaction was completely. The reaction solution was concentrated under reduced pressure to give a residue. The reaction solution was concentrated under reduced pressure to give a residue as yellow oil. The residue was purified by column chromatography (SiO₂, DCM/MeOH=30/1 to 20/1) to provide benzyl 12-((3R,4R,5R)-3,4-dihydroxy-5-(hydroxymethyl)piperidin-1-yl)-12-oxododecanoate (33-e-2) (2.30 g, yield: 90.2%). LCMS: calcd for [M+H]=450.3, found 450.3. ¹H NMR: (400 MHz, MeOD) (7.18-7.28 (m, 5H), 5.03 (s, 2H), 4.40-4.57 (m, 2H), 3.69-3.88 (m, 4H), 3.40-3.43 (m, 4H), 2.83-2.89 (m, 1H), 2.32-2.40 (m, 1H), 2.27 (t, J=8 Hz, 4H), 1.51-1.57 (m, 4H), 1.18-1.20 (m, 12H).

Step 2. To a solution of compound 33-e-2 (4.60 g, 10.2 mmol, 1.00 eq) in DCM (46.0 mL) was added tert-butyl propiolate (5.16 g, 40.9 mmol, 5.62 mL, 4.00 eq) and DMAP (750 mg, 6.14 mmol, 0.60 eq), the mixture was stirred at 20° C. for 12 hrs. TLC (petroleum ether/ethyl acetate=1/1, R_{f}=0.6) showed the starting materials was consumed and one new spot formed. The solvent was removed in vacuum, water (30.0 ml) was added to the residue. The aqueous layer was extracted with dichloromethane (3×50.0 ml), and the organic layer was dried with Na₂SO₄. The residue was purified by column chromatography (SiO₂, petroleum ether:ethyl acetate=30:1 to 8:1) to provide di-tert-butyl 3,3′-(((3R,4R,5R)-1-(12-(benzyloxy)-12-oxododecanoyl)-5-((((E)-3-(tert-butoxy)-3-oxoprop-1-en-1-yl)oxy)methyl)piperidine-3,4-diyl)bis(oxy))(2E,2′E)-diacrylate (33-e-3) (4.70 g; yield: 55.5%). LCMS: calcd for [M+H]=845.5, found 845.6. ¹H NMR: (400 MHz, CDCl₃) δ 7.45-7.50 (m, 1H), 7.27-7.37 (m, 7H), 5.25-5.31 (m, 2H), 5.14-5.17 (m, 1H), 5.12 (s, 2H), 4.43-4.83 (m, 1H), 3.81-4.08 (m, 5H), 3.08-3.19 (m, 1H), 2.65-2.93 (m, 1H), 2.30-2.37 (m, 4H), 1.61-1.68 (m, 6H), 1.47 (d, J=4 Hz, 27H), 1.27-1.28 (m, 12H).

Step 3. To a solution of compound 33-e-3 (2.70 g, 3.26 mmol, 1.00 eq) in EtOH (100 mL) was added Pd/(OH)₂ (2.70 g, 3.85 mmol, 20% purity, 1.18 eq), then stirred at 25° C. for 5 hrs under H₂ (15 psi). HNMR showed no starting material. The mixture was filtered and washed filter cake with MeOH (100.0 mL×3), concentrated under reduced pressure to give 12-((3R,4R,5R)-3,4-bis(3-(tert-butoxy)-3-oxopropoxy)-5-((3-(tert-butoxy)-3-oxopropoxy)methyl)piperidin-1-yl)-12-oxododecanoic acid (33-e-4) (2.30 g, crude).

Step 4. To a solution of compound 33-e-4 (2.30 g, 3.09 mmol, 1.00 eq) in DMF (23.0 mL) was added K₂CO₃ (1.28 g, 9.27 mmol, 3.00 eq) and BnBr (634.5 mg, 3.71 mmol, 440.63 uL, 1.20 eq), then stirred at 25° C. for 2 hrs. TLC (Petroleum ether:Ethyl acetate=2:1, R_{f} (product)=0.3) showed no starting material. The mixture was extracted with EtOAc (120 mL), washed with brine (120 mL), dried with Na₂SO₄, filtered and concentrated in vacuum. The residue was purified by column chromatography (SiO₂, Petroleum ether/Ethyl acetate=10/1 to 2/1) to provide compound 33-e (2.20 g; yield: 85.3%). ¹H NMR: (400 MHz, CDCl₃) δ 8.02 (s, 1H), 7.29-7.40 (m, 5H), 4.53-4.20 (m, 1H), 3.95-3.40 (m, 9H), 3.35-3.10 (m, 2H), 2.96 (s, 3H), 2.88 (s, 3H), 2.85-2.60 (m, 1H), 2.50-2.38 (m, 6H), 2.34-2.25 (m, 4H), 1.70-1.55 (m, 5H), 1.45 (s, 27H), 1.35-1.20 (m, 12H).

### Example 90

### Synthesis of GalNAc-siRNA Conjugate Compound 33-F

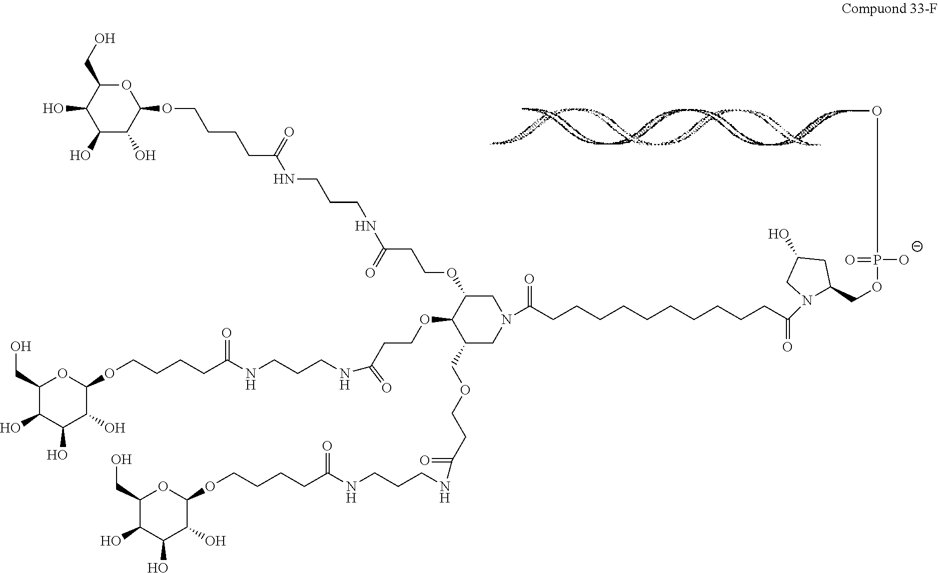 [SMILES: COP(=O)([O-])OC[C@@H]1C[C@@H](O)CN1C(=O)CCCCCCCCCCC(=O)N1C[C@H](COCCC(=O)NCCCNC(=O)CCCCO[C@@H]2O[C@H](CO)[C@H](O)[C@H](O)[C@H]2O)[C@@H](OCCC(=O)NCCCNC(=O)CCCCO[C@@H]2O[C@H](CO)[C@H](O)[C@H](O)[C@H]2O)[C@H](OCCC(=O)NCCCNC(=O)CCCCO[C@@H]2O[C@H](CO)[C@H](O)[C@H](O)[C@H]2O)C1]

Using the conjugate building block compound 33 described earlier (Example 89), RNA is synthesized with the ligand attached to 3′-end of the sense strand according to known procedures. This is annealed with an antisense strand. The product is shown above.

### Examples 91-92

(omitted)

### Example 93

### Synthesis of Compound 35

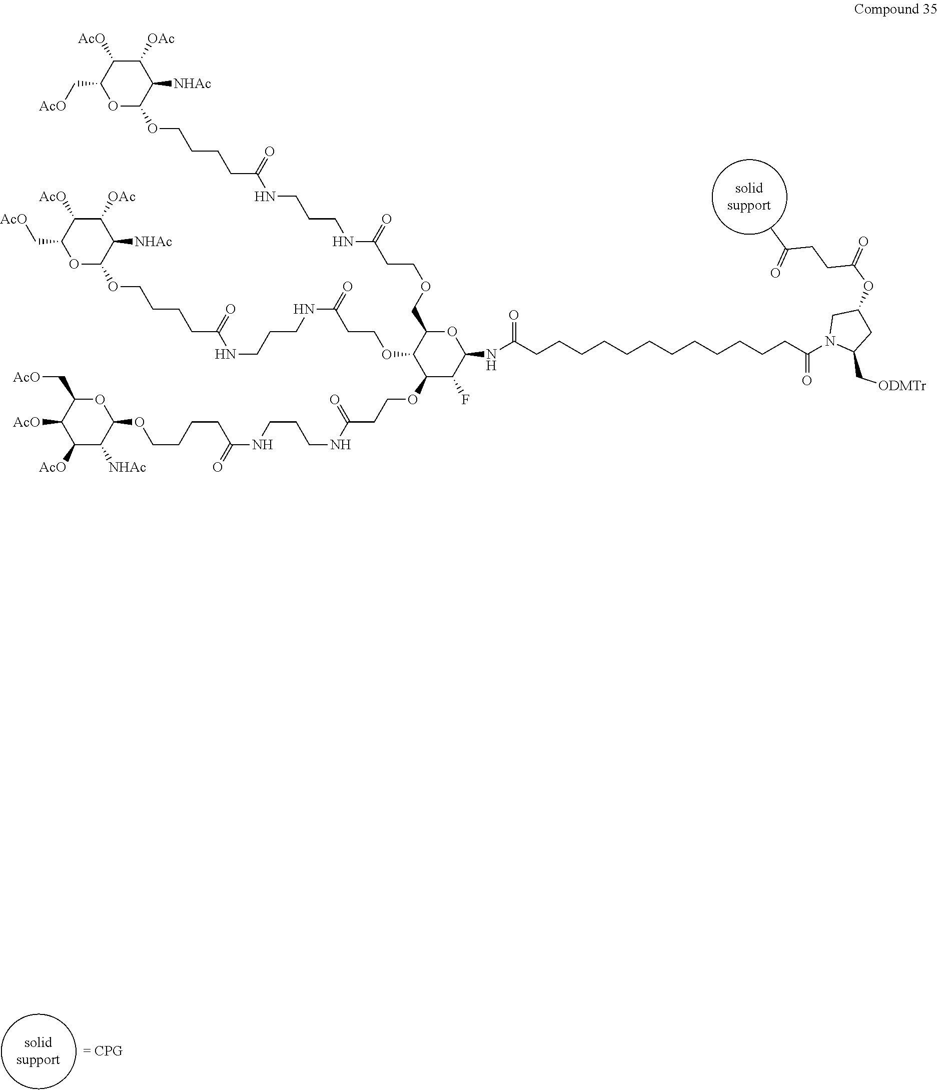

Compound 35 was synthesized following the procedure of compound 7 in example 30 by using 35-q as the starting material of which the synthesis was shown below. Loading: 24.0 μmol/g.

### 4-(((3R,5S)-1-(14-(((2R,3R,4S,5R,6R)-4,5-bis(3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)-6-((3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)methyl)-3-fluorotetrahydro-2H-pyran-2-yl)amino)-14-oxotetradecanoyl)-5-((bis(4-methoxyphenyl)(phenyl)methoxy)methyl)pyrrolidin-3-yl)oxy)-4-oxobutanoic acid (35-q)

The title compound was synthesized following the procedure of compound 7-q in example 30. LCMS: calcd for [M−2H]/2: 1296.1, found: 1296.7.

### Example 94

### Synthesis of GalNAc-siRNA Conjugate Compound 35-F

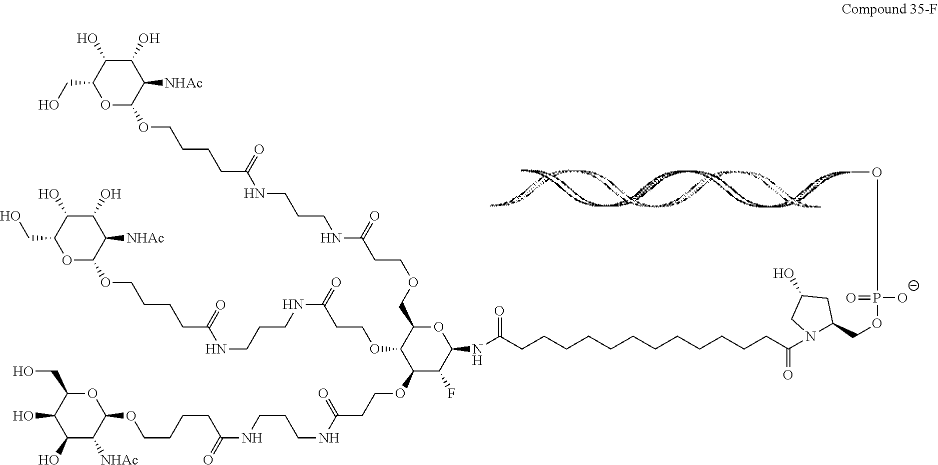 [SMILES: COP(=O)([O-])OC[C@@H]1C[C@@H](O)CN1C(=O)CCCCCCCCCCCCC(=O)N[C@@H]1O[C@H](COCCC(=O)NCCCNC(=O)CCCCO[C@@H]2O[C@H](CO)[C@H](O)[C@H](O)[C@H]2NC(C)=O)[C@@H](OCCC(=O)NCCCNC(=O)CCCCO[C@@H]2O[C@H](CO)[C@H](O)[C@H](O)[C@H]2NC(C)=O)[C@H](OCCC(=O)NCCCNC(=O)CCCCO[C@@H]2O[C@H](CO)[C@H](O)[C@H](O)[C@H]2NC(C)=O)[C@H]1F]

Using the conjugate building block compound 35 described earlier (Example 93), RNA is synthesized with the ligand attached to 3′-end of the sense strand according to known procedures. This is annealed with an antisense strand. The product is shown above.

### Example 95

### Synthesis of Compound 36

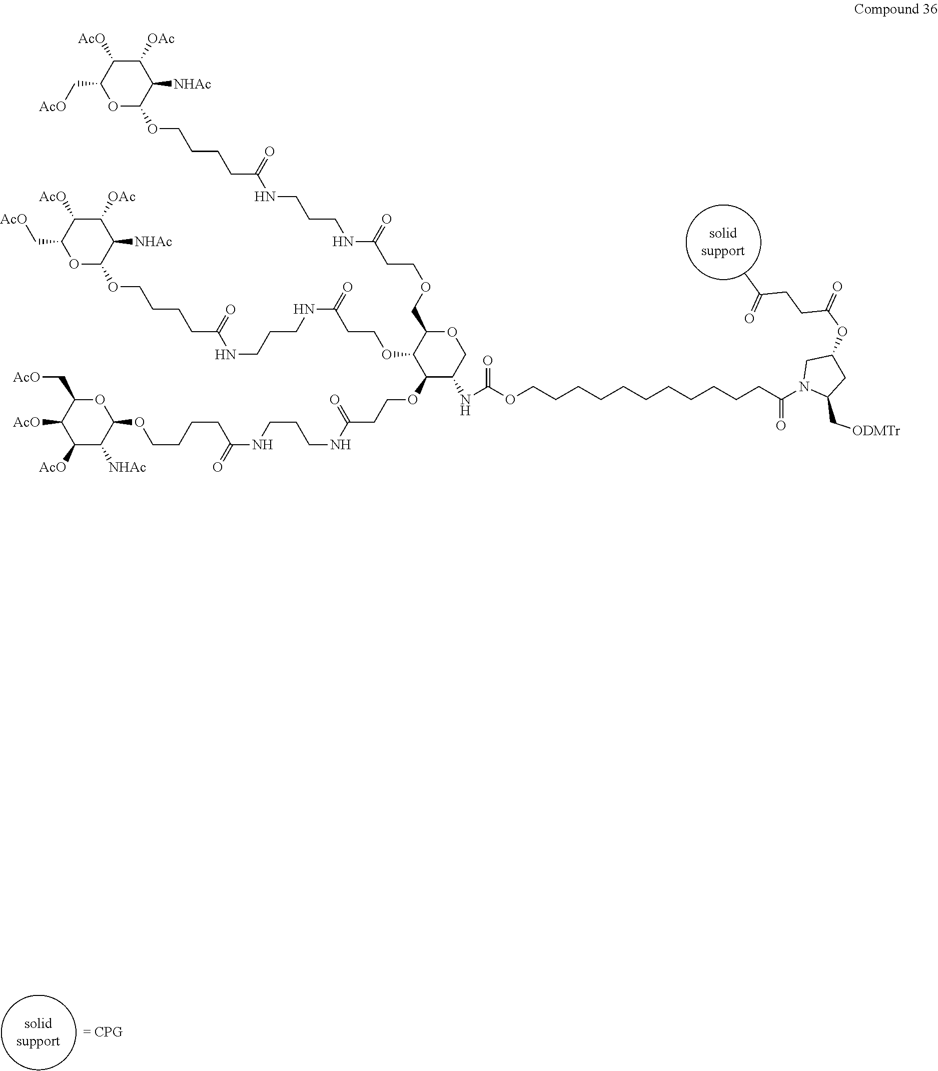

Compound 36 was synthesized following the procedure of compound 6 in example 29 by using 36-j as the starting material of which the synthesis was shown below. Loading: 28.0 μmol/g.

### 4-(((3R,5S)-1-(10-((((3S,4R,5S,6R)-4,5-bis(3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)-6-((3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)methyl)tetrahydro-2H-pyran-3-yl)carbamoyl)oxy)decanoyl)-5-((bis(4-methoxyphenyl)(phenyl)methoxy)methyl)pyrrolidin-3-yl)oxy)-4-oxobutanoic acid (36-j)

The title compound was synthesized following the procedure of compound 5-j in example 28 by using 36-f as the starting material of which the synthesis was shown below. LCMS: calcd for [M−2H]/2: 1274.1, found: 1274.7.

### The synthesis of 3,3′-(((2R,3S,4R,5S)-5-((((10-(benzyloxy)-10-oxodecyl)oxy)carbonyl)amino)-2-((2-carboxyethoxy)methyl)tetrahydro-2H-pyran-3,4-diyl)bis(oxy))dipropionic acid (36-f)

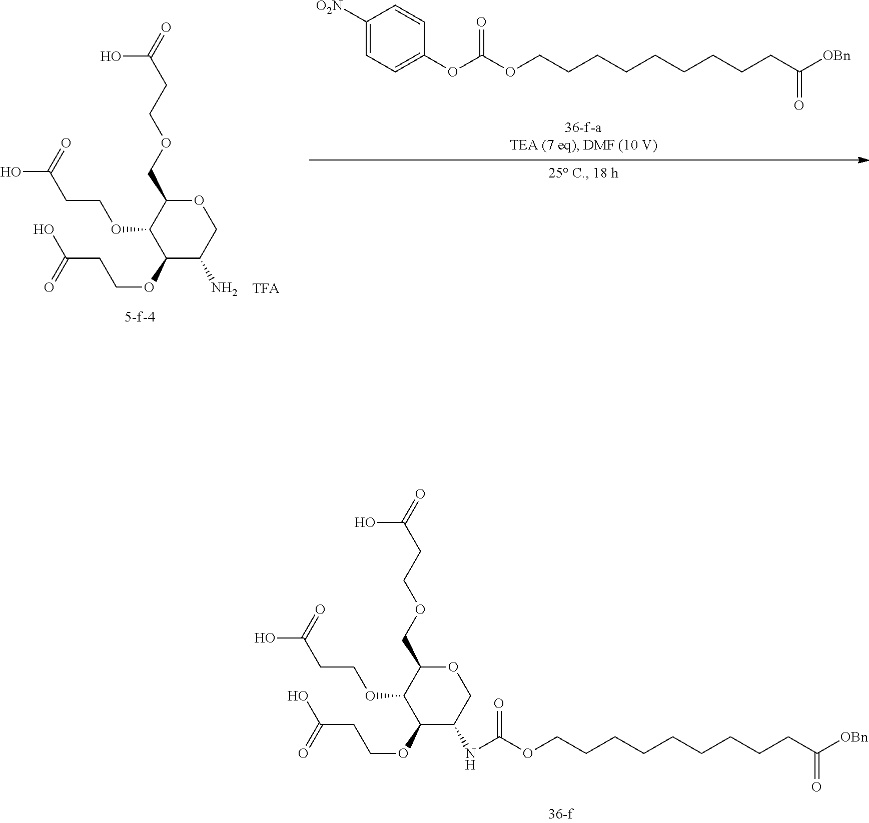 [SMILES: N[C@H]1CO[C@H](COCCC(=O)O)[C@@H](OCCC(=O)O)[C@@H]1OCCC(=O)O ; O=C(CCCCCCCCCOC(=O)Oc1ccc([N+](=O)[O-])cc1)OCc1ccccc1 ; "25° C., 18 h" ; "36-f-aTEA (7 eq), DMF (10 V)" ; O=C(O)CCOC[C@H]1OC[C@H](NC(=O)OCCCCCCCCCC(=O)OCc2ccccc2)[C@@H](OCCC(=O)O)[C@@H]1OCCC(=O)O]

Compound 5-f-4 (286.74 mg, 646.56 umol, 1.1 eq) was dissolved in anhydrous DMF (6 mL), TEA (416.34 mg, 4.11 mmol, 572.68 uL, 7 eq) was added, solid precipitated out from the mixture. Next, 36-f-a (0.29 g, 587.78 umol) was added and stirred at 25° C. (oil bath) for 16 h under N₂ to give a light yellow solution. LCMS indicated Compound 5-f-4 was consumed completely. The mixture was filtered and concentrated under vacuum. The crude product was precipitated with DCM/Hexane=1/5 at 25° C. for 60 min. The crude product was precipitated with ACN at 25° C. for 60 min to provide compound 36-f (0.49 g, brown power) which was used in the next step without further purification. LCMS: calcd for [M+NH₄]: 701.3, found: 701.3.

The synthesis of benzyl 10-(((4-nitrophenoxy)carbonyl)oxy)decanoate (36-f-a): To a stirred solution of benzyl 10-hydroxydecanoate (0.5 g, 1.80 mmol, 1 eq) in THE (6 mL) was added TEA (272.61 mg, 2.69 mmol, 374.98 uL, 1.5 eq) dropwise at 0° C. followed by the portion addition of 4-NO₂—C₆H₄O—COCl (615.44 mg, 3.05 mmol, 1.7 eq). The resulting solution was stirred at 20° C. 17 h. LCMS showed the starting material was consumed completely. The reaction mixture was concentrated under reduced pressure. The residue was purified by column chromatography (SiO₂, Petroleum ether/Ethyl acetate=50/1 to 0/1) to provide 36-f-a (0.6 g, yield: 75.33%). ¹H NMR: (400 MHz CDCl₃) δ=8.31-8.22 (m, 2H), 7.44-7.27 (m, 5H), 5.11 (s, 2H), 4.28 (t, J=6.8 Hz, 2H), 2.36 (t, J=7.2 Hz, 2H), 1.70-1.19 (m, 14H).

### Example 96

### Synthesis of GalNAc-siRNA Conjugate Compound 36-F

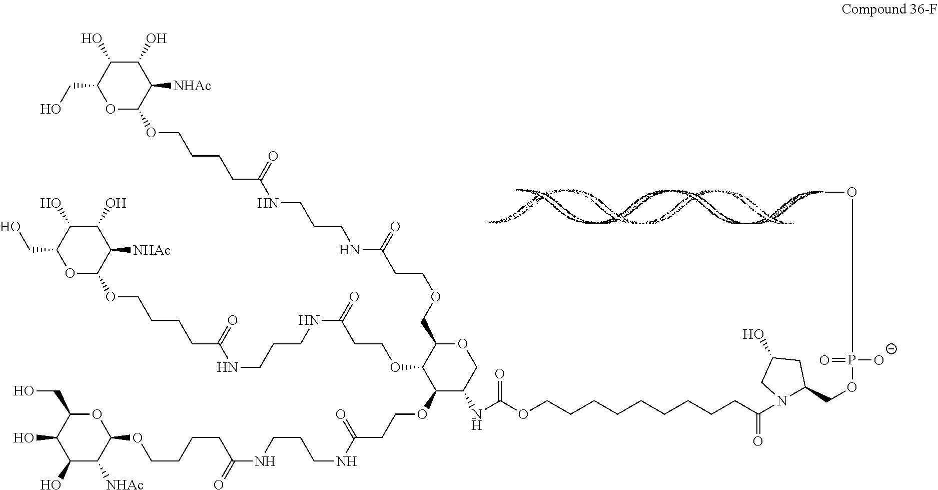 [SMILES: COP(=O)([O-])OC[C@@H]1C[C@@H](O)CN1C(=O)CCCCCCCCCOC(=O)N[C@H]1CO[C@H](COCCC(=O)NCCCNC(=O)CCCCO[C@@H]2O[C@H](CO)[C@H](O)[C@H](O)[C@H]2NC(C)=O)[C@@H](OCCC(=O)NCCCNC(=O)CCCCO[C@@H]2O[C@H](CO)[C@H](O)[C@H](O)[C@H]2NC(C)=O)[C@@H]1OCCC(=O)NCCCNC(=O)CCCCO[C@@H]1O[C@H](CO)[C@H](O)[C@H](O)[C@H]1NC(C)=O]

Using the conjugate building block compound 36 described earlier (Example 95), RNA is synthesized with the ligand attached to 3′-end of the sense strand according to known procedures. This is annealed with an antisense strand. The product is shown above.

### Example 97

### Synthesis of Compound 37

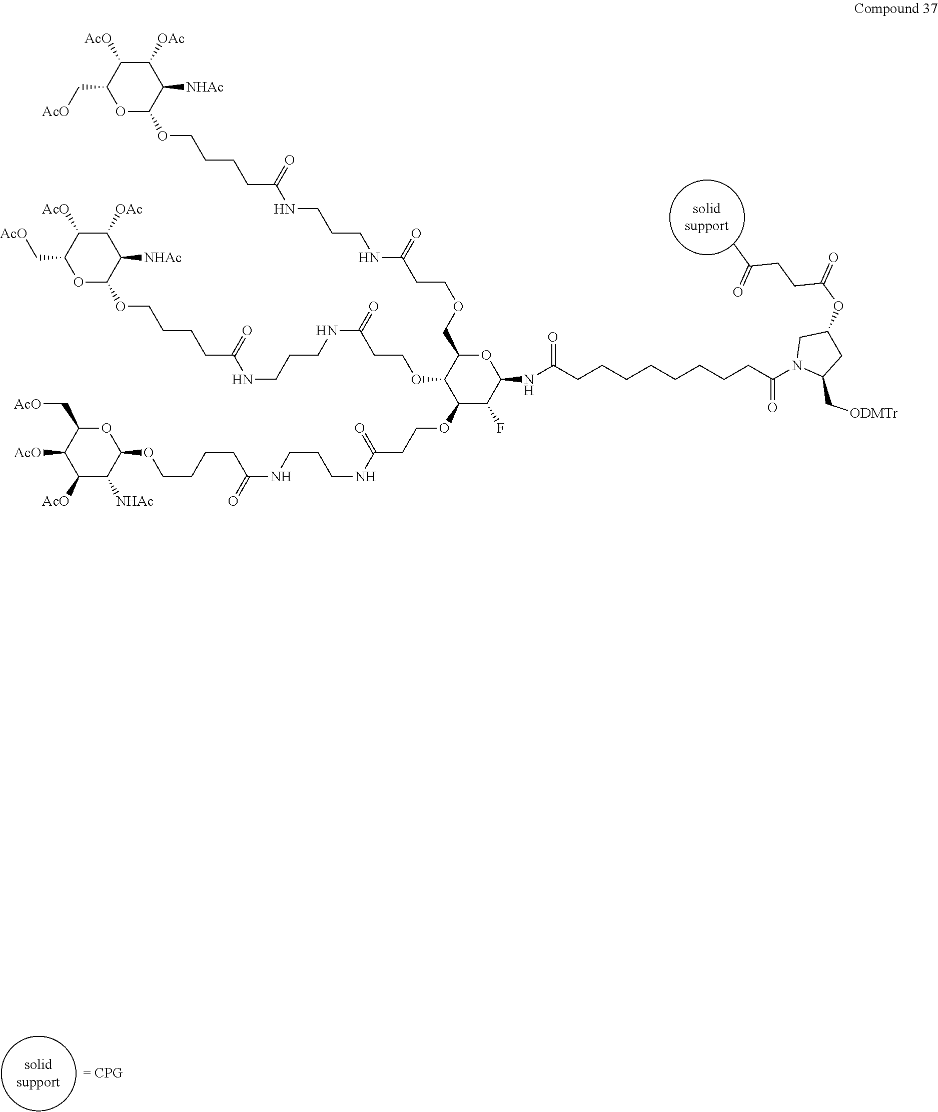

Compound 37 was synthesized following the procedure of compound 7 in example 30 by using 37-q as the starting material of which the synthesis was shown below. Loading: 37.0 μmol/g.

### 4-(((3R,5S)-1-(10-(((2R,3R,4S,5R,6R)-4,5-bis(3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)-6-((3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)methyl)-3-fluorotetrahydro-2H-pyran-2-yl)amino)-10-oxodecanoyl)-5-((bis(4-methoxyphenyl)(phenyl)methoxy)methyl)pyrrolidin-3-yl)oxy)-4-oxobutanoic acid (37-q)

The title compound was synthesized following the synthetic route of compound 7-q in example 30. LCMS: calcd for [M−2H]/2: 1268.1, found: 1268.6.

### Example 98

### Synthesis of GalNAc-siRNA Conjugate Compound 37-F

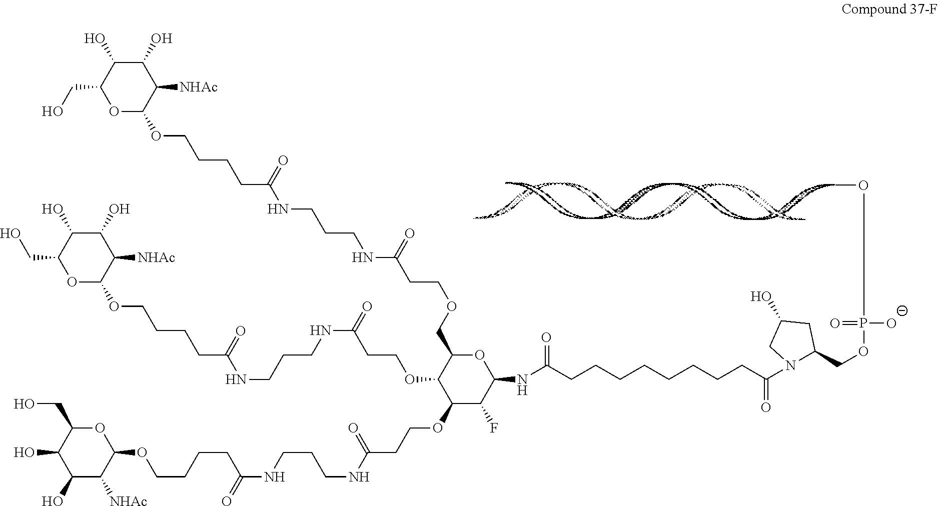 [SMILES: COP(=O)([O-])OC[C@@H]1C[C@@H](O)CN1C(=O)CCCCCCCCC(=O)N[C@@H]1O[C@H](COCCC(=O)NCCCNC(=O)CCCCO[C@@H]2O[C@H](CO)[C@H](O)[C@H](O)[C@H]2NC(C)=O)[C@@H](OCCC(=O)NCCCNC(=O)CCCCO[C@@H]2O[C@H](CO)[C@H](O)[C@H](O)[C@H]2NC(C)=O)[C@H](OCCC(=O)NCCCNC(=O)CCCCO[C@@H]2O[C@H](CO)[C@H](O)[C@H](O)[C@H]2NC(C)=O)[C@H]1F]

Using the conjugate building block compound 37 described earlier (Example 96), RNA is synthesized with the ligand attached to 3′-end of the sense strand according to known procedures. This is annealed with an antisense strand. The product is shown above.

### Example 99

### Synthesis of Compound 38

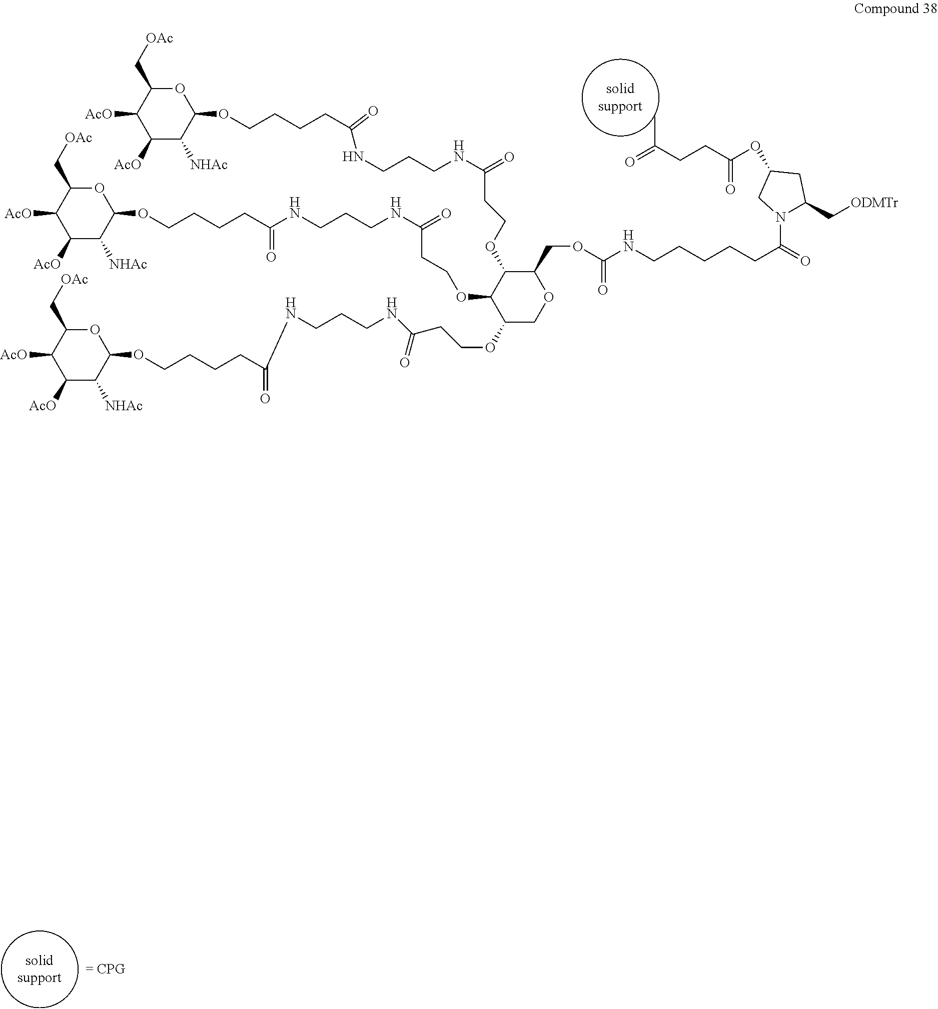

Compound 38 was synthesized following the procedure of compound 27 in example 77 by using 38-g as the starting material of which the synthesis was shown below. Loading: 27.0 μmol/g.

### 4-(((3R,5S)-5-((bis(4-methoxyphenyl)(phenyl)methoxy)methyl)-1-(6-(((((2R,3R,4R,5S)-3,4,5-tris(3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)tetrahydro-2H-pyran-2-yl)methoxy)carbonyl)amino)hexanoyl)pyrrolidin-3-yl)oxy)-4-oxobutanoic acid (38-g)

The title compound was synthesized following the procedure of compound 27-g in example 77. LCMS: calcd for [M−2H]/2: 1246.1, found: 1246.6.

### Example 100

### Synthesis of GalNAc-siRNA Conjugate Compound 38-F

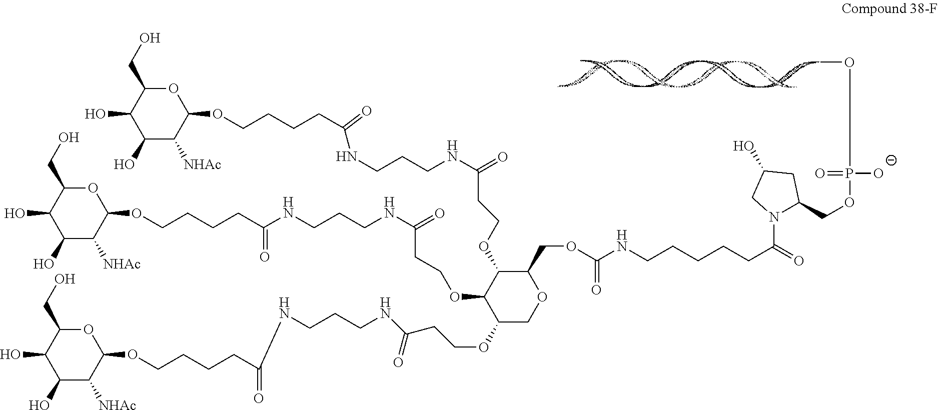 [SMILES: COP(=O)([O-])OC[C@@H]1C[C@@H](O)CN1C(=O)CCCCCNC(=O)OC[C@H]1OC[C@H](OCCC(=O)NCCCNC(=O)CCCCO[C@@H]2O[C@H](CO)[C@H](O)[C@H](O)[C@H]2NC(C)=O)[C@@H](OCCC(=O)NCCCNC(=O)CCCCO[C@@H]2O[C@H](CO)[C@H](O)[C@H](O)[C@H]2NC(C)=O)[C@@H]1OCCC(=O)NCCCNC(=O)CCCCO[C@@H]1O[C@H](CO)[C@H](O)[C@H](O)[C@H]1NC(C)=O]

Using the conjugate building block compound 38 described earlier (Example 99), RNA is synthesized with the ligand attached to 3′-end of the sense strand according to known procedures. This is annealed with an antisense strand. The product is shown above.

### Example 101

### Synthesis of Compound 39

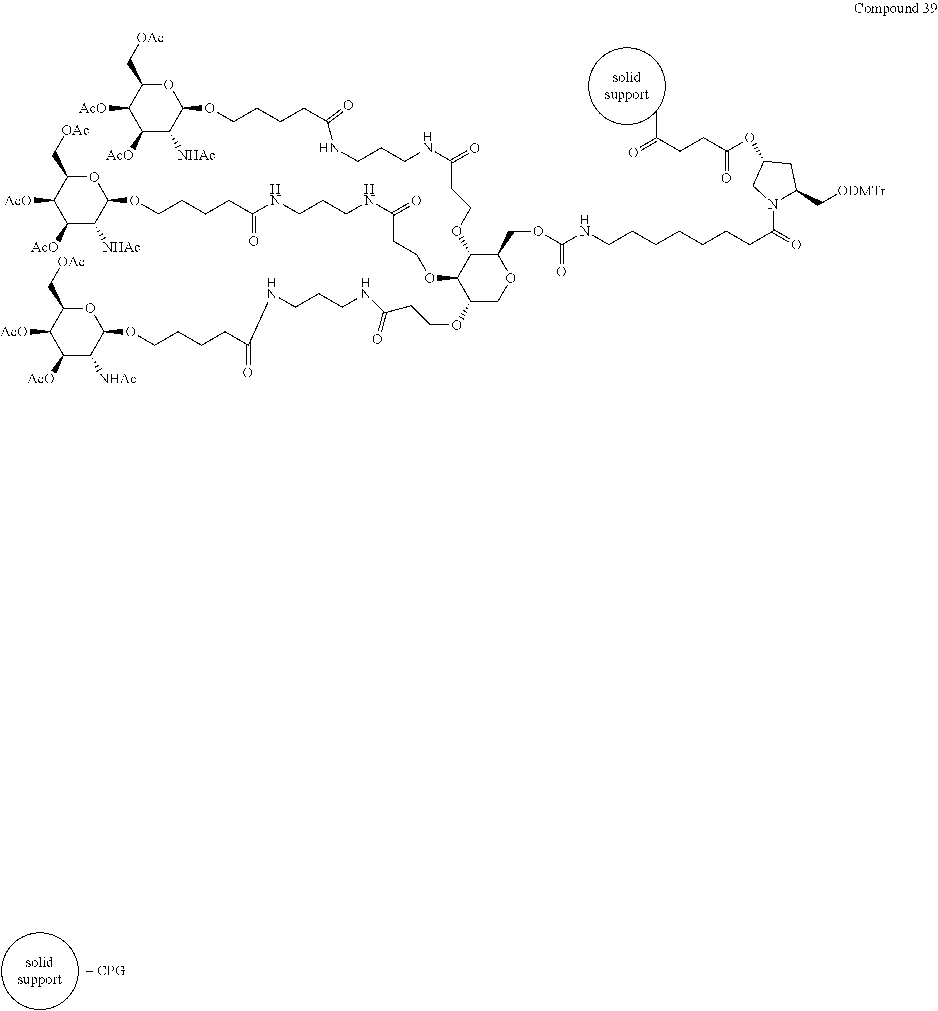

Compound 39 was synthesized following the procedure of compound 27 in example 77 by using 39-g as the starting material of which the synthesis was shown below. Loading: 31.0 μmol/g.

### 4-(((3R,5S)-5-((bis(4-methoxyphenyl)(phenyl)methoxy)methyl)-1-(8-(((((2R,3R,4R,5S)-3,4,5-tris(3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)tetrahydro-2H-pyran-2-yl)methoxy)carbonyl)amino)octanoyl)pyrrolidin-3-yl)oxy)-4-oxobutanoic acid (39-g)

The title compound was synthesized following the procedure of compound 27-g in example 77. LCMS: calcd for [M−2H]/2: 1260.1, found: 1260.6.

### Example 102

### Synthesis of GalNAc-siRNA conjugate compound 39-F

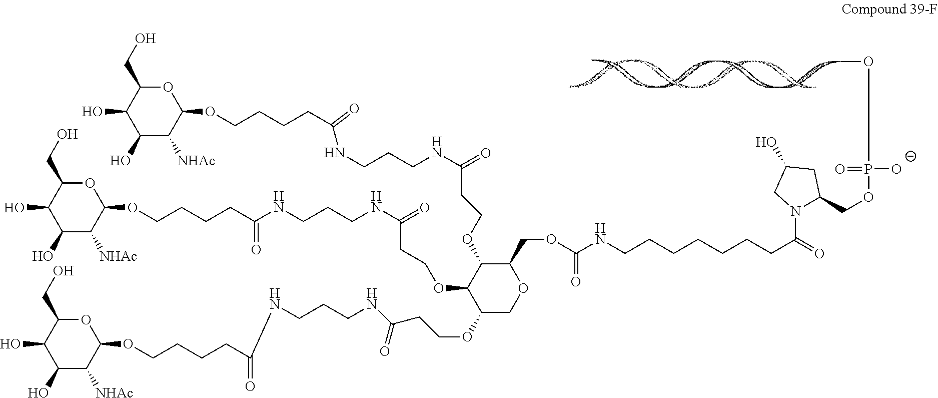 [SMILES: COP(=O)([O-])OC[C@@H]1C[C@@H](O)CN1C(=O)CCCCCCCNC(=O)OC[C@H]1OC[C@H](OCCC(=O)NCCCNC(=O)CCCCO[C@@H]2O[C@H](CO)[C@H](O)[C@H](O)[C@H]2NC(C)=O)[C@@H](OCCC(=O)NCCCNC(=O)CCCCO[C@@H]2O[C@H](CO)[C@H](O)[C@H](O)[C@H]2NC(C)=O)[C@@H]1OCCC(=O)NCCCNC(=O)CCCCO[C@@H]1O[C@H](CO)[C@H](O)[C@H](O)[C@H]1NC(C)=O]

Using the conjugate building block compound 39 described earlier (Example 101), RNA is synthesized with the ligand attached to 3′-end of the sense strand according to known procedures. This is annealed with an antisense strand. The product is shown above.

### Biological Example 1

### RNA Synthesis and Duplex Annealing

Oligonucleotides were synthesized by oligonucleotide synthesizer using commercially available nucleotides or chemically modified nucleotides with proper protecting groups.

Ligand conjugated strands were synthesized by using solid support containing the corresponding ligand. For example, the introduction of carbohydrate moiety/ligand (e.g., GalNAc) at the 3′-end of a sequence was achieved by starting the synthesis with the corresponding carbohydrate solid support following the standard oligonucleotide synthetic procedure by using oligonucleotide synthesizer.

After completion of synthesis, the support and the protecting groups were removed from the oligonucleotides by using proper deprotection system together or separately.

For the preparation of siRNA, equimolar amounts of sense and antisense strand were heated in 1×PBS at 95° C. for 5 min and slowly cooled to room temperature. Integrity of the duplex was confirmed by HPLC analysis.

Listed in Table 2 are the published siRNA sequences (Please refer to “miR-145 Antagonizes SNAI1-Mediated Stemness and Radiation Resistance in Colorectal Cancer”, Molecular Therapy, Vol. 26, No. 3, March 2018) used to provide the GalNAc-siRNA conjugates for biochemical characterization.

TABLE 2

| Target | S/AS | Sequence 5′-3′ |
|---|---|---|
| TTR | S | CmsAmsGmUmGfUmUfCfUfUmGmCmUmCmUmAmUmAmAm |
| | AS | UmsUfsAmUmAmGfAmGmCmAmAmGmAmAfCmAfCmUmGmsUmsUm |

Note:
lowercase s is PS linkage, lowercase m is 2′-O-methyl nucleotide, lowercase f is 2′-fluoro nucleotide; TTR is Transthyretin.

Listed in Table 3 are the GalNAc-siRNA conjugates and the corresponding quality characterizations.

TABLE 3

| | | Calculated | | |
|---|---|---|---|---|
| Compound ID | Chain ID | MS | Found MS | Purity (%) |
| Compound 2-F (TTR)¹ | S_moiety 2″ | 8043.1 | 8045.4 | 95.0 |
| | AS | 7015.8 | 7017.4 | 95.6 |
| Compound 6-F (TTR)¹ | S_ moiety 6″ | 8100.2 | 8101.6 | 96.4 |
| | AS | 7015.8 | 7017.4 | 95.6 |
| Compound 7-F (TTR)¹ | S_ moiety 7″ | 8061.1 | 8063.0 | 90.5 |
| | AS | 7015.8 | 7017.4 | 95.6 |
| Compound 27-F (TTR)¹ | S_ moiety 27″ | 8045.1 | 8046.6 | 96.22 |
| | AS | 7015.8 | 7017.4 | 95.6 |
| L96_TTR^{1,2} | S_L96 | 8000.1 | 8002.3 | 95.9 |
| | AS | 7015.8 | 7018.0 | 94.7 |
| TTR | S | 6211.1 | 6213.4 | 98.9 |
| | AS | 7015.8 | 7018.0 | 95.2 |
| Compound 32-F (TTR)¹ | S_moiety 32″ | 8039.0 | 8040.0 | 95.5 |
| | AS | 7015.8 | 7017.6 | 95.0 |
| Compound 33-F (TTR)¹ | S_moiety 33″ | 8027.0 | 8028.3 | 96.2 |
| | AS | 7015.8 | 7017.6 | 95.0 |
| Compound 5-F (TTR)¹ | S_moiety 5″ | 8043.0 | 8044.0 | 96.5 |
| | AS | 7015.8 | 7017.6 | 95.0 |
| Compound 30-F (TTR)¹ | S_moiety 30″ | 8142.3 | 8143.2 | 97.5 |
| | AS | 7015.8 | 7017.0 | 94.6 |
| Compound 28-F (TTR)¹ | S_moiety 28″ | 8072.0 | 8073.7 | 94.9 |
| | AS | 7015.8 | 7017.6 | 95.0 |
| Compound 29-F (TTR)¹ | S_moiety 29″ | 8128.0 | 8129.1 | 97.8 |
| | AS | 7015.8 | 7017.6 | 95.0 |

Note:
¹of which the conjugated siRNA is TTR; ² The structure of L96 was shown below.

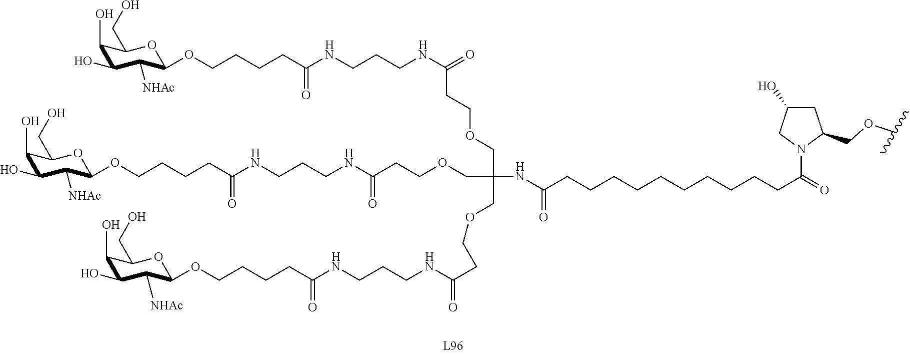

In Vitro Silencing Activity of GalNAc-siRNA Conjugates that Target TTR

### Isolation of Primary Mouse Hepatocytes

The C57BL/6 mice were anesthetized and the liver were perfused with 100 ml HBSS (GIBCO, 14025092) containing 1 mM EGTA (Aladdin, e104432) through an intravenous needle inserted into the inferior vena cava, followed by 100 ml collagenase-containing HBSS (collagenase type I, Solelybio, sy0535). Livers were excised and washed in PBS, and then disassociated in culture medium. The envelope of the liver was torn and the contents were released. Cells were filtered by 100 micron nylon mesh and centrifuge at 4° C., 200 g for 3 minutes. The yield and viability were determined using a trypan blue exclusion test (Sigma, 0.08%). The viability of hepatocytes above 85% were available for the subsequent operations.

### Free-Uptake

900 μL of complete growth media containing 8×10⁴ primary mouse hepatocytes (PMH) were added into a 24-well plate precoated with type I collagenase. Free-uptake was carried out by adding 10 μL of test articles/siRNA duplexes plus 90 μL of Opti-MEM into PMHs and mixed well. Cells were incubated for 24 hours at 37° C. in an atmosphere of 5% CO₂ prior to RNA purification. Four doses experiments were performed at 2 nM, 1 nM, 0.5 nM, and 0.25 nM. Eight points IC₅₀ curve fitting was based at 10 nM, 2.5 nM, 0.63 nM, 0.16 nM, 39 pM, 9.8 pM, 2.4 pM and 0.61 pM.

Total RNA Isolation (Invitrogen, 610-12) and cDNA Synthesis (TaKaRa, RR047A)

Cells were lysed in 300 μL of lysis buffer and transferred into a 96-deep-well plate (Plate 1). 20 μL of magnetic beads and 280 μL anhydrous ethanol were then added into each well in Plate 1. 500 μL/well of MW2 were added into Plate 2 and Plate 3 with the same arrangement. 50 μL/well of elution buffer were added into Plate 4 with the same typesetting. Place each plate into nucleic acid extraction instrument and run with isolation program. The concentration of each RNA sample was determined using NanoDrop. A master mix of 1 μL gDNA eraser and 2 μL 5× gDNA Eraser buffer were added into 7 μL total RNA. The RT1 program is: 42° C. 2 min, 4° C. hold. 4 μL 5× PrimeScript Buffer 2, 1 μLPrimeScript RT Enzyme Mix I, 1 μL RT Primer Mix and 4 μL RNase Free dH₂O were added into the above reaction and the RT2 program is: 37° C. 15 min, 85° C. 5 see, 4° C. hold. 180 μL of dilution buffer were added into each well for real time PCR.

### Real Time PCR

4 μL of cDNA were added to a master mix containing 1 μL TBP/GAPDH (mouse) primer or 1 μL TTR (mouse) primer and 5 μL PCR Buffer per well in a 384 well plate. Real time PCR was done in a Roche LC480 Real Time PCR machine. Each duplex was tested in at two independent free-uptakes and each free-uptake was assayed in three duplicates. To calculate relative fold change, real time PCR data were analyzed using ΔΔCt method and normalized to assays performed with mock cells.

### Activity Result

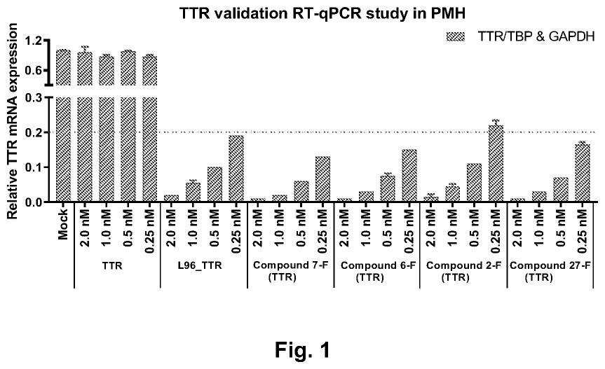

The activity of GalNAc-siRNA conjugates (Listed in Table 3) is shown in Table 4 and FIG. 1. Specifically, FIG. 1 shows in vitro TTR gene silencing by GalNac-siRNA Conjugates.

TABLE 4
GalNAc-siRNA conjugates IC₅₀ values

| | IC₅₀ | | IC₅₀ |
|---|---|---|---|
| Compound ID | (nM) | Compound ID | (nM) |
| Compound 28-F (TTR) | 0.08 | Compound 29-F (TTR) | 0.067 |
| Compound 33-F (TTR) | 0.08 | Compound 5-F (TTR) | 0.10 |
| Compound 32-F (TTR) | 0.07 | Compound 30-F (TTR) | 0.10 |
| L96_TTR | 0.10 | | |

The foregoing description is considered as illustrative only of the principles of the present invention. Further, since numerous modifications and changes will be readily apparent to those skilled in the art, it is not desired to limit the invention to the exact construction and process shown as described above. Accordingly, all suitable modifications and equivalents may be considered to fall within the scope of the invention as defined by the claims that follow.

All references, patents and patent applications and publications that are cited or referred to in this application are incorporated herein in their entirety herein by reference.

1. A compound having the structure shown in Formula (I) 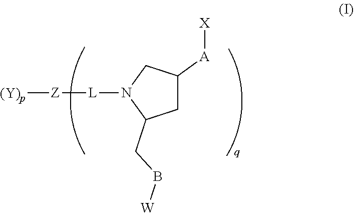
or a pharmaceutically acceptable salt or solvate thereof, wherein
A and B, for each occurrence, are each independently O, N(R^{N}), or S;
R^{N} is H or C₁₋₆ alkyl;
X and W, for each occurrence, are each independently H, a protecting group, a phosphate group, a phosphodiester group, an activated phosphate group, an activated phosphite group, a phosphoramidite, a solid support, —P(Z′)(Z″)O-nucleoside, —P(Z′)(Z″)O-oligonucleotide, a lipid, a PEG, a steroid, a polymer, a nucleotide, a nucleoside, an oligonucleotide, or a therapeutically active agent;
Z′ and Z″ are each independently O or S;
L is a covalent linker;
Z is a carbohydrate mimetic or a disaccharide, trisaccharide, or oligosaccharide;
each Y is independently -L′-T;
each T is independently a ligand selected from the group consisting of carbohydrate ligands, polypeptide ligands, and lipophile ligands;
each L′ is independently a covalent linker; and
p and q are each independently 1, 2, 3, 4, or 5.

2. (canceled)

3. The compound as claimed in claim 1 or a pharmaceutically acceptable salt or solvate thereof, wherein Z is a carbohydrate mimetic of a monosaccharide selected from the group consisting of deoxysugars, aminosugars, N-glycosides, iminosugars, unsaturated sugars, carboxylated sugars, amidated sugars, fused cyclic sugars, and carbasugars of a monosaccharide optionally, the monosaccharide is a terose, pentose, hexose, heptose, or octose.

4.-5. (canceled)

6. The compound as claimed in claim 1 or a pharmaceutically acceptable salt or solvate thereof, wherein Z has the structure: 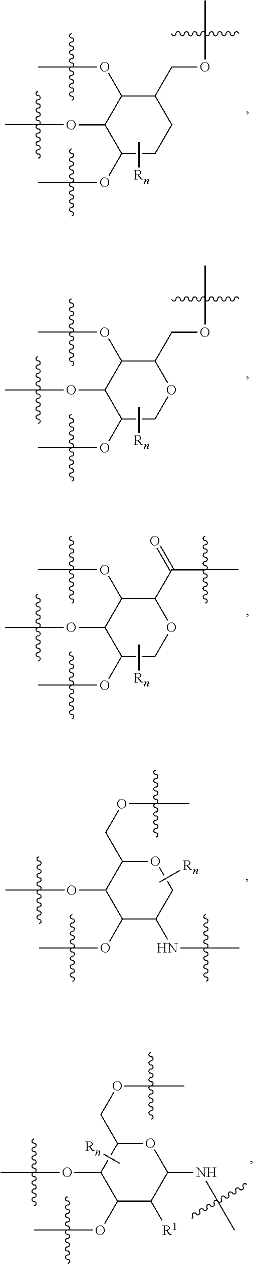 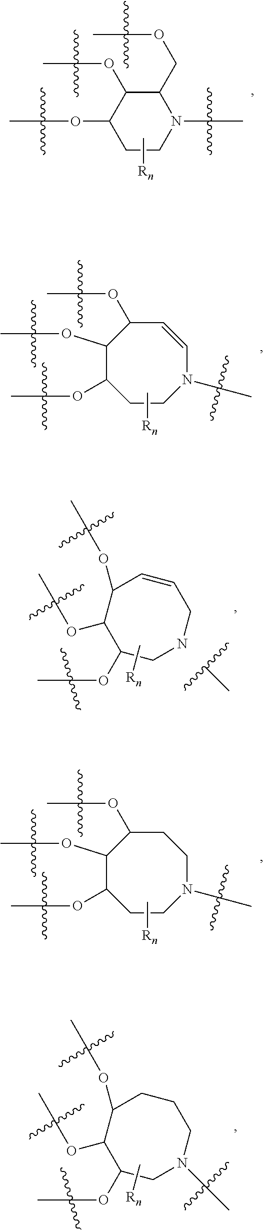 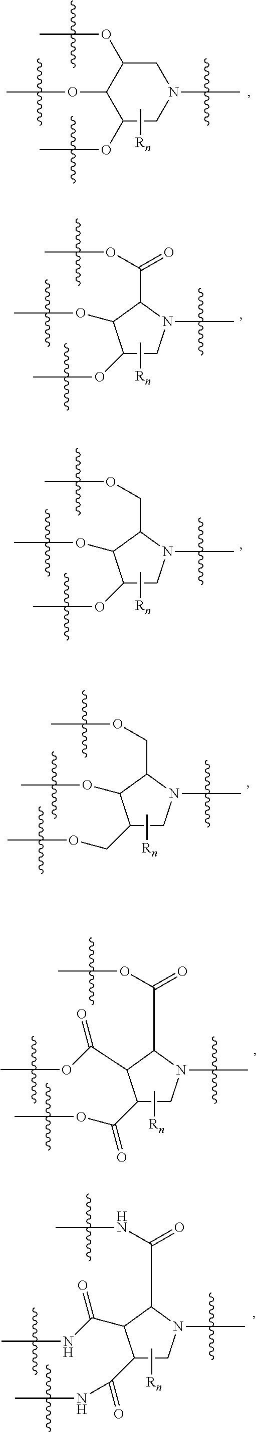 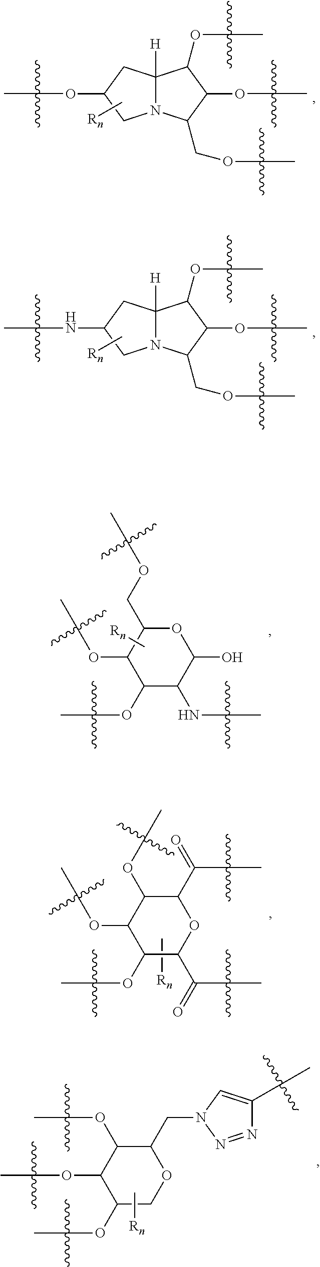 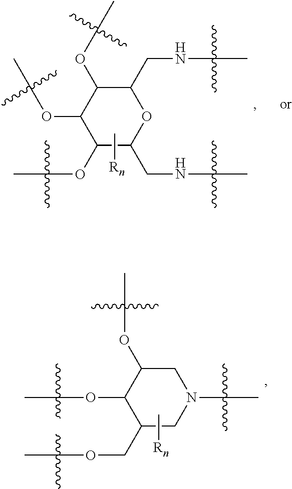
wherein
R¹ is H, C₁₋₆ alkyl, halogen or —NH(R²), wherein R² is H or acetyl;
each R is independently H, halogen, —CN, —C≡CH, —NH₂, —OC₁₋₆ alkyl, or C₁₋₆ alkyl, wherein said alkyl of —C₁₋₆alkyl and —OC₁₋₆alkyl is substituted with 0 to 5 halogen atoms; or two R together with the carbon to which they are attached form a C₃₋₆cycloalkyl or 3- to 6-membered heterocycloalkyl group, wherein said cycloalkyl of C₃₋₆cycloalkyl and heterocycloalkyl of 3- to 6-membered heterocycloalkyl is substituted with 0 to 5 halogen atoms; and
n is 0, 1, 2, or 3, as valency permits.

7.-8. (canceled)

9. The compound as claimed in claim 1 or a pharmaceutically acceptable salt or solvate thereof, wherein (Y)ₚ—Z— has the structure

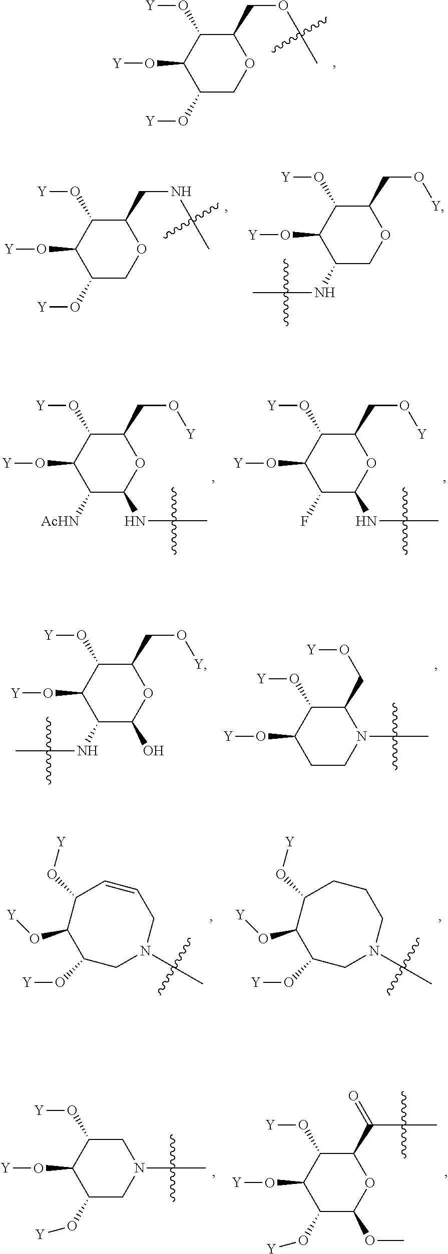

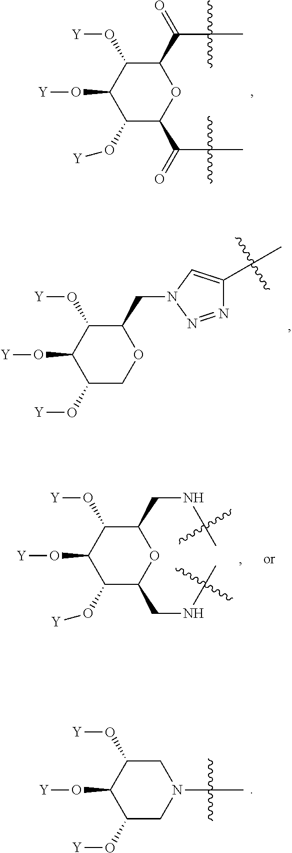

10.-11. (canceled)

12. The compound as claimed in claim 1 or a pharmaceutically acceptable salt or solvate thereof, wherein Z is a disaccharide selected from the group consisting of gentiobiose, isomaltose, melibiose, trehalose, sucrose, lactose, maltose, and cellobiose, or a carbohydrate mimetic thereof.

13. The compound as claimed in claim 1 or a pharmaceutically acceptable salt or solvate thereof, wherein Z has the following structure

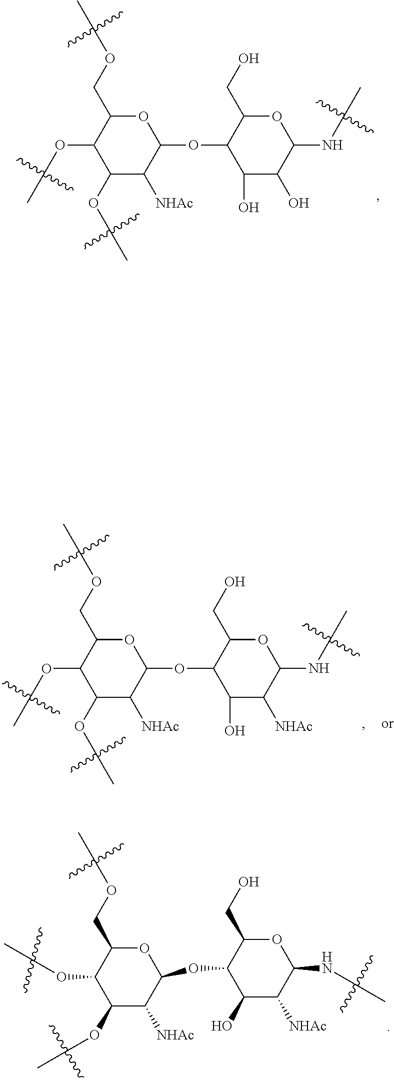 [SMILES: *NC1OC(CO)C(OC2OC(CO*)C(O*)C(O*)C2NC(C)=O)C(O)C1O ; *NC1OC(CO)C(OC2OC(CO*)C(O*)C(O*)C2NC(C)=O)C(O)C1NC(C)=O ; *N[C@@H]1O[C@H](CO)[C@@H](O[C@@H]2O[C@H](CO*)[C@@H](O*)[C@H](O*)[C@H]2NC(C)=O)[C@H](O)[C@H]1NC(C)=O]

14. (canceled)

15. The compound as claimed in claim 1 or a pharmaceutically acceptable salt or solvate thereof, wherein
each 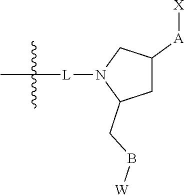
is independently a group having the following structure 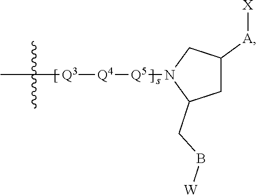
wherein
s is an integer from 1 to 20,
each Q³ is independently absent, —CO—, —NH—, —O—, —S—, —SO₂—, —OC(O)—, —C(O)O—, —NHC(O), —C(O)NH—, —CH₂—, —CH₂NH—, —NHCH₂—, —CH₂O—, or —OCH₂—,
each Q⁴ is independently absent, unsubstituted or substituted C₁₋₁₂ alkylene, unsubstituted or substituted C₂₋₁₂ alkenylene, unsubstituted or substituted C₂₋₁₂ alkynylene, unsubstituted or substituted C₂₋₁₂ heteroalkylene, unsubstituted or substituted 6- to 12-membered arylene, unsubstituted or substituted 5- to 12-membered heteroarylene, or unsubstituted or substituted 5- to 12-membered heterocyclylene, and
each Q⁵ is independently absent, —CO—, —NH—, —O—, —S—, —SO₂—, —CH₂—, —C(O)O—, —OC(O)—, —C(O)NH—, —NHC(O)—, —NH—CH(R^{a})—C(O)—, —C(O)—CH(R^{a})—NH—, —OP(O)(OH)O—, or —OP(S)(OH)O—, wherein each R^{a} is independently H or unsubstituted or substituted C₁₋₁₂ alkyl,
provided that at least one Q⁴ is present.

16.-17. (canceled)

18. The compound as claimed in claim 15 or a pharmaceutically acceptable salt or solvate thereof, wherein
each 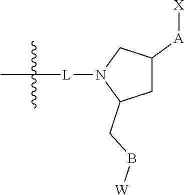
is independently a group having the structure 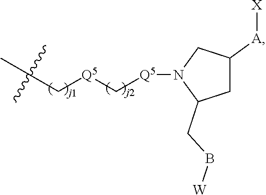
wherein
each Q⁵ is independently —CO—, —NH—, —O—, —CH₂—, —C(O)O—, —OC(O)—, —C(O)NH—, or —NHC(O)—, and
each of j1 and j2 is independently 0, 1, 2, 3, 4, 5, 6, 7, 8, 9, 10, 11, or 12.

19. The compound as claimed in claim 15 or a pharmaceutically acceptable salt or solvate thereof, wherein
each 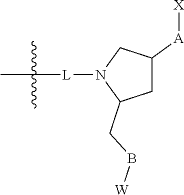
is independently a group having the following structure

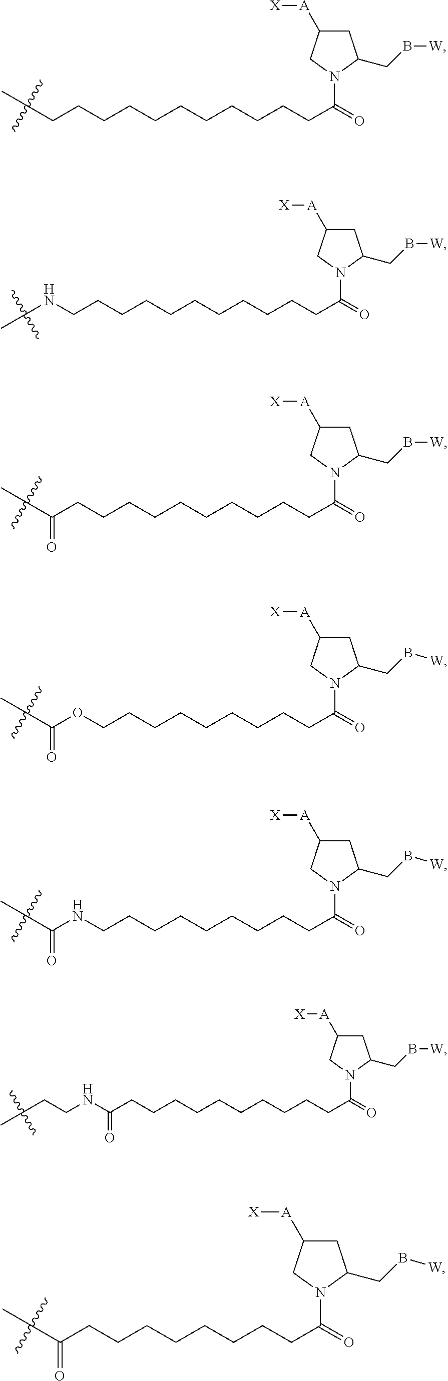

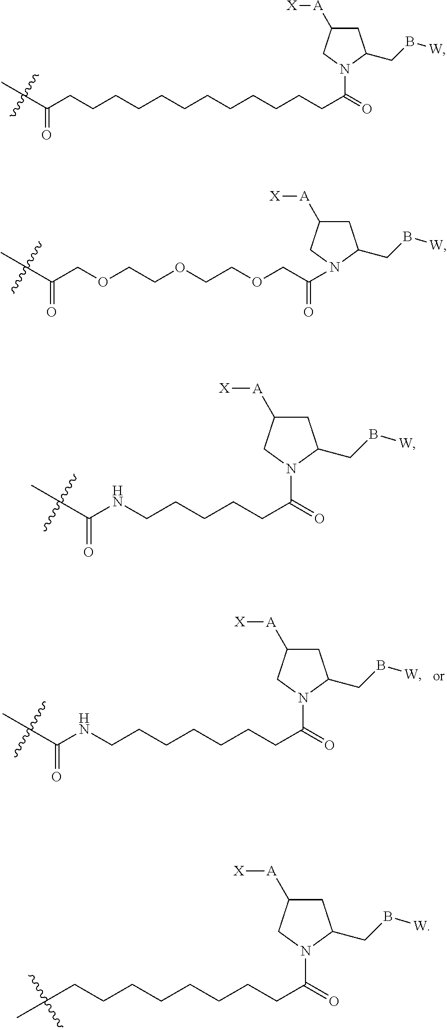

20. The compound as claimed in claim 1 or a pharmaceutically acceptable salt or solvate thereof, wherein each -L′-T is independently a group having the following structure:
[-Q³-Q⁴-Q⁵]ₛ-Q⁶-T,
wherein
s is an integer from 0 to 20,
each of Q³ and Q⁶ is independently absent, —CO—, —NH—, —O—, —S—, —SO₂—, —OC(O)—, —C(O)O—, —NHC(O)—, —C(O)NH—, —CH₂—, —CH₂NH—, —NHCH₂—, —CH₂O—, or —OCH₂—,
each Q⁴ is independently absent, unsubstituted or substituted C₁₋₁₂ alkylene, unsubstituted or substituted C₂₋₁₂ alkenylene, unsubstituted or substituted C₂₋₁₂ alkynylene, unsubstituted or substituted C₂₋₁₂ heteroalkylene, unsubstituted or substituted 6- to 12-membered arylene, unsubstituted or substituted 5- to 12-membered heteroarylene, or unsubstituted or substituted 5- to 12-membered heterocyclylene, and
each Q⁵ is independently absent, —CO—, —NH—, —O—, —S—, —SO₂—, —CH₂—, —C(O)O—, —OC(O)—, —C(O)NH—, —NHC(O)—, —NH—CH(R^{a})—C(O)—, —C(O)—CH(R^{a})—NH—, —OP(O)(OH)O—, or —OP(S)(OH)O—, wherein each R^{a} is independently H or unsubstituted or substituted C₁₋₁₂ alkyl,
provided that at least one of Q³, Q⁴, Q⁵, and Q⁶ is present.

21. (canceled)

22. The compound as claimed in claim 1 or a pharmaceutically acceptable salt or solvate thereof, wherein each -L′-T is independently a group of the following structure: 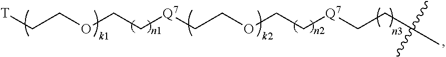
wherein
each Q⁷ is independently absent, —CO—, —NH—, —O—, —S—, —SO₂—, —OC(O)—, —C(O)O—, —NHC(O)—, —C(O)NH—, —CH₂—, —CH₂NH—, —NHCH₂—, —CH₂O—, or —OCH₂—,
each of k1 and k2 is independently 0, 1, 2, 3, 4, 5, 6, 7, 8, 9, or 10, and
each of n1, n2 and n3 is independently 1, 2, 3, 4, or 5.

23. (canceled)

24. The compound as claimed in claim 1 or a pharmaceutically acceptable salt or solvate thereof, wherein each -L′-T is independently a group of the following structure: 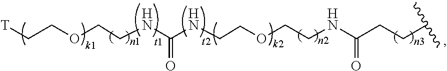
wherein
each of k1 and k2 is independently 0, 1, 2, or 3,
each of n1, n2 and n3 is independently 1, 2, 3, 4, or 5, and
one of t1 and t2 is 0 and anther of t1 and t2 is 1;
or 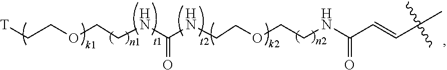
wherein
each of k1 and k2 is independently 0, 1, 2, or 3,
each of n1 and n2 is independently 1, 2, 3, 4, or 5, and
one of t1 and t2 is 0 and another of t1 and t2 is 1.

25.-28. (canceled)

29. The compound as claimed in claim 1 or a pharmaceutically acceptable salt or solvate thereof, wherein each T is independently a carbohydrate ligand selected from the group consisting of N-acetyl-galactosamine (GalNAc), allose, altrose, arabinose, cladinose, erythrose, erythrulose, fructose, D-fucitol, L-fucitol, fucosamine, fucose, fuculose, galactosamine, D-galactosaminitol, galactose, glucosamine, N-acetyl-glucosamine, glucosaminitol, glucose, glucose-6-phosphate, gulose glyceraldehyde, L-glycero-D-mannos-heptose, glycerol, glycerone, gulose, idose, lyxose, mannosamine, mannose, mannose-6-phosphate, psicose, quinovose, quinovosamine, rhamnitol, rhamnosamine, rhamnose, ribose, ribulose, sedoheptulose, sorbose, tagatose, talose, tartaric acid, threose, xylose, and xylulose, in an unprotected or protected form.

30. (canceled)

31. The compound as claimed in claim 1 or a pharmaceutically acceptable salt or solvate thereof, wherein the therapeutically active agent is selected from the group consisting of an antisense oligonucleotide (ASO), a small interfering RNA (siRNA), a microRNA (miRNA), a microRNA mimic, an anti-miRNA oligonucleotide (AMO), a long non-coding RNA, a peptide nucleic acid (PNA), a helper lipid, and a phosphorodiamidate morpholino oligomer (PMO), wherein the nucleic acid is unmodified or modified.

32. (canceled)

33. The compound as claimed in claim 1 or a pharmaceutically acceptable salt or solvate thereof, wherein said compound comprises a structure selected from the group consisting of:

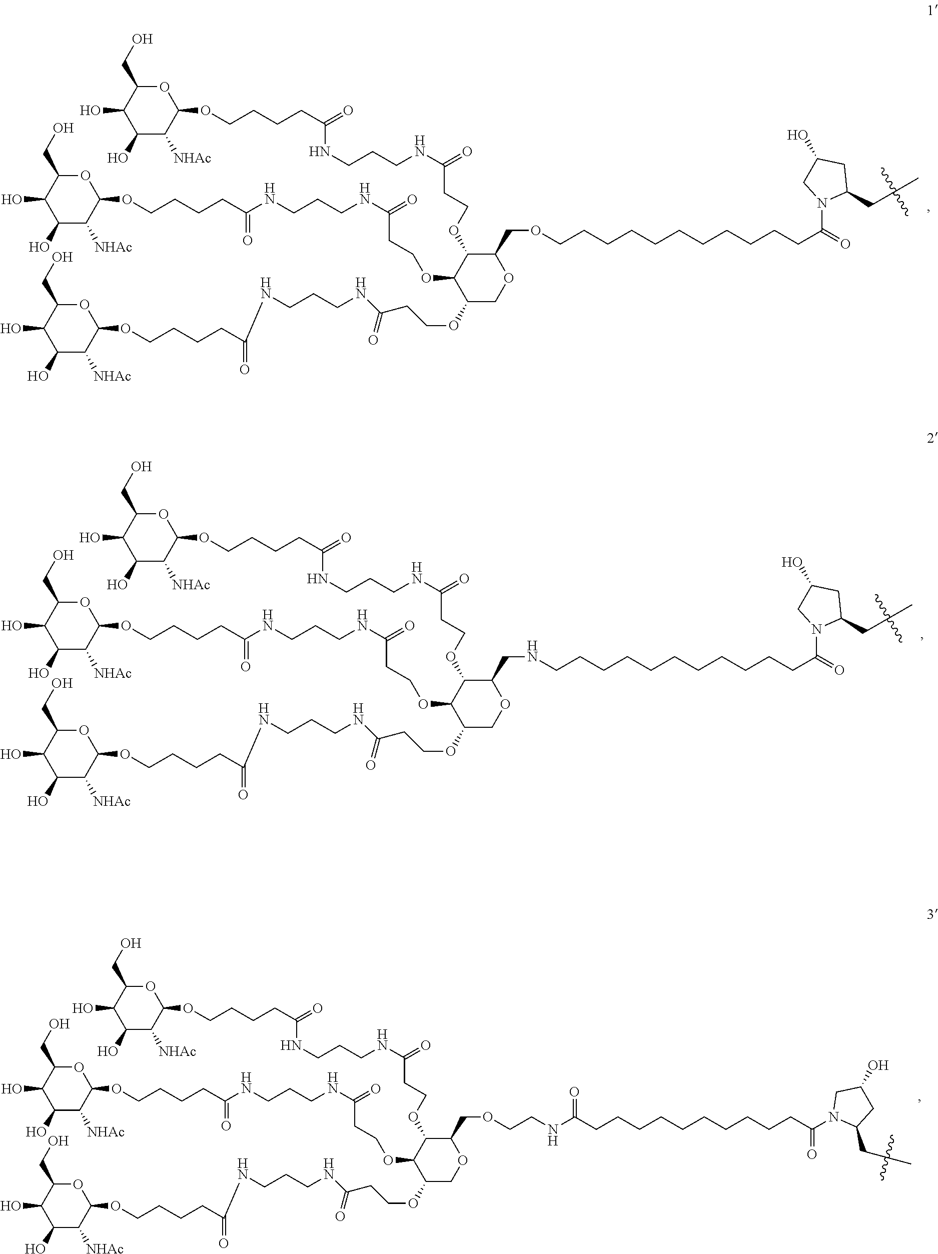

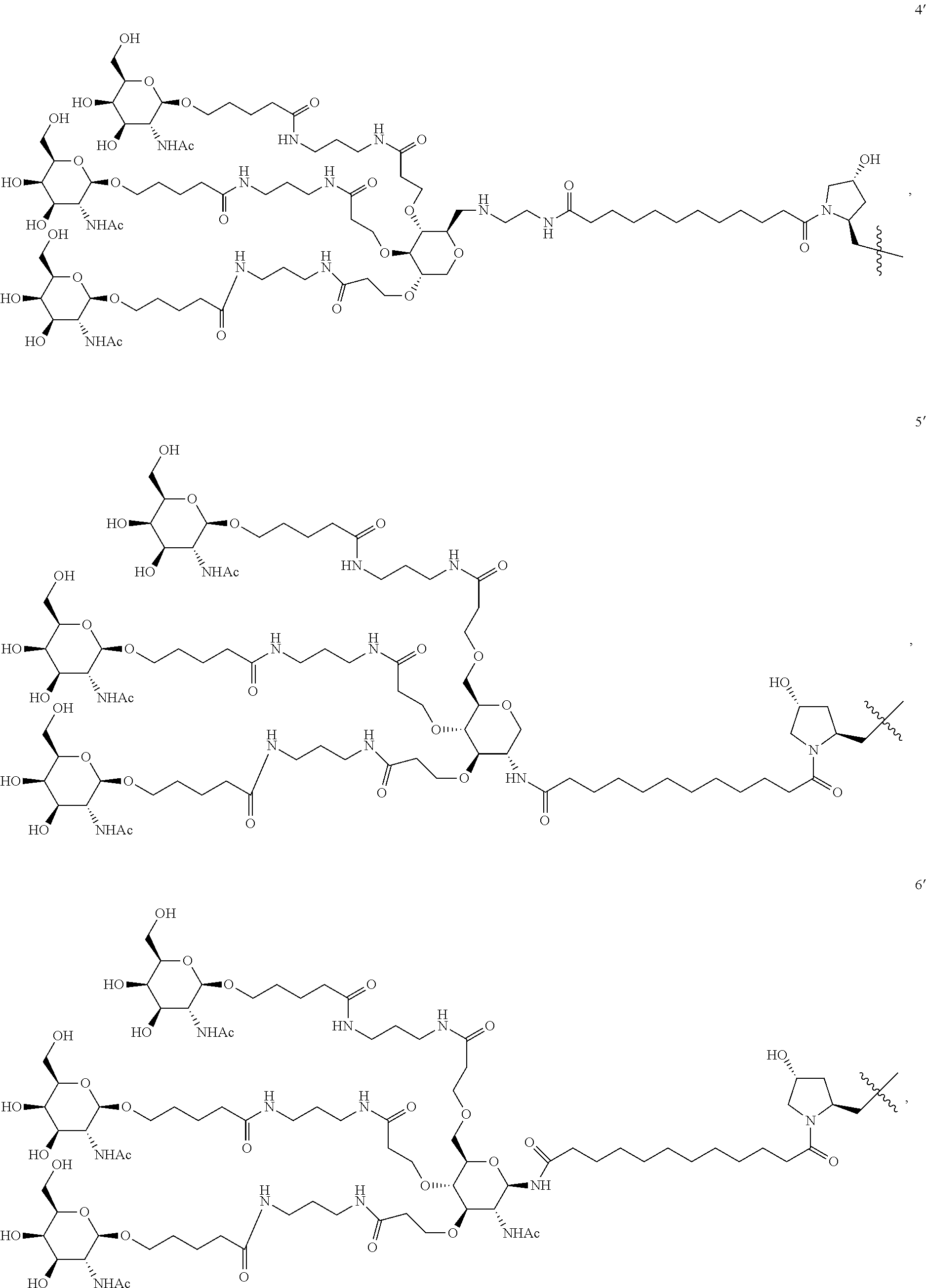

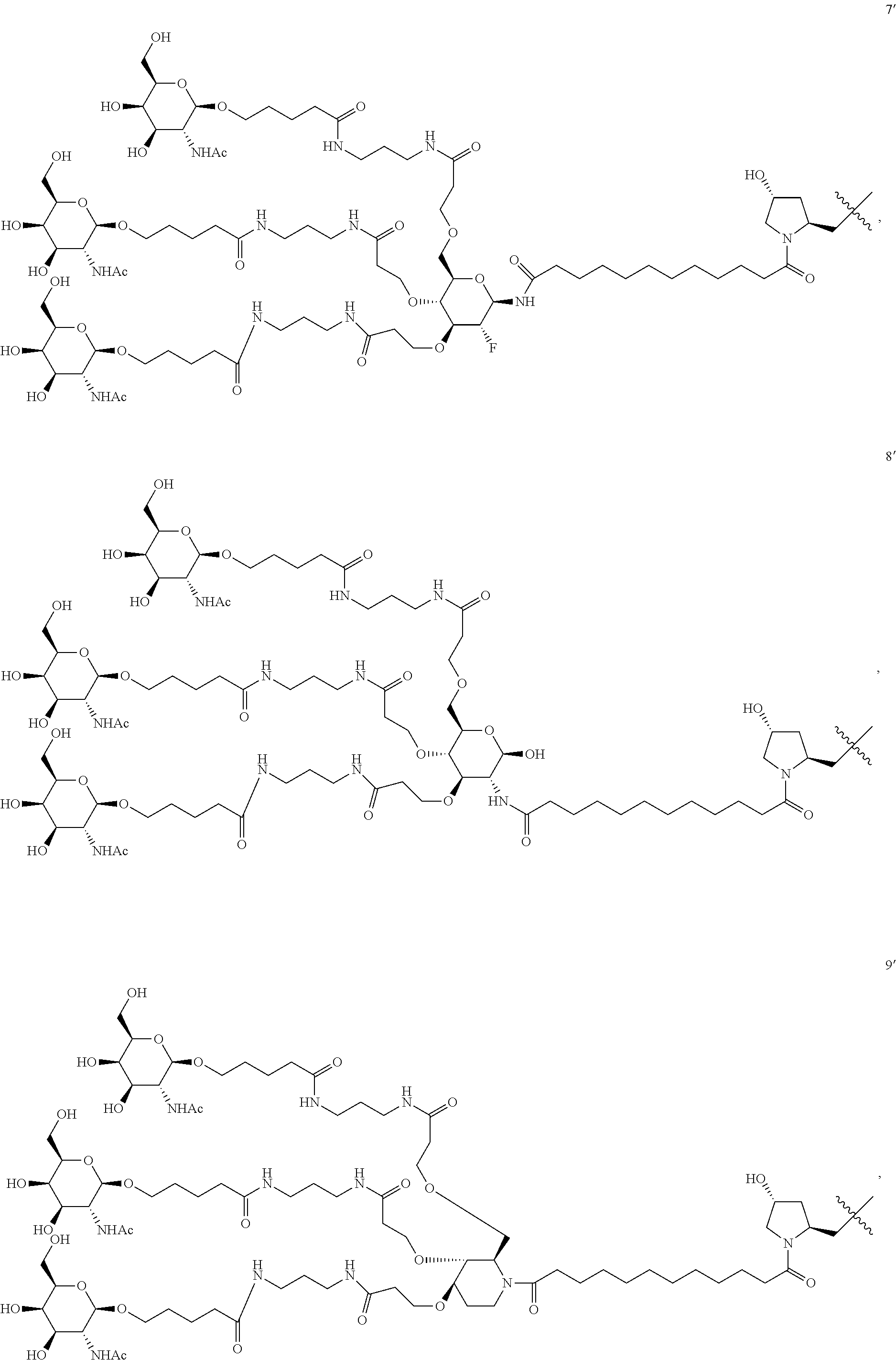

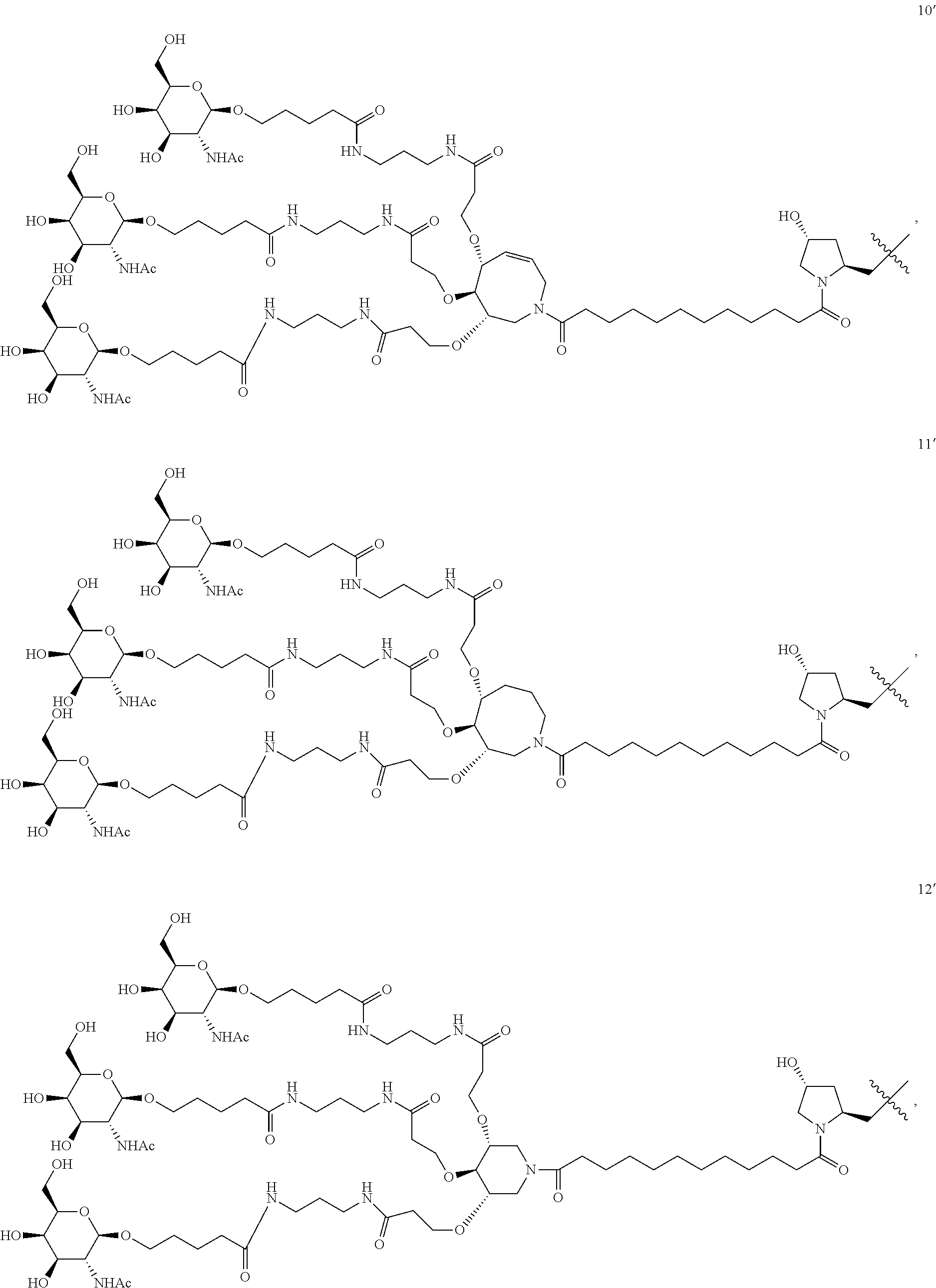

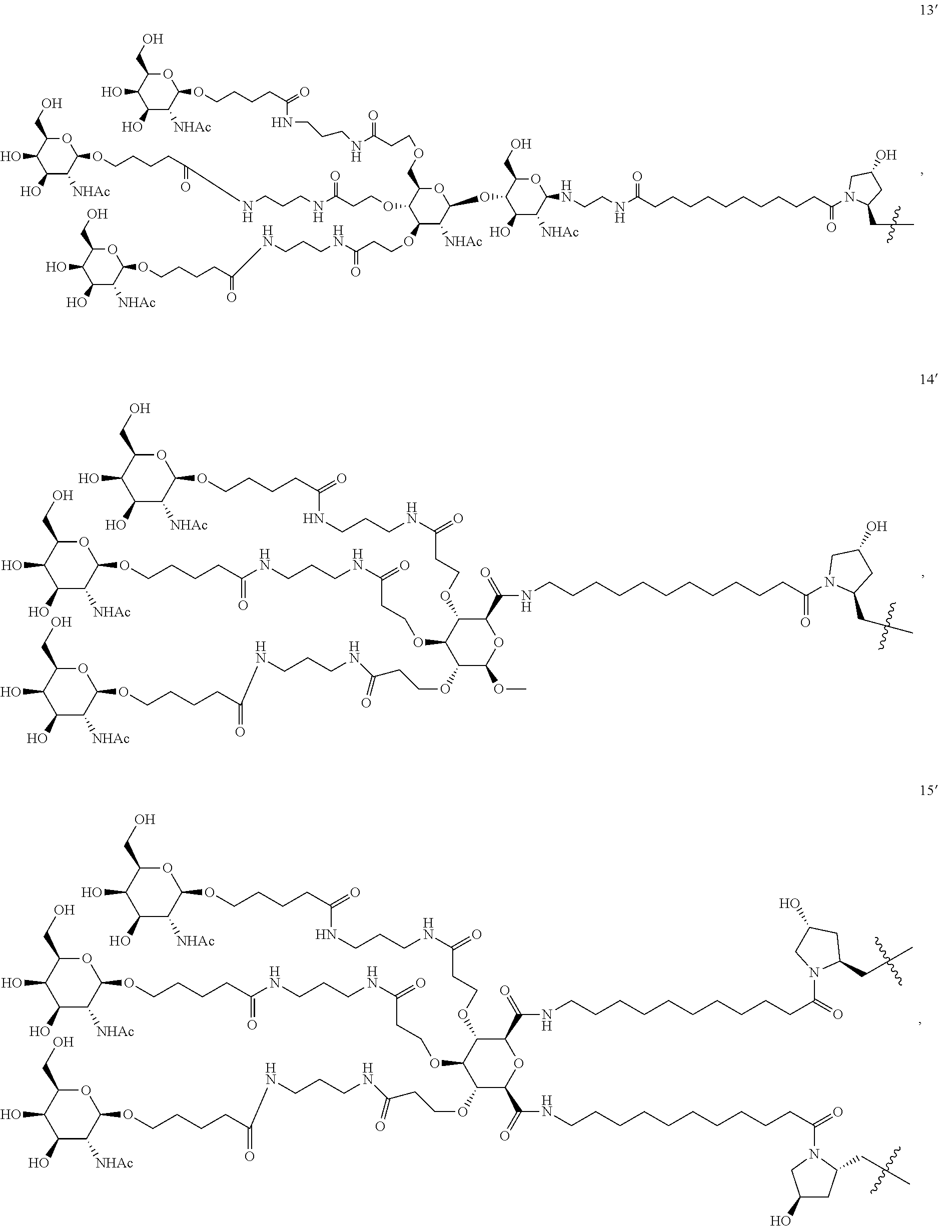

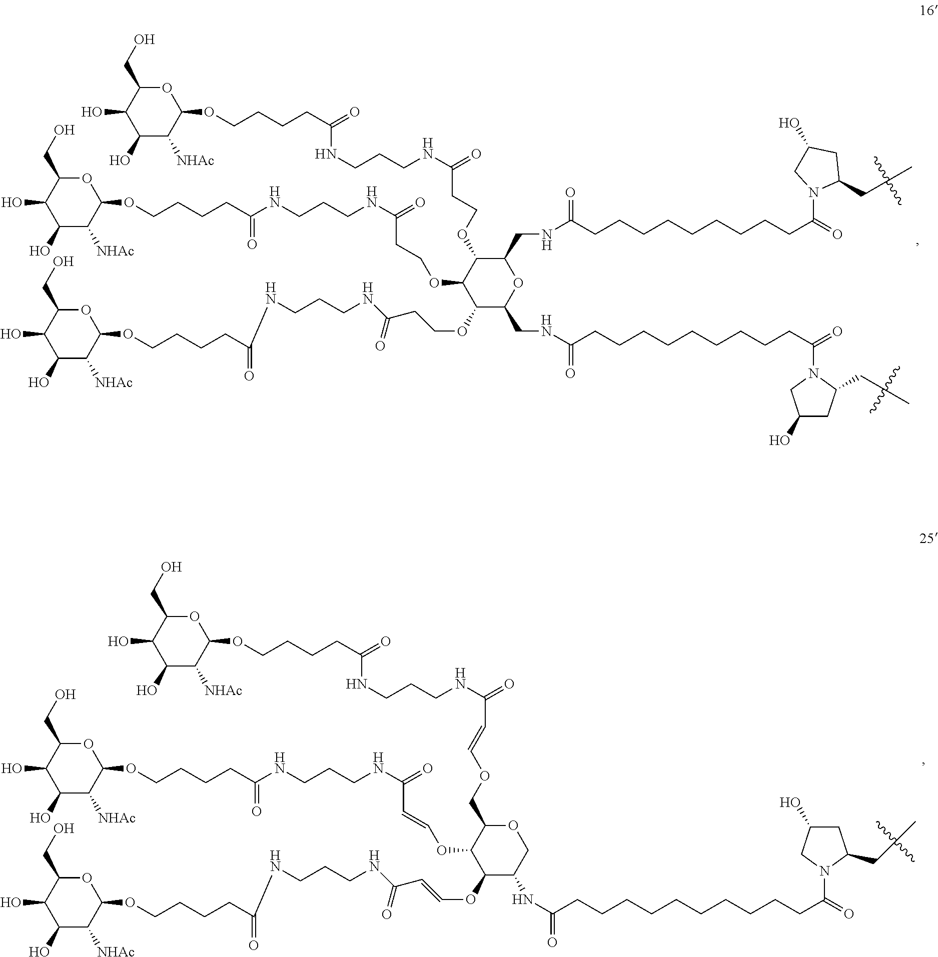

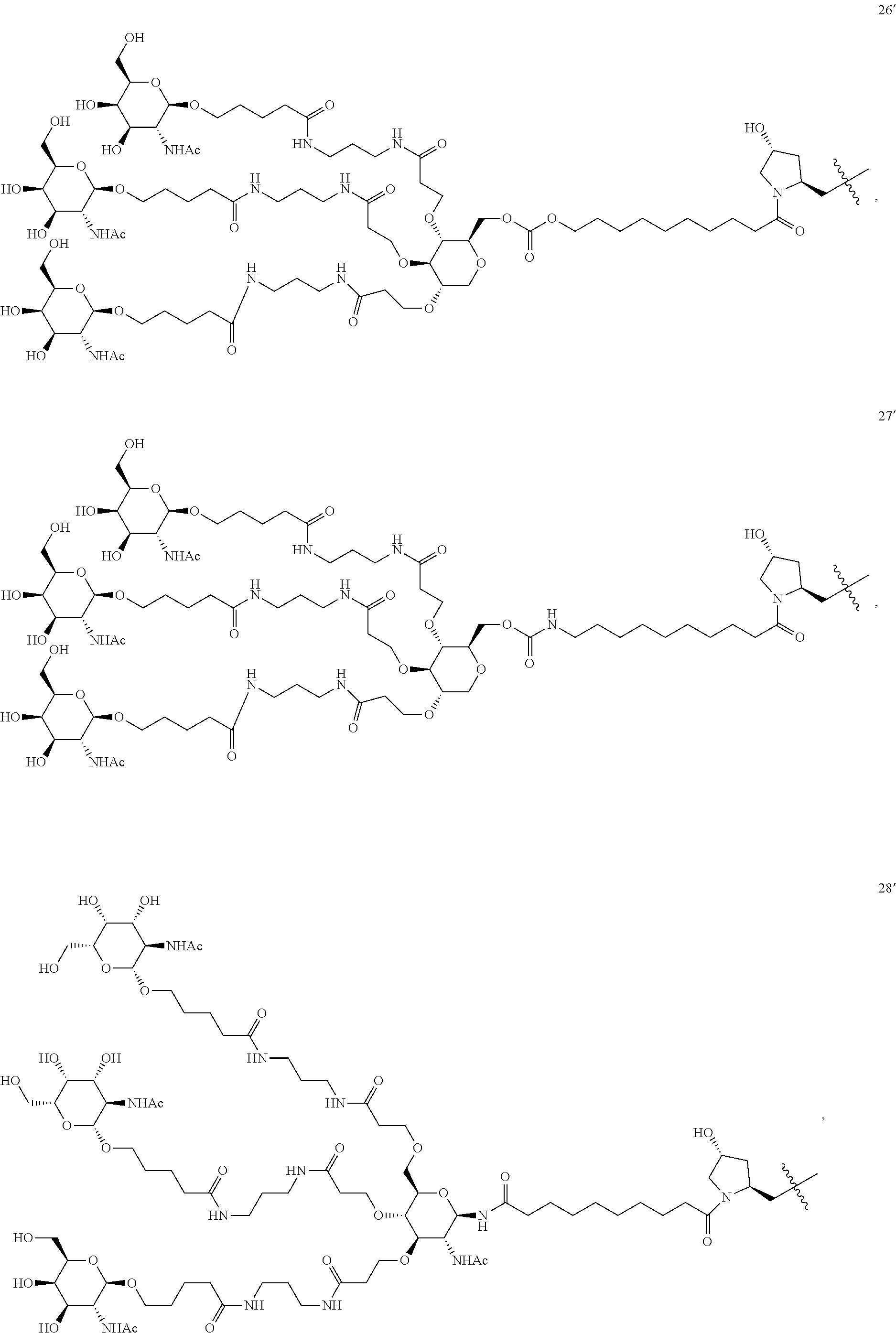

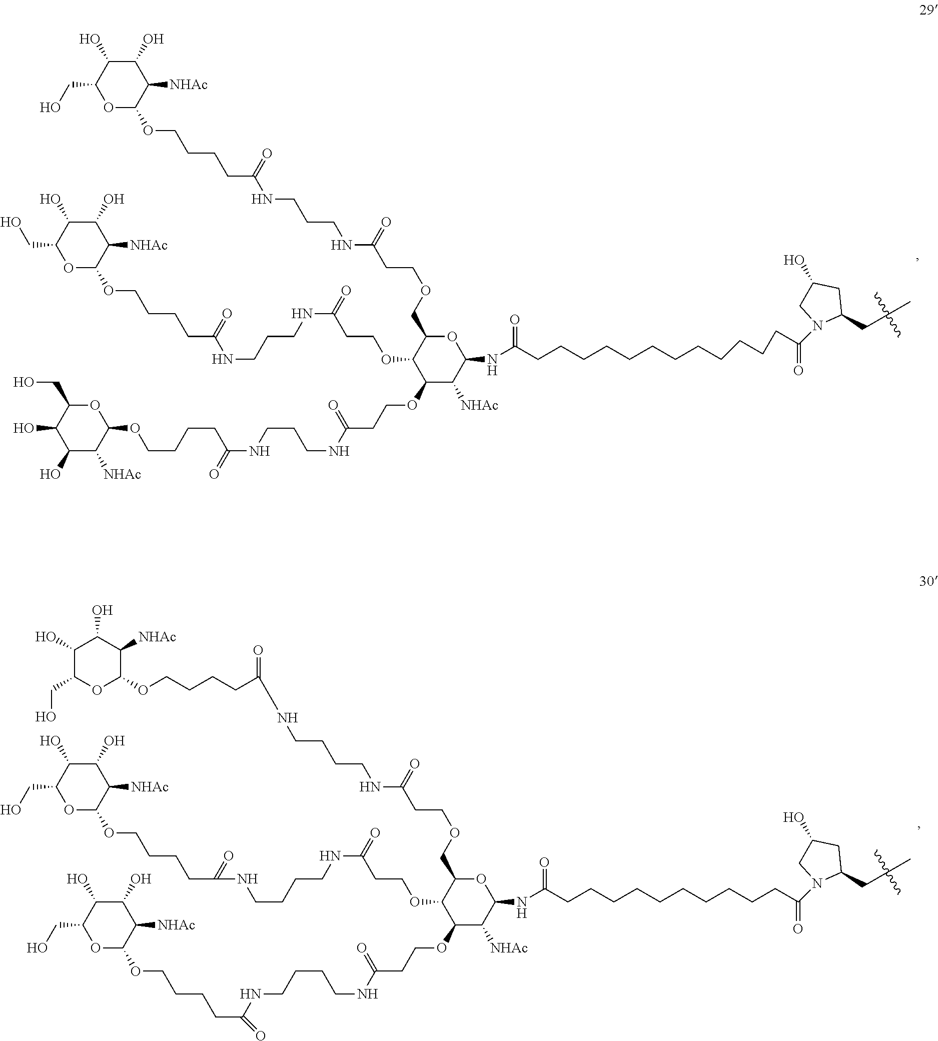

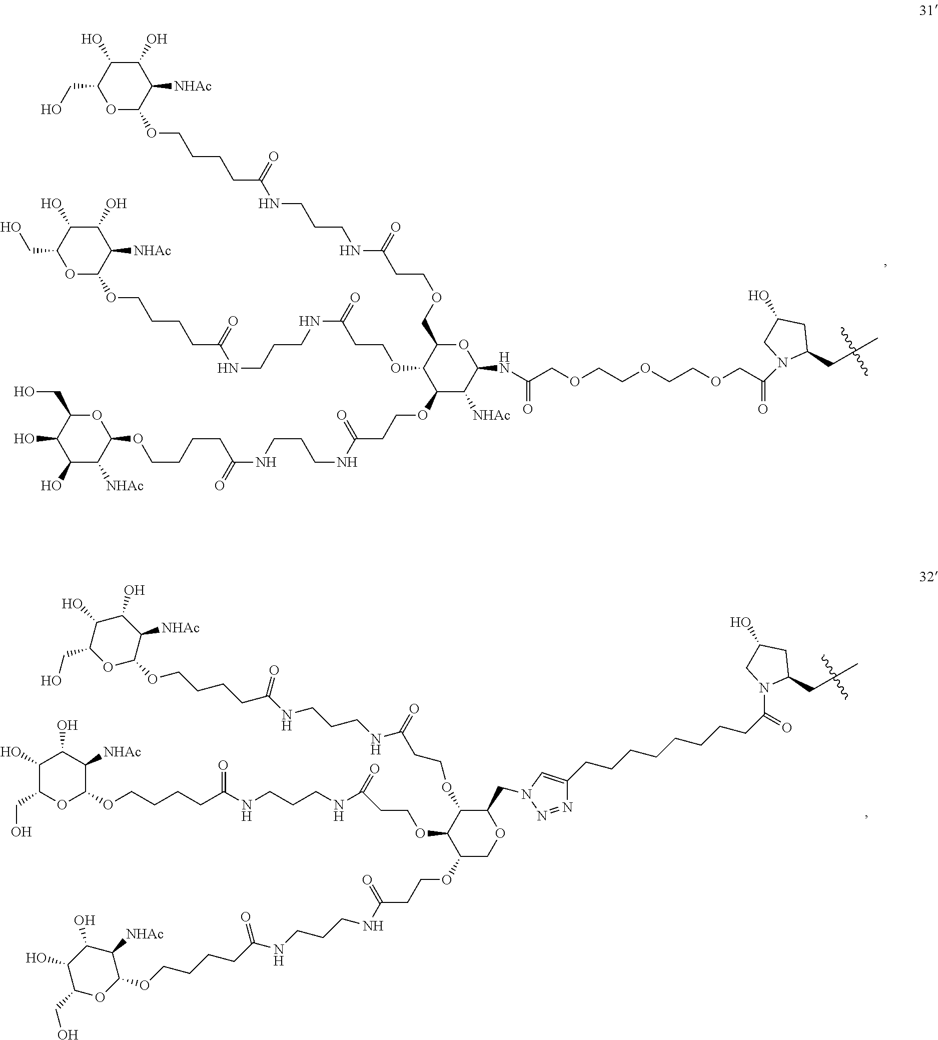

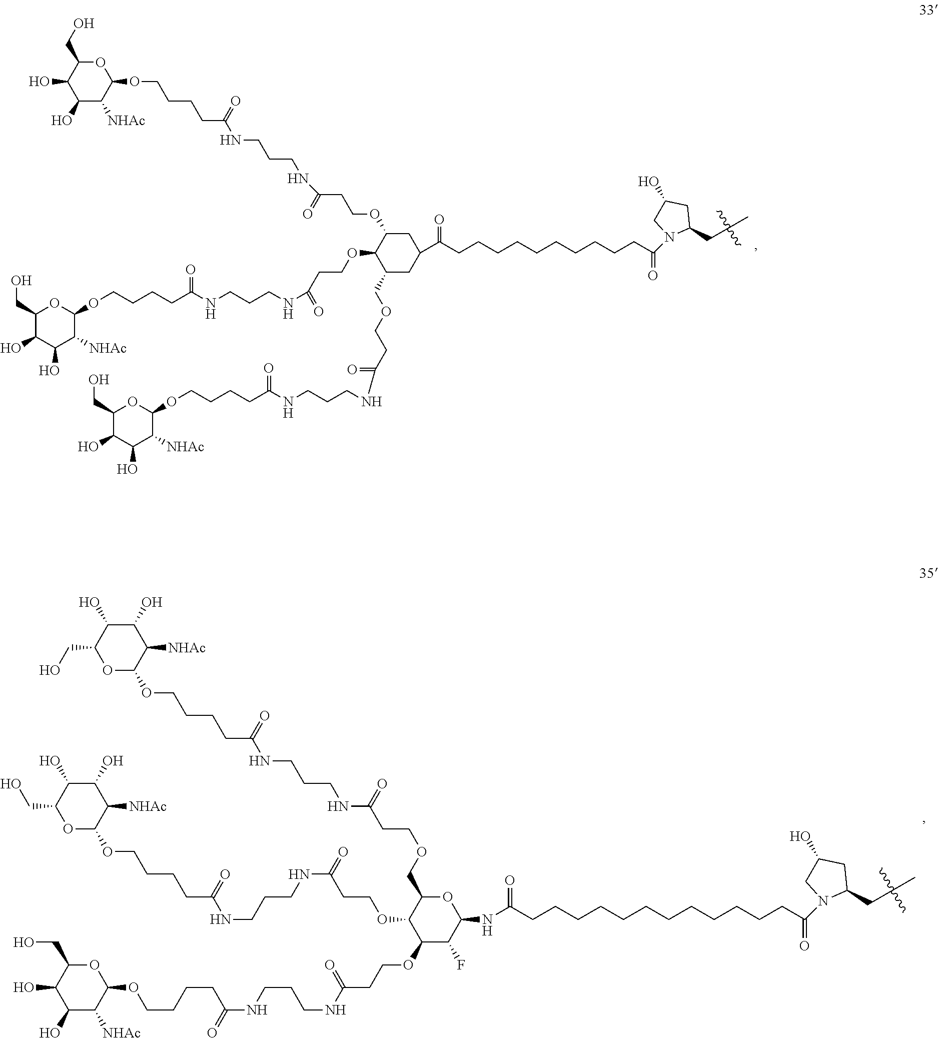

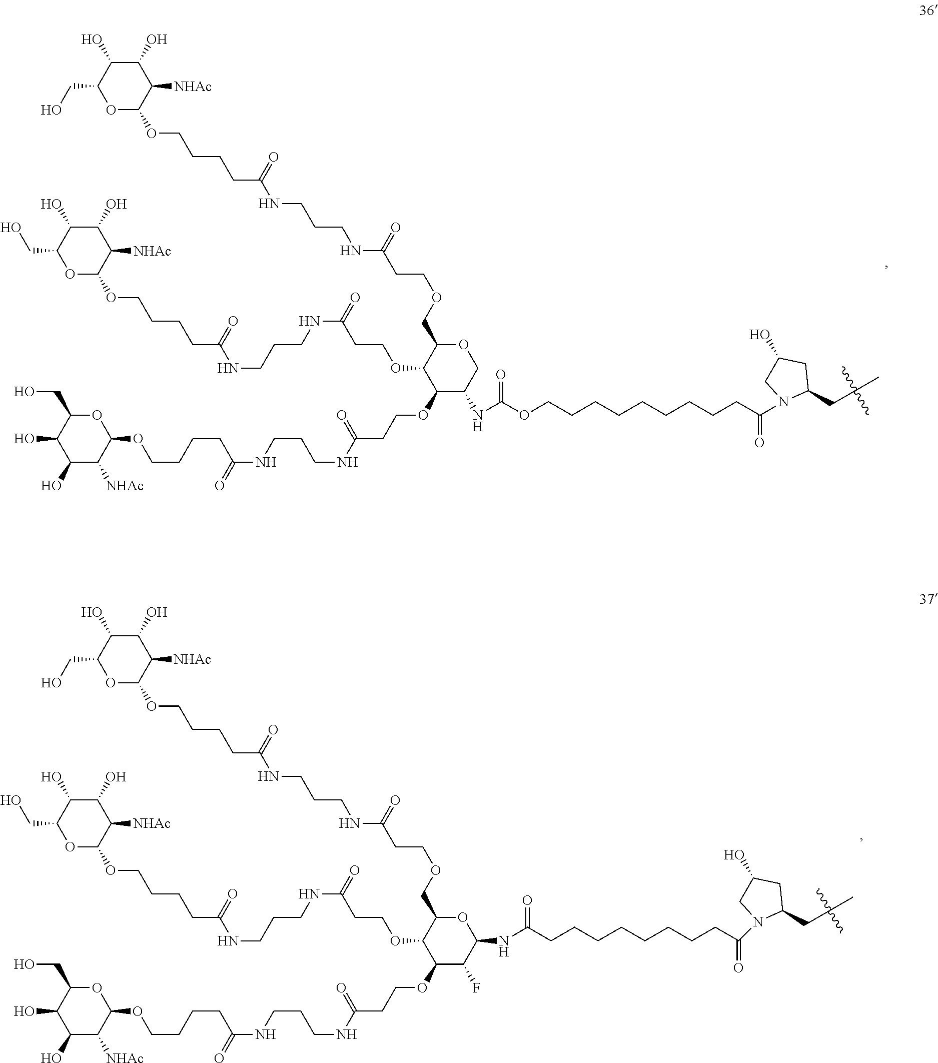

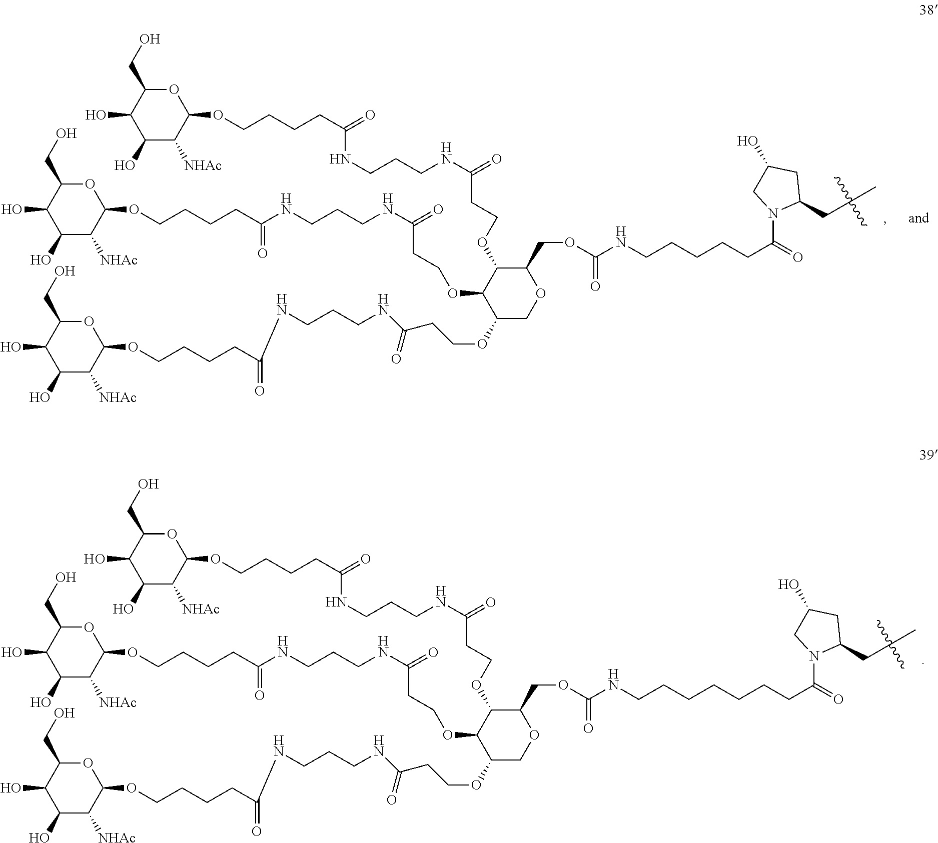

34. The compound as claimed in to claim 1 or a pharmaceutically acceptable salt or solvate thereof, wherein said compound comprises a structure selected from the group consisting of:

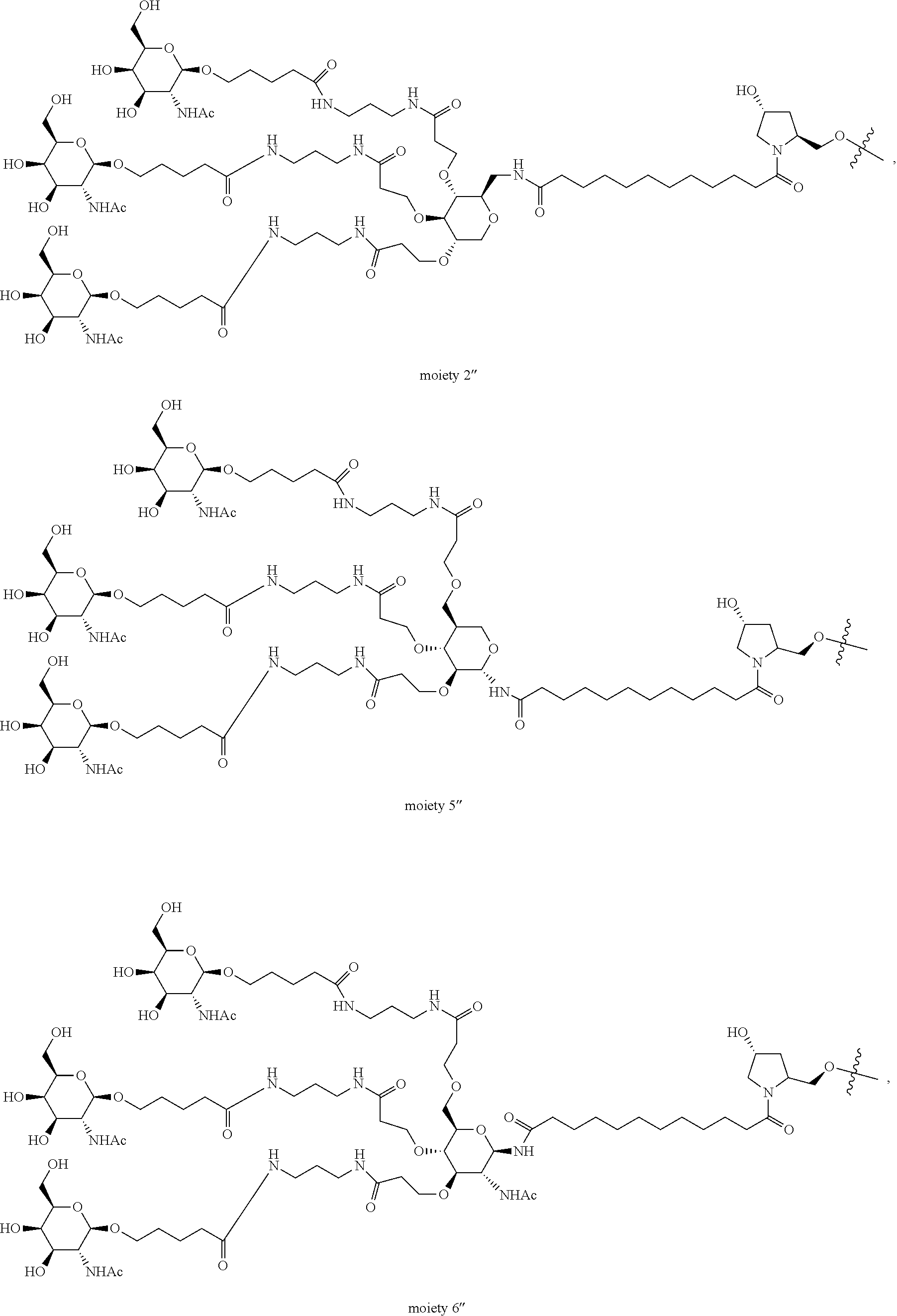

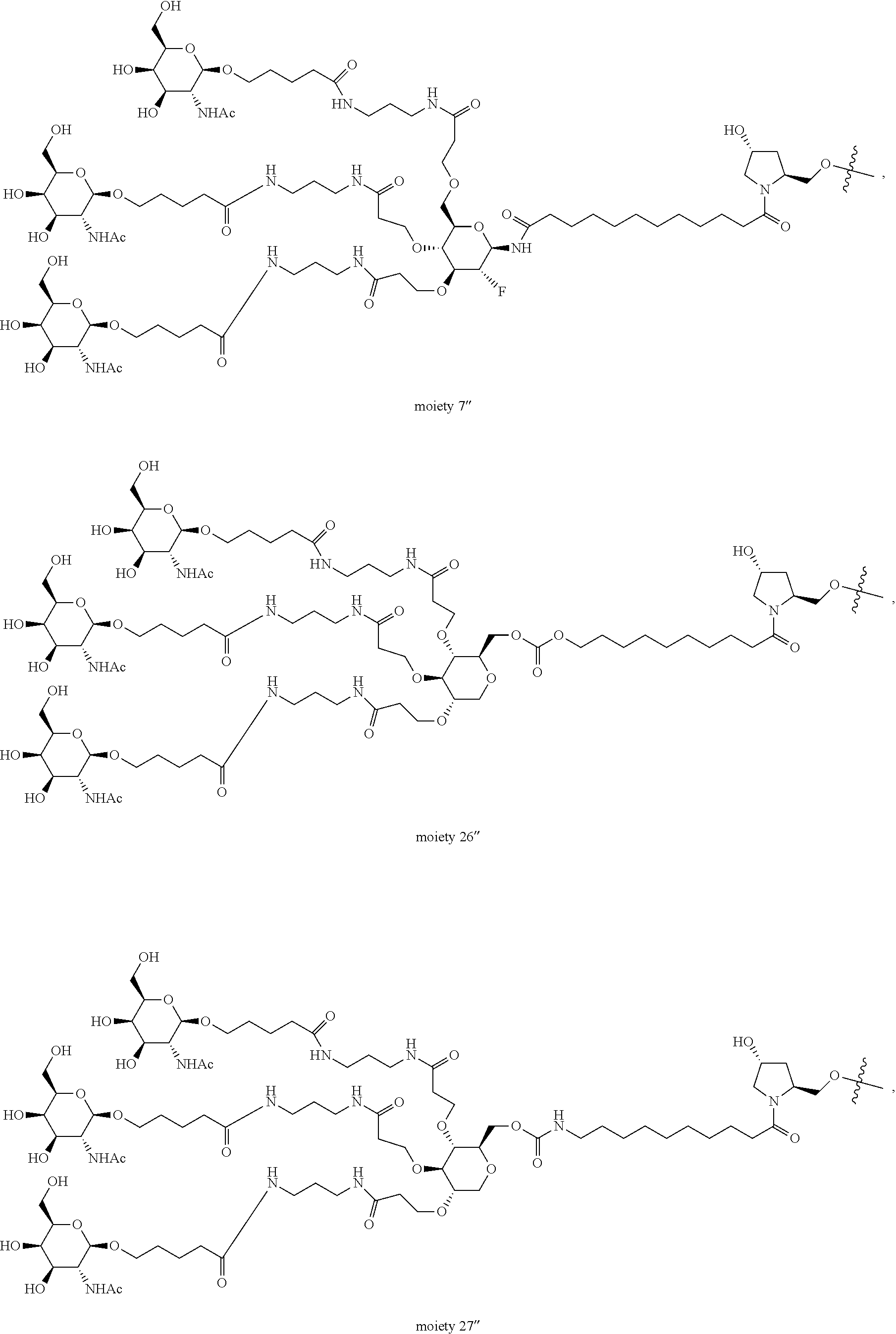

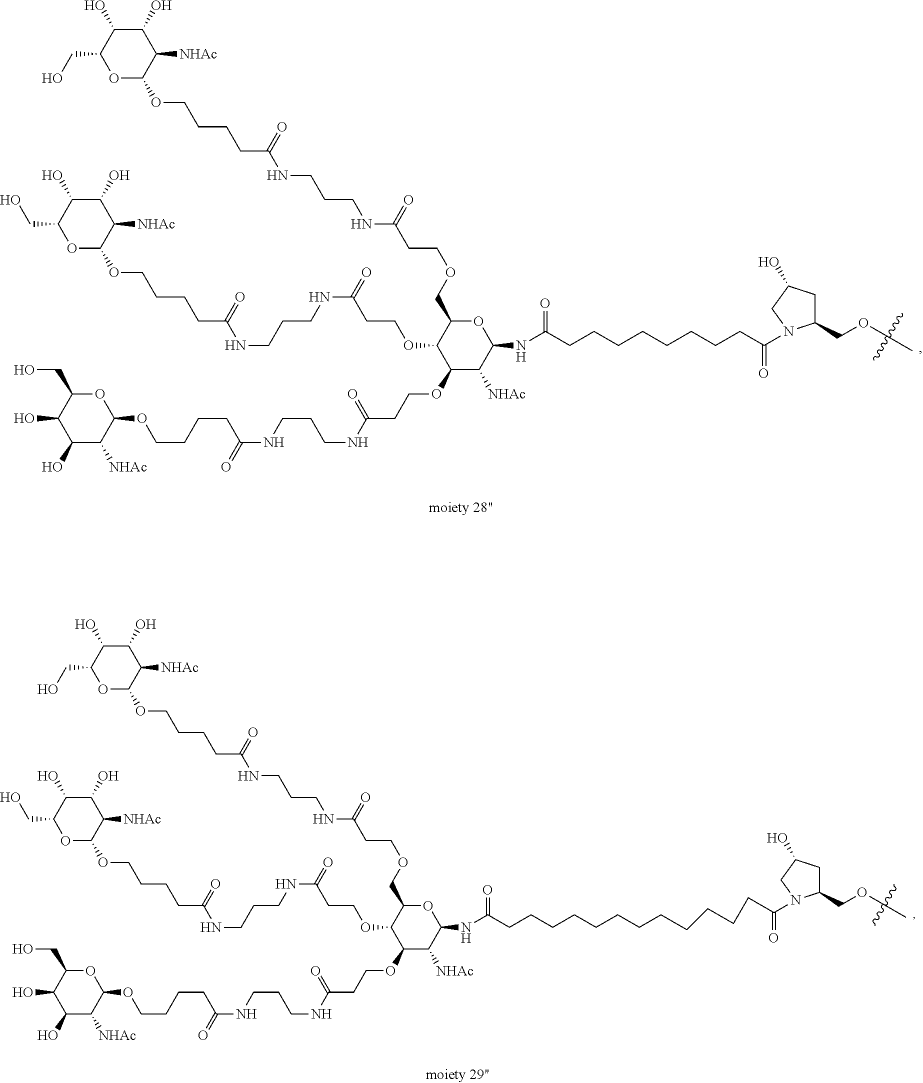

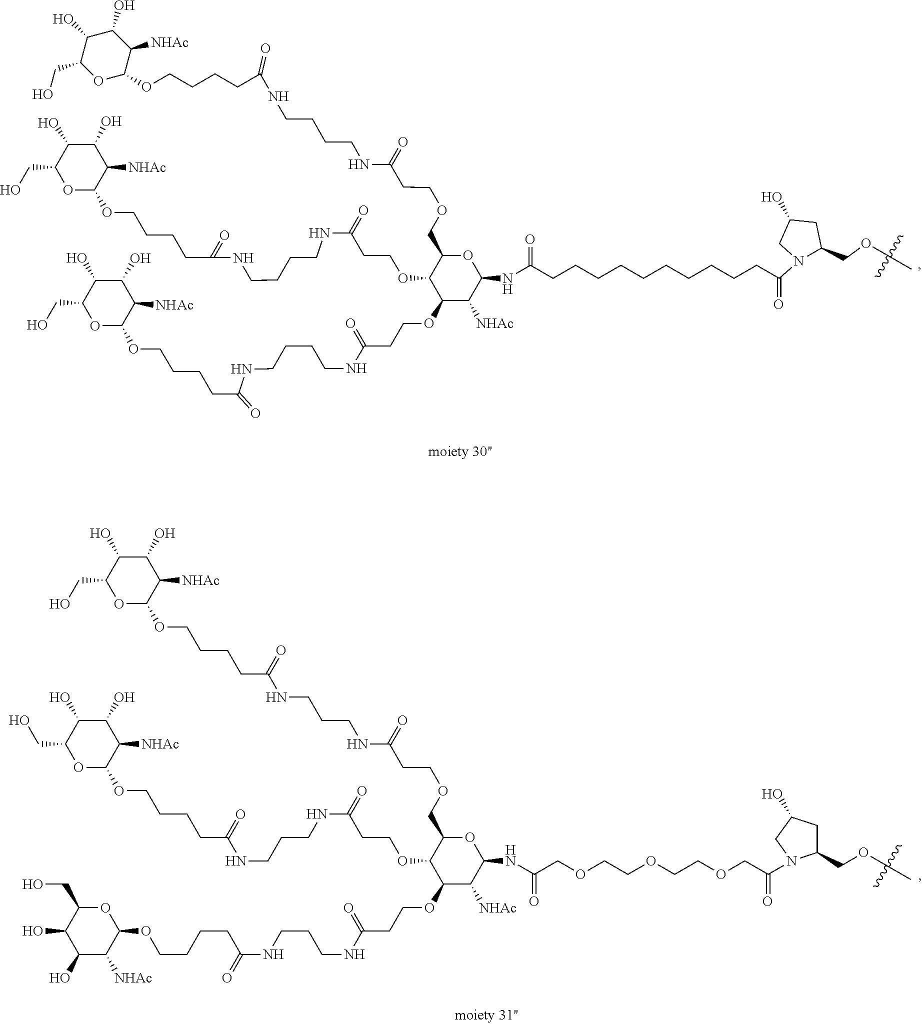

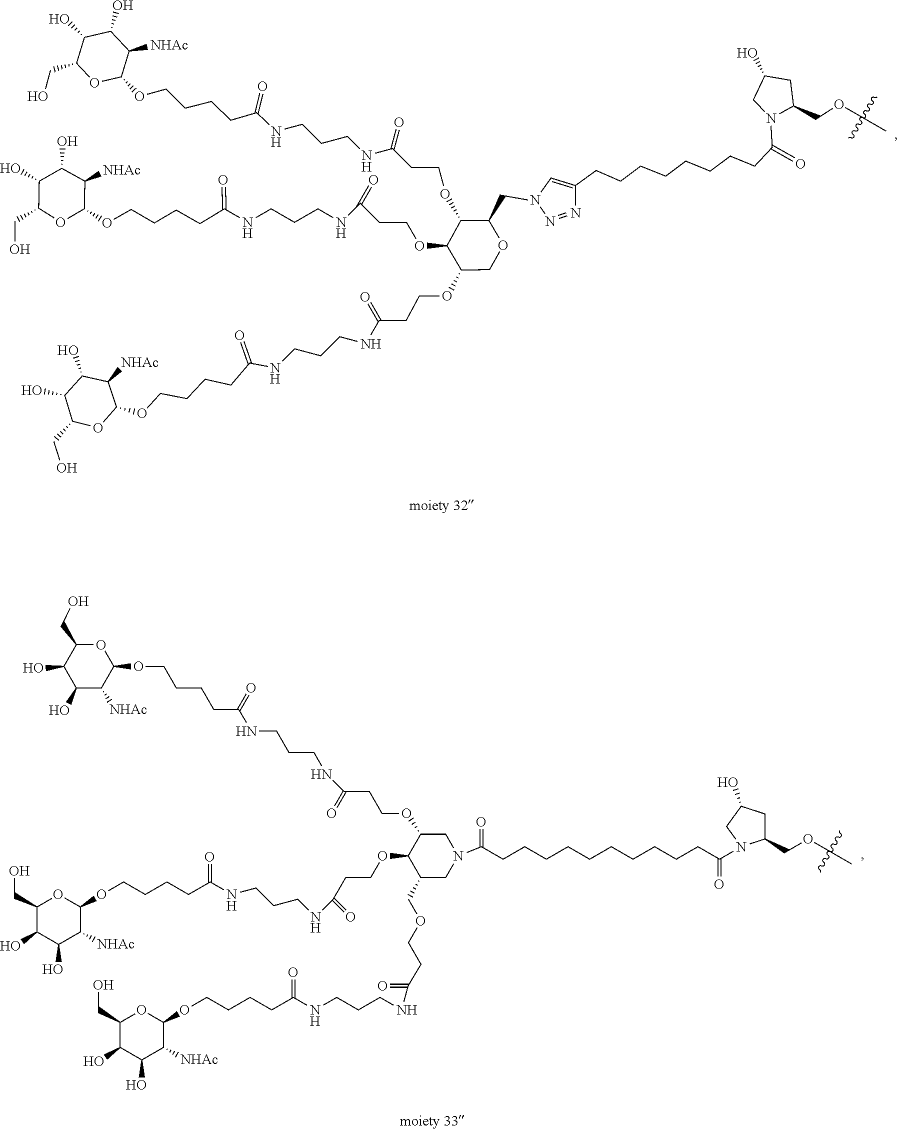

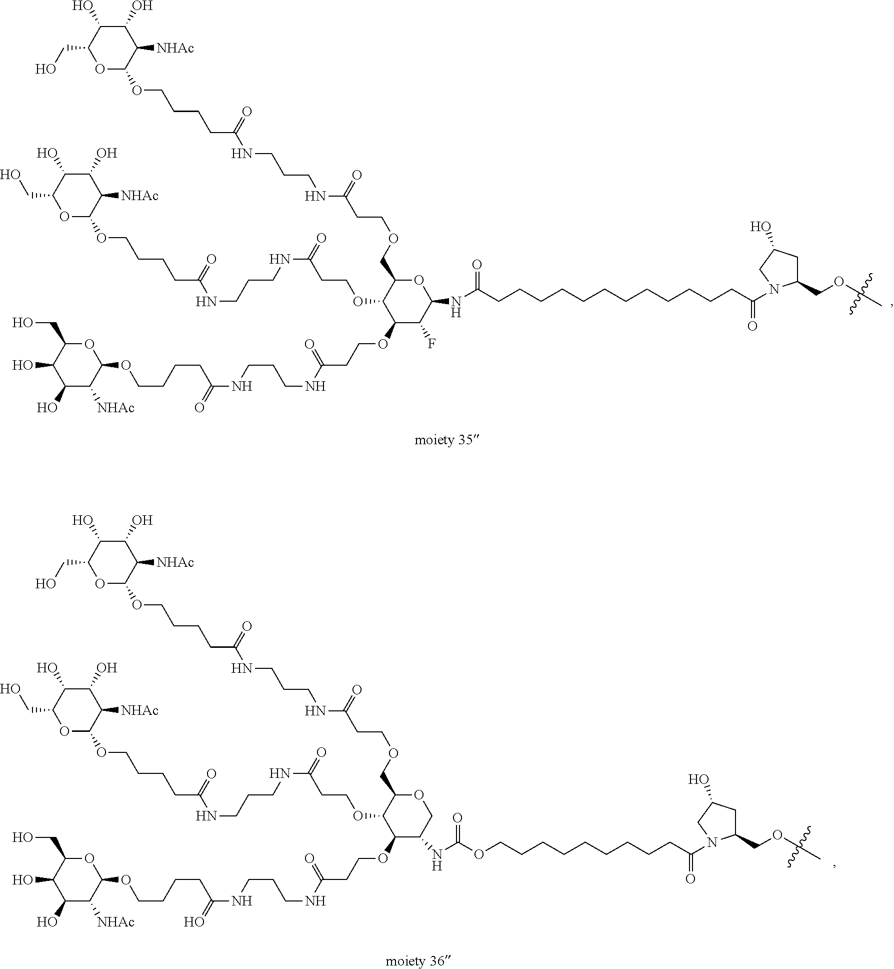

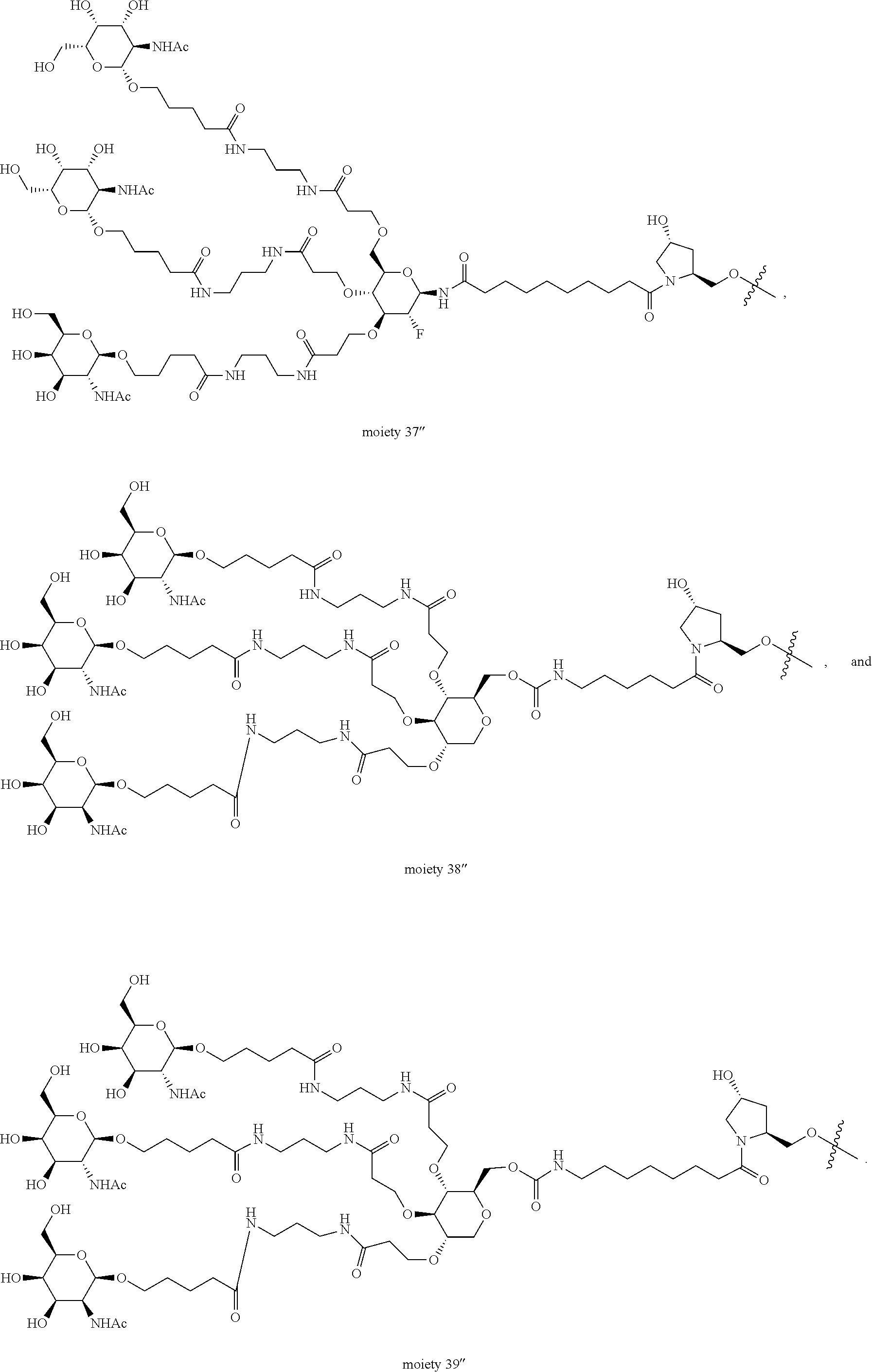

35. (canceled)

36. The compound as claimed in claim 1 or a pharmaceutically acceptable salt or solvate thereof, wherein said compound is selected from a group consisting of the following compounds:

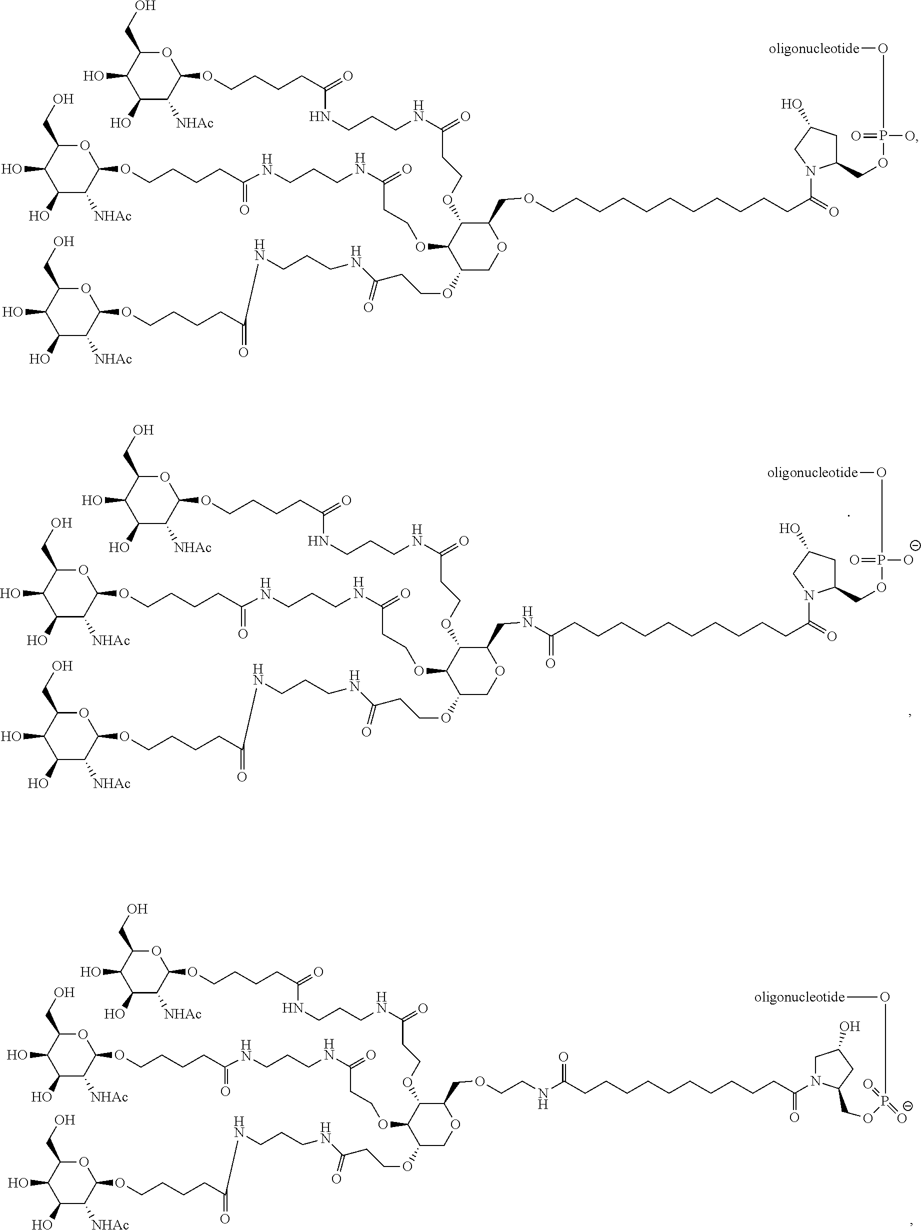

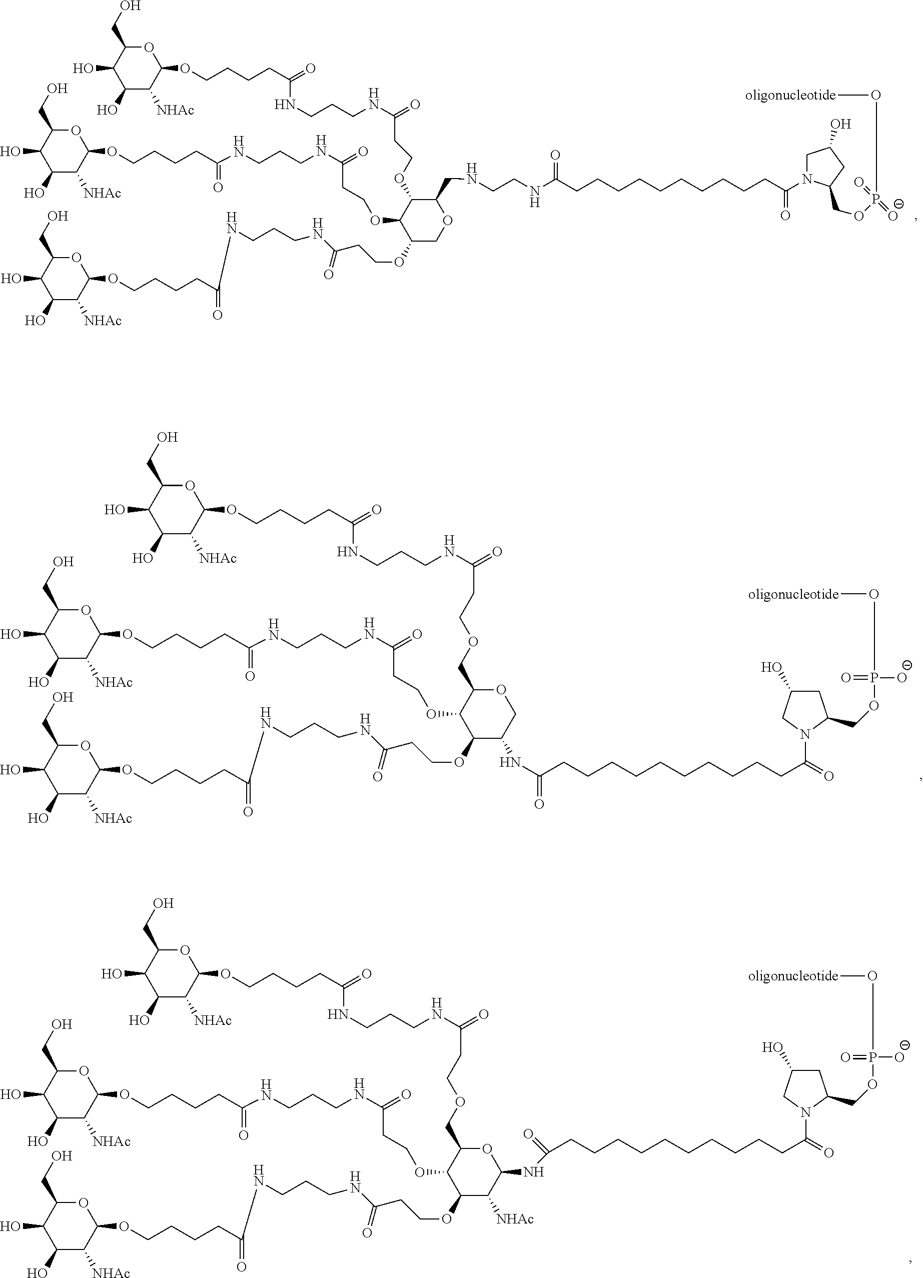

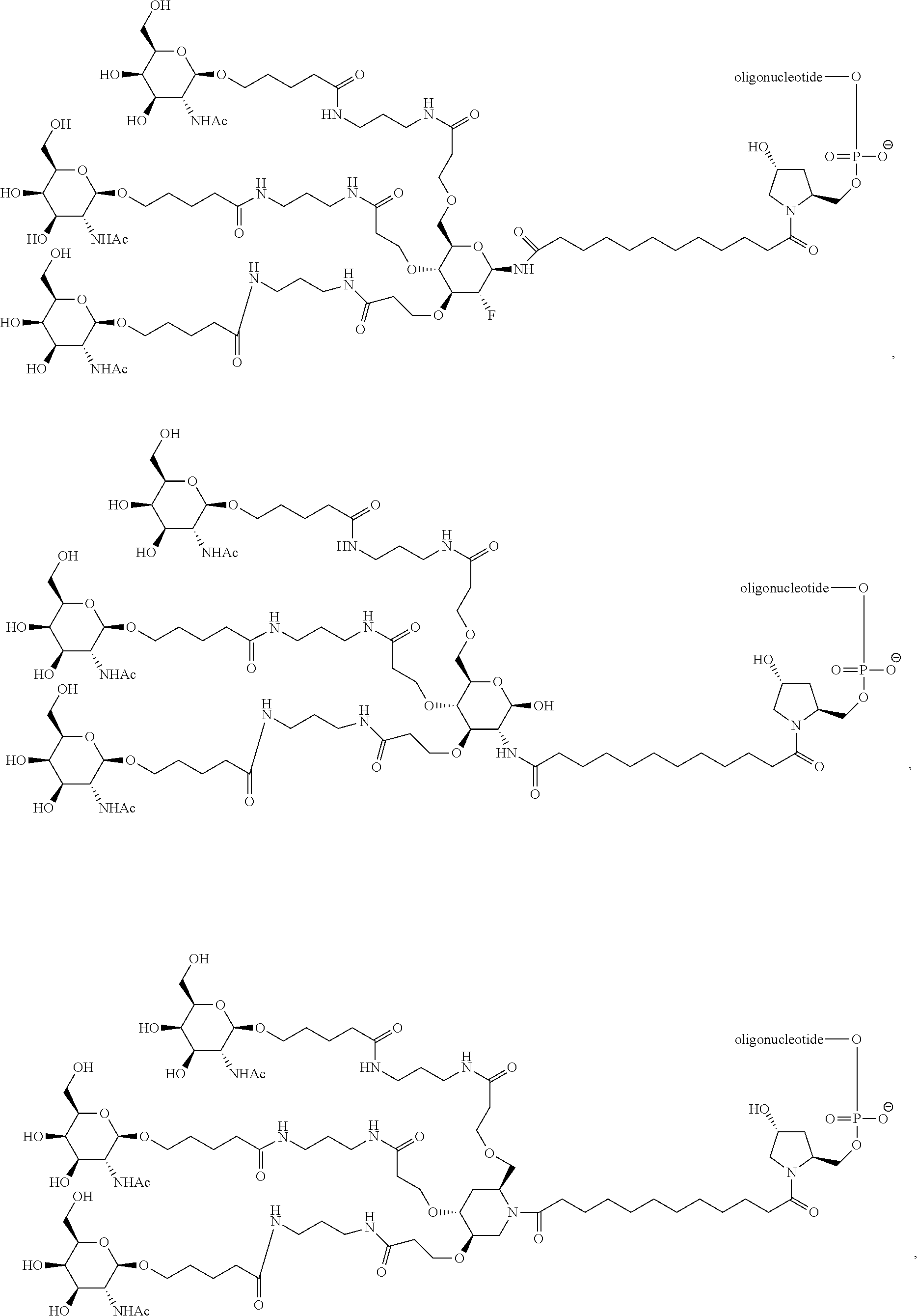

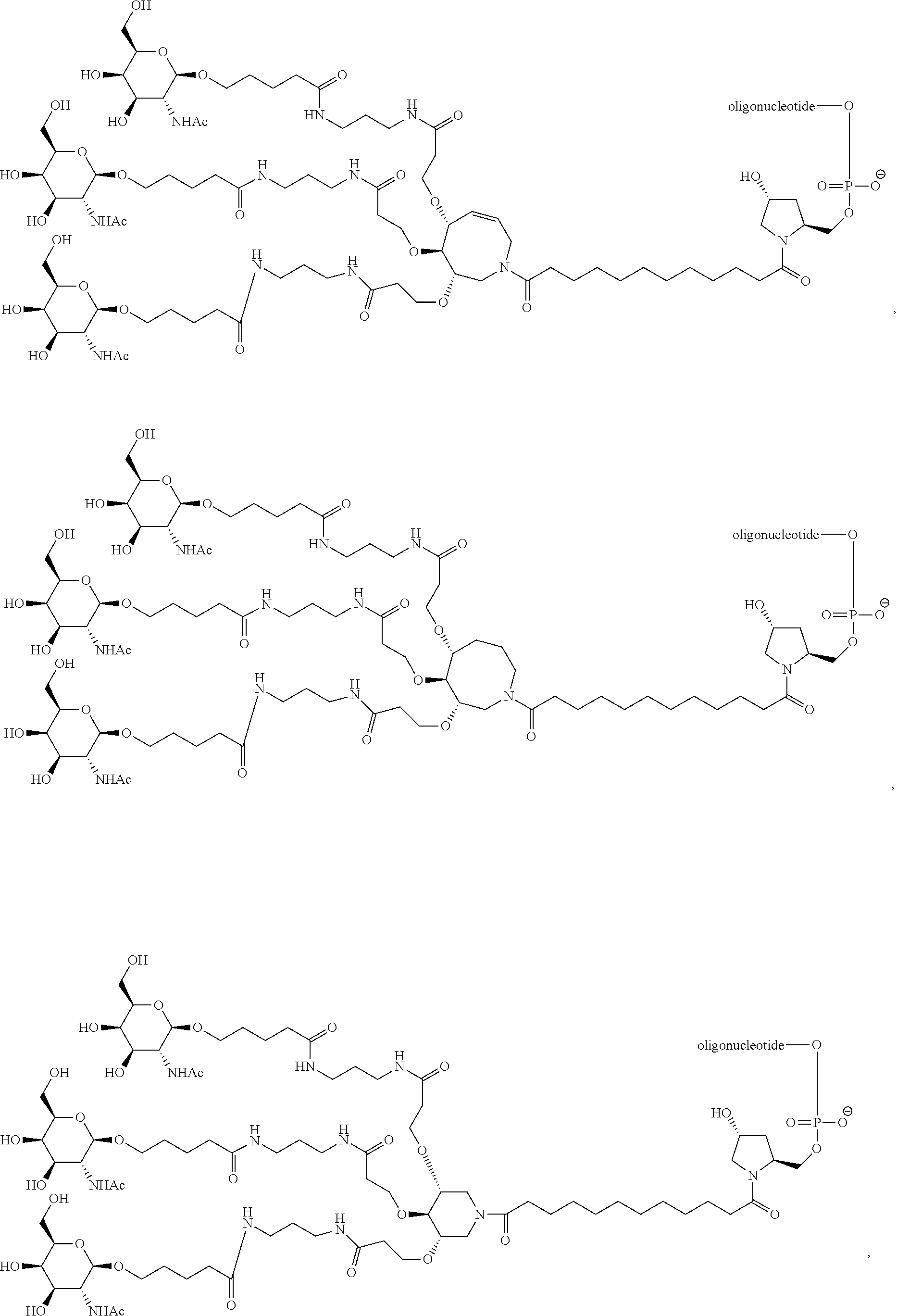

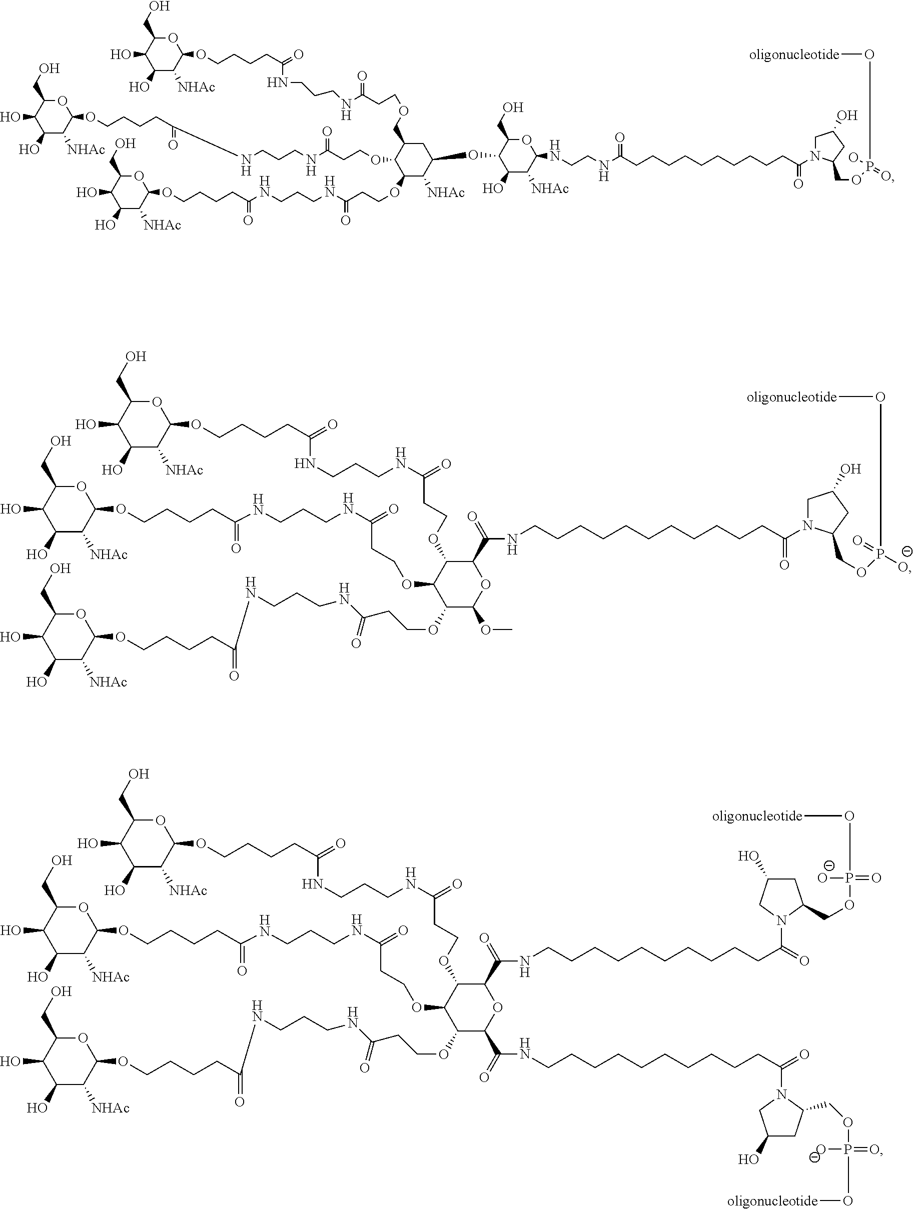

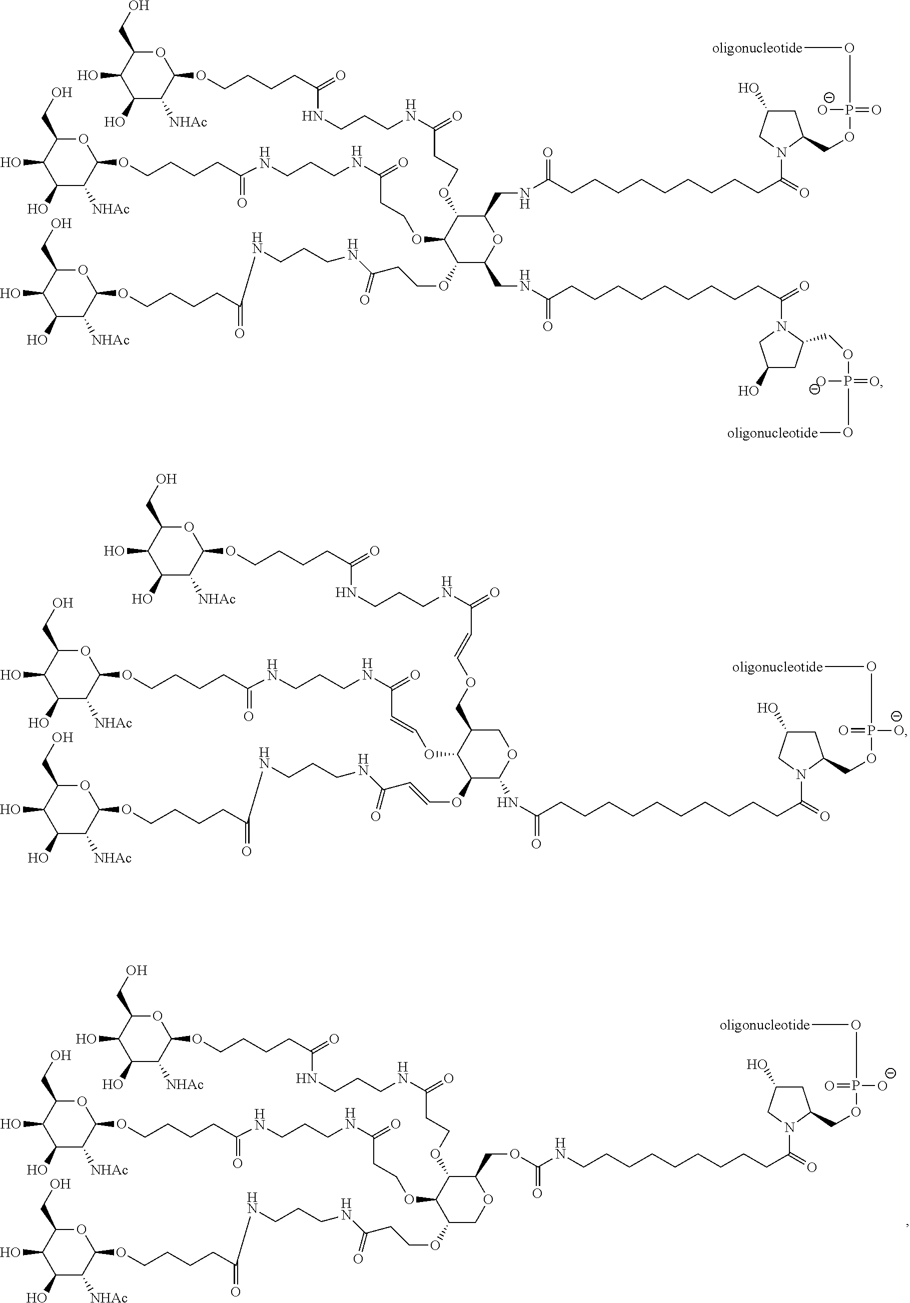

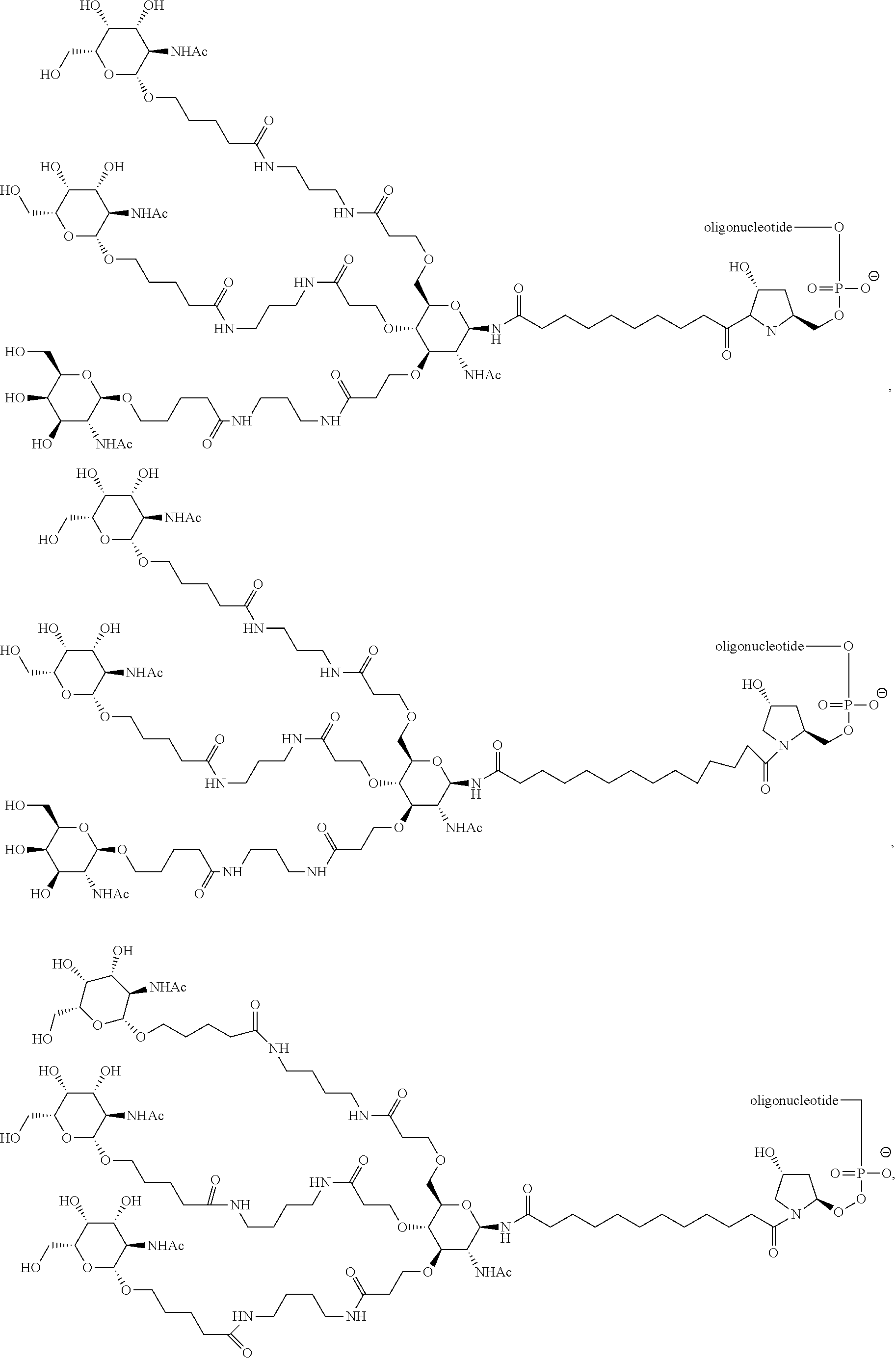

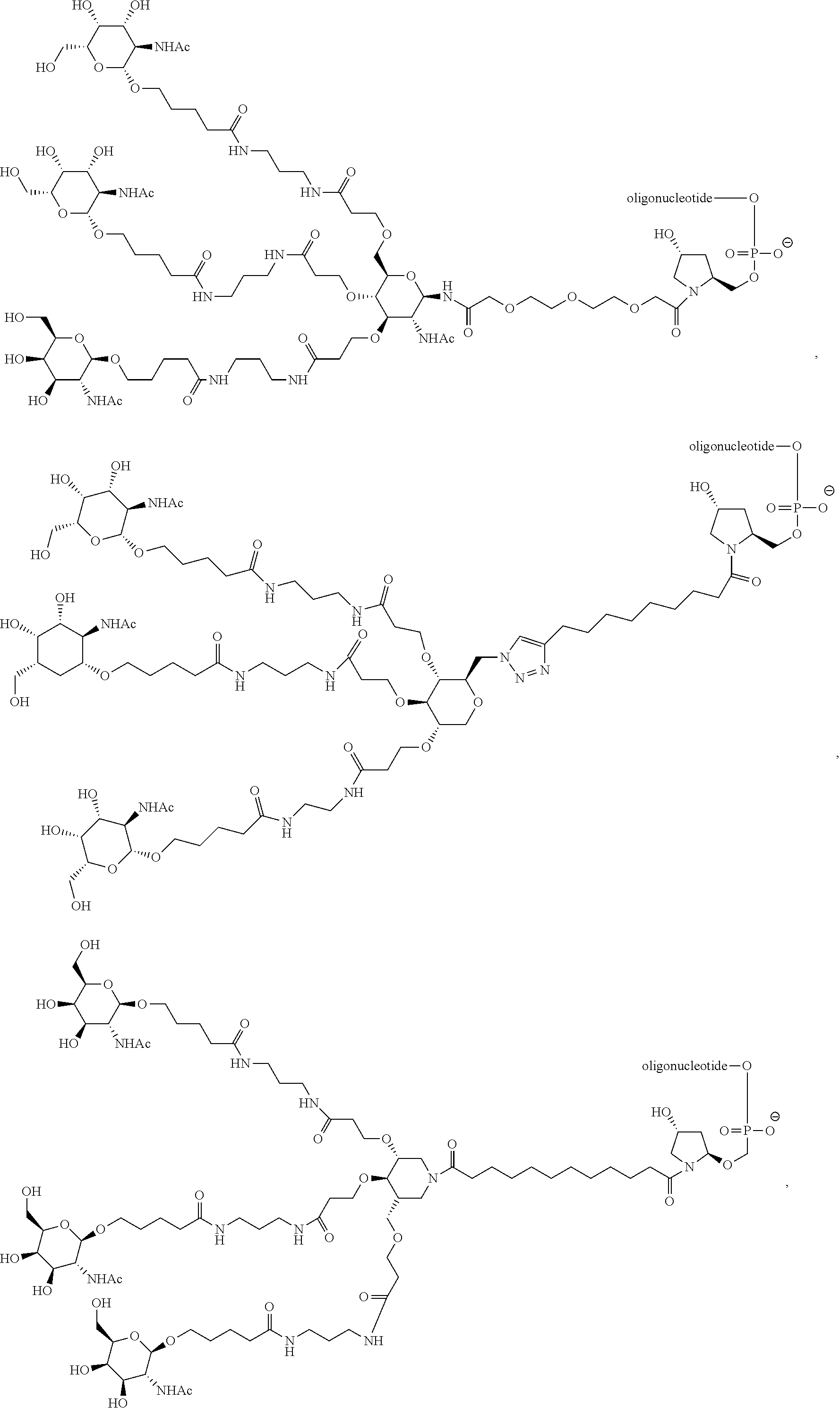

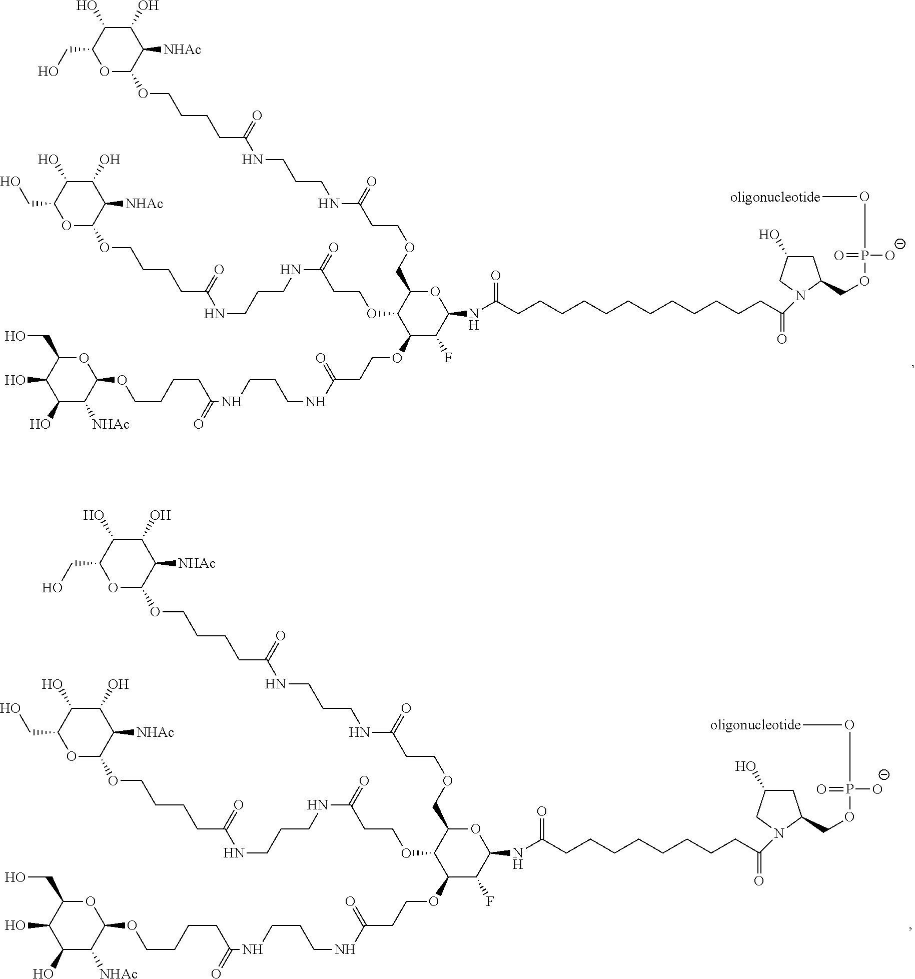

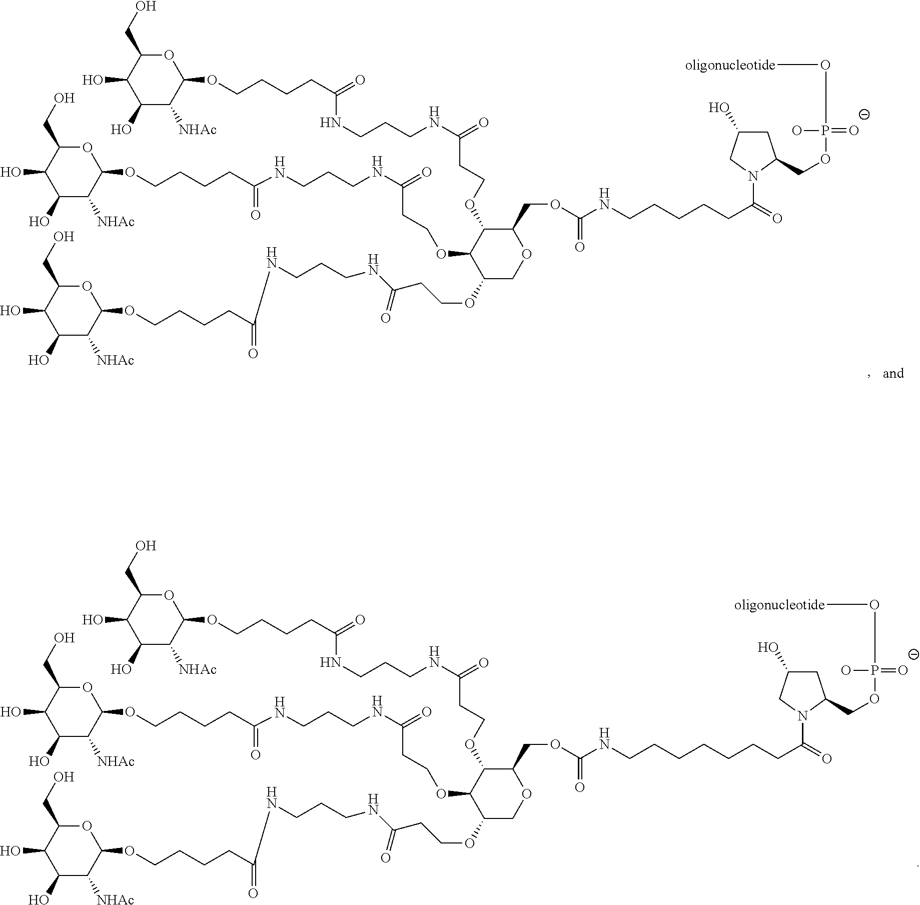

37. (canceled)

38. A method of modulating the expression of a target gene in a cell, comprising delivering to said cell a compound as claimed in claim 1 or a pharmaceutically acceptable salt or solvate thereof.

39. The method as claimed in claim 38, wherein the target gene is associated with a metabolic disease, or is relevant to a liver disease.

40.-45. (canceled)

46. A pharmaceutical composition comprising a compound as claimed claim 1 or a pharmaceutically acceptable salt or solvate thereof alone or in combination with a pharmaceutically acceptable carrier or excipient.
